(19) United States
(12) Patent Application Publication
(10) Pub. No.: US 2026/0167709 A1
(43) Pub. Date: Jun. 18, 2026

(54) ANTIBODIES TARGETING IL-31

(71) Applicant: NOVARTIS AG, Basel (CH)

(72) Inventors: Regis CEBE, Saint-Louis (FR); Hejun LIU, San Diego, CA (US); Frank KOLBINGER, Neuenburg am Rhein (DE); Jiri KOVARIK, Zurich (CH); Kathrin MÜLLER, Aarau (CH); Ben ROEDIGER, Basel (CH); Jean-Michel, René RONDEAU, Mulhouse (FR)

(21) Appl. No.: 19/325,292

(22) Filed: Sep. 10, 2025

Related U.S. Application Data
(60) Provisional application No. 63/693,617, filed on Sep. 11, 2024.
(60) Provisional application No. 63/848,833, filed on Jul. 22, 2025.

(51) Int. Cl. C07K 16/24

(52) U.S. Cl. CPC C07K 16/244; C07K 2317/24; C07K 2317/31; C07K 2317/55; C07K 2317/565; C07K 2317/73; C07K 2317/76; C07K 2317/92; C07K 2317/94

(57) ABSTRACT

The invention relates to antibodies that bind to IL-31, including monospecific and multispecific e.g., bispecific antibodies, methods of producing the antibodies, pharmaceutical compositions comprising the antibodies, and methods of using the antibodies.

### FIELD

The invention relates to antibodies that bind to IL-31, including monospecific and multispecific, e.g., bispecific antibodies, methods of producing the antibodies, pharmaceutical compositions comprising the antibodies, and methods of using the antibodies.

### SEQUENCE LISTING

The instant application contains a Sequence Listing which has been submitted electronically in XML format and is hereby incorporated by reference in its entirety. Said XML copy, created on 4 Aug. 2025, is named PAT059772-WO-PCT SQL_to file.xml and is 206,943 bytes in size.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present disclosure are intended.

### BACKGROUND

Interleukin-31 (IL-31) is an inflammatory cytokine implicated in numerous chronic inflammatory conditions including allergic asthma, allergic rhinitis, inflammatory bowel diseases, malignancies, osteoporosis, and dermatologic conditions including atopic dermatitis (Bagci et al., J Allergy Clin Immunol. 141 (2018): 858-866).

Atopic dermatitis is a chronic inflammatory skin disease that usually appears during infancy and often persists into adulthood. Symptoms include chronic or relapsing eczematous lesions, pruritus, and lichenification. Pruritus typically worsens at night causing sleep loss and having a substantial negative impact on the quality of life and mental health of the patient. (Bieber T., N Engl J Med 2008; 358: 1483-94). Typical treatments, such as topical agents e.g. corticosteroids, phototherapy, and systemic treatments, are often ineffective.

Studies show that transgenic mice overexpressing IL-31 develop features of the skin including hyperkeratosis, acanthosis, inflammatory cell infiltration and increased mast cells that closely resemble those of lesional skin from patients with atopic dermatitis (Dillon et al., Nat Immunol. 2004; 5: 752-760). Therapies that block, inhibit or reduce signal transduction of IL-31 on its cognate receptor have the potential to treat atopic dermatitis and other chronic inflammatory conditions. BMS-981164 is an IL-31 monoclonal antibody developed by Bristol-Myers Squibb for the treatment of atopic dermatitis. A Phase 1 clinical trial completed in 2015 however results were not published (https://clinicaltrials.gov/study/NCT01614756?term=BMS-981164) and no further clinical trials of BMS-981164 appear to be ongoing. Nemolizumab, a humanized monoclonal antibody targeting the IL-31 receptor was approved in Japan in 2022 and is a currently marketed therapeutic targeting IL-31 for the treatment of atopic dermatitis.

There is a need for further innovative therapeutics, particularly for those patients who do not achieve adequate speed and/or persistence of efficacy with currently available treatments.

In particular, there is a need for an IL-31 antibody having improved pharmacological activity, for example having high affinity for IL-31 and/or high potency for inhibiting IL-31 mediated signaling. Such IL-31 antibodies have use as a monotherapy for treating IL-31 mediated disorders, such as atopic dermatitis, as well as combination therapies for treating disorders mediated by IL-31 and one or more targets different from IL-31.

Interleukin-13 (IL-13) is another cytokine associated with the pathogenesis of inflammatory disorders such as atopic dermatitis. Serum levels of IL-13 have been shown to correlate with disease severity. Tralokinumab is an IL-13 antibody approved for human use for the treatment of moderate-to-severe atopic dermatitis. Lebrikizumab, an IgG4 monoclonal antibody that targets IL-13, has been shown in phase 3 trials to significantly improve skin clearance, itch, and quality of sleep-in patients with moderate-to-severe atopic dermatitis (Silverberg J et al., N Engl J Med. 2023; 388: 1080-1091) and has since been approved for human use.

The co-blockade of IL-13 and IL-31 using a multispecific antibody, e.g., bispecific antibody, according to the present disclosure has the potential to address unmet medical need in such diseases. Thus, there is a need for a high affinity and/or highly potent IL-31 antibody that can be used in a multispecific antibody, such as a bispecific IL-31/IL-13 antibody, for use in therapy, and in particular for use in treating chronic inflammatory disorders such as atopic dermatitis.

### SUMMARY

The disclosure provides novel antibodies that bind IL-31. The antibodies according to the disclosure are monospecific IL-31 antibodies, or multispecific e.g., bispecific antibodies that target IL-31 in combination with one or more targets that are different from IL-31, for example IL-13.

In an aspect, there is provided an antibody that binds to IL-31, wherein the antibody comprises:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23;
an LCDR1 comprising SEQ ID NO: 4 or SEQ ID NO: 24;
an LCDR2 comprising SEQ ID NO: 5 or SEQ ID NO: 25; and
an LCDR3 comprising SEQ ID NO: 6 or SEQ ID NO: 26;

or
an HCDR1 comprising SEQ ID NO: 48;
an HCDR2 comprising SEQ ID NO: 49 or SEQ ID NO: 61;
an HCDR3 comprising SEQ ID NO: 50 or SEQ ID NO: 63;
an LCDR1 comprising SEQ ID NO: 51 or SEQ ID NO: 65;
an LCDR2 comprising the amino acid sequence YAK; and
an LCDR3 comprising SEQ ID NO: 53 or SEQ ID NO: 64;

or
an HCDR1 comprising SEQ ID NO: 66 or SEQ ID NO: 72;
an HCDR2 comprising SEQ ID NO: 67 or SEQ ID NO: 73;
an HCDR3 comprising SEQ ID NO: 68 or SEQ ID NO: 77;
an LCDR1 comprising SEQ ID NO: 69 or SEQ ID NO: 76;
an LCDR2 comprising the amino acid sequence YAK; and
an LCDR3 comprising SEQ ID NO: 71 or SEQ ID NO: 75;

or
an HCDR1 comprising SEQ ID NO: 78 or SEQ ID NO: 90;
an HCDR2 comprising SEQ ID NO: 79 or SEQ ID NO: 91;
an HCDR3 comprising SEQ ID NO: 80 or SEQ ID NO: 96;
an LCDR1 comprising SEQ ID NO: 81 or SEQ ID NO: 95;
an LCDR2 comprising SEQ ID NO: 82 or SEQ ID NO: 93; and
an LCDR3 comprising SEQ ID NO: 83 or SEQ ID NO: 94.

In some embodiments, the antibody comprises:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23;
an LCDR1 comprising SEQ ID NO: 4 or SEQ ID NO: 24;
an LCDR2 comprising SEQ ID NO: 5 or SEQ ID NO: 25; and
an LCDR3 comprising SEQ ID NO: 6 or SEQ ID NO: 26,
   preferably wherein the HCDR1, HCDR2, HCDR3, LCDR1, LCDR2, and LCDR3 are defined according to the Kabat numbering scheme.

In some embodiments, the antibody comprises:
i) an HCDR1 comprising SEQ ID NO: 1
ii) an HCDR2 comprising SEQ ID NO: 21; and
iii) an HCDR3 comprising SEQ ID NO: 3;
or
i) an HCDR1 comprising SEQ ID NO: 1;
ii) an HCDR2 comprising SEQ ID NO: 21; and
iii) an HCDR3 comprising SEQ ID NO: 23;
or
i) an HCDR1 comprising SEQ ID NO: 1;
ii) an HCDR2 comprising SEQ ID NO: 2; and
iii) an HCDR3 comprising SEQ ID NO: 3,
   preferably wherein the HCDR1, HCDR2, and HCDR3 are defined according to the Kabat numbering scheme.

In some embodiments, the antibody comprises:
i) an LCDR1 comprising SEQ ID NO:4;
ii) an LCDR2 comprising SEQ ID NO:25; and
iii) an LCDR3 comprising SEQ ID NO:26
or
i) an LCDR1 comprising SEQ ID NO:4;
ii) an LCDR2 comprising SEQ ID NO:5; and
iii) an LCDR3 comprising SEQ ID NO:6
or
i) an LCDR1 comprising SEQ ID NO:4;
ii) an LCDR2 comprising SEQ ID NO:5; and
iii) an LCDR3 comprising SEQ ID NO:26
or
i) an LCDR1 comprising SEQ ID NO: 24;
ii) an LCDR2 comprising SEQ ID NO: 5; and
iii) an LCDR3 comprising SEQ ID NO: 26,
   preferably wherein the LCDR1, LCDR2, and LCDR3 are defined according to the Kabat numbering scheme.

In some embodiments, the antibody comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO:3;
iv) an LCDR1 comprising SEQ ID NO: 4;
v) an LCDR2 comprising SEQ ID NO: 5; and
vi) an LCDR3 comprising SEQ ID NO 6,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) a HCDR1 comprising SEQ ID NO: 1;
ii) a HCDR2 comprising SEQ ID NO: 2;
iii) a HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
   preferably wherein the HCDR1, HCDR2, HCDR3, LCDR1, LCDR2, and LCDR3 are defined according to the Kabat numbering scheme.

In some embodiments, the antibody comprises a heavy chain variable region (VH) comprising the amino acid sequence shown in SEQ ID NO: 7 or SEQ ID NO: 28 or SEQ ID NO: 29. In some embodiments, the antibody comprises a light chain variable region (VL) comprising the amino acid sequence shown in SEQ ID NO: 8 or SEQ ID NO: 30 or SEQ ID NO:31 or SEQ ID NO:32.

In some embodiments, the antibody comprises:
i) a heavy chain variable region (VH) comprising the amino acid sequence shown in SEQ ID NO: 28; and
ii) a light chain variable region (VL) comprising the amino acid sequence shown in SEQ ID NO: 31;
or
i) a heavy chain variable region (VH) comprising the amino acid sequence shown in SEQ ID NO: 28; and
ii) a light chain variable region (VL) comprising the amino acid sequence shown in SEQ ID NO: 32;
or
i) a heavy chain variable region (VH) comprising the amino acid sequence shown in SEQ ID NO: 28; and
ii) a light chain variable region (VL) comprising the amino acid sequence shown in SEQ ID NO: 30;
or
i) a heavy chain variable region (VH) comprising the amino acid sequence shown in SEQ ID NO: 28; and
ii) a light chain variable region (VL) comprising the amino acid sequence shown in SEQ ID NO: 8;
or
i) a heavy chain variable region (VH) comprising the amino acid sequence shown in SEQ ID NO: 29; and
ii) a light chain variable region (VL) comprising the amino acid sequence shown in SEQ ID NO: 31;
or
i) a heavy chain variable region (VH) comprising the amino acid sequence shown in SEQ ID NO: 29; and
ii) a light chain variable region (VL) comprising the amino acid sequence shown in SEQ ID NO: 32;
or
i) a heavy chain variable region (VH) comprising the amino acid sequence shown in SEQ ID NO: 29; and
ii) a light chain variable region (VL) comprising the amino acid sequence shown in SEQ ID NO: 30;
or
i) a heavy chain variable region (VH) comprising the amino acid sequence shown in SEQ ID NO: 29; and
ii) a light chain variable region (VL) comprising the amino acid sequence shown in SEQ ID NO: 8;
or
i) a heavy chain variable region (VH) comprising the amino acid sequence shown in SEQ ID NO: 7; and
ii) a light chain variable region (VL) comprising the amino acid sequence shown in SEQ ID NO: 31;
or
i) a heavy chain variable region (VH) comprising the amino acid sequence shown in SEQ ID NO: 7; and
ii) a light chain variable region (VL) comprising the amino acid sequence shown in SEQ ID NO: 32;
or
i) a heavy chain variable region (VH) comprising the amino acid sequence shown in SEQ ID NO: 7; and
ii) a light chain variable region (VL) comprising the amino acid sequence shown in SEQ ID NO: 30;
or
i) a heavy chain variable region (VH) comprising the amino acid sequence shown in SEQ ID NO: 7; and
ii) a light chain variable region (VL) comprising the amino acid sequence shown in SEQ ID NO: 8.

In some embodiments, the antibody comprises:
i) a light chain comprising the amino acid sequence shown in SEQ ID NO: 34; and
ii) a heavy chain comprising the amino acid sequence shown in SEQ ID NO: 37;
or
i) a light chain comprising the amino acid sequence shown in SEQ ID NO: 35; and
ii) a heavy chain comprising the amino acid sequence shown in SEQ ID NO: 37;
or
i) a light chain comprising the amino acid sequence shown in SEQ ID NO: 33; and
ii) a heavy chain comprising the amino acid sequence shown in SEQ ID NO: 37;
or
i) a light chain comprising the amino acid sequence shown in SEQ ID NO: 9; and
ii) a heavy chain comprising the amino acid sequence shown in SEQ ID NO: 37;
or
i) a light chain comprising the amino acid sequence shown in SEQ ID NO: 34; and
ii) a heavy chain comprising the amino acid sequence shown in SEQ ID NO: 38;
or
i) a light chain comprising the amino acid sequence shown in SEQ ID NO: 35; and
ii) a heavy chain comprising the amino acid sequence shown in SEQ ID NO: 38;
or
i) a light chain comprising the amino acid sequence shown in SEQ ID NO: 33; and
ii) a heavy chain comprising the amino acid sequence shown in SEQ ID NO: 38;
or
i) a light chain comprising the amino acid sequence shown in SEQ ID NO: 9; and
ii) a heavy chain comprising the amino acid sequence shown in SEQ ID NO: 38;
or
i) a light chain comprising the amino acid sequence shown in SEQ ID NO: 34; and
ii) a heavy chain comprising the amino acid sequence shown in SEQ ID NO: 39;
or
i) a light chain comprising the amino acid sequence shown in SEQ ID NO: 35; and
ii) a heavy chain comprising the amino acid sequence shown in SEQ ID NO: 39;
or
i) a light chain comprising the amino acid sequence shown in SEQ ID NO: 33; and
ii) a heavy chain comprising the amino acid sequence shown in SEQ ID NO: 39;
or
i) a light chain comprising the amino acid sequence shown in SEQ ID NO: 9; and
ii) a heavy chain comprising the amino acid sequence shown in SEQ ID NO: 39.

In some embodiments, the antibody further comprises an Fc polypeptide comprising the amino acid sequence shown in SEQ ID NO: 42 or a variant thereof. In some embodiments, the variant of SEQ ID NO: 42 comprises one or more mutations selected from:
a) one or more silencing mutations, e.g., an L234A and an L235A mutation (“LALA”); and/or
b) one or more half-life extension mutations, e.g., an M252Y, an S254T, and a T256E mutation (“YTE”); and/or
c) one or more K-i-H mutations, e.g., an S354C mutation and/or a T366W mutation or an Y349C, a T366S, an L368A, and/or a Y407V mutation, or one or more FAE mutations e.g., an F405L mutation or a K409R mutation,
according to EU numbering.

In some embodiments, the variant of SEQ ID NO: 42 comprises:
a) an L234A and an L235A mutation (“LALA”);
b) an M252Y, an S254T, and a T256E mutation (“YTE”); and
c) an F405L or a K409R mutation,
according to EU numbering.

In some embodiments, the Fc polypeptide comprises the amino acid sequence shown in SEQ ID NO: 44 or SEQ ID NO: 45.

In a further aspect there is provided an antibody that binds to IL-31, wherein the antibody comprises:
i) a light chain comprising the amino acid sequence shown in SEQ ID NO: 9; and
ii) a heavy chain comprising the amino acid sequence shown in SEQ ID NO: 10 or 109;
or
i) a light chain comprising the amino acid sequence shown in SEQ ID NO: 34; and
ii) a heavy chain comprising the amino acid sequence shown in SEQ ID NO: 104 or 107;
or
i) a light chain comprising the amino acid sequence shown in SEQ ID NO: 35; and
ii) a heavy chain comprising the amino acid sequence shown in SEQ ID NO: 104 or 107;
or
i) a light chain comprising the amino acid sequence shown in SEQ ID NO: 33; and
ii) a heavy chain comprising the amino acid sequence shown in SEQ ID NO: 104 or 107;
or
i) a light chain comprising the amino acid sequence shown in SEQ ID NO: 9; and
ii) a heavy chain comprising the amino acid sequence shown in SEQ ID NO: 104 or 107;
or
i) a light chain comprising the amino acid sequence shown in SEQ ID NO: 34; and
ii) a heavy chain comprising the amino acid sequence shown in SEQ ID NO: 105 or 108;
or
i) a light chain comprising the amino acid sequence shown in SEQ ID NO: 35; and
ii) a heavy chain comprising the amino acid sequence shown in SEQ ID NO: 105 or 108;
or
i) a light chain comprising the amino acid sequence shown in SEQ ID NO: 33; and
ii) a heavy chain comprising the amino acid sequence shown in SEQ ID NO: 105 or 108;
or
i) a light chain comprising the amino acid sequence shown in SEQ ID NO: 9; and
ii) a heavy chain comprising the amino acid sequence shown in SEQ ID NO: 105 or 108;
or
i) a light chain comprising the amino acid sequence shown in SEQ ID NO: 34; and
ii) a heavy chain comprising the amino acid sequence shown in SEQ ID NO: 10 or 109;
or
i) a light chain comprising the amino acid sequence shown in SEQ ID NO: 35; and
ii) a heavy chain comprising the amino acid sequence shown in SEQ ID NO: 10 or 109;
or
i) a light chain comprising the amino acid sequence shown in SEQ ID NO: 33; and
ii) a heavy chain comprising the amino acid sequence shown in SEQ ID NO: 10 or 109.

In a further aspect, there is provided an antibody that binds to IL-31, comprising:
(1) a heavy chain variable region comprising a variant of SEQ ID NO: 40, wherein the variant comprises relative to SEQ ID NO: 40 a substitution of the leucine (L) at position 29 (VH L29) by a different amino acid, wherein the glutamine (Q) at the first position of SEQ ID NO: 40 is denoted as position 1 (VH Q1); and
(2) a light chain variable region comprising SEQ ID NO: 8 or SEQ ID NO: 30 or SEQ ID NO:31 or SEQ ID NO:32. In some embodiments, the substitution of the leucine (L) at position 29 (VH L29) is a substitution by an arginine (R).

In a further aspect, there is provided an antibody that binds to IL-31, comprising:
(1) a heavy chain variable region comprising a variant of SEQ ID NO: 40, wherein the variant comprises relative to SEQ ID NO: 40 a substitution of the glycine (G) at position 54 (VH G54), a substitution of the aspartic acid (D) at position 55 (VH D55), and a substitution of the glutamine (Q) at position 65 (VH Q65) by a different amino acid, wherein the glutamine (Q) at the first position of SEQ ID NO: 40 is denoted as position 1 (VH Q1); and
(2) a light chain variable region comprising SEQ ID NO: 8 or SEQ ID NO: 30 or SEQ ID NO:31 or SEQ ID NO:32. In some embodiments, the substitution of the glycine (G) at position 54 (VH G54) is a substitution by an alanine (A), the substitution of the aspartic acid (D) at position 55 (VH D55) is a substitution by a glutamic acid (E), and the substitution of the glutamine (Q) at position 65 (VH Q65) is a substitution by a lysine (K).

In a further aspect, there is provided an antibody that binds to IL-31, comprising:
(1) a heavy chain variable region comprising a variant of SEQ ID NO: 40, wherein the variant comprises relative to SEQ ID NO: 40 a substitution of the aspartic acid (D) at position 100 (VH D100), a substitution of the tyrosine (Y) at position 102 (VH Y102), and a substitution of the aspartic acid (D) at position 110 (VH D110) by a different amino acid, wherein the glutamine (Q) at the first position of SEQ ID NO: 40 is denoted as position 1 (VH Q1); and
(2) a light chain variable region comprising SEQ ID NO: 8 or SEQ ID NO: 30 or SEQ ID NO:31 or SEQ ID NO:32. In some embodiments, the substitution of the aspartic acid (D) at position 100 (VH D100) is a substitution by a glutamic acid (E), the substitution of the tyrosine (Y) at position 102 (VH Y102) is a substitution by a proline (P), and the substitution of the aspartic acid (D) at position 110 (VH D110) is a substitution by a histidine (H).

In some embodiments, the variant of SEQ ID NO: 40 further comprises relative to SEQ ID NO: 40 a substitution of the alanine (A) at position 16 (VH A16), a substitution of the leucine (L) at position 70 (VH L70), and a substitution of the leucine (L) at position 117 (VH L117) by a different amino acid, wherein the glutamine (Q) at the first position of SEQ ID NO: 40 is denoted as position 1 (VH Q1). In some embodiments, the substitution of the alanine (A) at position 16 (VH A16) is a substitution by a serine (S), the substitution of the leucine (L) at position 70 (VH L70) is a substitution by an isoleucine (I), and the substitution of the leucine (L) at position 117 (VH L117) is a substitution by a threonine (T).

In a further aspect, there is provided an antibody that binds to IL-31, comprising:
(1) a heavy chain variable region comprising the amino acid sequence shown in SEQ ID NO: 7, SEQ ID NO: 28, or SEQ ID NO: 29; and
(2) a light chain variable region comprising a variant of SEQ ID NO: 41, wherein the variant comprises relative to SEQ ID NO: 41 a substitution of the glycine (G) at position 27 (VL G27) and a substitution of the histidine (H) at position 30 (VL H30) by a different amino acid, wherein the aspartic acid (D) at the first position of SEQ ID NO: 41 is denoted as position 1 (VL D1). In some embodiments, the substitution of the glycine (G) at position 27 (VL G27) is a substitution by an arginine (R) and the substitution of the histidine (H) at position 30 (VL H30) is a substitution by a glycine (G).

In a further aspect, there is provided an antibody that binds to IL-31, comprising:
(1) a heavy chain variable region comprising the amino acid sequence shown in SEQ ID NO: 7, SEQ ID NO: 28, or SEQ ID NO: 29; and
(2) a light chain variable region comprising a variant of SEQ ID NO: 41, wherein the variant comprises relative to SEQ ID NO: 41 a substitution of the asparagine (N) at position 50 (VL N50), a substitution of the leucine (L) at position 54 (VL L54), and a substitution of the aspartic acid (D) at position 56 (VL D56) by a different amino acid, wherein the aspartic acid (D) at the first position of SEQ ID NO: 41 is denoted as position 1 (VL D1). In some embodiments, the substitution of the asparagine (N) at position 50 (VL N50) is a substitution by a tyrosine (Y), the substitution of the leucine (L) at position 54 (VL L54) is a substitution by a serine (S), and the substitution of the aspartic acid (D) at position 56 (VL D56) is a substitution by a serine (S).

In some embodiments, the variant of SEQ ID NO: 41 further comprises relative to SEQ ID NO: 40 a substitution of the aspartic acid (D) at position 1 (VL D1), a substitution of the glutamic acid (E) at position 68 (VL E68), a substitution of the glutamine (Q) at position 70 (VL Q70), a substitution of the serine (S) at position 72 (VL S72), and a substitution of the glutamine (Q) at position 100 (VL Q100), wherein the aspartic acid (D) at the first position of SEQ ID NO: 41 is denoted as position 1 (VL D1). In some embodiments, the substitution of the aspartic acid (D) at position 1 (VL D1) is a substitution by an alanine (A), the substitution of the glutamic acid (E) at position 68 (VL E68) is a substitution by a glycine (G), the substitution of the glutamine (Q) at position 70 (VL Q70) is a substitution by an aspartic acid (D), the substitution of the serine (S) at position 72 (VL S72) is a substitution by a threonine (T), and the substitution of the glutamine (Q) at position 100 (VL Q100) is a substitution by a glycine (G).

In a further aspect, there is provided an antibody that binds to IL-31, comprising:
(1) a heavy chain variable region comprising a variant of SEQ ID NO: 40, wherein the variant comprises relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1), and;
(2) a light chain variable region comprising a variant of SEQ ID NO: 41, wherein the variant comprises relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

In some embodiments, the antibody described herein is monospecific for IL-31. In some embodiments, the antibody inhibits IL-31 activity, preferably inhibits IL-31 activity with an IC50 of less than 30 pM, less than 25 pM, less than 15 pM or less than about 15 pM, preferably as measured by an IL-31 dependent Baf3 cell proliferation assay. In some embodiments, the antibody binds IL-31 with a dissociation constant (KD) of less than 30 pM, less than 25 pM, less than 20 pM, less than 15 pM, less than 10 pM, less than 5 pM or less than about 5 pM as measured using surface plasmon resonance. In some embodiments, the antibody comprises a Fab domain having a melting temperature (Tm) of at least 70° C., such as 70° C., 71° C., 72° C., 73° C., 74° C., 75° C., 76° C., 77° C., preferably at least 75° C., more preferably 76° C. or about 76° C., as measured by DSF.

In some embodiments, the antibody described herein is monovalent with respect to binding IL-31. In some embodiments, the antibody is multivalent, preferably bivalent, with respect to binding IL-31.

In some embodiments, the antibody described herein is a humanized, preferably monoclonal, antibody. In some embodiments, the antibody is an IgG type, for example an IgG1 type. In some embodiments, the antibody is isolated.

In some embodiments, the IL-31 is human.

In a further aspect, there is provided a multispecific antibody, wherein the antibody comprises:
(i) a first part that binds to IL-31 comprising:
   an HCDR1 comprising SEQ ID NO: 1;
   an HCDR2 comprising SEQ ID NO: 2 or SEQ ID NO: 21;
   an HCDR3 comprising SEQ ID NO: 3 or SEQ ID NO: 23; and,
   an LCDR1 comprising SEQ ID NO: 4 or SEQ ID NO: 24;
   an LCDR2 comprising SEQ ID NO: 5 or SEQ ID NO: 25, and;
   an LCDR3 comprising SEQ ID NO: 6 or SEQ ID NO: 26, and
(ii) a second part that binds to a target different from IL-31, preferably wherein the HCDR1, HCDR2, HCDR3, LCDR1, LCDR2, and LCDR3 are defined according to the Kabat numbering scheme. In preferred embodiments, the second part binds to IL-13. In preferred embodiments, the multispecific antibody is bispecific.

In some embodiments, the first part that binds to IL-31 comprises:
i) an HCDR1 comprising SEQ ID NO: 1
ii) an HCDR2 comprising SEQ ID NO: 21, and;
iii) an HCDR3 comprising SEQ ID NO: 3;
or
i) an HCDR1 comprising SEQ ID NO: 1;
ii) an HCDR2 comprising SEQ ID NO: 21, and;
iii) an HCDR3 comprising SEQ ID NO: 23;
or
i) an HCDR1 comprising SEQ ID NO: 1;
ii) an HCDR2 comprising SEQ ID NO: 2, and;
iii) an HCDR3 comprising SEQ ID NO: 3,
   preferably wherein the HCDR1, HCDR2, and HCDR3 are defined according to the Kabat numbering scheme.

In some embodiments, the first part that binds to IL-31 comprises:
i) an LCDR1 comprising SEQ ID NO: 4;
ii) an LCDR2 comprising SEQ ID NO: 25; and
iii) an LCDR3 comprising SEQ ID NO: 26,
or
i) an LCDR1 comprising SEQ ID NO: 4;
ii) an LCDR2 comprising SEQ ID NO: 5; and
iii) an LCDR3 comprising SEQ ID NO: 6,
or
i) an LCDR1 comprising SEQ ID NO: 4;
ii) an LCDR2 comprising SEQ ID NO: 5; and
iii) an LCDR3 comprising SEQ ID NO: 26,
or
i) an LCDR1 comprising SEQ ID NO: 24;
ii) an LCDR2 comprising SEQ ID NO: 5; and
iii) an LCDR3 comprising SEQ ID NO: 26,
   preferably wherein the LCDR1, LCDR2, and LCDR3 are defined according to the Kabat numbering scheme.

In some embodiments, the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO:3;
iv) an LCDR1 comprising SEQ ID NO: 4;
v) an LCDR2 comprising SEQ ID NO: 5; and
vi) an LCDR3 comprising SEQ ID NO 6,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
   preferably wherein the HCDR1, HCDR2, HCDR3, LCDR1, LCDR2, and LCDR3 are defined according to the Kabat numbering scheme.

In some embodiments, the first part that binds to IL-31 comprises a heavy chain variable region (VH) comprising the amino acid sequence shown in SEQ ID NO: 7 or SEQ ID NO: 28 or SEQ ID NO: 29. In some embodiments, the first part that binds to IL-31 comprises a light chain variable region (VL) comprising the amino acid sequence shown in SEQ ID NO: 8 or SEQ ID NO: 30 or SEQ ID NO:31 or SEQ ID NO:32.

In some embodiments, the first part that binds to IL-31 comprises:
i) a heavy chain variable region (VH) comprising the amino acid sequence shown in SEQ ID NO: 28; and
ii) a light chain variable region (VL) comprising the amino acid sequence shown in SEQ ID NO: 31;
or
i) a heavy chain variable region (VH) comprising the amino acid sequence shown in SEQ ID NO: 28; and
ii) a light chain variable region (VL) comprising the amino acid sequence shown in SEQ ID NO: 32;
or
i) a heavy chain variable region (VH) comprising the amino acid sequence shown in SEQ ID NO: 28; and
ii) a light chain variable region (VL) comprising the amino acid sequence shown in SEQ ID NO: 30;
or
i) a heavy chain variable region (VH) comprising the amino acid sequence shown in SEQ ID NO: 28; and
ii) a light chain variable region (VL) comprising the amino acid sequence shown in SEQ ID NO: 8;
or
i) a heavy chain variable region (VH) comprising the amino acid sequence shown in SEQ ID NO: 29; and
ii) a light chain variable region (VL) comprising the amino acid sequence shown in SEQ ID NO: 31;
or
i) a heavy chain variable region (VH) comprising the amino acid sequence shown in SEQ ID NO: 29; and
ii) a light chain variable region (VL) comprising the amino acid sequence shown in SEQ ID NO: 32;
or
i) a heavy chain variable region (VH) comprising the amino acid sequence shown in SEQ ID NO: 29; and
ii) a light chain variable region (VL) comprising the amino acid sequence shown in SEQ ID NO: 30;
or
i) a heavy chain variable region (VH) comprising the amino acid sequence shown in SEQ ID NO: 29; and
ii) a light chain variable region (VL) comprising the amino acid sequence shown in SEQ ID NO: 8;
or
i) a heavy chain variable region (VH) comprising the amino acid sequence shown in SEQ ID NO: 7; and
ii) a light chain variable region (VL) comprising the amino acid sequence shown in SEQ ID NO: 31;
or
i) a heavy chain variable region (VH) comprising the amino acid sequence shown in SEQ ID NO: 7; and
ii) a light chain variable region (VL) comprising the amino acid sequence shown in SEQ ID NO: 32;
or
i) a heavy chain variable region (VH) comprising the amino acid sequence shown in SEQ ID NO: 7; and
ii) a light chain variable region (VL) comprising the amino acid sequence shown in SEQ ID NO: 30;
or
i) a heavy chain variable region (VH) comprising the amino acid sequence shown in SEQ ID NO: 7; and
ii) a light chain variable region (VL) comprising the amino acid sequence shown in SEQ ID NO: 8.

In some embodiments, the first part that binds to IL-31 comprises:
i) a light chain comprising the amino acid sequence shown in SEQ ID NO: 34; and
ii) a heavy chain comprising the amino acid sequence shown in SEQ ID NO: 37;
or
i) a light chain comprising the amino acid sequence shown in SEQ ID NO: 35; and
ii) a heavy chain comprising the amino acid sequence shown in SEQ ID NO: 37;
or
i) a light chain comprising the amino acid sequence shown in SEQ ID NO: 33; and
ii) a heavy chain comprising the amino acid sequence shown in SEQ ID NO: 37;
or
i) a light chain comprising the amino acid sequence shown in SEQ ID NO: 9; and
ii) a heavy chain comprising the amino acid sequence shown in SEQ ID NO: 37;
or
i) a light chain comprising the amino acid sequence shown in SEQ ID NO: 34; and
ii) a heavy chain comprising the amino acid sequence shown in SEQ ID NO: 38;
or
i) a light chain comprising the amino acid sequence shown in SEQ ID NO: 35; and
ii) a heavy chain comprising the amino acid sequence shown in SEQ ID NO: 38;
or
i) a light chain comprising the amino acid sequence shown in SEQ ID NO: 33; and
ii) a heavy chain comprising the amino acid sequence shown in SEQ ID NO: 38;
or
i) a light chain comprising the amino acid sequence shown in SEQ ID NO: 9; and
ii) a heavy chain comprising the amino acid sequence shown in SEQ ID NO: 38;
or
i) a light chain comprising the amino acid sequence shown in SEQ ID NO: 34; and
ii) a heavy chain comprising the amino acid sequence shown in SEQ ID NO: 39;
or
i) a light chain comprising the amino acid sequence shown in SEQ ID NO: 35; and
ii) a heavy chain comprising the amino acid sequence shown in SEQ ID NO: 39;
or
i) a light chain comprising the amino acid sequence shown in SEQ ID NO: 33; and
ii) a heavy chain comprising the amino acid sequence shown in SEQ ID NO: 39;
or
i) a light chain comprising the amino acid sequence shown in SEQ ID NO: 9; and
ii) a heavy chain comprising the amino acid sequence shown in SEQ ID NO: 39.

In some embodiments, the second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16,
   preferably wherein the HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
and
   the second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16,
   preferably wherein the HCDR1, HCDR2, HCDR3, LCDR1, LCDR2, and LCDR3 are defined according to the Kabat numbering scheme.

In some embodiments, the second part that binds to IL-13 comprises a heavy chain variable region (VH) comprising the amino acid sequence shown in SEQ ID NO: 17. In some embodiments, the second part that binds to IL-13 comprises a light chain variable region (VL) comprising the amino acid sequence shown in SEQ ID NO: 18.

In some embodiments,
the first part that binds to IL-31 comprises a heavy chain variable region (VH) comprising the amino acid sequence shown in SEQ ID NO: 7 and a light chain variable region (VL) comprising the amino acid sequence shown in SEQ ID NO: 8; and
the second part that binds to IL-13 comprises a heavy chain variable region (VH) comprising the amino acid sequence shown in SEQ ID NO: 17 and a light chain variable region (VL) comprising the amino acid sequence shown in SEQ ID NO: 18.

In some embodiments, the second part that binds to IL-13 comprises a light chain comprising an amino acid sequence shown in SEQ ID NO: 19. In some embodiments, the second part that binds to IL-13 comprises a heavy chain comprising an amino acid sequence shown in SEQ ID NO: 43.

In some embodiments,
the first part that binds to IL-31 comprises a light chain comprising the amino acid sequence shown in SEQ ID NO: 9 and a heavy chain comprising the amino acid sequence shown in SEQ ID NO: 39; and
the second part that binds to IL-13 comprises a light chain comprising the amino acid sequence shown in SEQ ID NO: 19 and a heavy chain comprising the amino acid sequence shown in SEQ ID NO: 43.

In some embodiments,
the first part that binds to IL-31 comprises a first Fc polypeptide, and
the second part that binds to IL-13 comprises a second Fc polypeptide, wherein the first and/or second Fc polypeptide comprises the amino acid sequence shown in SEQ ID NO: 42 or a variant thereof. In some embodiments, the variant of SEQ ID ON: 42 comprises one or more mutations selected from:
a. one or more silencing mutations e.g., an L234A and an L235A mutation (“LALA”); and/or
b. one or more half-life extension mutations e.g., an M252Y, an S254T and a T256E mutation (“YTE”); and/or
c. one or more K-i-H mutations e.g., an S354C and/or a T366W mutation or an Y349C, a T366S, an L368A, and/or Y407V mutation, or FAE mutations e.g., an F405L mutation or a K409R mutation,
according to EU numbering.

In some embodiments,
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) an F405L mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an K409R mutation;
or
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) an K409R mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an F405L mutation,
according to EU numbering.

In some embodiments,
i) the first part that binds to IL-31 comprises a first Fc polypeptide comprising the amino acid sequence shown in SEQ ID NO: 44, and the second part that binds to IL-13 comprises a second Fc polypeptide comprising the amino acid sequence shown in SEQ ID NO: 45; or
ii) the first part that binds to IL-31 comprises a first Fc polypeptide comprising the amino acid sequence shown in SEQ ID NO: 45, and the second part that binds to IL-13 comprises a second Fc polypeptide comprising the amino acid sequence shown in SEQ ID NO: 44.

In a further aspect, there is provided a multispecific, preferably bispecific, antibody comprising a first part that binds to IL-31 and a second part that binds to IL-13, wherein the first part that binds to IL-31 comprises:
i) a light chain comprising the amino acid sequence shown in SEQ ID NO: 9; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19; and iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 34; and
ii) a heavy chain comprising the amino acid sequence shown in SEQ ID NO: 104 or 107;
and the second part that binds to IL-13 comprises:
iii) a light chain comprising the amino acid sequence shown in SEQ ID NO: 19; and iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 35; and
ii) a heavy chain comprising the amino acid sequence shown in SEQ ID NO: 104 or 107;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 33; and
ii) a heavy chain comprising the amino acid sequence shown in SEQ ID NO: 104 or 107;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 9; and
ii) a heavy chain comprising the amino acid sequence shown in SEQ ID NO: 104 or 107;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 34; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 35; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 33; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 9; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 34; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 35; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19; and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 33; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110; In some embodiments, the first part that binds to IL-31 comprises:
i) a light chain comprising or consisting of the amino acid sequence shown in SEQ ID NO: 9; and
ii) a heavy chain comprising or consisting of the amino acid sequence shown in SEQ ID NO 10;
and the second part that binds to IL-13 comprises:
iii) a light chain comprising or consisting of the amino acid sequence shown in SEQ ID NO: 19, and
iv) a heavy chain comprising or consisting of the amino acid sequence shown in SEQ ID NO: 20.

In some embodiments, the first part that binds to IL-31 comprises:
i) a light chain comprising or consisting of the amino acid sequence shown in SEQ ID NO: 9; and
ii) a heavy chain comprising or consisting of the amino acid sequence shown in SEQ ID NO 109;
and the second part that binds to IL-13 comprises:
iii) a light chain comprising or consisting of the amino acid sequence shown in SEQ ID NO: 19, and
iv) a heavy chain comprising or consisting of the amino acid sequence shown in SEQ ID NO: 110.

In some embodiments, the multispecific antibody is monovalent with respect to binding IL-31 and monovalent with respect to binding IL-13. In some embodiments, the light chain of the first part that binds to IL-31 is a kappa light chain and the light chain of the second part that binds to IL-13 is a kappa light chain. In some embodiments, the multispecific antibody has an IgG, preferably an IgG1 isotype. In some embodiments, the multispecific antibody is isolated. In some embodiments, the multispecific antibody is bivalent, and is monovalent with respect to binding IL-31 and monovalent with respect to binding IL-13. In some embodiments, the multispecific antibody is humanized. In some embodiments, the first part that binds to IL-31 comprises a Fab and the second part that binds to IL-13 comprises a Fab. In some embodiments, the first part that binds to IL-31 is monoclonal and the second part that binds to IL-13 is monoclonal. In some embodiments, the IL-31 and IL-13 are human. In some embodiments, the multispecific antibody binds human IL-31 with a dissociation constant (KD) of less than 30 pM, less than 25 pM, less than 20 pM, less than 15 pM, less than 10 pM, or less than about 10 pM, as measured using surface plasmon resonance. In some embodiments, the multispecific antibody binds human IL-13 with a dissociation constant (KD) of less than 30 pM, less than 25 pM, less than 20 pM, less than 15 pM, less than 10 pM, less than 5 pM, less than 1 pM or less than about 1 pM, as measured using surface plasmon resonance. In some embodiments, the multispecific antibody inhibits human IL-31 activity, preferably inhibits human IL-31 activity with an IC50 of less than 30 pM, such as less than 25 pM, or less than 20 pM or less than about 20 pM, as measured by an IL-31 dependent Baf3 cell proliferation assay. In some embodiments, the multispecific antibody inhibits human IL-13 activity, preferably inhibits human IL-13 activity with an IC50 of less than 50 pM, less than 40 pM, less than 30 pM, less than 20 pM, less than 15 pM or less than about 15 pM, as measured in a HEK-Blue cell reporter assay.

In a further aspect, there is provided an ex vivo method for detecting the presence and/or concentration of IL-31 protein in a biological sample, comprising the steps of:
(i) providing a biological sample from a subject,
(ii) reacting said biological sample with at least one antibody described herein, under conditions sufficient for binding IL-31 protein present in said biological sample to said at least one antibody through antigen-antibody interactions, thus forming an IL-31-antibody complex; and
(iii) detecting a signal proportional to the level of antigen IL-31-antibody complex formed in step (ii),
wherein the intensity of the signal correlates with the concentration of IL-31 protein in the biological sample.

In a further aspect, there is provided a pharmaceutical composition comprising an antibody (e.g., a monospecific or multispecific antibody) described herein, optionally in combination with one or more pharmaceutically acceptable excipients, diluents, or carriers.

In a further aspect, there is provided an antibody (e.g., a monospecific or multispecific antibody) or pharmaceutical composition described herein, for use as a medicament. In a further aspect, there is provided the use of an antibody (e.g., a monospecific or multispecific antibody) or pharmaceutical composition described herein as a medicament.

In a further aspect, there is provided an antibody (e.g., a monospecific or multispecific antibody) or pharmaceutical composition described herein, for use in preventing and/or treating an IL-31-related disease or disorder. In a further aspect, there is provided the use of an antibody (e.g., a monospecific or multispecific antibody) or pharmaceutical composition described herein for the manufacture of a medicament for the treatment and/or prevention of an IL-31-related disease or disorder. In a further aspect, there is provided a method of preventing and/or treating an IL-31-related disease or disorder in a subject in need thereof, comprising administering to the subject a therapeutically effective amount of an antibody (e.g., a monospecific or multispecific antibody) or pharmaceutical composition described herein. In some embodiments, the IL-31-related disease or disorder is an inflammatory or immune disease or disorder. In some embodiments, the inflammatory or immune disease or disorder is a skin disease or disorder. In some embodiments, the IL-31 is human.

In a further aspect, there is provided a method of inhibiting the activity of IL-31, the method comprising contacting an effective amount of an antibody (e.g., a monospecific or multispecific antibody) or pharmaceutical composition described herein with a plurality of mammalian, preferably human cells.

In a further aspect, there is provided an ex vivo method for detecting the presence and/or concentration of IL-31 protein and/or IL-13 protein in a biological sample, comprising the steps of:
(i) providing a biological sample from a subject,
(ii) reacting said biological sample with at least one multispecific antibody, preferably bispecific antibody, described herein under conditions sufficient for binding IL-31 protein and/or IL-13 protein present in said biological sample to said at least one antibody through antigen-antibody interactions, thus forming an IL-31- and/or IL-13-antibody complex; and
(iii) detecting a signal proportional to the level of IL-31- and/or IL-13-antibody complex formed in step (ii),
wherein the intensity of the signal correlates with the concentration of IL-31 protein and/or IL-13 protein in the biological sample.

In a further aspect, there is provided an antibody (e.g., a monospecific or multispecific antibody) or pharmaceutical composition described herein, for use in preventing and/or treating an IL-31- and IL-13-related disease or disorder. In a further aspect, there is provided the use of an antibody (e.g., a monospecific or multispecific antibody) or pharmaceutical composition described herein for the manufacture of a medicament for the treatment and/or prevention of an IL-31 and IL-13-related disease or disorder. In a further aspect, there is provided a method of preventing and/or treating an IL-31- and IL-13-related disease or disorder in a subject in need thereof, comprising administering to the subject a therapeutically effective amount of an antibody (e.g., a monospecific or multispecific antibody) or pharmaceutical composition described herein. In some embodiments, the IL-31- and IL-13-related disease or disorder is an inflammatory or immune disease or disorder. In some embodiments, the inflammatory or immune disease or disorder is a skin disease or disorder. In some embodiments, the IL-31 and IL-13 are human.

In a further aspect, there is provided a method of simultaneously inhibiting the activity of IL-31 and IL-13, the method comprising contacting an effective amount of a multispecific antibody, preferably bispecific antibody, described herein with a plurality of mammalian, preferably human cells. In some embodiments, the method comprises administering the antibody to a subject, preferably a human subject. In some embodiments, the method is an ex vivo or an in vitro method.

In a further aspect, there is provided an isolated nucleic acid molecule or set of isolated nucleic acid molecules encoding an antibody (e.g., a monospecific or multispecific antibody) described herein. In a further aspect, there is provided a set of isolated nucleic acid molecules encoding a multispecific antibody, preferably bispecific antibody, described herein, wherein the first part binding to IL-31 and the second part binding to IL-13 are encoded by a separate nucleic acid molecule. In a further aspect, there is provided an expression vector or set of expression vectors comprising the isolated nucleic acid molecule or set of isolated nucleic acid molecules described herein. In a further aspect, there is provided a set of expression vectors comprising the set of isolated nucleic acid molecules described herein, wherein the nucleic acid molecule encoding the first part that binds to IL-31 and the nucleic acid molecule encoding the second part that binds to IL-13 are comprised by different vectors.

In a further aspect, there is provided a host cell comprising the nucleic acid molecule, set of nucleic acid molecules, expression vector, or set of expression vectors described herein. In a further aspect, there is provided a host cell comprising a nucleic acid molecule encoding the first part that binds to IL-31 of a multispecific antibody, preferably bispecific antibody, described herein, or an expression vector comprising said nucleic acid molecule. In a further aspect, there is provided a host cell comprising a nucleic acid molecule encoding the second part that binds to IL-13 of a multispecific antibody, preferably bispecific antibody, described herein, or an expression vector comprising said nucleic acid molecule. In some embodiments, the host cell is a HEK cell or a CHO cell.

In a further aspect, there is provided a mixture of host cells comprising a first and second host cell, wherein:
a. the first host cell comprises a nucleic acid molecule encoding the first part that binds to IL-31 of a multispecific antibody, preferably bispecific antibody, described herein, or an expression vector comprising said nucleic acid molecule; and
b. the second host cell comprises a nucleic acid molecule encoding the second part that binds to IL-13 of a multispecific antibody, preferably bispecific antibody described herein, or an expression vector comprising said nucleic acid molecule.

In a further aspect, there is provided a cell culture comprising a host cell or mixture of host cells described herein.

In a further aspect, there is provided an antibody (e.g., a monospecific or multispecific antibody) or pharmaceutical composition described herein, for use in treating and/or preventing atopic dermatitis. In a further aspect, there is provided the use of an antibody (e.g., a monospecific or multispecific antibody) or pharmaceutical composition described herein for treating and/or preventing atopic dermatitis. In a further aspect, there is provided the use of an antibody (e.g., a monospecific or multispecific antibody) or pharmaceutical composition described herein for the manufacture of a medicament for treating and/or preventing atopic dermatitis. In a further aspect, there is provided a method for treating and/or preventing atopic dermatitis in a subject in need thereof, comprising administering to the subject a therapeutically effective amount of an antibody (e.g., a monospecific or multispecific antibody) or pharmaceutical composition described herein.

In a further aspect, there is provided an antibody (e.g., a monospecific or multispecific antibody) or pharmaceutical composition described herein for use in preventing and/or treating pruritus, preferably pruritus associated with atopic dermatitis. In a further aspect, there is provided the use of an antibody (e.g., a monospecific or multispecific antibody) or pharmaceutical composition described herein in the prevention and/or treatment of pruritus, preferably pruritus associated with atopic dermatitis. In a further aspect, there is provided the use of an antibody (e.g., a monospecific or multispecific antibody) or pharmaceutical composition described herein for the manufacture of a medicament for the treatment and/or prevention of pruritus, preferably pruritus associated with atopic dermatitis. In a further aspect, there is provided a method for treating and/or preventing pruritus in a subject in need thereof, preferably pruritus associated with atopic dermatitis, comprising administering to the subject a therapeutically effective amount of an antibody (e.g., a monospecific or multispecific antibody) or pharmaceutical composition described herein.

In some embodiments, the atopic dermatitis is moderate to severe atopic dermatitis.

In a further aspect, there is provided a method of preventing and/or treating an inflammatory or immune condition in a subject in need thereof, the method comprising simultaneously or sequentially administering a therapeutically effective amount of an antibody described herein and an IL-13 antagonist to the subject. In some embodiments, the IL-13 antagonist is an antibody. In some embodiments, the inflammatory or immune condition is atopic dermatitis. In some embodiments, the subject is a human.

In a further aspect, there is provided a method of producing an antibody (e.g., a monospecific or multispecific antibody) described herein, comprising culturing a host cell described herein under conditions sufficient to express the antibody, and thereafter recovering and optionally purifying the antibody from the host cell culture.

In a further aspect, there is provided a method of producing a multispecific, preferably bispecific, antibody described herein, comprising culturing a host cell comprising a nucleic acid molecule encoding the first part that binds to IL-31 and a host cell comprising a nucleic acid molecule encoding the second part that binds to IL-13, or mixture of host cells, described herein, under conditions sufficient to express the first part that binds to IL-31 and the second part that binds to IL-13, and thereafter recovering and optionally purifying the first and second part from the host cell culture. In some embodiments, the host cell comprising the nucleic acid molecule encoding the first part that binds to IL-31 and the host cell comprising the nucleic acid molecule encoding the second part that binds to IL-13 are cultured in different host cell cultures. In some embodiments, the host cell comprising the nucleic acid molecule encoding the first part that binds to IL-31 and the host cell comprising the nucleic acid molecule encoding the second part that binds to IL-13 are cultured in the same host cell culture.

In a further aspect, there is provided a method of producing a multispecific, preferably bispecific, antibody described herein, comprising:
a) culturing a first host cell expressing the first part that binds to IL-31 in a culture under conditions sufficient to express the antibody, and a second host cell expressing the second part that binds to IL-13 in a culture under conditions sufficient to express the antibody, wherein the first and second host cell are cultured in the same or in different cultures;
b) recovering the first- and second-part from the host cell culture(s);
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part; and
e) obtaining the multispecific antibody; and optionally purifying the multispecific antibody.

In a further aspect there is provided a method of producing a pharmaceutical composition comprising a multispecific, preferably bispecific, antibody described herein, comprising:
a) culturing a first host cell expressing the first part that binds to IL-31 in a culture under conditions sufficient to express the antibody, and a second host cell expressing the second part that binds to IL-13 in a culture under conditions sufficient to express the antibody,
wherein the first and second host cell are cultured in the same or in different cultures;
b) recovering the first- and second-part from the host cell culture(s);
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part;
e) obtaining the multispecific antibody; and optionally purifying the multispecific antibody; and
f) formulating the multispecific antibody in a pharmaceutical composition, optionally in combination with one or more pharmaceutically acceptable excipients, diluents, or carriers.

In some embodiments, recovering the first- and second-part from the host cell culture(s) comprises centrifugation or depth filtration optionally followed by sterile filtration. In some embodiments, a purification step is performed prior to incubating the first- and second-part in step c). In some embodiments, the purification step is performed utilizing affinity chromatography, for example protein A affinity chromatography. In some embodiments, the first- and second-part are incubated in step c) and/or step d) in equimolar concentrations, in a molar ratio of first-to-second part from about 1:1.01 to about 1:2, or in a molar ratio of second-to-first part from about 1:1.01 to about 1:2.

In a further aspect, there is provided a method of producing a multispecific antibody, preferably bispecific antibody, described herein, comprising:
a) culturing a first host cell expressing the first part that binds to IL-31 in a culture under conditions sufficient to express the antibody, and a second host cell expressing the second part that binds to IL-13 in a culture under conditions sufficient to express the antibody, wherein the first and second host cell are cultured in the same culture;
b) incubating together the first- and second-part, preferably under reducing conditions that allow inter-chain disulfide bond reduction in the hinge region;
c) incubating the first- and second-part under conditions that allow oxidation of cysteines in the hinge region to form inter-chain disulfide bonds between the first- and second-part; and
d) obtaining the multispecific antibody; and optionally purifying the multispecific antibody, wherein incubating together the first- and second-part in step b) is done without any prior recovery of the first- and second-art from the culture.

In some embodiments, the produced multispecific antibody is formulated in a pharmaceutical composition, optionally in combination with one or more pharmaceutically acceptable excipients, diluents, or carriers.

In a further aspect, there is provided an antibody that competes for binding to an IL-31 epitope with an antibody comprising: an HCDR1 comprising SEQ ID NO: 1; an HCDR2 comprising SEQ ID NO: 2 or SEQ ID NO: 21; an HCDR3 comprising SEQ ID NO: 3 or SEQ ID NO: 23; an LCDR1 comprising SEQ ID NO: 4 or SEQ ID NO: 24; an LCDR2 comprising SEQ ID NO: 5 or SEQ ID NO: 25, and an LCDR3 comprising SEQ ID NO: 6 or SEQ ID NO: 26, preferably wherein the HCDR1, HCDR2, HCDR3, LCDR1, LCDR2, and LCDR3 are defined according to the Kabat numbering scheme.

In some embodiments, the antibody competes for binding to an IL-31 epitope with an antibody comprising:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6, preferably wherein the HCDR1, HCDR2, HCDR3, LCDR1, LCDR2, and LCDR3 are defined according to the Kabat numbering scheme.

In a further aspect, there is provided an antibody that binds to a human IL-31 epitope, wherein the epitope consists of amino acid residues S35, D37, V38, K40, I41, E44, N81, P86, R89, A90, K93, R96, K101, D105, E106, I108, E109, H110, D112, K113, L114, I115, F116, Q117, D118, A119, P120, E121, and T122 of human IL-31, preferably wherein human IL-31 is represented by SEQ ID NO: 143 or SEQ ID NO: 173. In some embodiments, the antibody comprises a heavy chain variable region (VH) comprising at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29 and a light chain variable region (VL) comprising at least 80%, at least 85%, at least 90%, at least 95%, at least 96%, at least 97%, at least 98%, or at least 99% sequence identity with SEQ ID NO: 8 or SEQ ID NO: 30 or SEQ ID NO:31 or SEQ ID NO:32. In some embodiments, the antibody comprises a heavy chain variable region (VH) comprising at least 80%, at least 85%, at least 90%, at least 95%, at least 96%, at least 97%, at least 98%, or at least 99% sequence identity with SEQ ID NO: 7 and a light chain variable region (VL) comprising at least 80%, at least 85%, at least 90%, at least 95%, at least 96%, at least 97%, at least 98%, or at least 99% sequence identity with SEQ ID NO: 8. In some embodiments, the heavy chain variable region comprises relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1). In some embodiments, the light chain variable region comprises relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

In a further aspect, there is provided a multispecific, preferably bispecific, antibody, comprising:
a first part that binds to a human IL-31 epitope, wherein the epitope consists of amino acid residues 535, D37, V38, K40, I41, E44, N81, P86, R89, A90, K93, R96, K101, D105, E106, I108, E109, H110, D112, K113, L114, I115, F116, Q117, D118, A119, P120, E121, and T122 of human IL-31, preferably wherein human IL-31 is represented by SEQ ID NO: 143 or SEQ ID NO: 173, and;
a second part that binds to IL-13 comprising a heavy chain variable region (VH) comprising the amino acid sequence shown in SEQ ID NO: 17 and a light chain variable region (VL) comprising the amino acid sequence shown in SEQ ID NO: 18. In some embodiments, the first part that binds to the human IL-31 epitope comprises a heavy chain variable region (VH) comprising at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29 and a light chain variable region (VL) comprising at least 80%, at least 85%, at least 90%, at least 95%, at least 96%, at least 97%, at least 98%, or at least 99% sequence identity with SEQ ID NO: 8 or SEQ ID NO: 30 or SEQ ID NO:31 or SEQ ID NO:32. In some embodiments, the first part that binds to the human IL-31 epitope comprises a heavy chain variable region (VH) comprising at least 80%, at least 85%, at least 90%, at least 95%, at least 96%, at least 97%, at least 98%, or at least 99% sequence identity with SEQ ID NO: 7 and a light chain variable region (VL) comprising at least 80%, at least 85%, at least 90%, at least 95%, at least 96%, at least 97%, at least 98%, or at least 99% sequence identity with SEQ ID NO: 8. In some embodiments, the first part that binds to the human IL-31 epitope comprises a heavy chain variable region comprising relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1). In some embodiments, the first part that binds to the human IL-31 epitope comprises a light chain variable region comprising relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

In some embodiments, the antibody or multispecific antibody is humanized. In some embodiments, the antibody or multispecific antibody is an IgG type, for example an IgG1 type. In some embodiments, the multispecific antibody binds human IL-31 with a dissociation constant (KD) of less than 30 pM, less than 25 pM, less than 20 pM, less than 15 pM, less than 10 pM or less than about 10 pM, as measured using surface plasmon resonance. In some embodiments, the multispecific antibody inhibits human IL-31 activity with an IC50 of less than 30 pM, less than 25 pM, or less than 20 pM or less than about 20 pM, as measured by an IL-31 dependent Baf3 cell proliferation assay. In some embodiments, the multispecific antibody binds human IL-13 with a dissociation constant (KD) of less than 30 pM, less than 25 pM, less than 20 pM, less than 15 pM, less than 10 pM, less than 5 pM, less than 1 pM or less than about 1 pM, as measured using surface plasmon resonance. In some embodiments, the multispecific antibody inhibits human IL-13 activity with an IC50 of less than 50 pM, less than 40 pM, less than 30 pM, less than 20 pM, less than 15 pM or less than about 15 pM, as measured in a HEK-Blue cell reporter assay.

The following detailed description provides further information on the aspects and embodiments of the invention.

### BRIEF DESCRIPTION OF DRAWINGS

FIG. 1A and FIG. 1B are schematic representations of an exemplary bispecific IgG antibody according to the invention. Interdomain and interchain disulfide bridges are indicated with a line. The exemplary bispecific antibody comprises a part binding to IL-31 and a part binding to IL-13. The introduction of e.g., Fab-arm exchange mutations enable assembly by heterodimerization of the Fc polypeptides. FIG. 1A and FIG. 1B show the two possible configurations of the Fab arm exchange of the Fc.

FIG. 2A and FIG. 2B show the structure of humanized anti-IL-31 Fab (TPP-13357 (SEQ ID NO: 147, 148) and TPP-17880 (SEQ ID NO: 9, 39)) in complex with human IL-31 (SEQ ID NO: 173). Glycosylation sites and a C133S mutation site are indicated on IL-31.

FIG. 3 shows the human IL-31 epitope surface of TPP-13357 (SEQ ID NO: 147, 148). Helices αA, αB, αC, and an extended loop region 115-122 between αC and αD are bound by the antibody TPP-13357.

FIG. 4A and FIG. 4B show the key human IL-31 (SEQ ID NO: 173) epitope residues recognized by TPP-13357 (FIG. 4A; SEQ ID NO: 147, 148) and TPP-17880 (FIG. 4B; SEQ ID NO: 9, 39). Observed variations in cynomolgus IL-31 are indicated in FIG. 4A.

FIG. 5A-FIG. 5B show the analysis of the crystal structure of humanized anti-IL-31 Fab (TPP-13357; FIG. 5A top and bottom panel, TPP-17880; FIG. 5B top and bottom panel) in complex with human IL-31. Paratope residues are plotted with their surface area buried due to IL-31 binding.

FIG. 6A-FIG. 6C shows some of the key interactions between humanized anti-IL-31 Fab TPP-13357 in complex with human IL-31. Dashed lines indicate hydrogen bonds or salt bridges.

FIG. 7, FIG. 8 and FIG. 9 show the impact of mutated residues in TPP-17880 (SEQ ID NO: 9, 39) on the binding to IL-31 compared to TPP-13357 (SEQ ID NO: 147, 148).

FIG. 10A-FIG. 10B and FIG. 11A-FIG. 11B show FACS analysis showing that the populations obtained from error prone PCR and structure guided libraries have improved affinity to IL-31.

FIG. 12 is a representative dose-response curve from an exemplary experiment showing human and cynomolgus monkey IL-31 induced cell growth of IL-31Ra/OSMRb expressing BaF3 cells.

FIG. 13 shows an exemplary schematic of a vector design used to generate antibodies of the disclosure.

FIG. 14 shows SET assay results against human and cynomolgus IL-13 and IL-31 using an exemplary anti-IL-31/IL-13 antibody of the invention (SEQ ID NO: 9, 10, 19, 20).

FIG. 15 shows concentration-dependent % inhibition (IC50) of human and cynomolgus IL-31 induced cell growth using an exemplary anti-IL-31/IL-13 antibody of the invention (SEQ ID NO: 9, 10, 19, 20) in an IL-31-dependent Baf3 cell growth assay (exemplary representative experiment).

FIG. 16A-FIG. 160 show kinetic constants of parental and affinity optimized Fabs in SPR binding sensorgrams. All sensorgrams were evaluated applying the fitting model for 1:1 binding interaction. One sensorgram per antibody/antigen interaction is shown: a) TPP-13357, b) TPP-13348, c) TPP-17877, d) TPP-17876, e) TPP-17878, f) TPP-17880, g) TPP-20012, h) TPP-17867, i) TPP-17868, j) TPP-17869, k) TPP-17871, l) TPP-17872, m) TPP-17873, n) TPP-17874, o) TPP-17875.

FIG. 17 shows the inhibition of human IL-31 induced Baf3 cell growth (IL-31 dependent) by different anti-IL-31 Fab variants (TPP-20012; TPP-13348; TPP-13357; TPP-17880).

FIG. 18 shows a human IL-31 SPR binding sensorgram of the anti-IL-31/IL-13 bispecific antibody generated in Example 9.

### DETAILED DESCRIPTION

The invention is based on the discovery of improved antibodies that bind to IL-31. The antibodies of the invention have high affinity for IL-31 and/or high potency for inhibiting IL-31 mediated signaling. The antibodies of the present invention may also exhibit one or more other favorable pharmaceutical properties relative to available antibodies in the art, including high thermostability and low aggregation propensities, to facilitate manufacturing and storage, lower effector function, and/or increased half-life.

The invention provides monospecific IL-31 antibodies. The invention also provides multispecific antibodies comprising a first part that binds to IL-31 and at least a second part that binds to a target different from IL-31. In some embodiments, there is provided a bispecific antibody comprising a first part that binds to IL-31 and a second part that binds to a target different from IL-31, preferably that binds to IL-13. In some embodiments, there is provided a bispecific antibody comprising a first part that binds to IL-31 and a second part that binds to IL-13, wherein the bispecific antibody is monovalent with respect to IL-31 and monovalent with respect to IL-13. The invention also provides methods of manufacturing the antibodies, pharmaceutical compositions comprising the antibodies, kits comprising the antibodies (or pharmaceutical compositions comprising the antibodies), and therapeutic or diagnostic uses comprising the antibodies or pharmaceutical compositions.

### Definitions

Unless defined otherwise, all technical and scientific terms used herein have the same meaning as commonly understood by those of ordinary skill in the art to which this disclosure pertains.

The term “IL-31”, “IL31”, “IL-31 polypeptide”, or “IL-31 protein”, refers to Interleukin-31, which is an inflammatory cytokine that helps trigger cell-mediated immunity against pathogens. The human and murine amino acid and nucleic acid sequences can be found in public databases, such as GenBank, UniProt and Swiss-Prot. For example, the amino acid sequence of human IL-31 can be found under UniProt Accession No. Q6EBC2 (SEQ ID NO: 143) and the nucleotide sequence encoding the human IL-31 can be found under Accession No. NM_001014336. The term “IL-31” or “IL31” refers in particular to human IL-31. The antibodies or fragments thereof of the present invention preferably specifically bind to IL-31, more preferably to human IL-31. In some embodiments, human IL-31 refers to IL-31 without a signal peptide corresponding to aa 1-23 of UniProt Accession No. Q6EBC2.

The term “IL-13”, “IL13” “IL-13 polypeptide”, or “IL-13 protein”, refers to Interleukin 13, a cytokine involved in regulating inflammatory and immune responses. The human and murine amino acid and nucleic acid sequences can be found in public databases, such as GenBank, UniProt and Swiss-Prot. For example, the amino acid sequence of human IL-13 can be found under UniProt Accession No. P35225 and the nucleotide sequence encoding human IL-13 can be found under Accession No. NM_002188. The term “IL-13” or “IL13” refers in particular to human IL-13. The anti-IL-31/IL-13 multispecific e.g., bispecific antibodies of the present invention preferably specifically bind to IL-13 and IL-31, more preferably to human IL-13 and human IL-31.

As used herein, the singular forms “a,” “an” and “the” include plural references unless the content clearly dictates otherwise. Thus, for example, reference to “an antibody” includes a mixture of two or more such antibodies. Thus, unless stated otherwise, the terms are to be understood as “at least one”.

Unless otherwise specifically stated or clear from context, as used herein, the term “about” in relation to a numerical value is understood as being within the normal tolerance in the art, e.g., within two standard deviations of the mean. Thus, “about” can e.g., be within +/−20%, 15%, 10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The term “comprise”, “comprises”, “comprising”, and the like, encompasses “including” as well as “consisting of”, e.g., a composition “comprising” X may consist exclusively of X or may include something additional, e.g., X+Y Similarly, a polypeptide or nucleic acid molecule “comprising” an amino acid sequence or a nucleotide sequence respectively, such as the ones described herein, may consist exclusively of the amino acid sequence or nucleotide sequence, or may include additional amino acid or nucleotide sequences. As used herein, the phrase “consisting essentially of” refers to the genera or species of active pharmaceutical agents included in a method or composition, as well as any excipients inactive for the intended purpose of the method or composition. In some cases, the phrase “consisting essentially of” expressly excludes the inclusion of one or more additional active agents other than an antibody of the present disclosure. In some cases, the phrase “consisting essentially of” expressly excludes the inclusion of one or more additional active agents other than an antibody of the present disclosure and a second co-administered active agent.

The term “binding specificity” as used herein refers to the ability of an individual antibody binding site to react with the greatest affinity to the specified antigenic determinant. E.g., an antibody may bind other antigens besides the specified antigen at a lower affinity (e.g., at a 10×, 100×, 1000× lower affinity).

The term “specifically binds” or “selectively binds”, when used in the context of describing the interaction of an antigen and an antibody described herein, refers to a binding reaction that is determinative of the presence of a cognate antigen (or epitope) in a heterogeneous population of proteins and other biologics, e.g., in a biological sample (such as a blood, serum, cell, cell lysate, cell supernatant, plasma or tissue sample). Binding may also be called specific when the antibody binds to its target antigen (e.g., IL-31 and/or IL-13 as described herein) with a binding affinity that is at least two-fold, at least five-fold, or at least ten-fold greater than its affinity for binding to a non-specific antigen, or against the background in an immunoassay. Binding affinity can be measured according to standard methods in the art, for example with surface plasmon resonance using commercially available instruments and protocols (e.g., using a Biacore instrument (Cytiva Life Sciences, Uppsala, Sweden) as e.g., shown in the experimental section herein). Other examples of suitable methods include enzyme-linked immunosorbent assay (ELISA), radioimmunoassay (REA), FACS analysis, bioassays (e.g., growth inhibition), and Western Blotting.

Typically, the antibodies of the invention bind to human IL-31 with dissociation constant (KD) of less than 100 pM, less than 90 pM, less than 80 pM, less than 70 pM, less than 60 pM, less than 50 pM, less than 30 pM, less than 25 pM, less than 20 pM, less than 15 pM, less than 10 pM, or less than 5 pM or less than about 5 pM as measured using surface plasmon resonance (SPR), preferably as measured using a Biacore instrument, for example a Biacore T200, Biacore T100, or Biacore 8K instrument. In some embodiments, the anti-IL-31 antibody binds to human IL-31 with a dissociation constant (KD) of less than 30 pM, less than 25 pM, less than 20 pM, less than 15 pM, less than 10 pM, or less than 5 pM or less than about 5 pM as measured using surface plasmon resonance, preferably as measured using a Biacore instrument, for example a Biacore T200, Biacore T100, or Biacore 8K instrument. SPR assays can be performed using standard conditions, for example following instrument manufacturer instructions. Further examples of SPR assays for assaying affinity to IL-31 are provided in the experimental section later herein. Measurements using Biacore instruments can be combined with standardized sensor chips for interaction analysis involving biotinylated molecules; exemplary assays are further described in the experimental section later herein. Non-limiting exemplary conditions include a temperature of 25° C., a running buffer of HBS-EP+ at a flow rate of about 40 μl/min, an analyte contact time of 240 s and a dissociation time of 900 s.

The term “KD” or “KD” refers to the dissociation equilibrium constant of a particular antibody-antigen interaction and provides a measurement of an antibody's affinity to its target. Comparison of affinity and/or binding properties between two antibodies can also be established without actually determining the KD value for each antibody, but based on a quantitative measurement of binding (e.g. by ELISA or FACS analysis) that is proportional to KD or on a qualitative measurement of affinity or on an inference of affinity (e.g., in functional assay or in vitro or in vivo assay with respect to the antibody exerting an effect associated with binding to its target).

The term “specifically binds” does not exclude cross-species reactivity. For example, an antibody that “specifically binds” to an antigen from one species may also “specifically bind” to that antigen in one or more other species. Thus, such cross-species reactivity does not itself alter the classification of an antibody as a “specific” binder. In certain embodiments, an antibody that specifically binds to human IL-31 and/or human IL-13 has cross-species reactivity with IL-31 and/or IL-13 from one or more non-human mammalian species, e.g., a primate species (including but not limited to one or more of Macaca fascicularis, Macaca mulatta, and Macaca nemestrina) or a rodent species, e.g., Mus musculus.

The term “antibody” as used herein refers to a polypeptide (or set of polypeptides) of the immunoglobulin family that is capable of binding an antigen non-covalently, reversibly, and typically, specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The variable regions of both the light (VL) and heavy (VH) chain portions determine antigen recognition and specificity. Conversely, the constant regions of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus of the molecule contains the variable region, and the C-terminus contains the constant region; the CH3 and CL domains comprise the carboxy-terminus of the heavy and light chain, respectively.

The term “antibody” includes, but is not limited to, monoclonal antibodies, human antibodies, humanized antibodies, camelid antibodies, chimeric antibodies, multispecific, e.g., bispecific antibodies and anti-idiotypic (anti-Id) antibodies (including, e.g., anti-Id antibodies to antibodies described herein), as well as parts, such as antigen-binding fragments, thereof. The antibodies can be of any isotype/class (e.g., IgG, IgE, IgM, IgD, IgA, or IgY) or subclass (e.g., IgG1, IgG2, IgG3, IgG4, IgAQ1, or IgA2). The term “antibody” also encompasses antibody fragments and antibody-like molecules i.e., molecules comprising an antibody or an antigen-binding fragment thereof e.g., a molecule comprising a first half antibody having a typical antibody structure (comprising two polypeptides, the first comprising a light chain, and the second comprising a heavy chain) and a second half antibody having e.g., an scFv-Fc format.

The term “antibody fragment” as used herein refers to one or more portions of an antibody. In some embodiments, these portions are part of the constant domain(s) of an antibody, e.g., fragment crystallizable (Fc), constant (C) domains, etc.

In other embodiments, these portions are antigen-binding fragments that retain the ability of binding an antigen. Thus, the term “antigen-binding fragment” as used herein refers to one or more portions of an antibody that retain the ability to interact with (e.g., by binding, steric hindrance, stabilizing/destabilizing, spatial distribution) an epitope of an antigen (e.g., IL-31 or IL-13), preferably specifically. Examples of antigen-binding fragments include, but are not limited to, a Fab fragment (comprising the VL, VH, CL and CH1 domains); a F(ab)2 fragment, a bivalent fragment comprising two Fab fragments linked by a disulfide bridge at the hinge region; an Fd fragment comprising the VH and CH1 domains; an Fv fragment comprising the VL and VH domains of a single arm of an antibody, a single chain Fv fragment (scFv); an (scFv′)2 (e.g., produced from scFv containing an additional C terminal cysteine by chemical coupling (Adams et al (1993) Can Res 53:4026-4034 and McCartney et al (1995) Protein Eng, 8:301-314) or by spontaneous site-specific dimerization of scFv containing an unpaired C terminal cysteine residue (Kipriyanov et al (1995) Cell. Bio Phys 26: 187-204)); a single domain antibody (dAb) fragment (Ward et al., 1989 Nature 341:544-546), that comprises a VH domain or a VL domain; an isolated complementarity determining region (CDR), or other epitope-binding fragments of an antibody. ScFv-sc-Fv tandems ((scFv)2) may also be produced by linking two scFv units by a third peptide linker, (see Kurucz et al (1995) J Immunol, 154:4576-4582).

Antibody fragments can be incorporated into e.g., single variable domain antibodies, maxibodies, minibodies, intrabodies, diabodies, triabodies, tetrabodies, v-NAR and bis-scFv (see, e.g., Hollinger and Hudson, (2005) Nature Biotechnology 23: 1126-1136). Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen binding regions (Zapata et al., (1995) Protein Eng. 8:1057-1062; and U.S. Pat. No. 5,641,870). A single variable domain antibody may e.g. comprise a VH, VL, or VHH (variable region of heavy-chain antibody, e.g., a camelid or shark antibody), and may be present in a format (e.g., homo- or hetero-multimer) with other, different variable regions or variable domains where the other regions or domains are not required for antigen binding by the single chain variable domain.

The term “single chain antibody”, “single chain Fv” or “scFv” refers to a molecule comprising an antibody heavy chain variable domain (or region; VH) and an antibody light chain variable domain (or region; VL) connected by a linker. Such scFv molecules can have the general structures: NH2-VL-linker-VH-COOH or NH2-VH-linker-VL-COOH. ScFvs can be prepared according to method known in the art (see, for example, Bird et al., (1988) Science 242:423-426 and Huston et al., (1988) Proc. Natl. Acad. Sci. USA 85:5879-5883). The scFv molecules typically comprise a linker (e.g., a Ser-Gly linker). Exemplary suitable linkers are described in, e.g., Holliger et al. (1993), Proc. Natl. Acad. Sci. USA 90:6444-6448; Alfthan et al. (1995), Protein Eng. 8:725-731; Choi et al. (2001), Eur. J. Immunol. 31: 94-106; Hu et al. (1996), Cancer Res. 56:3055-3061; Kipriyanov et al. (1999), J. Mol. Biol. 293:41-56, and Roovers et al. (2001), Cancer Immunol.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The positions of the CDRs and framework regions can be determined using various known definitions in the art, e.g., Kabat (“Kabat numbering scheme”), Chothia, IMGT, AbM, and combined definitions (see, e.g., Kabat et al. (1991), “Sequences of Proteins of Immunological Interest,” 5th Ed. Public Health Service, National Institutes of Health, Bethesda, MD; Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Lefranc, M. P., Nucleic Acids Res., 29:207-209 (2001); Al-Lazikani et al., J. Mol. Biol., 273:927-748 (1997)). Definitions of antigen combining sites are also described in the following: Ruiz et al., Nucleic Acids Res., 28:219-221 (2000); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). Under IMGT, the CDR regions of an antibody can be determined using the program IMGT/DomainGap Align.

Under the Kabat numbering scheme, the CDR amino acid residues in the heavy chain variable domain (VH) are typically numbered 31-35 (HCDR1), 50-65 (HCDR2), and 95-102 (HCDR3); and the CDR amino acid residues in the light chain variable domain (VL) are typically numbered 24-34 (LCDR1), 50-56 (LCDR2), and 89-97 (LCDR3). Under the Chothia numbering scheme, the CDR amino acids in the VH are typically numbered 26-32 (HCDR1), 52-56 (HCDR2), and 95-102 (HCDR3); and the CDR amino acid residues in the VL are typically numbered 26-32 (LCDR1), 50-52 (LCDR2), and 91-96 (LCDR3). In a combined Kabat and Chothia numbering scheme, the CDRs typically correspond to the amino acid residues that are part of a Kabat CDR, a Chothia CDR, or both. For instance, under the combined numbering scheme, the CDRs typically correspond to amino acid residues 26-35 (HCDR1), 50-65 (HCDR2), and 95-102 (HCDR3) in a human VH; and amino acid residues 24-34 (LCDR1), 50-56 (LCDR2), and 89-97 (LCDR3) in human VL, e.g., a mammalian VL, e.g., a human VL.

As used herein, the term “EU numbering” or “EU numbering scheme” refers to the Eu numbering convention for the constant regions of an antibody, as e.g., described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991.

The term “antibody framework” or “framework region” as used herein refers to the part of the variable domain, either VL or VH, which serves as a scaffold for the antigen binding loops (CDRs) of this variable domain.

The term “part antibody” or “half antibody” refers to a portion of an antibody, antibody fragment, antibody-like molecule or multispecific antibody that comprises a single antigen binding domain. In some embodiments, a part or half antibody refers to a heavy and a light chain pair of, for example, an IgG antibody. In some embodiments, a part or half antibody comprises a polypeptide comprising a VL and a CL domain and a polypeptide comprising a VH domain, a CH1 domain, a hinge domain, a CH2 domain, and a CH3 domain, wherein said VH and VL domains comprise an antigen-binding domain. In some embodiments, a part or half antibody refers to, e.g. a polypeptide comprising a scFv domain and an Fc polypeptide chain (comprising a CH2 domain and a CH3 domain, and optionally a hinge region). In some embodiments, a part or half antibody comprises or is a Fab.

The term “part antibody” or “half antibody” is intended for descriptive purposes only and is not intended to be limiting as to a particular configuration or method of production. Descriptions of a half antibody as a “first” half antibody, a “second” half antibody, a “left” half antibody, a “right” half antibody and the like are merely for convenience and descriptive purposes.

In some embodiments wherein the antibody is multispecific, e.g., bispecific, a first part or half antibody may be paired with another part or half antibody, such that the first part and second will associate, e.g., heterodimerize. This can, for example, be achieved via association of Fc polypeptide chains comprised by the first and second parts forming an Fc region heterodimer, as described later herein.

In some embodiments, a first part or half antibody will be covalently linked to a second part or half antibody. In some embodiments, either a first half antibody, a second part half antibody, or both a first and second half antibody may be linked to an additional antigen binding domain.

Preferably, a “part antibody” or “half antibody” described herein is a “half IgG antibody”. The term “part IgG antibody”, “half IgG antibody” or “half IgG antibody fragment” refers to a heavy and light chain pair of an IgG antibody. The light chain comprises a VL domain and a CL domain, and the heavy chain comprises a VH domain, a CH1 domain, a hinge domain, a CH2 domain, and a CH3 domain (i.e., an Fd and an Fc), wherein said VL and VH domains comprise an antigen-binding domain. Each part or half IgG antibody of the disclosure preferably comprises an Fc polypeptide chain as described later herein. As with all polypeptide chains, an Fc polypeptide chain contains an N-terminus and a C-terminus, each of which is capable of being linked to an antigen-binding domain. An example of a part or half IgG antibody is a part or half IgG1 antibody.

The terms “constant region” or “constant domain” refer to a carboxy terminal portion of the light and heavy chain which is not directly involved in binding of the antibody to antigen but exhibits various effector functions, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2, and CH3 domains of the heavy chain and the CL domain of the light chain.

The terms “variable region” or “variable domain” refer to a portion of the light and heavy chains, which differ extensively in sequence among antibodies and are determinant for the binding and specificity of each particular antibody for its particular antigen. The variability in sequence is concentrated in those regions called complementarity determining regions (CDRs), while the more highly conserved regions in the variable domain are called framework regions (FR). As used herein, VH refers to the heavy chain variable region without a constant region. As used herein, VL refers to a light chain variable region without a constant region.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosed sequences. The following exemplary eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” or “conservative modifications” are used to refer to amino acid modifications that do not significantly affect or alter the binding characteristics of the antibody containing the amino acid sequence.

The term “dose” refers to the quantity of pharmaceutical composition or antibody e.g., multispecific, such as bispecific, antibody described herein, which can be administered to a subject all at one time (unit dose), or in two or more administrations over a defined time interval. The interval between doses can be any desired amount of time and is referred to as the “dosing interval”.

The term “therapeutically effective” or “therapeutically effective amount” or “therapeutically efficacious dose” means a sufficient amount, e.g. of an antibody or composition described herein, or other pharmaceutically active agent, to provide the desired therapeutic effect (i.e., a reduction in disease activity, reduction in disease progression, reduction in disease signs and/or symptoms, etc.). A therapeutically effective amount is also one in which any toxic or detrimental effects of the antibody molecule are outweighed by the therapeutically beneficial effects. A “therapeutically effective dosage” preferably inhibits a measurable parameter (e.g., a symptom) by at least about 20%, more preferably by at least about 40%, even more preferably by at least about 60%, and still more preferably by at least about 80% relative to untreated subjects. The term “prophylactically effective” amount means a sufficient amount to provide a desired prophylactic effect. Typically, since a prophylactic dose is used in subjects prior to or at an earlier stage of disease, the prophylactically effective amount will be less than the therapeutically effective amount. The amount that is “effective” may vary from subject to subject, depending on the age and general condition of the individual, e.g., disease state, age, sex, weight, and the like, as well as the particular drug or pharmaceutically active agent. Thus, it is not always possible to specify an exact “effective” amount applicable for all patients. However, an appropriate “effective” dose in an individual case may be determined by a practicing physician. The ability of an antibody or fragment thereof to exert a therapeutic or prophylactic effect can e.g., be evaluated in an animal model system predictive of efficacy in the corresponding disease, or alternatively using in vitro assays, e.g., in vitro assays measuring inhibition of IL-31 and/or IL-13 activity described herein, examples of which are provided in the experimental section.

As used herein, “identity” refers to the sequence matching between two polypeptides, molecules or between two nucleic acids. When a respective position in both of the two compared sequences is occupied by the same base or amino acid (for instance, if a position in each of two polypeptides is occupied by a lysine), then the respective molecules are identical at that position. The “percentage identity” or “percent identity” between two sequences is a function of the number of identical positions shared by the sequences, taking into account the number of gaps, and the length of each gap, which need to be introduced for optimal alignment of the two sequences. Generally, a comparison is made when two sequences are aligned to give maximum identity. Such alignment can be provided using, for instance, the method of the Needleman and Wunsch (J. Mol. Biol. (48):444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. As another example, the percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. One suitable set of parameters are a Blossum 62 scoring matrix with a gap penalty of 12, a gap extend penalty of 4, and a frameshift gap penalty of 5.

The percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as e.g., described in the non-limiting examples below. Two sequences are “identical” if they have the same sequence of amino acids or nucleotides over the region being compared. Two sequences are “substantially identical” if two sequences have a specified percentage of amino acid residues or nucleotides that are the same (i.e., 80% identity, optionally 85%, 90%, 95%, 98% or 99% identity over a specified region, or, when not specified, over the entire sequence), when compared and aligned for maximum correspondence over a comparison window, or designated region as measured using one of the sequence comparison algorithms discussed herein or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window,” as used herein, includes reference to a segment corresponding to a number of contiguous positions in which a sequence may be compared to a reference sequence of the same number of contiguous positions after the two sequences are optimally aligned. The percent identity is typically calculated by comparing sequences of similar lengths.

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information, and be e.g., run using their default parameters. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the two nucleic acid sequences.

The term “isolated antibody” refers to an antibody that is removed from its original or native environment (e.g., the natural environment if it is naturally occurring). Moreover, an isolated antibody may be substantially free of other cellular material and/or chemicals, for example, an antibody isolated from a cell culture supernatant. Such an antibody may be part of a composition, and still be considered isolated in that such a composition is not part of the environment in which it was originally or natively found. Methods of isolation are known in the art and include electrophoretic (e.g., SDS-PAGE, isoelectric focusing, capillary electrophoresis), chromatographic (e.g., ion-exchange, reverse phase HPLC), and filtration methods. Other non-limiting examples include cation exchange (e.g., in bind-elute mode) in combination with multimodal anion exchange chromatography (MAC, e.g., in flow-through mode). In some embodiments, the antibody has been isolated and purified to greater than 95% or than about 95%, greater than 96% or than about 96%, greater than 97% or than about 97%, greater than 98% or than about 98%, or greater than 99% or than about 99% purity as determined by any suitable method in the art (e.g., the electrophoretic or chromatographic methods discussed above; see also Flatman et al., 2007, J Chromatogr B Analyt Technol Biomed Life Sci, 848: 79-87). Purity may also be e.g., determined using an intact UPLC-MS mass screening approach, e.g., following protein-A purification, or using an LC-MS approach. Purity may also be e.g., determined using size exclusion chromatography, e.g., on commercial columns like the BEH200 SEC column (Waters #186005225). Purity may also be e.g., determined using CE-SDS capillary electrophoresis, e.g., on commercial systems like the Beckman Coulter PA 800 system (Beckman Coulter, CA, USA). Further exemplary assays for antibody isolation, purification and purity analysis are provided in the experimental section later herein.

The term “epitope” refers to a site on an antigen (e.g., IL-31 or IL-13) to which an antibody of the disclosure interacts with and includes linear, discontinuous, or conformational epitopes. Epitopes can be formed both from contiguous amino acids and/or non-contiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)), or electron microscopy. A “paratope” is the part of the antibody which recognizes the epitope of the antigen.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

“Isolated” nucleic acid molecule or polynucleotide means a DNA or RNA, e.g., of genomic, mRNA, cDNA, or synthetic origin or a combination thereof which is not associated with all or a portion of a polynucleotide in which the isolated polynucleotide is found in nature, or is linked to a polynucleotide to which it is not linked in nature. Such nucleic acid molecules could be part of a vector and/or such nucleic acid molecules can be part of a composition, and still be isolated in that such a vector or composition is not part of the environment in which it is found in nature.

The term “operably linked” refers to a functional relationship between two or more polynucleotide (e.g., DNA) segments. Typically, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” or “codon optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell or a prokaryotic cell, for example an Escherichia coli cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as a retroviral vector, a lentiviral vector, an adenoviral vector, or an adeno-associated viral vector (AAV, e.g., AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, lentiviruses, adenoviruses and adeno-associated viruses), which serve equivalent functions.

The term “host cell” refers to a cell into which a recombinant nucleic acid molecule or expression vector has been introduced, e.g., a nucleic acid molecule or expression vector encoding an antibody described herein. Such a cell may alternatively be referred to as “recombinant host cell” or “engineered host cell”. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “protein” refers to any organic compound made of amino acids arranged in one or more linear chains and folded into a globular form. The amino acids in a polymer chain are joined together by the peptide bonds between the carboxyl and amino groups of adjacent amino acid residues. The term “protein” further includes, without limitation, peptides, polypeptides, single chain polypeptides or any complex molecules consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, and amino acid modification for chemical conjugation with another molecule. Unless otherwise indicated, a particular polypeptide sequence also implicitly encompasses conservatively modified variants thereof. Thus, in some embodiments, the term protein refers to an antibody or multispecific, e.g., bispecific, antibody described herein or to a fragment thereof.

The term “subject” includes human and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject” are used herein interchangeably. In a preferred embodiment, the “subject” or “patient” is a human. As used herein, the terms “cyno” or “cynomolgus” refer to the Cynomolgus monkey (Macaca fascicularis).

As used herein, the terms “treatment”, “treating” or “treat” used herein means any action that results in, or is contemplated to result in, cure (if applicable), or the alleviation, amelioration, improvement, or reduction of a disease or disorder (e.g., Atopic dermatitis (AD)), or any associated symptoms of the disease or disorder. “Prevention” or “preventing” or prevent” as used herein means any action that prevents or slows a worsening in the disease or disorder, or a parameter associated with the disease or disorder in a patient at risk for said worsening or at risk of developing the disease or disorder.

The term “subject in need of treatment” or “patient in need of treatment” includes subjects that would benefit from administration of an antibody or composition of the present disclosure used e.g., for a diagnostic procedure or treatment.

The term “IL-31-related (mediated) disease” or “IL-31-related (mediated) disorder” or disease or disorder that is “mediated by IL-31” or “related to IL-31” refers to any disorder, the onset, progression or the persistence of the symptoms or disease states of which requires or follows from the participation of IL-31. IL-31-related diseases include, but are not limited to, allergic, inflammatory and autoimmune diseases, such as atopic dermatitis, scleroderma, allergic contact dermatitis, chronic spontaneous urticaria, bullous pemphigoid, alopecia areata, dermatomyositis, prurigo nodularis, and psoriasis. In a preferred embodiment, the IL-31-related disease is atopic dermatitis. The term “IL-13-related (mediated) disease” or “IL-13-related (mediated) disorder” or disease or disorder that is “mediated by IL-13” or “related to IL-13” refers to any disorder, the onset, progression or the persistence of the symptoms or disease states of which requires or follows from the participation of IL-13. Exemplary IL-13-related diseases include, but are not limited to, allergy, asthma, autoimmune disease, inflammatory diseases, allergic rhinitis, food hypersensitivity, urticaria, bullous skin diseases, erythema multiform, contact dermatitis, psoriasis, rheumatoid arthritis, juvenile chronic arthritis, inflammatory bowel diseases (e.g., ulcerative colitis, Crohn's disease), idiopathic interstitial pneumonia, idiopathic pulmonary fibrosis, hypersensitivity pneumonitis, chronic obstructive pulmonary disease, RSV infection, uveitis, scleroderma, osteoporosis, Hodgkin's lymphoma, and atopic dermatitis. Other exemplary IL-13-related diseases include chronic sinusitis, systemic lupus erythematosus, systemic sclerosis, and Sjogren's syndrome. In a preferred embodiment, the IL-13-related disease is atopic dermatitis. More information on IL-31- and/or IL-13-related diseases or disorders is provided later herein.

The terms “pruritus” and “itch”, which are used synonymously herein, refer to a sensation that causes the desire or reflex to scratch the skin. Scratching can be problematic particularly in the case of chronic skin diseases that induce itching, such as atopic dermatitis, because the permanent itching stimulates patients to scratch the affected skin areas constantly and/or excessively, which often leads to skin injuries and further deterioration of the skin surface, as well as sleep deprivation, depression and low quality of life.

As used herein, the term “Fc” or “Fc polypeptide” includes a polypeptide comprising a CH2 and a CH3, and, optionally, any portion of an antibody hinge region. An Fc region is comprised of two Fc polypeptide chains, each comprising a CH2 and a CH3, that dimerize to form an Fc region. In some embodiments, an antibody described herein comprises an Fc polypeptide that is a variant of a human Fc polypeptide, such as a human IgG Fc polypeptide (for example a human IgG1 Fc polypeptide). A variant Fc polypeptide may comprise one or more amino acid modifications (mutations), preferably one or more amino acid substitutions, relative to a reference (“wild-type”) Fc polypeptide, for example an Fc polypeptide comprising the amino acid sequence shown in SEQ ID NO: 42. Examples of mutations include silencing mutations, half-life mutations, K-i-H (Knob-in-Hole), and FAE (Fab arm exchange) mutations, discussed later herein.

Each part or half antibody in the cases of multispecific antibodies described herein comprises an Fc polypeptide e.g., a first part antibody binding to IL-31 comprises a heavy chain comprising an Fc polypeptide and a second part antibody binding to a target different from IL-31 (e.g., IL-13) comprises a heavy chain comprising an Fc polypeptide, allowing for the two Fc polypeptide chains to dimerize into an Fc region of a multispecific (e.g., bispecific) antibody described herein. The dimerization may be promoted by the inclusion of pair K-i-H (Knob-in-Hole) or FAE (Fab arm exchange) mutations in the Fc polypeptides, discussed later herein.

As used herein, an “Fd” or “Fd region” comprises a VH and a CH1, and, optionally, any portion of an antibody hinge region.

The term “diabody” as used herein refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., (1993) Proc. Natl. Acad. Sci. USA 90:6444-6448.

The term “monospecific antibody,” as used herein, refers to an antibody that binds to a single target antigen.

The term “multispecific antibody” as used herein refers to an antibody that binds to two or more different (distinct) antigens or to an antibody that binds to two or more different (distinct) epitopes on the same antigen. Recognition of each antigen is generally accomplished via an “antigen-binding domain” (e.g., a “IL-31 antigen-binding domain”). In some aspects the multispecific antibodies comprise two, three, four or more polypeptide chains that together comprise a plurality, e.g., two or more, e.g., two, three or four antigen-binding domains. In some embodiments, the multispecific binding molecules comprise four polypeptide chains that together comprise a plurality, e.g., two or more, e.g., two, three or four, preferably two, antigen-binding domains that bind two or more different antigens. The term “multispecific” includes bispecific, trispecific, and tetraspecific molecules. Preferably, the multispecific antibodies of the invention are bispecific. Bispecific IgG molecules are typically bivalent and possess an asymmetric architecture due to the presence of, at least, different Fv regions. Depending on the method of preparation and the origin of the heavy and light chains, they may further differ in the constant regions of the heavy or light chains (e.g., via the introduction of “matching” (or pair) K-i-H or Fab arm exchange (FAE) mutations in the constant region of the heavy chains).

The term “bi-specific antibody” or “bispecific antibody” as used herein refers to a multispecific antibody that binds to two different antigens (e.g., IL-31 and IL-13) or to two different epitopes on the same antigen, preferably to two different antigens. A bispecific antibody is typically able to bind two different antigens or epitopes simultaneously or sequentially. The term “multivalent antibody” as used herein refers to an antibody that has at least two antigen binding domains. In some embodiments, a multivalent antibody is a bivalent antibody, trivalent antibody or a tetravalent antibody. The term “bivalent antibody” as used herein refers to an antibody that has two antigen-binding domains. The terms “trivalent antibody” and “tetravalent antibody” as used herein refer to an antibody that has three antigen-binding domains, or four antigen-binding domains, respectively. The term “monovalent antibody” means that the antibody has one antigen-binding domain.

The term “monovalent with respect to binding IL-13” as used herein means that the antibody has one IL-13 binding domain. The term “monovalent with respect to binding IL-31” as used herein means that the antibody has one IL-31-binding domain. In some embodiments, a bispecific antibody is provided that is monovalent with respect to binding IL-13 and monovalent with respect to binding IL-31, i.e., has one IL-31 binding domain and one IL-13 binding domain.

The term “monoclonal antibody” as used herein refers to polypeptides, including antibodies, antibody parts, and antigen-binding fragments, that have substantially identical amino acid sequence or are derived from the same genetic source. This term also includes preparations of antibody molecules of single molecular composition. A monoclonal antibody composition displays a single binding specificity and affinity for a particular epitope or antigen. Methods for generation of monoclonal antibodies using phage display technology are known in the art (Proetzel, G., Ebersbach, H. (Eds.) Antibody Methods and Protocols. Humana Press ISBN 978-1-61779-930-3; 2012).

The term “humanized” antibody as used herein, refers to an antibody that retains the reactivity of a non-human antibody or antibody variable domain while being less immunogenic in humans.

A “modification” or “mutation” of an amino acid residue/position, as used herein, refers to a change of a primary amino acid sequence as compared to a starting amino acid sequence, wherein the change results from a sequence alteration involving said amino acid residue/positions. The modification may e.g., be a substitution of the residue (or at said position) with another amino acid (e.g., a conservative or non-conservative substitution), an insertion, or a deletion of a residue. A “variant” or “mutant” of a given sequence comprises at least one amino acid modification relative to said sequence. In some embodiments, the amino acid modification is an amino acid substitution. In some embodiments, the amino acid modification is a conservative amino acid substitution.

The term “co-expression” means that different polypeptides are expressed together in a single host cell, common for all the polypeptides. Co-expression of a multispecific, e.g., bispecific, antibody means that the different parts forming the functional multispecific, e.g., bispecific, antibody are expressed in a single, common, host cell. Co-expression may be achieved by incorporating several expression vectors in the expression host cell, such as one for each of the parts of a bispecific antibody, or by incorporating one expression vector encoding all parts of the bispecific antibody.

As used herein, “silencing mutations” refer to mutations to residues in one or more constant domains, e.g., heavy chain constant regions, to silence, e.g., ADCC and/or CDC effector function. More than one mutation, for example amino acid substitutions, can be combined. Examples of combinations of amino acid substitutions for antibody effector silencing include the “DAPA” mutation (D265A, P329A in EU numbering) or the “LALA” mutation (L234A, L235A in EU numbering). Other exemplary silencing mutations include the “LALAGA” mutation (L234A, L235A, G237A in EU numbering), the “LALASKPA” mutation (L234A, L235A, S267K, P329A in EU numbering), the “DAPASK” mutation (D265A, P329A, S267K in EU numbering), the “GADAPA” mutation (G237A, D265A, P329A in EU numbering), the “GADAPASK” mutation (G237A, D265A, P329A, S267K in EU numbering), the “LALAPG” mutation (L234A, L235A, P329G in EU numbering), and the “LALAPA” mutation (L234A, L235A, P329A in EU numbering). Among silencing mutations, the “LALA” mutation (L234A and L235A in EU numbering) is preferred. Further examples of silencing mutations are disclosed in WO2022/097065.

As used herein, “half-life extension mutations” refer to mutations to residues in one or more constant domains, e.g., heavy chain constant regions, that result in half-life extension. More than one mutation, for example amino acid substitutions, can be combined. An example of a combination of amino acid substitutions for antibody half-life extension is the “YTE” mutation (M252Y, S254T, T256E in EU numbering). Another example is the “LS” mutation (M428L, N434S in EU numbering). Among half-life extension mutations, the “YTE” mutation (M252Y, S254T, T256E in EU Numbering) is preferred. Preferably, the term “half-life” as used herein refers to “in vivo half-life”, i.e., the half-life to the antibody circulating in the blood of a given mammal, preferably human.

As used herein, “K-i-H mutations” or “knob-in-hole mutations” refer to mutations to residues in one or more constant domains, e.g., heavy chain constant regions, that facilitate proper chain pairing, particularly for heterodimerization techniques for making multispecific e.g., bispecific antibodies. Exemplary knob mutations are (S354C, T366W) substitutions and exemplary hole mutations are (Y349C, T366S, L368A, Y407V) substitutions (in EU Numbering). Knob-in-hole technology is further discussed later herein.

As used herein, “Fab arm exchange mutations” or “FAE mutations” refer to mutations to residues in one or more constant domains, e.g., heavy chain constant regions, that facilitate heterodimerization by Fab arm exchange. An exemplary FAE mutation “pair” comprises a K409R mutation in one CH3 domain paired with an F405L mutation in the second CH3 domain, according to EU numbering. Fab arm exchange technology is discussed later herein.

The skilled person understands that in cases of a multispecific, e.g., a bispecific, antibody comprising a first part binding to IL-31 and a second part binding to a different antigen (e.g., IL-13) as described herein, the first part may in some embodiments comprise a heavy chain comprising a “knob” mutation in its constant region and the second part may comprise a heavy chain comprising a “hole” mutation in its constant region, or vice versa (the first part comprising the “hole” mutation and the second part comprising the “knob” mutation). Likewise, in the case of FAE mutations, the first part may in some embodiments comprise a heavy chain comprising a K409R or an F405L mutation in its constant region, and the second part may comprise a heavy chain comprising the respective “matching” (or pair) mutation (F405L or K409R as appropriate) in its constant region.

Bispecific antibodies described herein are heterodimers, which means that the first part binding to IL-31 comes from a first antibody (binding to IL-31) and the second part binding to a different target (e.g., IL-13) comes from a second antibody (binding to the second target). A “heterodimerization modification” is a modification to one or both parts of the antibodies forming the heterodimeric bispecific antibody, intended to facilitate such formation. Examples of heterodimerization modifications are the K-i-H and FAE mutations discussed above, and further described later herein.

The term “half-life” as used herein, refers to the time required for the concentration of a drug to fall by one-half (Rowland M and Towzer T N: Clinical Pharmacokinetics. Concepts and Applications. Third edition (1995); and Bonate P L and Howard D R (Eds): Pharmacokinetics in Drug Development, Volume 1 (2004)). Further details may also be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2nd Rev. ex edition (1982), which describes pharmacokinetic parameters such as alpha half-life and beta half-life and area under the curve (AUC). Optionally, all pharmacokinetic parameters and values quoted herein are to be read as being values in a human. Optionally, all pharmacokinetic parameters and values quoted herein are to be read as being values in a rabbit, mouse, rat, or Cynomolgus monkey.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immuno-adhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag). One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill. Examples of derivatized antibodies are antibody conjugates, described in more detail later herein.

Various aspects of the invention are described in further detail in the following sections and subsections.

### Anti IL-31 Antibodies

The invention provides antibodies that bind to IL-31, alternatively referred to herein as anti-IL-31 antibodies or IL-31 antibodies, or fragments thereof (that bind to IL-31), e.g., Fabs or any other fragment described herein. It is thus understood that the embodiments described herein referring to the anti-IL-31 antibodies are also applicable to their antigen-binding fragments (e.g., to Fabs) and any other antigen-binding fragment described herein. Preferably, the anti-IL-31 antibodies described herein, as well as their antigen-binding fragments, bind specifically to IL-31.

Preferably, the IL-31 is human IL-31. Preferably, the anti-IL-31 antibody is an IL-31 inhibitor.

In an embodiment, the anti-IL-31 antibody comprises:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23;
an LCDR1 comprising SEQ ID NO: 4 or SEQ ID NO: 24;
an LCDR2 comprising SEQ ID NO: 5 or SEQ ID NO: 25; and
an LCDR3 comprising SEQ ID NO: 6 or SEQ ID NO: 26;

or
an HCDR1 comprising SEQ ID NO: 48;
an HCDR2 comprising SEQ ID NO: 49 or SEQ ID NO: 61;
an HCDR3 comprising SEQ ID NO: 50 or SEQ ID NO: 63;
an LCDR1 comprising SEQ ID NO: 51 or SEQ ID NO: 65;
an LCDR2 comprising the amino acid sequence YAK; and
an LCDR3 comprising SEQ ID NO: 53 or SEQ ID NO: 64;

or
an HCDR1 comprising SEQ ID NO: 66 or SEQ ID NO: 72;
an HCDR2 comprising SEQ ID NO: 67 or SEQ ID NO: 73;
an HCDR3 comprising SEQ ID NO: 68 or SEQ ID NO: 77;
an LCDR1 comprising SEQ ID NO: 69 or SEQ ID NO: 76;
an LCDR2 comprising the amino acid sequence YAK; and
an LCDR3 comprising SEQ ID NO: 71 or SEQ ID NO: 75;

or
an HCDR1 comprising SEQ ID NO: 78 or SEQ ID NO: 90;
an HCDR2 comprising SEQ ID NO: 79 or SEQ ID NO: 91;
an HCDR3 comprising SEQ ID NO: 80 or SEQ ID NO: 96;
an LCDR1 comprising SEQ ID NO: 81 or SEQ ID NO: 95;
an LCDR2 comprising SEQ ID NO: 82 or SEQ ID NO: 93; and
an LCDR3 comprising SEQ ID NO: 83 or SEQ ID NO: 94.

In an embodiment, the anti-IL-31 antibody comprises: an HCDR1 comprising SEQ ID NO: 1; an HCDR2 comprising SEQ ID NO: 2 or SEQ ID NO: 21; an HCDR3 comprising SEQ ID NO: 3 or SEQ ID NO: 23; an LCDR1 comprising SEQ ID NO: 4 or SEQ ID NO: 24; an LCDR2 comprising SEQ ID NO: 5 or SEQ ID NO: 25, and an LCDR3 comprising SEQ ID NO: 6 or SEQ ID NO: 26, preferably wherein the HCDR1, HCDR2, HCDR3, LCDR1, LCDR2, and LCDR3 are defined according to the Kabat numbering scheme.

In an embodiment, the anti-IL-31 antibody comprises: an HCDR1 comprising SEQ ID NO: 48 or SEQ ID NO: 60; an HCDR2 comprising SEQ ID NO: 49 or SEQ ID NO: 61; an HCDR3 comprising SEQ ID NO: 50 or SEQ ID NO: 63; an LCDR1 comprising SEQ ID NO: 51 or SEQ ID NO: 65; an LCDR2 comprising the amino acid sequence YAK, and an LCDR3 comprising SEQ ID NO: 53 or SEQ ID NO: 64, preferably wherein the HCDR1, HCDR2, HCDR3, LCDR1, LCDR2, and LCDR3 are defined according to the Chothia numbering scheme.

In an embodiment, the anti-IL-31 antibody comprises: an HCDR1 comprising SEQ ID NO: 66 or SEQ ID NO: 72; an HCDR2 comprising SEQ ID NO: 67 or SEQ ID NO: 73; an HCDR3 comprising SEQ ID NO: 68 or SEQ ID NO: 77; an LCDR1 comprising SEQ ID NO: 69 or SEQ ID NO: 76; an LCDR2 comprising the amino acid sequence YAK, and an LCDR3 comprising SEQ ID NO: 71 or SEQ ID NO: 75, preferably wherein the HCDR1, HCDR2, HCDR3, LCDR1, LCDR2, and LCDR3 are defined according to the IMGT numbering scheme.

In an embodiment, the anti-IL-31 antibody comprises: an HCDR1 comprising SEQ ID NO: 78 or SEQ ID NO: 90; an HCDR2 comprising SEQ ID NO: 79 or SEQ ID NO: 91; an HCDR3 comprising SEQ ID NO: 80 or SEQ ID NO: 96; an LCDR1 comprising SEQ ID NO: 81 or SEQ ID NO: 95; an LCDR2 comprising SEQ ID NO: 82 or SEQ ID NO: 93, and an LCDR3 comprising SEQ ID NO: 83 or SEQ ID NO: 94, preferably wherein the HCDR1, HCDR2, HCDR3, LCDR1, LCDR2, and LCDR3 are defined according to the Combined numbering scheme.

In an embodiment, the anti-IL-31 antibody comprises i) an HCDR1 comprising SEQ ID NO: 1; ii) an HCDR2 comprising SEQ ID NO: 21; and iii) an HCDR3 comprising SEQ ID NO: 3. In an embodiment, the IL-31 antibody comprises i) an HCDR1 comprising SEQ ID NO: 1; ii) an HCDR2 comprising SEQ ID NO: 21; and iii) an HCDR3 comprising SEQ ID NO: 23. In an embodiment, the anti-IL-31 antibody comprises i) an HCDR1 comprising SEQ ID NO: 1; ii) an HCDR2 comprising SEQ ID NO: 2; and iii) an HCDR3 comprising SEQ ID NO: 3. Preferably, in these embodiments the HCDR1, HCDR2, and HCDR3 are defined according to the Kabat numbering scheme.

In an embodiment, the anti-IL-31 antibody comprises i) an LCDR1 comprising SEQ ID NO:4; ii) an LCDR2 comprising SEQ ID NO:25; and iii) an LCDR3 comprising SEQ ID NO:26. In an embodiment, the anti-IL-31 antibody comprises i) an LCDR1 comprising SEQ ID NO:4; ii) an LCDR2 comprising SEQ ID NO:5; and iii) an LCDR3 comprising SEQ ID NO:6. In an embodiment, the IL-31 antibody comprises i) an LCDR1 comprising SEQ ID NO:4; ii) an LCDR2 comprising SEQ ID NO:5; and iii) an LCDR3 comprising SEQ ID NO:26. In an embodiment, the IL-31 antibody comprises i) an LCDR1 comprising SEQ ID NO: 24; ii) an LCDR2 comprising SEQ ID NO: 5; and iii) an LCDR3 comprising SEQ ID NO: 26. Preferably, in these embodiments the LCDR1, LCDR2, and LCDR3 are defined according to the Kabat numbering scheme.

In an embodiment, the anti-IL-31 antibody comprises: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:26. In an embodiment, the anti-IL-31 antibody comprises: i) an HCDR1 comprising SEQ ID NO: 1; ii) an HCDR2 comprising SEQ ID NO:21; iii) an HCDR3 comprising SEQ ID NO:3; iv) an LCDR1 comprising SEQ ID NO:24; v) an LCDR2 comprising SEQ ID NO: 5; and vi) an LCDR3 comprising SEQ ID NO:26. In an embodiment, the anti-IL-31 antibody comprises: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:25; and vi) an LCDR3 comprising SEQ ID NO:26. In an embodiment, the anti-IL-31 antibody comprises: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:6. In an embodiment, the anti-IL-31 antibody comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:5; and vi) an LCDR3 comprising SEQ ID NO:26. In an embodiment, the anti-IL-31 antibody comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:24; v) an LCDR2 comprising SEQ ID NO:5; and vi) an LCDR3 comprising SEQ ID NO:26. In an embodiment, the anti-IL-31 antibody comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:25; and vi) an LCDR3 comprising SEQ ID NO:26. In an embodiment, the anti-IL-31 antibody comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:5; and vi) an LCDR3 comprising SEQ ID NO:6. In an embodiment, the anti-IL-31 antibody comprises: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:26. In an embodiment, the anti-IL-31 antibody comprises: i) an HCDR1 comprising SEQ ID NO: 1; ii) an HCDR2 comprising SEQ ID NO:2; iii) an HCDR3 comprising SEQ ID NO:3; iv) an LCDR1 comprising SEQ ID NO:24; v) an LCDR2 comprising SEQ ID NO:5; and vi) an LCDR3 comprising SEQ ID NO:26. In an embodiment, the anti-IL-31 antibody comprises: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:4; v) an LCDR2 comprising SEQ ID NO:25; and vi) an LCDR3 comprising SEQ ID NO:26. In an embodiment, the anti-IL-31 antibody comprises i) an HCDR1 comprising SEQ ID NO: 1; ii) an HCDR2 comprising SEQ ID NO: 2; iii) HCDR3 comprising SEQ ID NO: 3; iv) an LCDR1 comprising SEQ ID NO: 4; v) an LCDR2 comprising SEQ ID NO: 5; and vi) an LCDR3 comprising SEQ ID NO: 6. Preferably, in these embodiments the HCDR1, HCDR2, HCDR3, LCDR1, LCDR2, and LCDR3 are defined according to the Kabat numbering scheme.

In an embodiment, the anti-IL-31 antibody comprises i) an HCDR1 comprising SEQ ID NO: 48; ii) an HCDR2 comprising SEQ ID NO: 61; and iii) an HCDR3 comprising SEQ ID NO: 50. In an embodiment, the anti-IL-31 antibody comprises i) an HCDR1 comprising SEQ ID NO: 48; ii) an HCDR2 comprising SEQ ID NO: 61; and iii) an HCDR3 comprising SEQ ID NO: 63. In an embodiment, the anti-IL-31 antibody comprises i) an HCDR1 comprising SEQ ID NO: 48; ii) an HCDR2 comprising SEQ ID NO: 49; and iii) an HCDR3 comprising SEQ ID NO: 50. Preferably, in these embodiments the HCDR1, HCDR2, and HCDR3 are defined according to the Chothia numbering scheme.

In an embodiment, the anti-IL-31 antibody comprises i) an LCDR1 comprising SEQ ID NO: 51; ii) an LCDR2 comprising the amino acid sequence YAK; and iii) an LCDR3 comprising SEQ ID NO: 64. In an embodiment, the anti-IL-31 antibody comprises i) an LCDR1 comprising SEQ ID NO: 51; ii) an LCDR2 comprising the amino acid sequence YAK; and iii) an LCDR3 comprising SEQ ID NO: 53. In an embodiment, the anti-IL-31 antibody comprises i) an LCDR1 comprising SEQ ID NO: 65; ii) an LCDR2 comprising the amino acid sequence YAK; and iii) an LCDR3 comprising SEQ ID NO: 64. Preferably, in these embodiments the LCDR1, LCDR2, and LCDR3 are defined according to the Chothia numbering scheme.

In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 50; iv) an LCDR1 comprising SEQ ID NO: 51; v) an LCDR2 comprising the amino acid sequence YAK; and vi) an LCDR3 comprising SEQ ID NO: 64. In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 50; iv) an LCDR1 comprising SEQ ID NO: 65; v) an LCDR2 comprising the amino acid sequence YAK; and vi) an LCDR3 comprising SEQ ID NO: 64. In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 50; iv) an LCDR1 comprising SEQ ID NO: 51; v) an LCDR2 comprising the amino acid sequence YAK; and vi) an LCDR3 comprising SEQ ID NO: 64. In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 50; iv) an LCDR1 comprising SEQ ID NO: 51; v) an LCDR2 comprising the amino acid sequence YAK; and vi) an LCDR3 comprising SEQ ID NO: 53. In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 63; iv) an LCDR1 comprising SEQ ID NO: 51; v) an LCDR2 comprising the amino acid sequence YAK; and vi) an LCDR3 comprising SEQ ID NO: 64. In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 63; iv) an LCDR1 comprising SEQ ID NO: 65; v) an LCDR2 comprising the amino acid sequence YAK; and vi) an LCDR3 comprising SEQ ID NO: 64. In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 63; iv) an LCDR1 comprising SEQ ID NO: 51; v) an LCDR2 comprising the amino acid sequence YAK; and vi) an LCDR3 comprising SEQ ID NO: 64. In an embodiment, the anti-IL-31 antibody comprises: i) an HCDR1 comprising SEQ ID NO: 48; ii) an HCDR2 comprising SEQ ID NO: 61; iii) an HCDR3 comprising SEQ ID NO: 63; iv) an LCDR1 comprising SEQ ID NO: 51; v) an LCDR2 comprising the amino acid sequence YAK; and vi) an LCDR3 comprising SEQ ID NO: 53. In an embodiment, the anti-IL-31 antibody comprises: i) an HCDR1 comprising SEQ ID NO: 48; ii) an HCDR2 comprising SEQ ID NO: 49; iii) an HCDR3 comprising SEQ ID NO: 50; iv) an LCDR1 comprising SEQ ID NO: 51; v) an LCDR2 comprising the amino acid sequence YAK; and vi) an LCDR3 comprising SEQ ID NO: 64. In an embodiment, the IL-31 antibody comprises: i) an HCDR1 comprising SEQ ID NO: 48; ii) an HCDR2 comprising SEQ ID NO: 49; iii) an HCDR3 comprising SEQ ID NO: 50; iv) an LCDR1 comprising SEQ ID NO: 65; v) an LCDR2 comprising the amino acid sequence YAK; and vi) an LCDR3 comprising SEQ ID NO: 64. In an embodiment, the anti-IL-31 antibody comprises: i) an HCDR1 comprising SEQ ID NO: 48; ii) an HCDR2 comprising SEQ ID NO: 49; iii) an HCDR3 comprising SEQ ID NO: 50; iv) an LCDR1 comprising SEQ ID NO: 51; v) an LCDR2 comprising the amino acid sequence YAK; and vi) an LCDR3 comprising SEQ ID NO: 64. In an embodiment, the anti-IL-31 antibody comprises i) an HCDR1 comprising SEQ ID NO: 48; ii) an HCDR2 comprising SEQ ID NO: 49; iii) HCDR3 comprising SEQ ID NO: 50; iv) an LCDR1 comprising SEQ ID NO: 51; v) an LCDR2 comprising the amino acid sequence YAK; and vi) an LCDR3 comprising SEQ ID NO: 53. Preferably, in these embodiments the HCDR1, HCDR2, HCDR3, LCDR1, LCDR2, and LCDR3 are defined according to the Chothia numbering scheme.

In an embodiment, the anti-IL-31 antibody comprises i) an HCDR1 comprising SEQ ID NO: 66; ii) an HCDR2 comprising SEQ ID NO: 73; and iii) an HCDR3 comprising SEQ ID NO: 68. In an embodiment, the anti-IL-31 antibody comprises i) an HCDR1 comprising SEQ ID NO: 66; ii) an HCDR2 comprising SEQ ID NO: 73; and iii) an HCDR3 comprising SEQ ID NO: 77. In an embodiment, the anti-IL-31 antibody comprises i) an HCDR1 comprising SEQ ID NO: 66; ii) an HCDR2 comprising SEQ ID NO: 67; and iii) an HCDR3 comprising SEQ ID NO: 68. Preferably, in these embodiments the HCDR1, HCDR2, and HCDR3 are defined according to the IMGT numbering scheme.

In an embodiment, the anti-IL-31 antibody comprises i) an LCDR1 comprising SEQ ID NO: 69; ii) an LCDR2 comprising the amino acid sequence YAK; and iii) an LCDR3 comprising SEQ ID NO: 75. In an embodiment, the anti-IL-31 antibody comprises i) an LCDR1 comprising SEQ ID NO: 69; ii) an LCDR2 comprising the amino acid sequence YAK; and iii) an LCDR3 comprising SEQ ID NO: 71. In an embodiment, the anti-IL-31 antibody comprises i) an LCDR1 comprising SEQ ID NO: 69; ii) an LCDR2 comprising the amino acid sequence YAK; and iii) an LCDR3 comprising SEQ ID NO: 75. In an embodiment, the anti-IL-31 antibody comprises i) an LCDR1 comprising SEQ ID NO: 76; ii) an LCDR2 comprising the amino acid sequence YAK; and iii) an LCDR3 comprising SEQ ID NO: 75. Preferably, in these embodiments the LCDR1, LCDR2, and LCDR3 are defined according to the IMGT numbering scheme.

In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 68; iv) an LCDR1 comprising SEQ ID NO: 69; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 68; iv) an LCDR1 comprising SEQ ID NO: 76; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 68; iv) an LCDR1 comprising SEQ ID NO: 69; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 68; iv) an LCDR1 comprising SEQ ID NO: 69; v) an LCDR2 comprising the amino acid sequence YAK; and vi) an LCDR3 comprising SEQ ID NO: 71. In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 77; iv) an LCDR1 comprising SEQ ID NO: 69; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 77; iv) an LCDR1 comprising SEQ ID NO: 76; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 77; iv) an LCDR1 comprising SEQ ID NO: 69; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO: 66; ii) an HCDR2 comprising SEQ ID NO: 73; iii) an HCDR3 comprising SEQ ID NO: 77; iv) an LCDR1 comprising SEQ ID NO: 69; v) an LCDR2 comprising the amino acid sequence YAK; and vi) an LCDR3 comprising SEQ ID NO: 71. In an embodiment, the anti-IL-31 antibody comprises: i) an HCDR1 comprising SEQ ID NO: 66; ii) an HCDR2 comprising SEQ ID NO: 67; iii) an HCDR3 comprising SEQ ID NO: 68; iv) an LCDR1 comprising SEQ ID NO: 69; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO: 66; ii) an HCDR2 comprising SEQ ID NO: 67; iii) an HCDR3 comprising SEQ ID NO: 68; iv) an LCDR1 comprising SEQ ID NO: 76; v) an LCDR2 comprising the amino acid sequence YAK; and vi) an LCDR3 comprising SEQ ID NO: 75. In an embodiment, the anti-IL-31 antibody comprises: i) an HCDR1 comprising SEQ ID NO:66; ii) an HCDR2 comprising SEQ ID NO:67; iii) an HCDR3 comprising SEQ ID NO: 68; iv) an LCDR1 comprising SEQ ID NO: 69; v) an LCDR2 comprising the amino acid sequence YAK; and vi) an LCDR3 comprising SEQ ID NO: 75. In an embodiment, the anti-IL-31 antibody comprises i) an HCDR1 comprising SEQ ID NO: 66; ii) an HCDR2 comprising SEQ ID NO: 67; iii) HCDR3 comprising SEQ ID NO: 68; iv) an LCDR1 comprising SEQ ID NO: 69; v) an LCDR2 comprising the amino acid sequence YAK; and vi) an LCDR3 comprising SEQ ID NO: 71. Preferably, in these embodiments the HCDR1, HCDR2, HCDR3, LCDR1, LCDR2, and LCDR3 are defined according to the IMGT numbering scheme.

In an embodiment, the anti-IL-31 antibody comprises i) an HCDR1 comprising SEQ ID NO: 78; ii) an HCDR2 comprising SEQ ID NO: 91; and iii) an HCDR3 comprising SEQ ID NO: 80. In an embodiment, the anti-IL-31 antibody comprises i) an HCDR1 comprising SEQ ID NO: 78; ii) an HCDR2 comprising SEQ ID NO: 91; and iii) an HCDR3 comprising SEQ ID NO: 96. In an embodiment, the anti-IL-31 antibody comprises i) an HCDR1 comprising SEQ ID NO: 78; ii) an HCDR2 comprising SEQ ID NO: 79; and iii) an HCDR3 comprising SEQ ID NO: 80. Preferably, in these embodiments the HCDR1, HCDR2, and HCDR3 are defined according to the Combined numbering scheme.

In an embodiment, the anti-IL-31 antibody comprises i) an LCDR1 comprising SEQ ID NO: 81; ii) an LCDR2 comprising SEQ ID NO: 93; and iii) an LCDR3 comprising SEQ ID NO: 94. In an embodiment, the anti-IL-31 antibody comprises i) an LCDR1 comprising SEQ ID NO: 81; ii) an LCDR2 comprising SEQ ID NO: 82; and iii) an LCDR3 comprising SEQ ID NO: 83. In an embodiment, the anti-IL-31 antibody comprises i) an LCDR1 comprising SEQ ID NO: 81; ii) an LCDR2 comprising SEQ ID NO: 82; and iii) an LCDR3 comprising SEQ ID NO: 94. In an embodiment, the anti-IL-31 antibody comprises i) an LCDR1 comprising SEQ ID NO: 95; ii) an LCDR2 comprising SEQ ID NO: 82; and iii) an LCDR3 comprising SEQ ID NO: 94. Preferably, in these embodiments the LCDR1, LCDR2, and LCDR3 are defined according to the Combined numbering scheme.

In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 80; iv) an LCDR1 comprising SEQ ID NO: 81; v) an LCDR2 comprising SEQ ID NO: 82; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 80; iv) an LCDR1 comprising SEQ ID NO: 95; v) an LCDR2 comprising SEQ ID NO: 82; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 80; iv) an LCDR1 comprising SEQ ID NO: 81; v) an LCDR2 comprising SEQ ID NO: 93; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 80; iv) an LCDR1 comprising SEQ ID NO: 81; v) an LCDR2 comprising SEQ ID NO: 82; and vi) an LCDR3 comprising SEQ ID NO: 83. In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 96; iv) an LCDR1 comprising SEQ ID NO: 81; v) an LCDR2 comprising SEQ ID NO: 82; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 96; iv) an LCDR1 comprising SEQ ID NO: 95; v) an LCDR2 comprising SEQ ID NO: 82; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 96; iv) an LCDR1 comprising SEQ ID NO: 81; v) an LCDR2 comprising SEQ ID NO: 93; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 91; iii) an HCDR3 comprising SEQ ID NO: 96; iv) an LCDR1 comprising SEQ ID NO: 81; v) an LCDR2 comprising SEQ ID NO: 82; and vi) an LCDR3 comprising SEQ ID NO: 83. In an embodiment, the anti-IL-31 antibody comprises: i) an HCDR1 comprising SEQ ID NO: 78; ii) an HCDR2 comprising SEQ ID NO: 79; iii) an HCDR3 comprising SEQ ID NO: 80; iv) an LCDR1 comprising SEQ ID NO: 81; v) an LCDR2 comprising SEQ ID NO: 82; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 79; iii) an HCDR3 comprising SEQ ID NO: 80; iv) an LCDR1 comprising SEQ ID NO: 95; v) an LCDR2 comprising SEQ ID NO: 82; and vi) an LCDR3 comprising SEQ ID NO: 94. In an embodiment, the anti-IL-31 antibody comprises: i) an HCDR1 comprising SEQ ID NO: 78; ii) an HCDR2 comprising SEQ ID NO: 79; iii) an HCDR3 comprising SEQ ID NO: 80; iv) an LCDR1 comprising SEQ ID NO: 81; v) an LCDR2 comprising SEQ ID NO: 93; and vi) an LCDR3 comprising SEQ ID NO: 94. In an embodiment, the anti-IL-31 antibody comprises i) an HCDR1 comprising SEQ ID NO: 78; ii) an HCDR2 comprising SEQ ID NO: 79; iii) HCDR3 comprising SEQ ID NO: 80; iv) an LCDR1 comprising SEQ ID NO: 81; v) an LCDR2 comprising SEQ ID NO: 82; and vi) an LCDR3 comprising SEQ ID NO: 83. Preferably, in these embodiments the HCDR1, HCDR2, HCDR3, LCDR1, LCDR2, and LCDR3 are defined according to the Combined numbering scheme.

In some embodiments, the anti-IL-31 antibody comprises a heavy chain variable region (VH) comprising or consisting of the amino acid sequence shown in SEQ ID NO: 7 or SEQ ID NO: 28 or SEQ ID NO: 29. In some embodiments, the anti-IL-31 antibody comprises a heavy chain variable region (VH) comprising or consisting of an amino acid sequence having at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29.

In some embodiments, the anti-IL-31 antibody comprises a light chain variable region (VL) comprising or consisting of the amino acid sequence shown in SEQ ID NO: 8 or SEQ ID NO: 30 or SEQ ID NO:31 or SEQ ID NO:32. In some embodiments, the anti-IL-31 antibody comprises a light chain variable region (VL) comprising or consisting of an amino acid sequence having at least 80%, at least 85%, at least 90%, at least 95%, at least 96%, at least 97%, at least 98%, or at least 99% sequence identity with SEQ ID NO: 8 or SEQ ID NO: 30 or SEQ ID NO:31 or SEQ ID NO:32.

In an embodiment, the anti-IL-31 antibody comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith).

In an embodiment, the anti-IL-31 antibody comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith). In an embodiment, the anti-IL-31 antibody comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith).

In preferred embodiments, the anti-IL-31 antibody described herein further comprises an Fc polypeptide. In some embodiments, the Fc polypeptide comprises the amino acid sequence shown in SEQ ID NO: 42 or a variant thereof.

The Fc polypeptide may contain modifications e.g., to extend half-life, silence effector function, and/or to facilitate dimerization of Fc polypeptides to an Fc region (e.g., by introduction of K-i-H mutations or FAE mutations). In some embodiments, the Fc polypeptide comprises a variant of SEQ ID NO: 42 comprising (relative to SEQ ID NO: 42) one or more mutations selected from: a) one or more silencing mutations; b) one or more half-life extension mutations, and/or c) one or more K-i-H or FAE mutations.

In some embodiments, there is provided an antibody that binds to IL-31, wherein the antibody comprises a heavy chain comprising the amino acid sequence shown in SEQ ID NO: 39 or SEQ ID NO: 38 or SEQ ID NO: 37 or SEQ ID NO: 36; and an Fc polypeptide comprising the amino acid sequence shown in SEQ ID NO: 42 or a variant thereof, wherein the variant of SEQ ID NO: 42 comprises (relative to SEQ ID NO: 42) the silencing mutations L234A and L235A (“LALA”), and half-life extension mutations M252Y, S254T and T256E (“YTE”) (according to EU Numbering).

As is described in further detail herein, “knobs-into-holes” is a well-known heterodimerization technology used for the manufacture of antibodies, particularly bispecific antibodies. Introduction of either a “knob” or a “hole” in each antibody, for example in the CH3 region of the heavy chain of each antibody promotes the heterodimerization. The engineered antibodies having a “knob” preferentially partner with the engineered antibodies having a “hole”. This method is particularly effective for the production of IgG, for example IgG1, bispecific antibodies wherein production of a “knob” half IgG antibody preferentially partners with the “hole” half IgG antibody. Application of disulfide reduction/reoxidation covalently binds the two to generate a bispecific antibody with two different arms. This technology has for example been successfully used to manufacture the one-armed antibody onartuzumab that comprises a single antigen-binding arm and a human Fc region. Such “Fab arm exchange” heterodimerization technology, as described in further detail herein, is also well known to the skilled person for manufacture of antibodies, particularly bispecific antibodies. Example Fab arm exchange mutations, also termed herein “FAE mutations” include a K409R mutation and/or a F405L mutation (according to the EU numbering), for example in the CH3 region of the heavy chain of each antibody, to promote heterodimerization.

Therefore, in some embodiments, the anti-IL-31 antibody comprises an Fc polypeptide comprising one or more knob-in-hole (“K-i-H”) mutations or FAE mutations. Example K-i-H mutations include (S354C, T366W) and (Y349C, T366S, L368A, Y407V). Example FAE mutations include F405L and K409R.

In some embodiments, the anti-IL-31 antibody comprises an Fc polypeptide comprising a variant of SEQ ID NO: 42 comprising (relative to SEQ ID NO: 42) one or more mutations selected from: a) one or more silencing mutations, e.g., an L234A mutation and an L235A mutation, also known as “LALA”; and/or b) one or more half-life extension mutations, e.g., an M252Y, an S254T and a T256E mutation, also known as “YTE”; and/or c) one or more K-i-H mutations e.g., an S354C and/or a T366W) mutation or an Y349C, T366S, L368A, and/or Y407V mutation, or one or more FAE mutations, e.g., an F405L mutation or a K409R mutation, according to EU Numbering.

In some embodiments, the anti-IL-31 antibody comprises an Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) an F405L or a K409R mutation,
according to EU numbering.

In some embodiments, the anti-IL-31 antibody comprises an Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) an F405L mutation,
according to EU numbering.

In some embodiments, the anti-IL-31 antibody comprises an Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) a K409R mutation,
according to EU numbering.

In some embodiments, the anti-IL-31 antibody comprises an Fc polypeptide comprising SEQ ID NO: 44 or SEQ ID NO: 45, preferably SEQ ID NO: 44. In some embodiments, the anti-IL-31 antibody comprises an Fc polypeptide comprising SEQ ID NO: 46 or SEQ ID NO: 47.

In some embodiments, there is provided an antibody that binds to IL-31, wherein the antibody comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith); or wherein the antibody comprises: i) a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith). In some embodiments, there is provided an antibody that binds to IL-31, wherein the antibody comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith). In some embodiments, there is provided an antibody that binds to IL-31, wherein the antibody comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 10 (or an amino acid sequence having at least 80%, at least 85%, at least 90%, at least 95%, at least 96%, at least 97%, at least 98%, or at least 99% sequence identity therewith). In some embodiments, there is provided an antibody that binds to IL-31, wherein the antibody comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO: 109 (or an amino acid sequence having at least 80%, at least 85%, at least 90%, at least 95%, at least 96%, at least 97%, at least 98%, or at least 99% sequence identity therewith). Preferably, the antibody binds specifically to IL-31.

In another aspect, there is provided an antibody that binds to IL-31 comprising:
(1) a heavy chain variable region comprising or consisting of the amino acid sequence shown in SEQ ID NO: 40 or any variant thereof wherein 1 to 10 amino acids, 3 to 10 amino acids, or 5, 6, 7, 8, 9, or 10 amino acids of said sequence are substituted by a different amino acid; and
(2) a light chain variable region comprising or consisting of the amino acid sequence shown in SEQ ID NO: 41 or any variant thereof wherein 1 to 11 amino acids, 3 to 11 amino acids, or 5, 6, 7, 8, 9, 10 or 11 amino acids of said sequence are substituted by a different amino acid. Preferably, the antibody binds IL-31 with a dissociation constant (KD) of less than 100 pM, less than 90 pM, less than 80 pM, less than 70 pM, less than 60 pM, less than 50 pM, less than 30 pM, such as a kD less than 25 pM, less than 20 pM, less than 15 pM, less than 10 pM, less than 5 pM or less than about 5 pM as measured using surface plasmon resonance, preferably as measured using a Biacore instrument following the manufacturer's instructions. In some embodiments, it binds to human IL-31 with a dissociation (KD) of less than 30 pM, less than 25 pM, less than 20 pM, less than 15 pM, less than 10 pM, or less than 5 pM or less than about 5 pM as measured using surface plasmon resonance, preferably as measured using a Biacore instrument following the manufacturer's instructions.

In some embodiments, the variant of SEQ ID NO: 40 has at least 80%, at least 85%, at least 86%, at least 87%, at least 88%, at least 89%, at least 90%, at least 91%, at least 92%, at least 93%, at least 94%, or at least 95% identity with SEQ ID NO: 40; and the variant of SEQ ID NO: 41 has at least 80%, at least 85%, at least 86%, at least 87%, at least 88%, at least 89%, at least 90%, at least 91%, at least 92%, at least 93%, at least 94%, or at least 95% identity with SEQ ID NO: 41.

In some embodiments:
(1) the heavy chain variable region comprises a substitution by a different amino acid in comparison to SEQ ID NO: 40 selected from the group consisting of:
   a substitution of the alanine (A) at position 16 (VH A16),
   a substitution of the leucine (L) at position 29 (VH L29),
   a substitution of the glycine (G) at position 54 (VH G54),
   a substitution of the aspartic acid (D) at position 55 (VH D55),
   a substitution of the glutamine (Q) at position 65 (VH Q65),
   a substitution of the leucine (L) at position 70 (VH L70),
   a substitution of the aspartic acid (D) at position 100 (VH D100),
   a substitution of the tyrosine (Y) at position 102 (VH Y102),
   a substitution of the aspartic acid (D) at position 110 (VH D110),
   a substitution of the leucine (L) at position 117 (VH L117), and
   a combination thereof,
wherein the glutamine (Q) at the first position of SEQ ID NO: 40 is denoted as position 1 (VH Q1); and
(2) the light chain variable region comprises a substitution by a different amino acid in comparison to SEQ ID NO: 41 selected from the group consisting of:
   a substitution of the aspartic acid (D) at position 1 (VL D1),
   a substitution of the glycine (G) at position 27 (VL G27),
   a substitution of the histidine (H) at position 30 (VL H30),
   a substitution of the asparagine (N) at position 50 (VL N50),
   a substitution of the leucine (L) at position 54 (VL L54),
   a substitution of the aspartic acid (D) at position 56 (VL D56),
   a substitution of the glutamic acid (E) at position 68 (VL E68),
   a substitution of the glutamine (Q) at position 70 (VL Q70),
   a substitution of the serine (S) at position 72 (VL S72),
   a substitution of the serine (S) at position 93 (VL S93),
   a substitution of the glutamine (Q) at position 100 (VL Q100), and
   a combination thereof,
wherein the aspartic acid (D) at the first position of SEQ ID NO: 41 is denoted as position 1 (VL D1).

In some embodiments:
(1) the heavy chain variable region comprises a substitution by a different amino acid in comparison to SEQ ID NO: 40 selected from the group consisting of:
   a substitution of the alanine (A) at position 16 (VH A16) by a serine (S),
   a substitution of the leucine (L) at position 29 (VH L29) by an arginine (R),
   a substitution of the glycine (G) at position 54 (VH G54) by an alanine (A),
   a substitution of the aspartic acid (D) at position 55 (VH D55) by a glutamic acid (E),
   a substitution of the glutamine (Q) at position 65 (VH Q65) by a lysine (K),
   a substitution of the leucine (L) at position 70 (VH L70) by an isoleucine (I),
   a substitution of the aspartic acid (D) at position 100 (VH D100) by a glutamic acid (E),
   a substitution of the tyrosine (Y) at position 102 (VH Y102) by a proline (P) or a glutamine (Q),
   a substitution of the aspartic acid (D) at position 110 (VH D110) by a histidine (H),
   a substitution of the leucine (L) at position 117 (VH L117) by a threonine (T), and
   a combination thereof,
wherein the glutamine (Q) at the first position of SEQ ID NO: 40 is denoted as position 1 (VH Q1); and
(2) the light chain variable region comprises a substitution by a different amino acid in comparison to SEQ ID NO: 41 selected from the group consisting of:
   a substitution of the aspartic acid (D) at position 1 (VL D1) by an alanine (A),
   a substitution of the glycine (G) at position 27 (VL G27) by an arginine (R),
   a substitution of the histidine (H) at position 30 (VL H30) by a glycine (G),
   a substitution of the asparagine (N) at position 50 (VL N50) by a tyrosine (Y)
   a substitution of the leucine (L) at position 54 (VL L54) by a serine (S),
   a substitution of the aspartic acid (D) at position 56 (VL D56) by a serine (S) or a glutamic acid (E),
   a substitution of the glutamic acid (E) at position 68 (VL E68) by a glycine (G),
   a substitution of the glutamine (Q) at position 70 (VL Q70) by an aspartic acid (D),
   a substitution of the serine (S) at position 72 (VL S72) by a threonine (T),
   a substitution of the serine (S) at position 93 (VL S93) by a lysine (K),
   a substitution of the glutamine (Q) at position 100 (VL Q100) by a glycine (G), and
   a combination thereof,
wherein the aspartic acid (D) at the first position of SEQ ID NO: 41 is denoted as position 1 (VL D1).

In some embodiments, the heavy chain variable region comprises the following substitutions by different amino acids in comparison to SEQ ID NO: 40:
a substitution of the alanine (A) at position 16 (VH A16) by a serine (S),
a substitution of the leucine (L) at position 29 (VH L29) by an arginine (R),
a substitution of the glycine (G) at position 54 (VH G54) by an alanine (A),
a substitution of the aspartic acid (D) at position 55 (VH D55) by a glutamic acid (E),
a substitution of the glutamine (Q) at position 65 (VH Q65) by a lysine (K),
a substitution of the leucine (L) at position 70 (VH L70) by an isoleucine (I),
a substitution of the aspartic acid (D) at position 100 (VH D100) by a glutamic acid (E),
a substitution of the tyrosine (Y) at position 102 (VH Y102) by a proline (P),
a substitution of the aspartic acid (D) at position 110 (VH D110) by a histidine (H), and
a substitution of the leucine (L) at position 117 (VH L117) by a threonine (T),
wherein the glutamine (Q) at the first position of SEQ ID NO: 40 is denoted as position 1 (VH Q1), and preferably
the light chain variable region comprises the following substitutions by different amino acids in comparison to SEQ ID NO: 41:
   a substitution of the aspartic acid (D) at position 1 (VL D1) by an alanine (A),
   a substitution of the glycine (G) at position 27 (VL G27) by an arginine (R),
   a substitution of the histidine (H) at position 30 (VL H30) by a glycine (G),
   a substitution of the asparagine (N) at position 50 (VL N50) by a tyrosine (Y),
   a substitution of the leucine (L) at position 54 (VL L54) by a serine (S),
   a substitution of the aspartic acid (D) at position 56 (VL D56) by a serine (S),
   a substitution of the glutamic acid (E) at position 68 (VL E68) by a glycine (G),
   a substitution of the glutamine (Q) at position 70 (VL Q70) by an aspartic acid (D),
   a substitution of the serine (S) at position 72 (VL S72) by a threonine (T), and
   a substitution of the glutamine (Q) at position 100 (VL Q100) by a glycine (G),
wherein the aspartic acid (D) at the first position of SEQ ID NO: 41 is denoted as position 1 (VL D1).

In some embodiments, the antibody that binds to IL-31 comprises:
(1) a heavy chain variable region comprising or consisting of a variant of SEQ ID NO: 40, wherein the variant comprises relative to SEQ ID NO: 40 a substitution of the leucine (L) at position 29 (VH L29) by a different amino acid, wherein the glutamine (Q) at the first position of SEQ ID NO: 40 is denoted as position 1 (VH Q1); and
(2) a light chain variable region comprising or consisting of SEQ ID NO: 8 or SEQ ID NO: 30 or SEQ ID NO:31 or SEQ ID NO:32.

Preferably, the substitution of the leucine (L) at position 29 (VH L29) is a substitution by an arginine (R).

In some embodiments, the antibody that binds to IL-31 comprises:
(1) a heavy chain variable region comprising or consisting of a variant of SEQ ID NO: 40, wherein the variant comprises relative to SEQ ID NO: 40 a substitution of the glycine (G) at position 54 (VH G54), a substitution of the aspartic acid (D) at position 55 (VH D55), and a substitution of the glutamine (Q) at position 65 (VH Q65) by a different amino acid, wherein the glutamine (Q) at the first position of SEQ ID NO: 40 is denoted as position 1 (VH Q1); and
(2) a light chain variable region comprising or consisting of SEQ ID NO: 8 or SEQ ID NO: 30 or SEQ ID NO:31 or SEQ ID NO:32.

Preferably, the substitution of the glycine (G) at position 54 (VH G54) is a substitution by an alanine (A), the substitution of the aspartic acid (D) at position 55 (VH D55) is a substitution by a glutamic acid (E), and the substitution of the glutamine (Q) at position 65 (VH Q65) is a substitution by a lysine (K).

In some embodiments, the antibody that binds to IL-31 comprises:
(1) a heavy chain variable region comprising or consisting of a variant of SEQ ID NO: 40, wherein the variant comprises relative to SEQ ID NO: 40 a substitution of the aspartic acid (D) at position 100 (VH D100), a substitution of the tyrosine (Y) at position 102 (VH Y102), and a substitution of the aspartic acid (D) at position 110 (VH D110) by a different amino acid, wherein the glutamine (Q) at the first position of SEQ ID NO: 40 is denoted as position 1 (VH Q1); and
(2) a light chain variable region comprising or consisting of SEQ ID NO: 8 or SEQ ID NO: 30 or SEQ ID NO:31 or SEQ ID NO:32.

Preferably, the substitution of the aspartic acid (D) at position 100 (VH D100) is a substitution by a glutamic acid (E), the substitution of the tyrosine (Y) at position 102 (VH Y102) is a substitution by a proline (P), and the substitution of the aspartic acid (D) at position 110 (VH D110) is a substitution by a histidine (H).

In some embodiments, the variant of SEQ ID NO: 40 further comprises relative to SEQ ID NO: 40 a substitution of the alanine (A) at position 16 (VH A16), a substitution of the leucine (L) at position 70 (VH L70), and a substitution of the leucine (L) at position 117 (VH L117) by a different amino acid, wherein the glutamine (Q) at the first position of SEQ ID NO: 40 is denoted as position 1 (VH Q1). Preferably, the substitution of the alanine (A) at position 16 (VH A16) is a substitution by a serine (S), the substitution of the leucine (L) at position 70 (VH L70) is a substitution by an isoleucine (I), and the substitution of the leucine (L) at position 117 (VH L117) is a substitution by a threonine (T).

In some embodiments, the antibody that binds to IL-31 comprises:
(1) a heavy chain variable region comprising or consisting of the amino acid sequence shown in SEQ ID NO: 7, SEQ ID NO: 28, or SEQ ID NO: 29; and
(2) a light chain variable region comprising or consisting of a variant of SEQ ID NO: 41, wherein the variant comprises relative to SEQ ID NO: 41 a substitution of the glycine (G) at position 27 (VL G27) and a substitution of the histidine (H) at position 30 (VL H30) by a different amino acid, wherein the aspartic acid (D) at the first position of SEQ ID NO: 41 is denoted as position 1 (VL D1).

Preferably, the substitution of the glycine (G) at position 27 (VL G27) is a substitution by an arginine (R) and the substitution of the histidine (H) at position 30 (VL H30) is a substitution by a glycine (G).

In some embodiments, the antibody that binds to IL-31 comprises:
(1) a heavy chain variable region comprising or consisting of the amino acid sequence shown in SEQ ID NO: 7, SEQ ID NO: 28, or SEQ ID NO: 29; and
(2) a light chain variable region comprising or consisting of a variant of SEQ ID NO: 41, wherein the variant comprises relative to SEQ ID NO: 41 a substitution of the asparagine (N) at position 50 (VL N50), a substitution of the leucine (L) at position 54 (VL L54), and a substitution of the aspartic acid (D) at position 56 (VL D56) by a different amino acid, wherein the aspartic acid (D) at the first position of SEQ ID NO: 41 is denoted as position 1 (VL D1).

Preferably, the substitution of the asparagine (N) at position 50 (VL N50) is a substitution by a tyrosine (Y), the substitution of the leucine (L) at position 54 (VL L54) is a substitution by a serine (S), and the substitution of the aspartic acid (D) at position 56 (VL D56) is a substitution by a serine (S).

In some embodiments, the variant of SEQ ID NO: 41 further comprises relative to SEQ ID NO: 40 a substitution of the aspartic acid (D) at position 1 (VL D1), a substitution of the glutamic acid (E) at position 68 (VL E68), a substitution of the glutamine (Q) at position 70 (VL Q70), a substitution of the serine (S) at position 72 (VL S72), and a substitution of the glutamine (Q) at position 100 (VL Q100), wherein the aspartic acid (D) at the first position of SEQ ID NO: 41 is denoted as position 1 (VL D1). Preferably, the substitution of the aspartic acid (D) at position 1 (VL D1) is a substitution by an alanine (A), the substitution of the glutamic acid (E) at position 68 (VL E68) is a substitution by a glycine (G), the substitution of the glutamine (Q) at position 70 (VL Q70) is a substitution by an aspartic acid (D), the substitution of the serine (S) at position 72 (VL S72) is a substitution by a threonine (T), and the substitution of the glutamine (Q) at position 100 (VL Q100) is a substitution by a glycine (G).

In some embodiments, the antibody that binds to IL-31 comprises:
(1) a heavy chain variable region comprising or consisting of a variant of SEQ ID NO: 40, wherein the variant comprises relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1), and;
(2) a light chain variable region comprising or consisting of a variant of SEQ ID NO: 41, wherein the variant comprises relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

In some embodiments, the antibody binds IL-31 with a dissociation constant (KD) of less than 30 pM, less than 25 pM, less than 20 pM, less than 15 pM, less than 10 pM, or less than 5 pM or less than about 5 pM, as measured using surface plasmon resonance.

In an aspect, there is provided an antibody that competes for binding to an IL-31 epitope with an antibody comprising:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23;
an LCDR1 comprising SEQ ID NO: 4 or SEQ ID NO: 24;
an LCDR2 comprising SEQ ID NO: 5 or SEQ ID NO: 25; and
an LCDR3 comprising SEQ ID NO: 6 or SEQ ID NO: 26;

or
an HCDR1 comprising SEQ ID NO: 48;
an HCDR2 comprising SEQ ID NO: 49 or SEQ ID NO: 61;
an HCDR3 comprising SEQ ID NO: 50 or SEQ ID NO: 63;
an LCDR1 comprising SEQ ID NO: 51 or SEQ ID NO: 65;
an LCDR2 comprising the amino acid sequence YAK; and
an LCDR3 comprising SEQ ID NO: 53 or SEQ ID NO: 64;

or
an HCDR1 comprising SEQ ID NO: 66 or SEQ ID NO: 72;
an HCDR2 comprising SEQ ID NO: 67 or SEQ ID NO: 73;
an HCDR3 comprising SEQ ID NO: 68 or SEQ ID NO: 77;
an LCDR1 comprising SEQ ID NO: 69 or SEQ ID NO: 76;
an LCDR2 comprising the amino acid sequence YAK; and
an LCDR3 comprising SEQ ID NO: 71 or SEQ ID NO: 75;

or
an HCDR1 comprising SEQ ID NO: 78 or SEQ ID NO: 90;
an HCDR2 comprising SEQ ID NO: 79 or SEQ ID NO: 91;
an HCDR3 comprising SEQ ID NO: 80 or SEQ ID NO: 96;
an LCDR1 comprising SEQ ID NO: 81 or SEQ ID NO: 95;
an LCDR2 comprising SEQ ID NO: 82 or SEQ ID NO: 93; and
an LCDR3 comprising SEQ ID NO: 83 or SEQ ID NO: 94.

In some embodiments, the antibody competes for binding to an IL-31 epitope with an antibody comprising: an HCDR1 comprising SEQ ID NO: 1; an HCDR2 comprising SEQ ID NO: 2 or SEQ ID NO: 21; an HCDR3 comprising SEQ ID NO: 3 or SEQ ID NO: 23; an LCDR1 comprising SEQ ID NO: 4 or SEQ ID NO: 24; an LCDR2 comprising SEQ ID NO: 5 or SEQ ID NO: 25, and an LCDR3 comprising SEQ ID NO: 6 or SEQ ID NO: 26, preferably wherein the HCDR1, HCDR2, HCDR3, LCDR1, LCDR2, and LCDR3 are defined according to the Kabat numbering scheme.

In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:26. In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:24; v) an LCDR2 comprising SEQ ID NO:5; and vi) an LCDR3 comprising SEQ ID NO:26. In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:25; and vi) an LCDR3 comprising SEQ ID NO:26. In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:6. In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:5; and vi) an LCDR3 comprising SEQ ID NO:26. In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:24; v) an LCDR2 comprising SEQ ID NO:5; and vi) an LCDR3 comprising SEQ ID NO:26.

In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:25; and vi) an LCDR3 comprising SEQ ID NO:26. In an embodiment, the antibody competes for binding to an IL-31 epitope with an antibody comprising: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:5; and vi) an LCDR3 comprising SEQ ID NO:6. In an embodiment, the antibody competes for binding to an IL-31 epitope with an antibody comprising: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:26. In an embodiment, the antibody competes for binding to an IL-31 epitope with an antibody comprising: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:24; v) an LCDR2 comprising SEQ ID NO:5; and vi) an LCDR3 comprising SEQ ID NO:26. In an embodiment, the antibody competes for binding to an IL-31 epitope with an antibody comprising: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:4; v) an LCDR2 comprising SEQ ID NO:25; and vi) an LCDR3 comprising SEQ ID NO:26. In an embodiment, the antibody competes for binding to an IL-31 epitope with an antibody comprising: i) an HCDR1 comprising SEQ ID NO: 1; ii) an HCDR2 comprising SEQ ID NO: 2; iii) an HCDR3 comprising SEQ ID NO: 3; iv) an LCDR1 comprising SEQ ID NO: 4; v) an LCDR2 comprising SEQ ID NO: 5; and vi) an LCDR3 comprising SEQ ID NO: 6. Preferably, in these embodiments the HCDR1, HCDR2, HCDR3, LCDR1, LCDR2, and LCDR3 are defined according to the Kabat numbering scheme.

In some embodiments, the anti-IL-31 antibody described herein inhibits IL-31 activity, preferably human IL-31 activity, for example inhibits IL-31 activity with an IC50 of less than 100 pM, less than 90 pM, less than 80 pM, less than 70 pM, less than 60 pM, less than 50 pM, less than 30 pM, less than 25 pM, less than 20 pM, or less than 15 pM or less than about 15 pM. In some embodiments, it inhibits IL-31 activity, preferably human IL-31 activity, with an IC50 of less than 30 pM, less than 25 pM, less than 15 pM or less than about 15 pM, e.g., about 10 pM or less. Preferably, inhibition of IL-31 activity is measured using an IL-31 dependent Baf3 cell proliferation assay.

A non-limiting example of IL-31 activity is induction of cell proliferation, particularly of IL-31 dependent cells. IL-31 dependent Baf3 cell proliferation assays are known to the skilled person. As a non-limiting example, the human IL-31Ra (Uniprot Ref: Q8NI17) and human OSMRb (Gene ID: 9180) genes can be overexpressed in the mouse Baf3 cell line to generate human IL-31 growth dependent Baf3 cells. Stable pools of cells expressing hIL-31Ra and hOSMRb can be e.g., selected by replacing msIL-3 with human IL-31 in the growth medium. Different concentrations of human IL-31 can be used for screening and the identification of IL-31 dependent Baf3 cells that are dependent on low IL-31 concentrations, e.g., from 0.05 to 1 ng/ml, from 0.1 to 0.5 ng/ml, from 0.15 to 0.35 ng/ml, from 0.2 to 0.3 ng/ml, e.g., on 0.3 ng/ml or about 0.3 ng/ml. Confirmation of expression of hIL-31Ra and hOSMRb can be e.g., performed by flow cytometry after surface staining with antibodies against hIL-31Ra and hOSMRb, commercially available (examples of which being nemolizumab and Alexa Fluor® 647 AffiniPure™ F(ab′)₂ Fragment Goat Anti-Human IgG, F(ab′)₂ fragment specific secondary antibody (Jackson ImmunoResearch, #109-606-097)). The human IL-31 growth dependent Baf3 cells can be used to determine the potency (IC50) value of the anti-IL-31 antibody described herein in assays in which cytokine induced cell proliferation is blocked by the anti-IL-31 antibody. A further example of a Baf3 cell proliferation assay is provided in the experimental section later herein.

In some embodiments, a preferred IL-31 dependent Baf3 cell proliferation assay utilizes mouse Baf3 cells expressing human IL-31Ra and OSMRb, wherein the Baf3 cells are dependent on at least 0.3 ng/ml or about 0.3 ng/ml human IL-31 for their growth.

In some embodiments, the anti-IL-31 antibody inhibits IL-31, preferably human IL-31, activity to a greater extent compared to an anti-IL-31 antibody comprising:
i) a VH comprising SEQ ID NO: 157 and a VL comprising SEQ ID NO: 158;
ii) a VH comprising SEQ ID NO: 159 and a VL comprising SEQ ID NO: 160, and/or;
iii) a VH comprising SEQ ID NO: 40 and a VL comprising SEQ ID NO: 41, preferably as measured using an IL-31 dependent Baf3 cell proliferation assay.

In some embodiments, the anti-IL-31 antibody inhibits IL-31, preferably human IL-31, activity to a greater extent compared to an anti-IL-31 antibody comprising:
i) a heavy chain comprising SEQ ID NO: 147 and a light chain comprising SEQ ID NO: 148;
ii) a heavy chain comprising SEQ ID NO: 155 and a light chain comprising SEQ ID NO: 156;
iii) a heavy chain comprising SEQ ID NO: 153 and a light chain comprising SEQ ID NO: 154, and/or;
iv) a heavy chain comprising SEQ ID NO: 174 and a light chain comprising SEQ ID NO: 175, preferably as measured using an IL-31 dependent Baf3 cell proliferation assay.

Improved inhibition (potency) can manifest as a lower IC50 value for IL-31, preferably human IL-31, for example at least a two-fold, at least a three-fold, at least a four-fold, at least a five-fold, at least a six-fold, at least a seven-fold, at least an eight-fold, at least a nine-fold, at least a ten-fold, or lower, IC50 value relative to a reference anti-IL-31 antibody.

In some embodiments, the anti-IL-31 antibody described herein binds to IL-31, preferably human IL-31, with a dissociation constant (KD) of less than 100 pM, less than 90 pM, less than 80 pM, less than 70 pM, less than 60 pM, less than 50 pM, less than 30 pM, such as a kD less than 25 pM, less than 20 pM, less than 15 pM, less than 10 pM, less than 5 pM or less than about 5 pM as measured using surface plasmon resonance, preferably as measured using a Biacore instrument.

In some embodiments, it binds to IL-31, preferably human IL-31, with a dissociation constant (KD) of less than 30 pM, less than 25 pM, less than 20 pM, less than 15 pM, less than 10 pM, or less than 5 pM or less than about 5 pM as measured using surface plasmon resonance, preferably as measured using a Biacore instrument. Non-limiting examples of Biacore instruments include Biacore T200, T100 and 8K.

In some embodiments, the anti-IL-31 antibody has an improved affinity for IL-31, preferably human IL-31, compared to an anti-IL-31 antibody comprising:
i) a VH comprising SEQ ID NO: 157 and a VL comprising SEQ ID NO: 158;
ii) a VH comprising SEQ ID NO: 159 and a VL comprising SEQ ID NO: 160, and/or;
iii) a VH comprising SEQ ID NO: 40 and a VL comprising SEQ ID NO: 41, as measured using surface plasmon resonance, preferably as measured using a Biacore instrument.

In some embodiments, the anti-IL-31 antibody has an improved affinity for IL-31, preferably human IL-31, compared to an anti-IL-31 antibody comprising:
i) a heavy chain comprising SEQ ID NO: 147 and a light chain comprising SEQ ID NO: 148;
ii) a heavy chain comprising SEQ ID NO: 155 and a light chain comprising SEQ ID NO: 156, and/or;
iii) a heavy chain comprising SEQ ID NO: 153 and a light chain comprising SEQ ID NO: 154,
as measured using surface plasmon resonance, preferably as measured using a Biacore instrument.

Improved affinity can manifest as a lower dissociation constant (KD) for IL-31, preferably human IL-31, for example at least a two-fold, at least a three-fold, at least a four-fold, at least a five-fold, at least a six-fold, at least a seven-fold, at least an eight-fold, at least a nine-fold, at least a ten-fold, or lower, dissociation constant relative to a reference anti-IL-31 antibody.

In some embodiments, the anti-IL-31 antibody comprises a Fab domain having a melting temperature (Tm) of at least 70° C., such as 70° C., 71° C., 72° C., 73° C., 74° C., 75° C., 76°, 77° C. preferably at least 75° C., more preferably 76° C. or about 76° C., preferably as measured by DSF (differential scanning fluorimetry). Non-limiting examples of suitable DSF conditions are a buffer of 20 mM His/His-HCl at a pH of 5.5 or about 5.5. A further example of a DSF assay is provided in the experimental section later herein.

In some embodiments, the anti-IL-31 antibody described herein binds, preferably specifically binds, to a human IL-31 epitope consisting of amino acid residues S35, D37, V38, K40, I41, E44, N81, P86, R89, A90, K93, R96, K101, D105, E106, I108, E109, H110, D112, K113, L114, I115, F116, Q117, D118, A119, P120, E121, and T122 of human IL-31, preferably human IL-31 represented by SEQ ID NO: 143 or SEQ ID NO: 173. Such antibodies can be readily generated by the skilled person on the basis of the disclosure. For example, the skilled person may vary the heavy chain variable region and light chain variable region sequences by substituting specific positions described earlier herein, and further discussed in the experimental section, using standard molecular toolbox techniques. Binding to the epitope can be confirmed using any of the assays described herein, in combination with X-ray crystallography using standard protocols (an example of an X-ray crystallography assay of IL-31 complexed with anti-IL31 antibody is provided in the experimental section herein).

Non-limiting exemplary positions in the light chain variable region that can be substituted by a different amino acid to generate anti-IL-31 antibodies (VL positions are numbered starting from the aspartic acid (D) at position 1 of the VL represented by SEQ ID NO: 41) are Asp 1, Gly27, Asn28, His30, Asn50, Lys52, Thr53, Leu54, Ala55, Asp56, Glu68, and Ser93. In some embodiments the VL position that is substituted by a different amino acid is selected from the group consisting of Asp1, Gly27, His30, Asn50, Leu54, Asp56, and Glu68, or from a combination thereof. Non-limiting exemplary positions in the heavy chain variable region that can be substituted by a different amino acid to generate anti-IL-31 antibodies (VH positions are numbered starting from the glutamine (Q) at position 1 of the VH represented by SEQ ID NO: 40) are Leu29, Trp33, Gly54, Asp55, Gly56, Lys65, Asp100, Gly101, Tyr102, Ala104, Ala105, and Asp110. In some embodiments, the VH position that is substituted by a different amino acid is selected from the group consisting of Leu29, Gly54, Asp55, Asp100, Tyr102, and Asp110, or from a combination thereof.

In some embodiments, the anti-IL-31 antibody comprises a heavy chain variable region (VH) comprising at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29 and a light chain variable region (VL) comprising at least 80%, at least 85%, at least 90%, at least 95%, at least 96%, at least 97%, at least 98%, or at least 99% sequence identity with SEQ ID NO: 8 or SEQ ID NO: 30 or SEQ ID NO:31 or SEQ ID NO:32. In some embodiments, the anti-IL-31 antibody comprises a heavy chain variable region (VH) comprising at least 80%, at least 85%, at least 90%, at least 95%, at least 96%, at least 97%, at least 98%, or at least 99% sequence identity with SEQ ID NO: 7 and a light chain variable region (VL) comprising at least 80%, at least 85%, at least 90%, at least 95%, at least 96%, at least 97%, at least 98%, or at least 99% sequence identity with SEQ ID NO: 8. In some embodiments, the heavy chain variable region comprises relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1). In some embodiments, the light chain variable region comprises relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

In some embodiments, the anti-IL-31 antibody described herein binds to Cynomolgus monkey IL-31.

In some embodiment, the anti-IL-31 antibody described herein is monospecific for IL-31.

In some embodiments, the anti-IL-31 antibody is monovalent with respect to binding IL-31. In other embodiments, the anti-IL-31 antibody is multivalent, preferably bivalent, with respect to binding IL-31.

In some embodiments, the anti-IL-31 antibody is a humanized, human, or chimeric antibody. In some embodiments, the anti-IL-31 antibody is monoclonal. In some embodiments, the anti-IL-31 antibody is a humanized antibody, preferably a humanized monoclonal antibody.

In some embodiments, the anti-IL-31 antibody is an IgG type antibody, for example a human IgG1 type.

In some embodiments, the anti-IL-31 antibody is a humanized, monoclonal, monospecific for IL-31, bivalent with respect to binding IL-31, antibody of IgG1 type.

In some embodiments, the anti-IL-31 antibody is an isolated antibody.

### Multispecific IL-31 Binding Antibodies

Multispecific (multi-targeting) antibodies represent a promising avenue for treatments of diseases and disorders that are not adequately treated by or are resistant to monotherapies. However, multispecific e.g., bispecific antibodies, often suffer from relatively poor physicochemical stability, pharmacokinetic properties, immunogenicity, and/or scalability of manufacturing and purification, whereas the multispecific antibodies provided herein overcome one or more of these challenges. In particular, the multispecific antibodies, preferably bispecific antibodies, of the present invention provide surprisingly high binding affinity and/or potency. The multispecific antibodies of the present invention may surprisingly retain a high binding affinity and/or potency relative to monospecific antibodies binding to the same targets or antigens. The multispecific antibodies of the invention may also exhibit one or more further favorable pharmaceutical properties as demonstrated herein relative to available antibodies in the art, including high thermostability and low aggregation propensities, to facilitate manufacturing and storage, lower effector function, and/or increased half-life.

Aspects and embodiments with respect to the multispecific antibodies described herein are also applicable to their antigen-binding fragments.

Thus, in a further aspect, there is provided a multispecific antibody comprising;
a first part that binds to IL-31 as described earlier herein, and;
a second part that binds to a target different from IL-31.

In some embodiments, the second part binds to a target selected from the group consisting of IL-2, IL-4, IL-5, IL-6, IL-7, IL-10, IL-12, IL-13, IL-15, IL-17, IL-18, IL-21, IL-22, IL-23, IL-33, IL-36, IL-37, IL-38, and their receptors. Preferably, the second part binds to IL-13.

Preferably, the first part specifically binds to IL-31, and the second part specifically binds to a target different from IL-31, preferably to IL-13. Preferably, the multispecific antibody is a bispecific antibody.

In some embodiments, the first part that binds to IL-31 comprises:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23;
an LCDR1 comprising SEQ ID NO: 4 or SEQ ID NO: 24;
an LCDR2 comprising SEQ ID NO: 5 or SEQ ID NO: 25; and
an LCDR3 comprising SEQ ID NO: 6 or SEQ ID NO: 26;

or
an HCDR1 comprising SEQ ID NO: 48;
an HCDR2 comprising SEQ ID NO: 49 or SEQ ID NO: 61;
an HCDR3 comprising SEQ ID NO: 50 or SEQ ID NO: 63;
an LCDR1 comprising SEQ ID NO: 51 or SEQ ID NO: 65;
an LCDR2 comprising the amino acid sequence YAK; and
an LCDR3 comprising SEQ ID NO: 53 or SEQ ID NO: 64;

or
an HCDR1 comprising SEQ ID NO: 66 or SEQ ID NO: 72;
an HCDR2 comprising SEQ ID NO: 67 or SEQ ID NO: 73;
an HCDR3 comprising SEQ ID NO: 68 or SEQ ID NO: 77;
an LCDR1 comprising SEQ ID NO: 69 or SEQ ID NO: 76;
an LCDR2 comprising the amino acid sequence YAK; and
an LCDR3 comprising SEQ ID NO: 71 or SEQ ID NO: 75;

or
an HCDR1 comprising SEQ ID NO: 78 or SEQ ID NO: 90;
an HCDR2 comprising SEQ ID NO: 79 or SEQ ID NO: 91;
an HCDR3 comprising SEQ ID NO: 80 or SEQ ID NO: 96;
an LCDR1 comprising SEQ ID NO: 81 or SEQ ID NO: 95;
an LCDR2 comprising SEQ ID NO: 82 or SEQ ID NO: 93; and
an LCDR3 comprising SEQ ID NO: 83 or SEQ ID NO: 94.

In some embodiments, the first part that binds to IL-31 comprises: an HCDR1 comprising SEQ ID NO: 1; an HCDR2 comprising SEQ ID NO: 2 or SEQ ID NO: 21; an HCDR3 comprising SEQ ID NO: 3 or SEQ ID NO: 23; an LCDR1 comprising SEQ ID NO: 4 or SEQ ID NO: 24; an LCDR2 comprising SEQ ID NO: 5 or SEQ ID NO: 25, and an LCDR3 comprising SEQ ID NO: 6 or SEQ ID NO: 26, preferably wherein the HCDR1, HCDR2, HCDR3, LCDR1, LCDR2, and LCDR3 are defined according to the Kabat numbering scheme.

In some embodiments, the first part that binds to IL-31 comprises: an HCDR1 comprising SEQ ID NO: 48 or SEQ ID NO: 60; an HCDR2 comprising SEQ ID NO: 49 or SEQ ID NO: 61; an HCDR3 comprising SEQ ID NO: 50 or SEQ ID NO: 63; an LCDR1 comprising SEQ ID NO: 51 or SEQ ID NO: 65; an LCDR2 comprising the amino acid sequence YAK, and an LCDR3 comprising SEQ ID NO: 53 or SEQ ID NO: 64, preferably wherein the HCDR1, HCDR2, HCDR3, LCDR1, LCDR2, and LCDR3 are defined according to the Chothia numbering scheme.

In some embodiments, the first part that binds to IL-31 comprises: an HCDR1 comprising SEQ ID NO: 66 or SEQ ID NO: 72; an HCDR2 comprising SEQ ID NO: 67 or SEQ ID NO: 73; an HCDR3 comprising SEQ ID NO: 68 or SEQ ID NO: 77; an LCDR1 comprising SEQ ID NO: 69 or SEQ ID NO: 76; an LCDR2 comprising the amino acid sequence YAK, and an LCDR3 comprising SEQ ID NO: 71 or SEQ ID NO: 75, preferably wherein the HCDR1, HCDR2, HCDR3, LCDR1, LCDR2, and LCDR3 are defined according to the IMGT numbering scheme.

In some embodiments, the first part that binds to IL-31 comprises: an HCDR1 comprising SEQ ID NO: 78 or SEQ ID NO: 90; an HCDR2 comprising SEQ ID NO: 79 or SEQ ID NO: 91; an HCDR3 comprising SEQ ID NO: 80 or SEQ ID NO: 96; an LCDR1 comprising SEQ ID NO: 81 or SEQ ID NO: 95; an LCDR2 comprising SEQ ID NO: 82 or SEQ ID NO: 93, and an LCDR3 comprising SEQ ID NO: 83 or SEQ ID NO: 94, preferably wherein the HCDR1, HCDR2, HCDR3, LCDR1, LCDR2, and LCDR3 are defined according to the Combined numbering scheme.

In some embodiments, the first part that binds to IL-31 comprises i) an HCDR1 comprising SEQ ID NO: 1; ii) an HCDR2 comprising SEQ ID NO: 21; and iii) an HCDR3 comprising SEQ ID NO: 3. In some embodiments, the first part that binds to IL-31 comprises i) an HCDR1 comprising SEQ ID NO: 1; ii) an HCDR2 comprising SEQ ID NO: 21; and iii) an HCDR3 comprising SEQ ID NO: 23. In some embodiments, the first part that binds to IL-31 comprises i) an HCDR1 comprising SEQ ID NO: 1; ii) an HCDR2 comprising SEQ ID NO: 2; and iii) an HCDR3 comprising SEQ ID NO: 3. Preferably, in these embodiments the HCDR1, HCDR2, and HCDR3 are defined according to the Kabat numbering scheme.

In some embodiments, the first part that binds to IL-31 comprises i) an LCDR1 comprising SEQ ID NO:4; ii) an LCDR2 comprising SEQ ID NO:25; and iii) an LCDR3 comprising SEQ ID NO:26. In some embodiments, the first part that binds to IL-31 comprises i) an LCDR1 comprising SEQ ID NO:4; ii) an LCDR2 comprising SEQ ID NO:5; and iii) an LCDR3 comprising SEQ ID NO:6. In some embodiments, the first part that binds to IL-31 comprises i) an LCDR1 comprising SEQ ID NO:4; ii) an LCDR2 comprising SEQ ID NO:5; and iii) an LCDR3 comprising SEQ ID NO:26. In some embodiments, the first part that binds to IL-31 comprises i) an LCDR1 comprising SEQ ID NO: 24; ii) an LCDR2 comprising SEQ ID NO: 5; and iii) an LCDR3 comprising SEQ ID NO: 26. Preferably, in these embodiments the LCDR1, LCDR2, and LCDR3 are defined according to the Kabat numbering scheme.

In some embodiments, the first part that binds to IL-31 comprises: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:26. In some embodiments, the first part that binds to IL-31 comprises: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:24; v) an LCDR2 comprising SEQ ID NO:5; and vi) an LCDR3 comprising SEQ ID NO:26. In some embodiments, the first part that binds to IL-31, comprises: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:25; and vi) an LCDR3 comprising SEQ ID NO:26. In some embodiments, the first part that binds to IL-31 comprises: i) an HCDR1 comprising SEQ ID NO:1; ii) an HCDR2 comprising SEQ ID NO:21; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:6. In some embodiments, the first part that binds to IL-31 comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:5; and vi) an LCDR3 comprising SEQ ID NO:26. In some embodiments, the first part that binds to IL-31 comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:24; v) an LCDR2 comprising SEQ ID NO:5; and vi) an LCDR3 comprising SEQ ID NO:26. In some embodiments, the first part that binds to IL-31 comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:25; and vi) an LCDR3 comprising SEQ ID NO:26. In some embodiments, the first part that binds to IL-31 comprises: i) an HCDR1 comprising SEQ ID NO:1; ii) an HCDR2 comprising SEQ ID NO:21; iii) an HCDR3 comprising SEQ ID NO:23; iv) an LCDR1 comprising SEQ ID NO:4; v) an LCDR2 comprising SEQ ID NO:5; and vi) an LCDR3 comprising SEQ ID NO:6. In some embodiments, the first part that binds to IL-31 comprises: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:4; v) an LCDR2 comprising SEQ ID NO:5; and vi) an LCDR3 comprising SEQ ID NO:26. In some embodiments, the first part that binds to IL-31 comprises: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:24; v) an LCDR2 comprising SEQ ID NO:5; and vi) an LCDR3 comprising SEQ ID NO:26. In some embodiments, the first part that binds to IL-31 comprises: i) an HCDR1 comprising SEQ ID NO:1; ii) an HCDR2 comprising SEQ ID NO:2; iii) an HCDR3 comprising SEQ ID NO:3; iv) an LCDR1 comprising SEQ ID NO:4; v) an LCDR2 comprising SEQ ID NO:25; and vi) an LCDR3 comprising SEQ ID NO:26. In some embodiments, the first part that binds to IL-31 comprises i) an HCDR1 comprising SEQ ID NO: 1; ii) an HCDR2 comprising SEQ ID NO: 2; iii) HCDR3 comprising SEQ ID NO: 3; iv) an LCDR1 comprising SEQ ID NO: 4; v) an LCDR2 comprising SEQ ID NO: 5; and vi) an LCDR3 comprising SEQ ID NO: 6. Preferably, in these embodiments the HCDR1, HCDR2, HCDR3, LCDR1, LCDR2, and LCDR3 are defined according to the Kabat numbering scheme.

In some embodiments, the first part that binds to IL-31 comprises a heavy chain variable region (VH) comprising or consisting of the amino acid sequence shown in SEQ ID NO: 7 or SEQ ID NO: 28 or SEQ ID NO: 29. In some embodiments, the first part that binds to IL-31 comprises a heavy chain variable region (VH) comprising or consisting of an amino acid sequence having at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29.

In some embodiments, the first part that binds to IL-31 comprises a light chain variable region (VL) comprising or consisting of the amino acid sequence shown in SEQ ID NO: 8 or SEQ ID NO: 30 or SEQ ID NO:31 or SEQ ID NO:32. In some embodiments, the first part that binds to IL-31 comprises a light chain variable region (VL) comprising or consisting of an amino acid sequence having at least 80%, at least 85%, at least 90%, at least 95%, at least 96%, at least 97%, at least 98%, or at least 99% sequence identity with SEQ ID NO: 8 or SEQ ID NO: 30 or SEQ ID NO:31 or SEQ ID NO:32.

In some embodiments, the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith); and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith).

In some embodiments, the first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith). In some embodiments, the first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith).

Preferably, the second part comprised in the multispecific antibody, preferably bispecific antibody, binds to IL-13. Preferably, the first part and the second part comprised in the multispecific antibody, preferably bispecific antibody, specifically bind to IL-31 and IL-13, respectively.

Preferably, the IL-31 and IL-13 are human IL-31 and IL-13. In preferred embodiments, a multispecific, preferably bispecific, antibody comprising a first part that binds to IL-31 and a second part that binds to IL-13 is an IL-31/IL-13 inhibitor.

In some embodiments, the second part binds to IL-13 and comprises: i) an HCDR1 comprising SEQ ID NO: 11; ii) an HCDR2 comprising SEQ ID NO: 12; iii) an HCDR3 comprising SEQ ID NO: 13; iv) an LCDR1 comprising SEQ ID NO: 14; v) an LCDR2 comprising SEQ ID NO: 15; and vi) an LCDR3 comprising SEQ ID NO: 16, preferably wherein the HCDR1, HCDR2, HCDR3, LCDR1, LCDR2, and LCDR3 are defined according to the Kabat numbering scheme.

In some embodiments, the second part binds to IL-13 and comprises: i) an HCDR1 comprising SEQ ID NO: 54; ii) an HCDR2 comprising SEQ ID NO: 55; iii) an HCDR3 comprising SEQ ID NO: 56; iv) an LCDR1 comprising SEQ ID NO: 57; v) an LCDR2 comprising the amino acid sequence LAS; and vi) an LCDR3 comprising SEQ ID NO: 59, preferably wherein the HCDR1, HCDR2, HCDR3, LCDR1, LCDR2, and LCDR3 are defined according to the Chothia numbering scheme.

In some embodiments, the second part binds to IL-13 and comprises: i) an HCDR1 comprising SEQ ID NO: 97; ii) an HCDR2 comprising SEQ ID NO: 98; iii) an HCDR3 comprising SEQ ID NO: 99; iv) an LCDR1 comprising SEQ ID NO: 100; v) an LCDR2 the amino acid sequence LAS; and vi) an LCDR3 comprising SEQ ID NO: 102, preferably wherein the HCDR1, HCDR2, HCDR3, LCDR1, LCDR2, and LCDR3 are defined according to the IMGT numbering scheme.

In some embodiments, the second part binds to IL-13 and comprises: i) an HCDR1 comprising SEQ ID NO: 84; ii) an HCDR2 comprising SEQ ID NO: 85; iii) an HCDR3 comprising SEQ ID NO: 86; and/or iv) an LCDR1 comprising SEQ ID NO: 87; v) an LCDR2 comprising SEQ ID NO: 88; and vi) an LCDR3 comprising SEQ ID NO: 89, preferably wherein the HCDR1, HCDR2, HCDR3, LCDR1, LCDR2, and LCDR3 are defined according to the Combined numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO:3;
iv) an LCDR1 comprising SEQ ID NO: 4;
v) an LCDR2 comprising SEQ ID NO: 5; and
vi) an LCDR3 comprising SEQ ID NO 6, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6, and
the second part binds to IL-13 and comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments, the second part binds to IL-13 and comprises a heavy chain variable region (VH) comprising or consisting of the amino acid sequence shown in SEQ ID NO: 17. In some embodiments, the second part binds to IL-13 comprises a heavy chain variable region (VH) comprising or consisting of an amino acid sequence having at least 80%, at least 85%, at least 90%, at least 95%, at least 96%, at least 97%, at least 98%, or at least 99% sequence identity with SEQ ID NO: 17.

In some embodiments, the second part binds to IL-13 and comprises a light chain variable region (VL) comprising or consisting of the amino acid sequence shown in SEQ ID NO: 18. In some embodiments, the second part binds to IL-13 comprises a light chain variable region (VL) comprising or consisting of an amino acid sequence having at least 80%, at least 85%, at least 90%, at least 95%, at least 96%, at least 97%, at least 98%, or at least 99% sequence identity with SEQ ID NO: 18.

In some embodiments,
the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith), and;
the second part binds to IL-13 and comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments, the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith).

In some embodiments, the second part binds to IL-13 and comprises a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the second part binds to IL-13 and comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In preferred embodiments, the multispecific, preferably bispecific, antibody comprises an Fc region. An exemplary such region can be formed by dimerization of a first Fc polypeptide comprised by the first part that binds to IL-31, and of a second Fc polypeptide comprised by the second part, said second part preferably binding to IL-13 as described herein.

Thus, in preferred embodiments,
the first part that binds to IL-31 comprises a first Fc polypeptide, and;
the second part binds to IL-13 and comprises a second Fc polypeptide, wherein the first and second Fc polypeptide associate to form an Fc region.

In some embodiments, the first and/or second polypeptide comprises the amino acid sequence shown in SEQ ID NO: 42 or a variant thereof.

It is understood that the first Fc polypeptide comprised by the first part and the second Fc polypeptide comprised by the second part of a multispecific, preferably bispecific, antibody described herein may differ in at least one amino acid residue, e.g., to promote dimerization and formation of the antibody by introduction of K-i-H or FAE mutation pairs described herein.

The first and/or second Fc polypeptide comprised by the first and second parts of the antibody may contain modifications e.g., to extend half-life, silence effector function, and/or to facilitate dimerization of Fc polypeptides to an Fc region (e.g., by introduction of K-i-H mutations or FAE mutations). In some embodiments, the first and/or second Fc polypeptide comprises a variant of SEQ ID NO: 42 comprising (relative to SEQ ID NO: 42) one or more mutations (e.g., one, two, three, four, five, six, seven, eight, nine, or more mutations) selected from: a. one or more silencing mutations (e.g., an L234A and an L235A mutation “LALA”); and/or b. one or more half-life extension mutations (e.g., an M252Y, an S254T, and a T256E mutation “YTE”); and/or c, one or more K-i-H (e.g., an S354C and/or a T366W mutation or an Y349C, a T366S, an L368A, and/or Y407V mutation) or FAE mutations (e.g., an F405L mutation or a K409R mutation).

In some embodiments,
the first part that binds to IL-31 comprises a first Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) an F405L mutation, and
the second part binds to IL-13 and comprises a second Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) an K409R mutation,
according to EU numbering.

In some embodiments,
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) a K409R mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an F405L mutation,
according to EU numbering.

In some embodiments,
the first part that binds to IL-31 comprises a first Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) an S354C and a T366W mutation, and
the second part binds to IL-13 and comprises a second Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) a Y349C, a T366S, an L368A, and Y407V mutation,
according to EU numbering.

In some embodiments,
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) a Y349C, a T366S, an L368A, and Y407V mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an S354C and a T366W mutation,
according to EU numbering.

In some embodiments,
the first part that binds to IL-31 comprises a first Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) a T366W mutation, and
the second part binds to IL-13 and comprises a second Fc polypeptide comprising a variant of SEQ ID NO: 42 comprising (relative to SEQ ID NO: 42):
a) an L234A and an L235A mutation (“LALA”);
b) an M252Y, an S254T, and a T256E mutation (“YTE”); and
c) a T366S, an L368A, and Y407V mutation,
according to EU numbering.

In some embodiments,
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) a T366S, an L368A, and Y407V mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) a T366W mutation,
according to EU numbering.

In some embodiments,
i) the first part that binds to IL-31 comprises a first Fc polypeptide comprising the amino acid sequence shown in SEQ ID NO: 44, and the second part binds to IL-13 and comprises a second Fc polypeptide comprising the amino acid sequence shown in SEQ ID NO: 45; or
ii) the first part that binds to IL-31 comprises a first Fc polypeptide comprising the amino acid sequence shown in SEQ ID NO: 45, and the second part binds to IL-13 and comprises a second Fc polypeptide comprising the amino acid sequence shown in SEQ ID NO: 44.

In some embodiments,
i) the first part that binds to IL-31 comprises a first Fc polypeptide comprising the amino acid sequence shown in SEQ ID NO: 46, and the second part binds to IL-13 and comprises a second Fc polypeptide comprising the amino acid sequence shown in SEQ ID NO: 47; or
ii) the first part that binds to IL-31 comprises a first Fc polypeptide comprising the amino acid sequence shown in SEQ ID NO: 47, and the second part binds to IL-13 and comprises a second Fc polypeptide comprising the amino acid sequence shown in SEQ ID NO: 46.

In some embodiments, the first part that binds to IL-31 comprises a heavy chain comprising the amino acid sequence shown in SEQ ID NO: 39 or SEQ ID NO: 38 or SEQ ID NO: 37; and a first Fc polypeptide comprising the amino acid sequence shown in SEQ ID NO: 42 or a variant thereof, wherein the variant of SEQ ID NO: 42 comprises (relative to SEQ ID NO: 42) the silencing mutations L234A and L235A (“LALA”), half-life extension mutations M252Y, S254T and T256E (“YTE”), and a F405L or K409R mutation, preferably a F405L mutation, according to EU numbering.

In some embodiments, the second part binds to IL-13 and comprises a heavy chain comprising the amino acid sequence shown in SEQ ID NO: 43; and a second Fc polypeptide comprising the amino acid sequence shown in SEQ ID NO: 42 or a variant thereof, wherein the variant of SEQ ID NO: 42 comprises (relative to SEQ ID NO: 42) the silencing mutations L234A and L235A (“LALA”), half-life extension mutations M252Y, S254T and T256E (“YTE”), and an F405L or K409R mutation, preferably a K409R mutation, according to EU numbering.

In some embodiments, the first part that binds to IL-31 comprises a heavy chain comprising the amino acid sequence shown in SEQ ID NO: 39 or SEQ ID NO: 38 or SEQ ID NO: 37; and a first Fc polypeptide comprising the amino acid sequence shown in SEQ ID NO: 42 or a variant thereof, wherein the variant of SEQ ID NO: 42 comprises (relative to SEQ ID NO: 42) the silencing mutations L234A and L235A (“LALA”), half-life extension mutations M252Y, S254T and T256E (“YTE”), and an S354C and a T366W mutation or a Y349C, a T366S, an L368A, and a Y407V mutation, according to EU Numbering.

In some embodiments, the second part binds to IL-13 and comprises a heavy chain comprising the amino acid sequence shown in SEQ ID NO: 43; and a second Fc polypeptide comprising the amino acid sequence shown in SEQ ID NO: 42 or a variant thereof, wherein the variant of SEQ ID NO: 42 comprises (relative to SEQ ID NO: 42) the silencing mutations L234A and L235A (“LALA”), half-life extension mutations M252Y, S254T and T256E (“YTE”), and an S354C and a T366W mutation or a Y349C, a T366S, an L368A, and a Y407V mutation, according to EU numbering.

In some embodiments, the multispecific, preferably bispecific, antibody comprises a first part that binds to IL-31 that is an IgG antibody or a half IgG antibody or an IL-31 binding domain e.g., an anti-IL-31 Fab, an anti-IL-31 Fv, an anti-IL-31 scFv, an anti-IL-31 scFv-Fc or fragment thereof e.g., a VL or VH domain or CDRs, preferably an anti-IL-31 antibody heavy and light chain pair (i.e., half IgG antibody). Preferably, the first part specifically binds to IL-31, preferably human IL-31.

In some embodiments, the multispecific, preferably bispecific, antibody comprises a second part that binds to IL-13 that is an IgG antibody or a half IgG antibody or an IL-13 binding domain e.g., an anti-IL-13 Fab, an anti-IL-13 Fv, an anti-IL-13 scFv, an anti-IL-13 scFv-Fc or fragment thereof e.g., a VL or VH domain or CDRs, preferably an anti-IL-13 antibody heavy and light chain pair (i.e., half IgG antibody). Preferably, the second part specifically binds to IL-13, preferably human IL-13.

In preferred embodiments, the multispecific, preferably bispecific, antibody comprises a first part that binds to IL-31 comprising a heavy and light chain pair, and a second part that binds to IL-13 comprising a heavy and light chain pair. In some embodiments, the light chain of the first part that binds to IL-31 is a kappa light chain, and the light chain of the second part that binds to IL-13 is a kappa light chain.

In some embodiments, the first part that binds to IL-31 comprises a Fab and the second part that binds to IL-13 comprises a Fab. In some embodiments, the first part that binds to IL-31 is monoclonal and the second part that binds to IL-13 is monoclonal.

In some embodiments, there is provided a multispecific, preferably bispecific antibody, comprising i) a first part that binds to IL-31 and ii) a second part that binds to IL-13, wherein the first part that binds to IL-31 comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO 10 or 109 (or an amino acid sequence having at least 80%, at least 85%, at least 90%, at least 95%, at least 96%, at least 97%, at least 98%, or at least 99% sequence identity therewith); and wherein the second part that binds to IL-13 comprises: iii) a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and iv) a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 104 or 107 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 104 or 107 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 104 or 107 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 104 or 107 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 105 or 108 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 105 or 108 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 105 or 108 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 105 or 108 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 10 or 109 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 10 or 109 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO 10 or 109 (or an amino acid sequence having at least 80%, at least 85%, at least 90%, at least 95%, at least 96%, at least 97%, at least 98%, or at least 99% sequence identity therewith); and wherein the second part that binds to IL-13 comprises: a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO 10 (or an amino acid sequence having at least 80%, at least 85%, at least 90%, at least 95%, at least 96%, at least 97%, at least 98%, or at least 99% sequence identity therewith); and wherein the second part that binds to IL-13 comprises: iii) a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and iv) a heavy chain comprising or consisting of the amino acid sequence shown in SEQ ID NO: 20 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO 109 (or an amino acid sequence having at least 80%, at least 85%, at least 90%, at least 95%, at least 96%, at least 97%, at least 98%, or at least 99% sequence identity therewith); and wherein the second part that binds to IL-13 comprises: iii) a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith), and iv) a heavy chain comprising or consisting of the amino acid sequence shown in SEQ ID NO: 110 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, there is provided a multispecific, preferably bispecific antibody, comprising i) a first part that binds to IL-31 and ii) a second part that binds to IL-13, wherein the first part that binds to IL-31 comprises: i) a light chain comprising or consisting of the amino acid sequence shown in SEQ ID NO: 177 (or an amino acid sequence having at least 80%, at least 85%, at least 90%, at least 95%, at least 96%, at least 97%, at least 98%, or at least 99% sequence identity therewith); and ii) a heavy chain comprising or consisting of the amino acid sequence shown in SEQ ID NO 176 (or an amino acid sequence having at least 80%, at least 85%, at least 90%, at least 95%, at least 96%, at least 97%, at least 98%, or at least 99% sequence identity therewith); and wherein the second part that binds to IL-13 comprises: iii) a light chain comprising or consisting of the amino acid sequence shown in SEQ ID NO: 179 (or an amino acid sequence having at least 80%, at least 85%, at least 90%, at least 95%, at least 96%, at least 97%, at least 98%, or at least 99% sequence identity therewith), and iv) a heavy chain comprising or consisting of the amino acid sequence shown in SEQ ID NO: 178 (or an amino acid sequence having at least 80%, at least 85%, at least 90%, at least 95%, at least 96%, at least 97%, at least 98%, or at least 99% sequence identity therewith). In some embodiments, the multispecific, preferably bispecific, antibody is monovalent with respect to binding IL-31 and monovalent with respect to binding IL-13. In some embodiments, the multispecific, preferably bispecific, antibody has an IgG, preferably an IgG1 isotype. Preferably, the first part and the second part specifically bind to, respectively, IL-31 and IL-13, preferably human IL-31 and human IL-13.

In some embodiments, the multispecific, preferably bispecific, antibody is multivalent, preferably bivalent. In some embodiments, the multispecific, preferably bispecific, antibody is monovalent with respect to binding IL-31 and monovalent with respect to binding to IL-13.

In some embodiments, the multispecific, preferably bispecific, antibody is multivalent, e.g., bivalent, with respect to binding IL-31. In some embodiments, the multispecific, preferably bispecific, antibody is multivalent, e.g., bivalent, with respect to binding IL-13.

In preferred embodiments, the multispecific, preferably bispecific, antibody has an IgG, preferably an IgG1 isotype.

In some embodiments, the multispecific, preferably bispecific, antibody is a humanized, chimeric, or human antibody. In some embodiments, the multispecific, preferably bispecific, antibody is a humanized antibody.

In preferred embodiments, the IL-31 and IL-13 are human.

In some embodiments, the multispecific, preferably bispecific, antibody comprises:
a first part that binds, preferably specifically binds, to a human IL-31 epitope, wherein the epitope consists of amino acid residues 535, D37, V38, K40, I41, E44, N81, P86, R89, A90, K93, R96, K101, D105, E106, I108, E109, H110, D112, K113, L114, I115, F116, Q117, D118, A119, P120, E121, and T122 of human IL-31, preferably wherein human IL-31 is represented by SEQ ID NO: 143 or SEQ ID NO: 173, and;
a second part that binds, preferably specifically binds, to IL-13, comprising a heavy chain variable region (VH) comprising or consisting of the amino acid sequence shown in SEQ ID NO: 17 and a light chain variable region (VL) comprising or consisting of the amino acid sequence shown in SEQ ID NO: 18.

Methods for generating the first part that binds to the human IL-31 epitope described herein and assays for binding confirmation are discussed elsewhere herein, and further discussed in the experimental section.

Non-limiting exemplary positions in the light chain variable region of the first part that binds to human IL-31 that can be substituted by a different amino acid (VL positions are numbered starting from the aspartic acid (D) at position 1 of the VL represented by SEQ ID NO: 41) are Asp1, Gly27, Asn28, His30, Asn50, Lys52, Thr53, Leu54, Ala55, Asp56, Glu68, and Ser93. In some embodiments the VL position that is substituted by a different amino acid is selected from the group consisting of Asp1, Gly27, His30, Asn50, Leu54, Asp56, and Glu68, or from a combination thereof. Non-limiting exemplary positions in the heavy chain variable region of the first part that binds to human IL-31 that can be substituted (VH positions are numbered starting from the glutamine (Q) at position 1 of the VH represented by SEQ ID NO: 40) are Leu29, Trp33, Gly54, Asp55, Gly56, Lys65, Asp100, Gly101, Tyr102, Ala104, Ala105, and Asp110. In some embodiments, the VH position that is substituted by a different amino acid is selected from the group consisting of Leu29, Gly54, Asp55, Asp100, Tyr102, and Asp110, or from a combination thereof.

In some embodiments, the first part that binds to the human IL-31 epitope comprises a heavy chain variable region (VH) comprising at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29 and a light chain variable region (VL) comprising at least 80%, at least 85%, at least 90%, at least 95%, at least 96%, at least 97%, at least 98%, or at least 99% sequence identity with SEQ ID NO: 8 or SEQ ID NO: 30 or SEQ ID NO:31 or SEQ ID NO:32. In some embodiments, the first part that binds to the human IL-31 epitope comprises a heavy chain variable region (VH) comprising at least 80%, at least 85%, at least 90%, at least 95%, at least 96%, at least 97%, at least 98%, or at least 99% sequence identity with SEQ ID NO: 7 and a light chain variable region (VL) comprising at least 80%, at least 85%, at least 90%, at least 95%, at least 96%, at least 97%, at least 98%, or at least 99% sequence identity with SEQ ID NO: 8. In some embodiments, the heavy chain variable region comprises relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1). In some embodiments, the light chain variable region comprises relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

In some embodiments, the multispecific, preferably bispecific, antibody binds to human IL-31 with dissociation constant (KD) of less than 100 pM, less than 90 pM, less than 80 pM, less than 70 pM, less than 60 pM, less than 50 pM, less than 30 pM, less than 25 pM, less than 20 pM, less than 15 pM, less than 10 pM, or less than 5 pM or less than about 5 pM as measured using surface plasmon resonance, preferably as measured using a Biacore instrument following the manufacturer's instructions. In some embodiments, the multispecific, preferably bispecific, antibody binds to human IL-31 with a dissociation constant (KD) of less than 30 pM, less than 25 pM, less than 20 pM, less than 15 pM, less than 10 pM or less than about 10 pM, e.g., about 5 pM, as measured using surface plasmon resonance, preferably as measured using a Biacore instrument (e.g., a Biacore T200, T100, or 8K instrument as discussed elsewhere herein).

In some embodiments, the multispecific, preferably bispecific, antibody binds to human IL-13 with a dissociation constant (KD) of less than 30 pM, less than 25 pM, less than 20 pM, less than 15 pM, less than 10 pM, less than 5 pM, less than 1 pM or less than about 1 pM, as measured using surface plasmon resonance, preferably as measured using a Biacore instrument (e.g., a Biacore T200, T100, or 8K instrument as discussed elsewhere herein).

In some embodiments, the multispecific, preferably bispecific, antibody binds to human IL-31 and human IL-13 with a dissociation constant of less than 30 pM, less than 25 pM, less than 20 pM, less than 15 pM, less than 10 pM, less than 5 pM, less than 1 pM or less than about 1 pM, as measured using SET (solution equilibrium titration), e.g., as described by Friguet et al., J Immunol Methods; 77(2): 305-319, 1985. SET allows the determination of antibody-antigen interaction affinities for tight binders. This technique does not require immobilization or labeling of either interaction partner. A constant concentration of antibody (at or below the expected KD) is co-incubated with serial antigen dilutions until equilibrium is reached. The fraction of unbound antibody is determined by transferring the antigen-antibody mixtures on antigen-coated plates for a brief incubation. The free antibody consequently binds to the coated antigen and is, after a washing step to remove antibody-antigen complexes, detected with an electrochemiluminescence-labeled detection antibody. The resulting signal is plotted versus the antigen concentration. The KD is determined by non-linear curve fitting with a 1:2 binding model for bivalent monospecific antibodies, and with a 1:1 binding model for monovalent bispecific antibodies. To measure KD values down to the low picomolar level, a sensitive read-out technology such as electrochemiluminescence (ECL) is required (Haenel et al., Anal Biochem; 339(1):182-184, 2005). A further example of application of SET for determination of KD of a multispecific antibody described herein is provided in the experimental section.

In some embodiments, the multispecific, preferably bispecific, antibody has an improved affinity for human IL-31 compared to an anti-IL-31 antibody comprising:
i) a VH comprising SEQ ID NO: 157 and a VL comprising SEQ ID NO: 158;
ii) a VH comprising SEQ ID NO: 159 and a VL comprising SEQ ID NO: 160, and/or;
iii) a VH comprising SEQ ID NO: 40 and a VL comprising SEQ ID NO: 41, as measured using surface plasmon resonance, preferably as measured using a Biacore instrument.

In some embodiments, the multispecific, preferably bispecific, antibody has an improved affinity for human IL-31 compared to an anti-IL-31 antibody comprising:
i) a heavy chain comprising SEQ ID NO: 147 and a light chain comprising SEQ ID NO: 148;
ii) a heavy chain comprising SEQ ID NO: 155 and a light chain comprising SEQ ID NO: 156, and/or;
iii) a heavy chain comprising SEQ ID NO: 153 and a light chain comprising SEQ ID NO: 154,
as measured using surface plasmon resonance, preferably as measured using a Biacore instrument.

Improved affinity can manifest as a lower dissociation constant (KD) for IL-31, preferably human IL-31, for example at least a two-fold, at least a three-fold, at least a four-fold, at least a five-fold, at least a six-fold, at least a seven-fold, at least an eight-fold, at least a nine-fold, at least a ten-fold, or lower, dissociation constant relative to a reference multispecific antibody.

In some embodiments, the multispecific, preferably bispecific, antibody inhibits human IL-31 activity, for example inhibits human IL-31 activity with an IC50 of less than 100 pM, less than 90 pM, less than 80 pM, less than 70 pM, less than 60 pM, less than 50 pM, less than 30 pM, less than 25 pM, less than 20 pM, or less than 15 pM or less than about 15 pM, e.g., about 11 pM or less. In some embodiments, it inhibits human IL-31 activity with an IC50 of less than 30 pM, less than 25 pM, less than 20 pM or less than about 20 pM. Preferably, inhibition of IL-31 activity is measured using an IL-31 dependent Baf3 cell proliferation assay as described elsewhere herein, a further example of which is provided in the experimental section herein.

In some embodiments, the multispecific, preferably bispecific, antibody inhibits human IL-31 activity to a greater extent compared to an anti-IL-31 antibody comprising:
i) a VH comprising SEQ ID NO: 157 and a VL comprising SEQ ID NO: 158;
ii) a VH comprising SEQ ID NO: 159 and a VL comprising SEQ ID NO: 160, and/or;
iii) a VH comprising SEQ ID NO: 40 and a VL comprising SEQ ID NO: 41, preferably as measured using an IL-31 dependent Baf3 cell proliferation assay.

In some embodiments, the multispecific, preferably bispecific, antibody inhibits human IL-31 activity to a greater extent compared to an anti-IL-31 antibody comprising:
i) a heavy chain comprising SEQ ID NO: 147 and a light chain comprising SEQ ID NO: 148;
ii) a heavy chain comprising SEQ ID NO: 155 and a light chain comprising SEQ ID NO: 156,
iii) a heavy chain comprising SEQ ID NO: 153 and a light chain comprising SEQ ID NO: 154, and/or;
iv) a heavy chain comprising SEQ ID NO: 174 and a light chain comprising SEQ ID NO: 175,
preferably as measured using an IL-31 dependent Baf3 cell proliferation assay.

Improved inhibition (potency) can manifest as a lower IC50 value for human IL-31, for example at least a two-fold, at least a three-fold, at least a four-fold, at least a five-fold, at least a six-fold, at least a seven-fold, at least an eight-fold, at least a nine-fold, at least a ten-fold, or lower, IC50 value relative to a reference multispecific antibody.

In some embodiments, the multispecific, preferably bispecific, antibody inhibits human IL-13 activity, for example inhibits human IL-13 activity with an IC50 of less than 50 pM, less than 40 pM, less than 30 pM, less than 20 pM, less than 15 pM or less than about 15 pM, or less than 10 pM or less than about 10 pM, as measured in a HEK-Blue cell reporter assay. Such assays are known to the skilled person. For example, HEK-Blue reporter cells can be incubated with recombinant or native (e.g., contained in supernatants of MyLa cells) human IL-13 in the presence of increasing concentrations of multispecific antibody, for example for about 24 hours, and inhibition of the secretion of secreted alkaline phosphatase (SEAP) can be determined using the QUANTI-Blue™ method or any other suitable method available to the skilled person.

In some embodiments, the multispecific, preferably bispecific, antibody described herein binds to cynomolgus monkey IL-31 and IL-13.

In other embodiments, an anti-IL-13 part comprised by a multispecific e.g., bispecific antibody described herein, has a lower binding affinity and/or potency than a monospecific IL-13 antibody disclosed herein, but it nevertheless provides a biological response e.g., inhibits IL-13 signaling.

In some embodiments, the multispecific, preferably bispecific, antibody is an isolated antibody.

### Formats of Multispecific Antibodies

Multispecific antibody formats to be used in accordance with the disclosure are well known to the skilled person. Multispecific antibodies may be multivalent and/or monovalent with respect to binding each antigen. Bispecific antibodies, for example, include but are not limited to bivalent (monovalent with respect to binding each antigen), trivalent (bivalent with respect to binding one antigen and monovalent with respect to binding the other antigen), or tetravalent (bivalent with respect to binding both antigens). In some embodiments, bispecific antibodies are bivalent and are monovalent with respect to binding IL-31 and monovalent with respect to binding IL-13.

Non-limiting exemplary multispecific antibody formats include, but are not limited to, bivalent bispecific F(ab)2 format, bivalent bispecific Fab-scFv format, bivalent bispecific scFv-scFv format, bivalent bispecific IgG format, trivalent bispecific IgG format e.g., a whole antibody molecule specific to one antigen having e.g., a single chain Fab (scFab) linked to the C-terminal end of the molecule specific to another antigen, tetravalent bispecific IgG format, trivalent bispecific scFv3 format, trivalent bispecific (scFv)2-Fab format, trivalent bispecific F(ab)3 format, trivalent trispecific scFv3 format, trivalent trispecific Fab-scFv2 format (e.g., a Fab fragment specific to one antigen C-terminally linked to two scFvs specific to another antigen, one on each chain), trivalent trispecific F(ab)3 format, or trivalent trispecific IgG format, or tetravalent trispecific IgG format. Other exemplary formats incorporate sdAbs or fragments thereof such as a VHH, for example a Fab-VHH or scFv-VHH format.

Optionally, the multispecific antibody may be incorporated into an antibody-drug conjugate.

In some embodiments, a multispecific antibody comprises an antibody, scFv or Fab, or half IgG antibody, or fragments thereof, having binding specificity for IL-31; and one or more of an antibody, scFv or Fab, or half IgG antibody, or fragments thereof, having binding specificity for IL-13.

In some embodiments, the multispecific antibody comprises an scFv or Fab, or fragment thereof, having binding specificity for IL-31; and an scFv or Fab, or fragment thereof, having binding specificity for IL-13.

In some embodiments, the multispecific antibody comprises two scFvs or Fabs, or fragments thereof, having binding specificity for IL-31; and an antibody, or fragment thereof, having binding specificity for IL-13.

In some embodiments, the multispecific antibody comprises an scFv, or fragment thereof, having binding specificity for IL-31; and a Fab, or fragment thereof, having binding specificity for IL-13.

In some embodiments, the multispecific antibody has an antibody:scFv (“X style”) format e.g., with an IL-31 antibody joined to two IL-13 scFvs (e.g. VL-VH format), each scFv being attached via an external linker to the C-terminus of one of the polypeptide chains of the IL-31 antibody heavy chain (in particular, the C-terminus of the Fc region).

In some embodiments, the scFv-Fab (“bottle opener”) format may be used e.g., with a IL-31 binding part (anti-IL-31 half antibody, having an anti-IL-31 antibody heavy chain and light chain, i.e., a IL-31 antigen-binding Fab and an Fc polypeptide chain) pairing with an IL-13 binding arm (anti-IL-13 half antibody, having an anti-IL-13 scFv and an Fc polypeptide chain) via knob and hole or FAE mutations that favor heterodimerization of the Fc domains.

In some embodiments, the multispecific antibody is an IgG1 isotype comprising an IL-31 half antibody (an anti-IL-31 antibody heavy chain and light chain, i.e., an anti-IL-31 antigen-binding Fab and an Fc polypeptide chain) paired with an anti-IL-13 half antibody (an IL-13 antibody heavy chain and light chain, i.e., an IL-13 antigen-binding Fab and an Fc polypeptide chain).

Protocols for generating multispecific e.g., bispecific/heterodimeric antibodies are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bispecific structure using a heterobifunctional reagent having an amine-reactive group and a sulfhydryl reactive group as described in, e.g., U.S. Pat. No. 4,433,059; bispecific antibodies generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions (Fd regions) of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multi-specific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VL and VH domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; string of VH domains (or VL domains in family members) connected by peptide linkages with crosslinkable groups at the C-terminus further associated with VL domains to form a series of FVs (or scFvs), as described in, e.g., U.S. Pat. No. 5,864,019; VL and VH domains, scFvs, or Fabs wherein one of the antigens is bound monovalently and one of the antigens is bound bivalently, optionally comprising heterodimeric Fc regions, as described in, e.g., WO2011/028952; and single chain binding polypeptides with both a VL and VH domain linked through a peptide linker are combined into multivalent structures through non-covalent or chemical crosslinking to form, e.g., homobivalent, heterobivalent, trivalent, and tetravalent structures using both scFv or diabody type format, as described in, e.g., U.S. Pat. No. 5,869,620. Additional exemplary multispecific and bispecific antibodies and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1.

Accordingly, in some embodiments, the multispecific antibodies of the present invention comprise an IL-31 binding domain and an IL-13 binding domain in any one of the multispecific or bispecific formats known in the art, e.g., as described above. A preferred format for the multispecific antibodies described herein is described in more detail below.

### IgG Format

In preferred embodiments, a bispecific antibody has an IgG format comprising an anti-IL-31 half IgG antibody and an anti-IL-13 half IgG antibody, for example an anti-IL-31 half IgG1 antibody and an anti-IL-13 half IgG1 antibody.

An anti-IL-31 half IgG antibody comprises a polypeptide comprising a VL domain and a CL domain, and a second polypeptide comprising a VH domain, a CH1 domain, a hinge domain, a CH2 domain, and a CH3 domain (i.e., an Fd and an Fc), wherein said VL and VH domains comprise an anti-IL-31 antigen-binding domain. The anti-IL-13 half IgG antibody comprises a polypeptide comprising a VL domain and a CL domain, and a second polypeptide comprising a VH domain, a CH1 domain, a hinge domain, a CH2 domain, and a CH3 domain (i.e., an Fd and an Fc), wherein said VL and VH domains comprise an anti-IL-13 antigen-binding domain.

The Fc region (CH2 domains and/or CH3 domains) preferably comprises one or more mutations that favor heterodimerization of the two Fc polypeptide chains forming the Fc region of the mixed chain multispecific antibody, relative to unmodified polypeptide chains. Mutations for favoring heterodimerization are described in detail elsewhere herein, and include the K-i-H and FAE mutations described earlier herein.

It will also be appreciated by the skilled artisan that a multispecific, such as bispecific, antibody may alternatively be stabilized by covalently linking one or more of the polypeptide chains of the antibody. As used herein, “linking” refers to one portion of a multispecific, such as bispecific, antibody, or fragment thereof, being attached to another portion of the molecule. Direct attachment is a form of linkage, also referred to as “fusion”. Attachment may also be indirect and be mediated via linkers. For example, using an scFv in the format VH-internal linker-VL, the VH is linked indirectly to the VL, and directly to the internal linker (the linker may also be described in this example as being fused to both the VH and VL). Linkers are known in the art at may be chemical or peptidic. The linking may be along the polypeptide backbone (e.g., terminus to terminus), amino acid side chain to side chain, or amino acid side chain to terminus. Attachment may be covalent. Thus, in some embodiments, the part that binds to IL-31 and the part that binds to IL-31 in the multispecific, preferably bispecific, antibody is linked. In some embodiments, the linkage is direct linkage, and the parts are thus fused to each other. In some embodiments, an anti-IL-31 binding domain and/or an anti-IL-13 binding domain are fused to an Fc polypeptide chain. In some embodiments, an anti-IL-31 binding domain (e.g., an anti-IL-31 Fab) is linked, e.g., via a polypeptide linker, to the N-terminus of an anti-IL-13 binding domain. In some embodiments, an anti-IL-13 binding domain (e.g., an anti-IL-13 Fab) is linked, e.g., via a polypeptide linker, to the N-terminus of an anti-IL-31 binding domain.

Optionally, one or more chains of a multispecific, preferably bispecific, antibody may further comprise another antigen recognition domain, e.g., at least one additional VHH, scFv or Fab, resulting in a trivalent, tetravalent antibody etc. The at least one additional VHH, scFv or Fab may be disposed at the C-terminus of a polypeptide chain or disposed at the N-terminus of a polypeptide chain, for example disposed at the C-terminus of a polypeptide comprising a heavy chain constant domain, e.g., a CH2 domain or CH3 domain or disposed C-terminally to the CH3 domain of a one or more polypeptides of the multispecific antibody. The least one additional VHH, scFv or Fab may be disposed at the C-terminus of a polypeptide comprising the anti-IL-31 half antibody heavy chain or disposed at the C-terminus of a polypeptide comprising the anti-IL-31 half antibody light chain. The at least one additional VHH, scFv or Fab may be disposed at the N-terminus of a polypeptide comprising the anti-IL-31 half antibody heavy chain. The at least one additional VHH, scFv or Fab may be disposed at the N-terminus of a polypeptide comprising the anti-IL-31 antibody light chain. The at least one additional VHH, scFv or Fab may be disposed at the C-terminus of a polypeptide comprising the anti-IL-13 half antibody heavy chain or disposed at the C-terminus of a polypeptide comprising the anti-IL-13 half antibody light chain. The at least one additional VHH, scFv or Fab may be disposed at the N-terminus of a polypeptide comprising the anti-IL-13 half antibody heavy chain. The at least one additional VHH, scFv or Fab may be disposed at the N-terminus of a polypeptide comprising the anti-IL-13 antibody light chain. These additional antigen-binding domains, e.g., VHHs, scFvs or Fabs, may bind to IL-13, IL-31 or one or different antigens. Where the one or more additional antigen-binding domains, e.g., VHHs, scFvs or Fabs, recognizes IL-13 or IL-31, the antibody is bispecific. Where the one or more additional antigen-binding domains, e.g., VHHs, scFvs or Fabs, recognizes one antigen different from IL-13 or IL-31, the antibody is trispecific. The additional antigen-binding domain e.g., VHHs, scFvs or Fabs, may recognize one or more anti-inflammatory antigens.

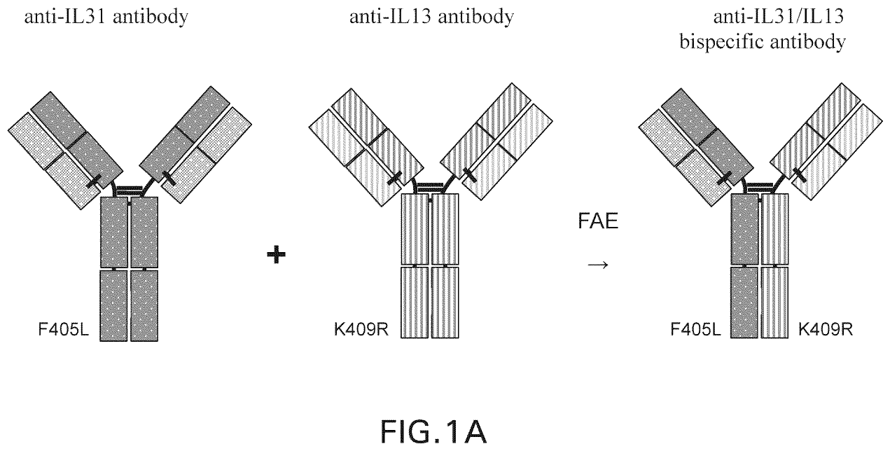

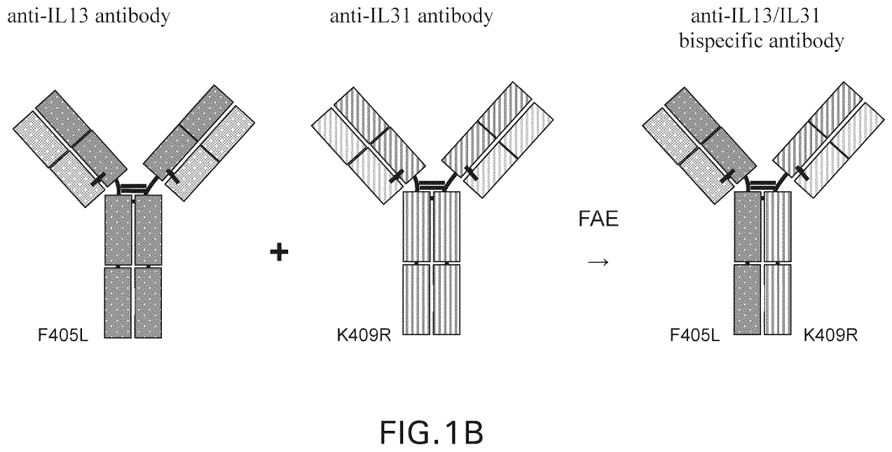

In preferred embodiments, a multispecific antibody of the disclosure is bispecific and has the structure depicted in FIG. 1A or 1B.

The amino acid sequences shown in Table 1 are non-limiting examples of sequences that can be comprised by anti-IL-31 monospecific anti-IL-31/IL-13 multispecific, preferably bispecific, antibodies described herein, as well as by fragments thereof. In the case of anti-IL-31/IL-13 bispecific antibodies, they can for example be generated in IgG format (for example IgG1 format) and can comprise a part binding to IL-31 (anti-IL-31 half antibody, having an anti-IL-31 antibody heavy chain and light chain, i.e., an IL-31 antigen-binding Fab and an Fc polypeptide chain) paired with a part binding to IL-13 (anti-IL-13 half antibody, having an anti-IL-13 antibody heavy chain and light chain, i.e., an IL-13 antigen-binding Fab and an Fc polypeptide chain) via mutations that favor heterodimerization of the Fc domains. In such embodiments, the anti-IL-31/IL-13 bispecific antibody preferably specifically binds to IL-31 and IL-13, preferably human IL-31 and human IL-13.

TABLE 1
Non-limiting exemplary anti-IL-31 monospecific and anti-IL-31/IL-13
multispecific Antibody Sequences.

| | | |
|---|---|---|
| SEQ ID NUMBER | Binding Region | Sequence |

IL-31 binding portions

| | | |
|---|---|---|
| SEQ ID NO: 1 | HCDR1 | RYWMQ |
| (Kabat) | | |
| SEQ ID NO: 2 | HCDR2 | AIYPAEGDTRYSQKFKG |
| (Kabat) | | |
| SEQ ID NO: 3 | HCDR3 | PEGPYAAPYGMHY |
| (Kabat) | | |
| SEQ ID NO: 4 | LCDR1 | RASRNIGNYLA |
| (Kabat) | | |
| SEQ ID NO: 5 | LCDR2 | YAKTSAS |
| (Kabat) | | |
| SEQ ID NO: 6 | LCDR3 | QHFWSTPWT |
| (Kabat) | | |
| SEQ ID NO: 7 | V_{H} | QVQLVQSGAEVKKPGSSVKVSCKAS |
| | | GYTRTRYWMQWVRQAPGQGLEWMGAI |
| | | YPAEGDTRYSQKFKGRVTITADKSTSTAY |
| | | MELSSLRSEDTAVYYCAFPEGPYAAPYG |
| | | MHYWGQGTTVTVSS |
| SEQ ID NO: 8 | V_{L} | AIQMTQSPSSLSASVGDRVTITCRASR |
| | | NIGNYLAWYQQKPGKVPKLLIYYAKTSA |
| | | SGVPSRFSGSRSGTDYTLTISSLQPEDVAT |
| | | YYCQHFWSTPWTFGGGTKVEIK |
| SEQ ID NO: 9 | Light Chain | AIQMTQSPSSLSASVGDRVTITCRASR |
| | | NIGNYLAWYQQKPGKVPKLLIYYAKTSA |
| | | SGVPSRFSGSRSGTDYTLTISSLQPEDVAT |
| | | YYCQHFWSTPWTFGGGTKVEIKRTVAAP |
| | | SVFIFPPSDEQLKSGTASVVCLLNNFYPRE |
| | | AKVQWKVDNALQSGNSQESVTEQDSKD |
| | | STYSLSSTLTLSKADYEKHKVYACEVTH |
| | | QGLSSPVTKSFNRGEC |
| SEQ ID NO: 10 | IL-31 antibody | QVQLVQSGAEVKKPGSSVKVSCKAS |
| | Heavy Chain | GYTRTRYWMQWVRQAPGQGLEWMGAI |
| | (LALA; YTE; | YPAEGDTRYSQKFKGRVTITADKSTSTAY |
| | F405L) | MELSSLRSEDTAVYYCAFPEGPYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSCDKTHTCPPCPAPEAAGGPSVFLFPPK |
| | | PKDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFLLYSKLTVDKSR |
| | | WQQGNVFSCSVMHEALHNHYTQKSLSL |
| | | SPGK |
| SEQ ID NO: 21 | HCDR2 | AIYPGEGDTRYSQKFKG |
| (Kabat) | | |
| SEQ ID NO: 23 | HCDR3 | PEGQYAAPYGMHY |
| (Kabat) | | |
| SEQ ID NO: 24 | LCDR1 | RASRNIHNYLA |
| (Kabat) | | |
| SEQ ID NO: 25 | LCDR2 | YAKTSAE |
| (Kabat) | | |
| SEQ ID NO: 26 | LCDR3 | QHFWKTPWT |
| (Kabat) | | |
| SEQ ID NO: 28 | V_{H} | QVQLVQSGAEVKKPGSSVKVSCKAS |
| | | GYTRTRYWMQWVRQAPGQGLEWMGAI |
| | | YPGEGDTRYSQKFKGRVTITADKSTSTAY |
| | | MELSSLRSEDTAVYYCAFPEGPYAAPYG |
| | | MHYWGQGTTVTVSS |
| SEQ ID NO: 29 | V_{H} | QVQLVQSGAEVKKPGSSVKVSCKAS |
| | | GYTRTRYWMQWVRQAPGQGLEWMGAI |
| | | YPGEGDTRYSQKFKGRVTITADKSTSTAY |
| | | MELSSLRSEDTAVYYCAFPEGQYAAPYG |
| | | MHYWGQGTTVTVSS |
| SEQ ID NO: 30 | V_{L} | AIQMTQSPSSLSASVGDRVTITCRASR |
| | | NIGNYLAWYQQKPGKVPKLLIYYAKTSA |
| | | EGVPSRFSGSRSGTDYTLTISSLQPEDVAT |
| | | YYCQHFWKTPWTFGGGTKVEIK |
| SEQ ID NO: 31 | V_{L} | AIQMTQSPSSLSASVGDRVTITCRASR |
| | | NIGNYLAWYQQKPGKVPKLLIYYAKTSA |
| | | SGVPSRFSGSRSGTDYTLTISSLQPEDVAT |
| | | YYCQHFWKTPWTFGGGTKVEIK |
| SEQ ID NO: 32 | V_{L} | AIQMTQSPSSLSASVGDRVTITCRASR |
| | | NIHNYLAWYQQKPGKVPKLLIYYAKTSA |
| | | SGVPSRFSGSRSGTDYTLTISSLQPEDVAT |
| | | YYCQHFWKTPWTFGGGTKVEIK |
| SEQ ID NO: 33 | Light Chain | AIQMTQSPSSLSASVGDRVTITCRASR |
| | | NIGNYLAWYQQKPGKVPKLLIYYAKTSA |
| | | EGVPSRFSGSRSGTDYTLTISSLQPEDVAT |
| | | YYCQHFWKTPWTFGGGTKVEIKRTVAAP |
| | | SVFIFPPSDEQLKSGTASVVCLLNNFYPRE |
| | | AKVQWKVDNALQSGNSQESVTEQDSKD |
| | | STYSLSSTLTLSKADYEKHKVYACEVTH |
| | | QGLSSPVTKSFNRGEC |
| SEQ ID NO: 34 | Light Chain | AIQMTQSPSSLSASVGDRVTITCRASR |
| | | NIGNYLAWYQQKPGKVPKLLIYYAKTSA |
| | | SGVPSRFSGSRSGTDYTLTISSLQPEDVAT |
| | | YYCQHFWKTPWTFGGGTKVEIKRTVAAP |
| | | SVFIFPPSDEQLKSGTASVVCLLNNFYPRE |
| | | AKVQWKVDNALQSGNSQESVTEQDSKD |
| | | STYSLSSTLTLSKADYEKHKVYACEVTH |
| | | QGLSSPVTKSFNRGEC |
| SEQ ID NO: 35 | Light Chain | AIQMTQSPSSLSASVGDRVTITCRASR |
| | | NIHNYLAWYQQKPGKVPKLLIYYAKTSA |
| | | SGVPSRFSGSRSGTDYTLTISSLQPEDVAT |
| | | YYCQHFWKTPWTFGGGTKVEIKRTVAAP |
| | | SVFIFPPSDEQLKSGTASVVCLLNNFYPRE |
| | | AKVQWKVDNALQSGNSQESVTEQDSKD |
| | | STYSLSSTLTLSKADYEKHKVYACEVTH |
| | | QGLSSPVTKSFNRGEC |
| SEQ ID NO: 37 | Fd | QVQLVQSGAEVKKPGSSVKVSCKAS |
| | | GYTRTRYWMQWVRQAPGQGLEWMGAI |
| | | YPGEGDTRYSQKFKGRVTITADKSTSTAY |
| | | MELSSLRSEDTAVYYCAFPEGPYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSC |
| SEQ ID NO: 38 | Fd | QVQLVQSGAEVKKPGSSVKVSCKAS |
| | | GYTRTRYWMQWVRQAPGQGLEWMGAI |
| | | YPGEGDTRYSQKFKGRVTITADKSTSTAY |
| | | MELSSLRSEDTAVYYCAFPEGQYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSC |
| SEQ ID NO: 39 | Fd | QVQLVQSGAEVKKPGSSVKVSCKAS |
| | | GYTRTRYWMQWVRQAPGQGLEWMGAI |
| | | YPAEGDTRYSQKFKGRVTITADKSTSTAY |
| | | MELSSLRSEDTAVYYCAFPEGPYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSC |
| SEQ ID NO: 42 | Fc (wild-type) | DKTHTCPPCPAPELLGGPSVFLFPPKP |
| | | KDTLMISRTPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFFLYSKLTVDKSR |
| | | WQQGNVFSCSVMHEALHNHYTQKSLSL |
| | | SPGK |
| SEQ ID NO: 44 | Fc (LALA; | DKTHTCPPCPAPEAAGGPSVFLFPPKP |
| | YTE; F405L) | KDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFLLYSKLTVDKSR |
| | | WQQGNVFSCSVMHEALHNHYTQKSLSL |
| | | SPGK |
| SEQ ID NO: 45 | Fc (LALA; | DKTHTCPPCPAPEAAGGPSVFLFPPKP |
| | YTE; K409R) | KDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFFLYSRLTVDKSR |
| | | WQQGNVFSCSVMHEALHNHYTQKSLSL |
| | | SPGK |
| SEQ ID NO: 46 | Fc with Knob | DKTHTCPPCPAPELLGGPSVFLFPPKP |
| | mutations (S354C, | KDTLMISRTPEVTCVVVDVSHEDPEVKF |
| | T366W) | NWYVDGVEVHNAKTKPREEQYNSTYRV |
| | | VSVLTVLHQDWLNGKEYKCKVSNKALP |
| | | APIEKTISKAKGQPREPQVYTLPPCREEM |
| | | TKNQVSLWCLVKGFYPSDIAVEWESNGQ |
| | | PENNYKTTPPVLDSDGSFFLYSKLTVDKS |
| | | RWQQGNVFSCSVMHEALHNHYTQKSLS |
| | | LSPGK |
| SEQ ID NO: 47 | Fc with Hole | DKTHTCPPCPAPELLGGPSVFLFPPKP |
| | mutations (Y349C, | KDTLMISRTPEVTCVVVDVSHEDPEVKF |
| | T366S, L368A, | NWYVDGVEVHNAKTKPREEQYNSTYRV |
| | Y407V) | VSVLTVLHQDWLNGKEYKCKVSNKALP |
| | | APIEKTISKAKGQPREPQVCTLPPSREEM |
| | | TKNQVSLSCAVKGFYPSDIAVEWESNGQ |
| | | PENNYKTTPPVLDSDGSFFLVSKLTVDKS |
| | | RWQQGNVFSCSVMHEALHNHYTQKSLS |
| | | LSPGK |
| SEQ ID NO: 48 | HCDR1 | GYTRTRY |
| (Chothia) | | |
| SEQ ID NO: 49 | HCDR2 | YPAEGD |
| (Chothia) | | |
| SEQ ID NO: 50 | HCDR3 | PEGPYAAPYGMHY |
| (Chothia) | | |
| SEQ ID NO: 51 | LCDR1 | SRNIGNY |
| (Chothia) | | |
| SEQ ID NO: 53 | LCDR3 | FWSTPW |
| (Chothia) | | |
| SEQ ID NO: 61 | HCDR2 | YPGEGD |
| (Chothia) | | |
| SEQ ID NO: 63 | HCDR3 | PEGQYAAPYGMHY |
| (Chothia) | | |
| SEQ ID NO: 64 | LCDR3 | FWKTPW |
| (Chothia) | | |
| SEQ ID NO: 65 | LCDR1 | SRNIHNY |
| (Chothia) | | |
| SEQ ID NO: 66 | HCDR1 | GYTRTRYW |
| (IMGT) | | |
| SEQ ID NO: 67 | HCDR2 | IYPAEGDT |
| (IMGT) | | |
| SEQ ID NO: 68 | HCDR3 | AFPEGPYAAPYGMHY |
| (IMGT) | | |
| SEQ ID NO: 69 | LCDR1 | RNIGNY |
| (IMGT) | | |
| SEQ ID NO: 71 | LCDR3 | QHFWSTPWT |
| (IMGT) | | |
| SEQ ID NO: 73 | HCDR2 | IYPGEGDT |
| (IMGT) | | |
| SEQ ID NO: 75 | LCDR3 | QHFWKTPWT |
| (IMGT) | | |
| SEQ ID NO: 76 | LCDR1 | RNIHNY |
| (IMGT) | | |
| SEQ ID NO: 77 | HCDR3 | AFPEGQYAAPYGMHY |
| (IMGT) | | |
| SEQ ID NO: 78 | HCDR1 | GYTRTRYWMQ |
| (Combined) | | |
| SEQ ID NO: 79 | HCDR2 | AIYPAEGDTRYSQKFKG |
| (Combined) | | |
| SEQ ID NO: 80 | HCDR3 | PEGPYAAPYGMHY |
| (Combined) | | |
| SEQ ID NO: 81 | LCDR1 | RASRNIGNYLA |
| (Combined) | | |
| SEQ ID NO: 82 | LCDR2 | YAKTSAS |
| (Combined) | | |
| SEQ ID NO: 83 | LCDR3 | QHFWSTPWT |
| (Combined) | | |
| SEQ ID NO: 91 | HCDR2 | AIYPGEGDTRYSQKFKG |
| (Combined) | | |
| SEQ ID NO: 93 | LCDR2 | YAKTSAE |
| (Combined) | | |
| SEQ ID NO: 94 | LCDR3 | QHFWKTPWT |
| (Combined) | | |
| SEQ ID NO: 95 | LCDR1 | RASRNIHNYLA |
| (Combined) | | |
| SEQ ID NO: 96 | HCDR3 | PEGQYAAPYGMHY |
| (Combined) | | |
| SEQ ID NO: 104 | IL-31 antibody | QVQLVQSGAEVKKPGSSVKVSCKAS |
| | heavy chain (LALA; | GYTRTRYWMQWVRQAPGQGLEWMGAI |
| | YTE; F405L) | YPGEGDTRYSQKFKGRVTITADKSTSTAY |
| | | MELSSLRSEDTAVYYCAFPEGPYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSCDKTHTCPPCPAPEAAGGPSVFLFPPK |
| | | PKDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFLLYSKLTVDKSR |
| | | WQQGNVFSCSVMHEALHNHYTQKSLSL |
| | | SPGK |
| SEQ ID NO: 105 | IL-31 antibody | QVQLVQSGAEVKKPGSSVKVSCKAS |
| | heavy chain (LALA; | GYTRTRYWMQWVRQAPGQGLEWMGAI |
| | YTE; F405L) | YPGEGDTRYSQKFKGRVTITADKSTSTAY |
| | | MELSSLRSEDTAVYYCAFPEGQYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSCDKTHTCPPCPAPEAAGGPSVFLFPPK |
| | | PKDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFLLYSKLTVDKSR |
| | | WQQGNVFSCSVMHEALHNHYTQKSLSL |
| | | SPGK |
| SEQ ID NO: 107 | IL-31 antibody | QVQLVQSGAEVKKPGSSVKVSCKAS |
| | heavy chain (LALA; | GYTRTRYWMQWVRQAPGQGLEWMGAI |
| | YTE; K409R) | YPGEGDTRYSQKFKGRVTITADKSTSTAY |
| | | MELSSLRSEDTAVYYCAFPEGPYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSCDKTHTCPPCPAPEAAGGPSVFLFPPK |
| | | PKDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFFLYSRLTVDKSR |
| | | WQQGNVFSCSVMHEALHNHYTQKSLSL |
| | | SPGK |
| SEQ ID NO: 108 | IL-31 antibody | QVQLVQSGAEVKKPGSSVKVSCKAS |
| | heavy chain (LALA; | GYTRTRYWMQWVRQAPGQGLEWMGAI |
| | YTE; K409R) | YPGEGDTRYSQKFKGRVTITADKSTSTAY |
| | | MELSSLRSEDTAVYYCAFPEGQYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSCDKTHTCPPCPAPEAAGGPSVFLFPPK |
| | | PKDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFFLYSRLTVDKSR |
| | | WQQGNVFSCSVMHEALHNHYTQKSLSL |
| | | SPGK |
| SEQ ID NO: 109 | IL-31 antibody | QVQLVQSGAEVKKPGSSVKVSCKAS |
| | heavy chain (LALA; | GYTRTRYWMQWVRQAPGQGLEWMGAI |
| | YTE; K409R) | YPAEGDTRYSQKFKGRVTITADKSTSTAY |
| | | MELSSLRSEDTAVYYCAFPEGPYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSCDKTHTCPPCPAPEAAGGPSVFLFPPK |
| | | PKDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFFLYSRLTVDKSR |
| | | WQQGNVFSCSVMHEALHNHYTQKSLSL |
| | | SPGK |

IL-13 binding portions

| | | |
|---|---|---|
| SEQ ID NO: 11 | HCDR1 | AYSVN |
| (Kabat) | | |
| SEQ ID NO: 12 | HCDR2 | MIWGDGKIVYNSALKS |
| (Kabat) | | |
| SEQ ID NO: 13 | HCDR3 | DGYYPYAMDN |
| (Kabat) | | |
| SEQ ID NO: 14 | LCDR1 | RASKSVDSYGNSFMH |
| (Kabat) | | |
| SEQ ID NO: 15 | LCDR2 | LASNLES |
| (Kabat) | | |
| SEQ ID NO: 16 | LCDR3 | QQNNEDPRT |
| (Kabat) | | |
| SEQ ID NO: 17 | V_{H} | QVTLRESGPALVKPTQTLTLTCTVSGF |
| | | SLSAYSVNWIRQPPGKALEWLAMIWGD |
| | | GKIVYNSALKSRLTISKDTSKNQVVLTMT |
| | | NMDPVDTATYYCAGDGYYPYAMDNWG |
| | | QGSLVTVSS |
| SEQ ID NO: 18 | V_{L} | DIVMTQSPDSLSVSLGERATINCRAS |
| | | KSVDSYGNSFMHWYQQKPGQPPKLLIYL |
| | | ASNLESGVPDRFSGSGSGTDFTLTISSLQA |
| | | EDVAVYYCQQNNEDPRTFGGGTKVEIK |
| SEQ ID NO: 19 | Light Chain | DIVMTQSPDSLSVSLGERATINCRAS |
| | | KSVDSYGNSFMHWYQQKPGQPPKLLIYL |
| | | ASNLESGVPDRFSGSGSGTDFTLTISSLQA |
| | | EDVAVYYCQQNNEDPRTFGGGTKVEIKR |
| | | TVAAPSVFIFPPSDEQLKSGTASVVCLLN |
| | | NFYPREAKVQWKVDNALQSGNSQESVT |
| | | EQDSKDSTYSLSSTLTLSKADYEKHKVY |
| | | ACEVTHQGLSSPVTKSFNRGEC |
| SEQ ID NO: 20 | IL-13 antibody | QVTLRESGPALVKPTQTLTLTCTVSGF |
| | Heavy Chain | SLSAYSVNWIRQPPGKALEWLAMIWGD |
| | (LALA; YTE; | GKIVYNSALKSRLTISKDTSKNQVVLTMT |
| | K409R) | NMDPVDTATYYCAGDGYYPYAMDNWG |
| | | QGSLVTVSSASTKGPSVFPLAPSSKSTSG |
| | | GTAALGCLVKDYFPEPVTVSWNSGALTS |
| | | GVHTFPAVLQSSGLYSLSSVVTVPSSSLGT |
| | | QTYICNVNHKPSNTKVDKRVEPKSCDKT |
| | | HTCPPCPAPEAAGGPSVFLFPPKPKDTLYI |
| | | TREPEVTCVVVDVSHEDPEVKFNWYVD |
| | | GVEVHNAKTKPREEQYNSTYRVVSVLTV |
| | | LHQDWLNGKEYKCKVSNKALPAPIEKTI |
| | | SKAKGQPREPQVYTLPPSREEMTKNQVS |
| | | LTCLVKGFYPSDIAVEWESNGQPENNYK |
| | | TTPPVLDSDGSFFLYSRLTVDKSRWQQG |
| | | NVFSCSVMHEALHNHYTQKSLSLSPGK |
| SEQ ID NO: 42 | Fc (wild-type) | DKTHTCPPCPAPELLGGPSVFLFPPKP |
| | | KDTLMISRTPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFFLYSKLTVDKSR |
| | | WQQGNVFSCSVMHEALHNHYTQKSLSL |
| | | SPGK |
| SEQ ID NO: 43 | Fd | QVTLRESGPALVKPTQTLTLTCTVSGF |
| | | SLSAYSVNWIRQPPGKALEWLAMIWGD |
| | | GKIVYNSALKSRLTISKDTSKNQVVLTMT |
| | | NMDPVDTATYYCAGDGYYPYAMDNWG |
| | | QGSLVTVSSASTKGPSVFPLAPSSKSTSG |
| | | GTAALGCLVKDYFPEPVTVSWNSGALTS |
| | | GVHTFPAVLQSSGLYSLSSVVTVPSSSLGT |
| | | QTYICNVNHKPSNTKVDKRVEPKSC |
| SEQ ID NO: 44 | Fc (LALA; | DKTHTCPPCPAPEAAGGPSVFLFPPKP |
| | YTE; F405L) | KDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFLLYSKLTVDKSR |
| | | WQQGNVFSCSVMHEALHNHYTQKSLSL |
| | | SPGK |
| SEQ ID NO: 45 | Fc (LALA; | DKTHTCPPCPAPEAAGGPSVFLFPPKP |
| | YTE; K409R) | KDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLTCLVKGFYPSDIAVEWESNGQP |
| | | ENNYKTTPPVLDSDGSFFLYSRLTVDKSR |
| | | WQQGNVFSCSVMHEALHNHYTQKSLSL |
| | | SPGK |
| SEQ ID NO: 46 | Fc with Knob | DKTHTCPPCPAPELLGGPSVFLFPPKP |
| | mutations (S354C, | KDTLMISRTPEVTCVVVDVSHEDPEVKF |
| | T366W) | NWYVDGVEVHNAKTKPREEQYNSTYRV |
| | | VSVLTVLHQDWLNGKEYKCKVSNKALP |
| | | APIEKTISKAKGQPREPQVYTLPPCREEM |
| | | TKNQVSLWCLVKGFYPSDIAVEWESNGQ |
| | | PENNYKTTPPVLDSDGSFFLYSKLTVDKS |
| | | RWQQGNVFSCSVMHEALHNHYTQKSLS |
| | | LSPGK |
| SEQ ID NO: 47 | Fc with Hole | DKTHTCPPCPAPELLGGPSVFLFPPKP |
| | mutations (Y349C, | KDTLMISRTPEVTCVVVDVSHEDPEVKF |
| | T366S, L368A, | NWYVDGVEVHNAKTKPREEQYNSTYRV |
| | Y407V) | VSVLTVLHQDWLNGKEYKCKVSNKALP |
| | | APIEKTISKAKGQPREPQVCTLPPSREEM |
| | | TKNQVSLSCAVKGFYPSDIAVEWESNGQ |
| | | PENNYKTTPPVLDSDGSFFLVSKLTVDKS |
| | | RWQQGNVFSCSVMHEALHNHYTQKSLS |
| | | LSPGK |
| SEQ ID NO: 54 | HCDR1 | GFSLSAY |
| (Chothia) | | |
| SEQ ID NO: 55 | HCDR2 | WGDGK |
| (Chothia) | | |
| SEQ ID NO: 56 | HCDR3 | DGYYPYAMDN |
| (Chothia) | | |
| SEQ ID NO: 57 | LCDR1 | SKSVDSYGNSF |
| (Chothia) | | |
| SEQ ID NO: 59 | LCDR3 | NNEDPR |
| (Chothia) | | |
| SEQ ID NO: 84 | HCDR1 | GFSLSAYSVN |
| (Combined) | | |
| SEQ ID NO: 85 | HCDR2 | MIWGDGKIVYNSALKS |
| (Combined) | | |
| SEQ ID NO: 86 | HCDR3 | DGYYPYAMDN |
| (Combined) | | |
| SEQ ID NO: 87 | LCDR1 | RASKSVDSYGNSFMH |
| (Combined) | | |
| SEQ ID NO: 88 | LCDR2 | LASNLES |
| (Combined) | | |
| SEQ ID NO: 89 | LCDR3 | QQNNEDPRT |
| (Combined) | | |
| SEQ ID NO: 97 | HCDR1 | GFSLSAYS |
| (IMGT) | | |
| SEQ ID NO: 98 | HCDR2 | IWGDGKI |
| (IMGT) | | |
| SEQ ID NO: 99 | HCDR3 | AGDGYYPYAMDN |
| (IMGT) | | |
| SEQ ID NO: 100 | LCDR1 | KSVDSYGNSF |
| (IMGT) | | |
| SEQ ID NO: 102 | LCDR3 | QQNNEDPRT |
| (IMGT) | | |
| SEQ ID NO: 110 | IL-13 antibody | QVTLRESGPALVKPTQTLTLTCTVSGF |
| | Heavy Chain | SLSAYSVNWIRQPPGKALEWLAMIWGD |
| | (LALA; YTE; | GKIVYNSALKSRLTISKDTSKNQVVLTMT |
| | F405L) | NMDPVDTATYYCAGDGYYPYAMDNWG |
| | | QGSLVTVSSASTKGPSVFPLAPSSKSTSG |
| | | GTAALGCLVKDYFPEPVTVSWNSGALTS |
| | | GVHTFPAVLQSSGLYSLSSVVTVPSSSLGT |
| | | QTYICNVNHKPSNTKVDKRVEPKSCDKT |
| | | HTCPPCPAPEAAGGPSVFLFPPKPKDTLYI |
| | | TREPEVTCVVVDVSHEDPEVKFNWYVD |
| | | GVEVHNAKTKPREEQYNSTYRVVSVLTV |
| | | LHQDWLNGKEYKCKVSNKALPAPIEKTI |
| | | SKAKGQPREPQVYTLPPSREEMTKNQVS |
| | | LTCLVKGFYPSDIAVEWESNGQPENNYK |
| | | TTPPVLDSDGSFLLYSKLTVDKSRWQQG |
| | | NVFSCSVMHEALHNHYTQKSLSLSPGK |

Additional sequences

| | | |
|---|---|---|
| SEQ ID NO: 176 | anti-IL-31 | QVQLVQSGAEVKKPGSSVKVSCKAS |
| | Heavy Chain | GYTRTRYWMQWVRQAPGQGLEWMGAI |
| | | YPAEGDTRYSQKFKGRVTITADKSTSTAY |
| | | MELSSLRSEDTAVYYCAFPEGPYAAPYG |
| | | MHYWGQGTTVTVSSASTKGPSVFPLAPS |
| | | SKSTSGGTAALGCLVKDYFPEPVTVSWN |
| | | SGALTSGVHTFPAVLQSSGLYSLSSVVTV |
| | | PSSSLGTQTYICNVNHKPSNTKVDKRVEP |
| | | KSCDKTHTCPPCPAPEAAGGPSVFLFPPK |
| | | PKDTLYITREPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSREEM |
| | | TKNQVSLSCAVKGFYPSDIAVEWESNGQ |
| | | PENNYKTTPPVLDSDGSFFLVSKLTVDKS |
| | | RWQQGNVFSCSVMHEALHNHYTQKSLS |
| | | LSPGK |
| SEQ ID NO: 177 | anti-IL-31 Light | AIQMTQSPSSLSASVGDRVTITCR |
| | Chain | ASRNIGNYLAWYQQKPGKVPKLLIY |
| | | YAKTSASGVPSRFSGSRSGTDYTLTIS |
| | | SLQPEDVATYYCQHFWSTPWTFGGG |
| | | TKVEIKRTVAAPSVFIFPPSDEQLKSGT |
| | | ASVVCLLNNFYPREAKVQWKVDNA |
| | | LQSGNSQESVTEQDSKDSTYSLSSTLT |
| | | LSKADYEKHKVYACEVTHQGLSSPV |
| | | TKSFNRGEC |
| SEQ ID NO: 178 | anti-IL-13 | QVTLRESGPALVKPTQTLTLTCTVSGF |
| | Heavy Chain | SLSAYSVNWIRQPPGKALEWLAMIWGD |
| | | GKIVYNSALKSRLTISKDTSKNQVVLTMT |
| | | NMDPVDTATYYCAGDGYYPYAMDNWG |
| | | QGSLVTVSSASTKGPSVFPLAPSSKSTSG |
| | | GTAALGCLVKDYFPEPVTVSWNSGALTS |
| | | GVHTFPAVLQSSGLYSLSSVVTVPSSSLGT |
| | | QTYICNVNHKPSNTKVDKRVEPKSCDKT |
| | | HTCPPCPAPEAAGGPSVFLFPPKPKDTLYI |
| | | TREPEVTCVVVDVSHEDPEVKFNWYVD |
| | | GVEVHNAKTKPREEQYNSTYRVVSVLTV |
| | | LHQDWLNGKEYKCKVSNKALPAPIEKTI |
| | | SKAKGQPREPQVYTLPPSREEMTKNQVS |
| | | LWCLVKGFYPSDIAVEWESNGQPENNYK |
| | | TTPPVLDSDGSFFLYSKLTVDKSRWQQG |
| | | NVFSCSVMHEALHNHYTQKSLSLSPGK |
| SEQ ID NO: 179 | anti-IL-13 Light | DIVMTQSPDSLSVSLGERATINCRAS |
| | Chain | KSVDSYGNSFMHWYQQKPGQPPKLLIYL |
| | | ASNLESGVPDRFSGSGSGTDFTLTISSLQA |
| | | EDVAVYYCQQNNEDPRTFGGGTKVEIKR |
| | | TVAAPSVFIFPPSDEQLKSGTASVVCLLN |
| | | NFYPREAKVQWKVDNALQSGNSQESVT |
| | | EQDSKDSTYSLSSTLTLSKADYEKHKVY |
| | | ACEVTHQGLSSPVTKSFNRGEC |

### Modification of the Antibodies of the Present Disclosure

The present application includes variants of the antibodies described herein and/or fragments thereof having various modifications in variable regions and/or constant regions. For example, an Fc polypeptide comprised by the disclosed antibodies may be wild-type, such as shown or comprising SEQ ID NO:42, or it may be modified to achieve various outcomes.

For example, the part that binds to IL-31 may comprise a heavy chain comprising an Fc polypeptide having “knob” mutations, e.g., ((S354C, T366W); EU numbering), and the part that binds to IL-13 may comprise a heavy chain comprising an Fc polypeptide having “hole” mutations, e.g., ((Y349C, T366S, L368A, Y407V); (EU numbering), or vice versa, to facilitate proper pairing of a bispecific antibody described herein.

Alternatively, and preferably, the part that binds to IL-31 may comprise a heavy chain comprising an Fc polypeptide having an F405L mutation (EU numbering) and the part that binds to IL-13 may comprise a heavy chain comprising an Fc polypeptide having a K409R mutation (EU numbering), or vice versa, to facilitate proper pairing of a bispecific antibody described herein.

In some embodiments, the heavy chain constant region domains of the multispecific binding antibody, e.g., bispecific antibody, comprise the “LALA” mutation (L234A and L235A in EU numbering) for Fc silencing, the “YTE” mutation (M252Y, S254T, and T256E (EU Numbering)) for half-life extension and the knob-in-hole mutations (e.g., knob S354C and T366W; hole Y349C, T366S, L368A, and Y407V (EU Numbering)) that facilitate proper chain pairing.

In some embodiments, the modified Fc region comprised in the multispecific, e.g., bispecific, antibody described herein, comprises two Fc polypeptide chains, wherein the first Fc polypeptide chain comprises an amino acid sequence as set forth in SEQ ID NO: 46, having knob mutations S354C and T366W (EU Numbering), and the second Fc polypeptide chain comprises an amino acid sequence as set forth in SEQ ID NO: 47, having hole mutations (Y349C, T366S, L368A, and Y407V (EU Numbering).

In some embodiments, the heavy chain constant region domains of the multispecific binding antibody, e.g., bispecific antibody, comprise the “LALA” mutation (L234A and L235A in EU numbering) for Fc silencing, the “YTE” mutation (M252Y, S254T, and T256E (EU Numbering)) for half-life extension and the Fab arm exchange “FAE” mutations (e.g., K409R; F405L (EU Numbering)) that facilitate proper chain pairing.

A preferred modified Fc region comprised in the multispecific e.g., bispecific, antibody described herein comprises two Fc polypeptide chains, wherein the first Fc polypeptide chain comprises an amino acid sequence as set forth in SEQ ID NO: 44, comprising the “LALA” mutation (L234A and L235A in EU numbering); “YTE” mutation (M252Y, S254T, and T256E (EU Numbering)); and FAE mutation (F405L (EU Numbering)), and the second Fc polypeptide chain comprises an amino acid sequence as set forth in SEQ ID NO:45, comprising the “LALA” mutation (L234A and L235A in EU numbering); “YTE” mutation (M252Y, S254T, and T256E (EU Numbering)); and FAE mutation (K409R (EU Numbering)).

Other exemplary antibody effector silencing mutations that can be considered for the Fc polypeptides comprised in the multispecific antibodies described herein include the “DAPA” mutation (D265A, P329A in EU numbering), the “LALAGA” mutation (L234A, L235A, G237A in EU numbering), the “LALASKPA” mutation (L234A, L235A, S267K, P329A in EU numbering), the “DAPASK” mutation (D265A, P329A, S267K in EU numbering), the “GADAPA” mutation (G237A, D265A, P329A in EU numbering), the “GADAPASK” mutation (G237A, D265A, P329A, S267K in EU numbering), the “LALAPG” mutation (L234A, L235A, P329G in EU numbering), and the “LALAPA” mutation (L234A, L235A, P329A in EU numbering). Further examples of silencing mutations are disclosed in WO2022/097065.

Another exemplary antibody “half-life extension” mutation that can be considered for the Fc polypeptide comprised in the multispecific antibodies described herein is the “LS” mutation (M428L, N434S in EU numbering).

Modifications that may be made to the disclosed multispecific e.g., bispecific antibodies are discussed further below.

### A. Antibodies With Variable Region Modifications

Each of the antibodies described herein comprises CDR region sequences. In some embodiments, one or more of these CDR region sequences have conservative modifications relative to starting (parent) antibody amino acid sequences, and wherein the modified antibodies comprising the modified CDR sequences retain or have enhanced binding properties as compared to the parent antibodies. Conservative modifications (mutations) (as discussed above) can be introduced. The mutations may be amino acid substitutions, additions, deletions, or combinations thereof.

Amino acid sequence variants of the antibodies described herein can be prepared by introducing appropriate nucleotide changes into the encoding DNAs, or by synthesis of the desired variants. Such variants include, for example, deletions from, or insertions or substitutions of, residues within the amino acid sequences of present antibodies. Any combination of deletion, insertion, and substitution is made to arrive at the final construct, provided that the final construct possesses the desired antigen-binding characteristics. The amino acid changes also may alter post-translational processes of the antibodies, such as changing the number or position of glycosylation sites. The present application includes variants of the antibodies described herein and/or fragments thereof having amino acid conservative modifications in variable regions and/or constant regions.

In some embodiments, a multispecific, preferably bispecific, antibody described herein comprises an HCDR1, HCDR2, HCDR3, LCDR1, LCDR2, and/or LCDR3 region with a sequence shown in Table 1, or an HCDR1, HCDR2, HCDR3, LCDR1, LCDR2, and/or LCDR3 region having at least 80%, at least 85%, at least 90%, at least 95%, at least 96%, at least 97%, at least 98%, or at least 99% sequence identity with the sequence of an HCDR1, HCDR2, HCDR3, LCDR1, LCDR2, and/or LCDR3 region shown in Table 1. In some embodiments, a multispecific, preferably bispecific, antibody described herein comprises an HCDR1, HCDR2, HCDR3, LCDR1, LCDR2, and/or LCDR3 region having 1, 2, or 3 substitutions, deletions, or insertions relative to the sequence of an HCDR1, HCDR2, HCDR3, LCDR1, LCDR2, and/or LCDR3 region shown in Table 1.

In some embodiments, a multispecific, preferably bispecific, antibody described herein comprises a VH and/or VL region comprising or consisting of a sequence shown in Table 1, or a VL and/or VH region comprising or consisting of a sequence comprising at least 80%, at least 85%, at least 90%, at least 95%, at least 96%, at least 97%, at least 98%, or at least 99% sequence identity with the sequence of a VL and/or VH region shown in Table 1. In some embodiments, a multispecific, preferably bispecific, antibody described herein comprises a VL and/or VH region comprising 1, 2, or 3 substitutions, deletions, or insertions relative to the sequence of a VL and/or VH region shown in Table 1.

In some embodiments, a multispecific, preferably bispecific, antibody described herein comprises a heavy chain and/or light chain comprising or consisting of a sequence shown in Table 1, or a heavy chain and/or light chain comprising or consisting of a sequence comprising at least 80%, at least 85%, at least 90%, at least 95%, at least 96%, at least 97%, at least 98%, or at least 99% sequence identity with the sequence of a heavy chain and/or light chain shown in Table 1. In some embodiments, a multispecific, preferably bispecific, antibody described herein comprises a heavy chain and/or light chain comprising 1, 2, or 3 substitutions, deletions, or insertions relative to the sequence of a heavy chain and/or light chain shown in Table 1.

B. Antibodies with Enhanced Heterodimerization

Inadequate heterodimerization of two antibody heavy chain domains can be an obstacle for increasing the yield of desired multispecific antibodies and represents challenges for purification. A variety of approaches available can be used in to enhance dimerization of the two heavy chain domains of bispecific or multispecific antibodies, e.g., as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1.

The present disclosure provides methods of enhancing dimerization (hetero-dimerization) of two interacting heterologous polypeptides and/or reducing dimerization (homo-dimerization) of two identical polypeptides. Typically, each of the two interacting polypeptides comprises an Fc polypeptide having a CH2 and a CH3 domain of an antibody. The CH3 domains are derived from the constant region of an antibody of any isotype, class or subclass, and preferably of IgG (IgG1, IgG2, IgG3, or IgG4) class.

Typically, the polypeptides of the disclosure comprise other antibody fragments in addition to CH3 domains, such as, CH1 domains, CH2 domains, hinge domain, VH domain(s), VL domain(s), CDR(s), and/or antigen-binding fragments described herein, such as scFv or Fab. These antibody fragments are derived from various types of antibodies described herein, for example, polyclonal antibody, monoclonal antibodies, chimeric antibodies, humanized antibodies, human antibodies, bispecific or multispecific antibodies, camelized antibodies, anti-idiotypic (anti-Id) antibodies and antibody conjugates. Heterodimerization of two different heavy chains can give rise to the desired antibody or antibody-like molecule, while homodimerization of identical heavy chains will reduce yield of the desired antibody or molecule. In an exemplary embodiment, the two or more hetero-polypeptide chains comprise two chains comprising CH3 domains and forming the molecules of any of the multispecific antibody (e.g., bispecific antibody) or fragments thereof formats described above of the present disclosure.

In an embodiment, the two hetero-polypeptide chains comprising CH3 domains comprise modifications that favor heterodimeric association of the polypeptides, relative to unmodified chains. Various examples of modification strategies are described earlier herein and are further provided below.

### Knob-in-Hole (KIH)

Multispecific, e.g., bispecific, antibodies or fragments thereof of the present disclosure may comprise one or more mutations to one or more of the constant domains, e.g., to the CH3 domains. In some embodiments, the multispecific e.g., bispecific, antibody or fragment thereof of the present disclosure comprises two polypeptides that each comprise a heavy chain constant domain of an antibody, e.g., a CH2 or CH3 domain or CH2-CH3 domain. In some embodiments, the two heavy chain constant domains, e.g., the CH2-CH3 domains of the multispecific, e.g., bispecific, antibody or fragment thereof comprise one or more mutations that allow for a heterodimeric association between the two chains. In some embodiments, the one or more mutations are disposed on the CH3 domains of at least two polypeptides of the multispecific, e.g., bispecific, antibody or fragment thereof. In some embodiments, the one or more mutations to a first polypeptide of the multispecific, e.g., bispecific, antibody or fragment thereof comprising a heavy chain constant domain creates a “knob” and the one or more mutations to a second polypeptide of the multispecific, e.g., bispecific, antibody or fragment thereof comprising a heavy chain constant domain creates a “hole,” such that heterodimerization of the polypeptide of the multispecific, e.g., bispecific, antibody or fragment thereof comprising a heavy chain constant domain causes the “knob” to interface (e.g., interact, e.g., a CH3 domain of a first polypeptide interacting with a CH3 domain of a second polypeptide) with the “hole.”

As the term is used herein, a “knob” refers to at least one amino acid side chain which projects from the interface of a first polypeptide of the multispecific, e.g., bispecific, antibody or fragment thereof comprising a heavy chain constant domain and is therefore positionable in a compensatory “hole” in the interface with a second polypeptide of the multispecific, e.g., bispecific antibody or fragment thereof comprising a heavy chain constant domain so as to stabilize the heterodimerization of polypeptide chains (e.g., of the half antibodies) of multispecific, e.g., bispecific, antibodies or fragments thereof over homomultimer formation, for example. The knob may exist in the original interface or may be introduced synthetically (e.g. by altering nucleic acid encoding the interface). Typical import residues for the formation of a knob are generally naturally occurring amino acid residues and are typically selected from arginine (R), phenylalanine (F), tyrosine (Y) and tryptophan (W). Most typical are tryptophan and tyrosine. In some embodiments, the original residue for the formation of the protuberance has a small side chain volume, such as alanine, asparagine, aspartic acid, glycine, serine, threonine or valine.

A “hole” refers to at least one amino acid side chain which is recessed from the interface of a second polypeptide of the multispecific, e.g., bispecific, antibody or fragment thereof comprising a heavy chain constant domain and therefore accommodates a corresponding knob on the adjacent interfacing surface of a first polypeptide of the multispecific, e.g., bispecific, antibody or fragment thereof comprising a heavy chain constant domain. The hole may exist in the original interface or may be introduced synthetically (e.g. by altering nucleic acid encoding the interface). The typical import residues for the formation of a hole are usually naturally occurring amino acid residues and are typically selected from alanine (A), serine (S), threonine (T) and valine (V). Most typical are serine, alanine or threonine. In some embodiments, the original residue for the formation of the hole has a large side chain volume, such as tyrosine, arginine, phenylalanine or tryptophan.

In some embodiments, a first CH3 domain is mutated at residue 366, 405 and/or 407 according to the EU numbering scheme (Edelman et al., PNAS, 1969 May, 63(1):78-85; Kabat et al. (pp. 688-696 in Sequences of proteins or immunological interest, 5^{th} ed., Vol. 1 (1991; NIH, Bethesda, Md.)) to create either a “knob” or a “hole” (as described above), and the second CH3 domain that heterodimerizes with the first CH3 domain is mutated at: residue 407 if residue 366 is mutated in the first CH3 domain, residue 349 if residue 405 is mutated in the first CH3 domain, or residue 366 if residue 407 is mutated in the first CH3 domain (EU numbering), to create a “hole” or “knob” complementary to the “knob” or “hole” of the first CH3 domain.

In another embodiment, a first CH3 domain is mutated at residue 366 (EU numbering) to create either a “knob” or a hole” (as described above), and the second CH3 domain that heterodimerizes with the first CH3 domain is mutated at residues 366, 368 and/or 407 (EU numbering). In one embodiment, the mutation to the first CH3 domain introduces a tyrosine (Y) residue at position 366 (EU numbering). In an embodiment, the mutation to the first CH3 is T366Y. In one embodiment, the mutation to the first CH3 domain introduces a tryptophan (W) residue at position 366 (EU numbering). In an embodiment, the mutation to the first CH3 is T366W. In some embodiments, the mutation to the second CH3 domain that heterodimerizes with the first CH3 domain mutated at position 366 (e.g., has a tyrosine (Y) or tryptophan (W) introduced at position 366, e.g., comprises the mutation T366Y or T366W), comprises a mutation at position 366, a mutation at position 368 and a mutation at position 407 (EU numbering). In some embodiments, the mutation at position 366 introduces a serine (S) residue, the mutation at position 368 introduces an alanine (A), and the mutation at position 407 introduces a valine (V). In some embodiments, the mutations comprise T366S, L368A and Y407V. In one embodiment the first CH3 domain of the multispecific, e.g., bispecific, antibody or fragment thereof comprises the mutation T366Y, and the second CH3 domain that heterodimerizes with the first CH3 domain comprises the mutations T366S, L368A and Y407V, or vice versa. In one embodiment, the first CH3 domain of the multispecific, e.g., bispecific, antibody or fragment thereof comprises the mutation T366W, and the second CH3 domain that heterodimerizes with the first CH3 domain comprises the mutations T366S, L368A and Y407V, or vice versa.

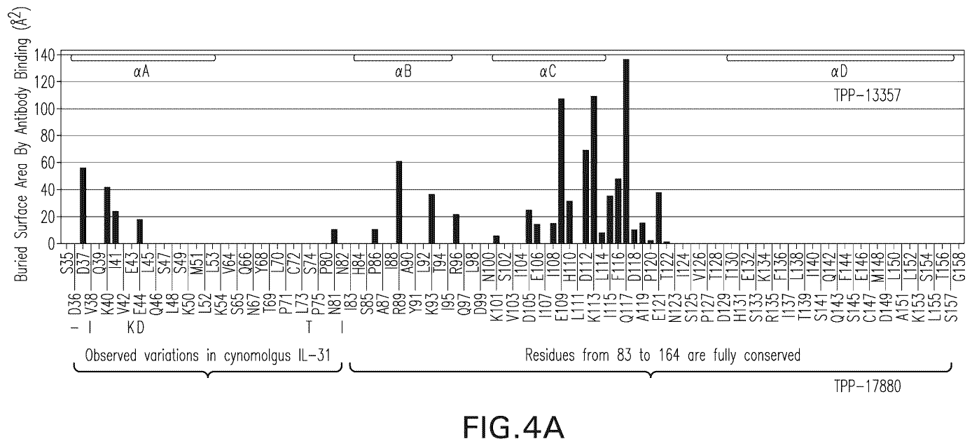

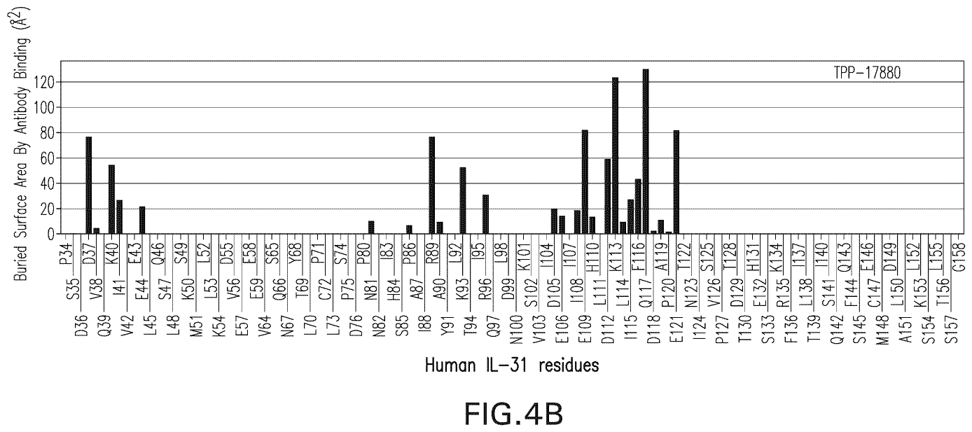

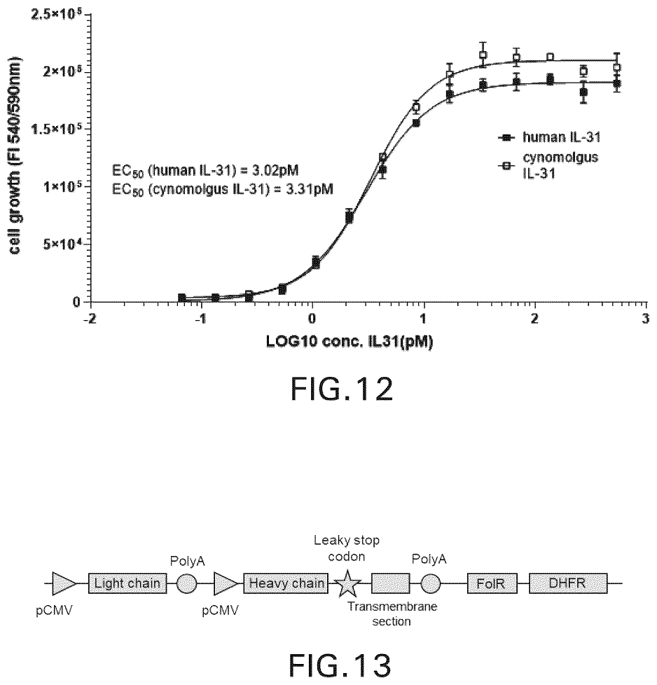

Additional knob in hole mutations suitable for use in multispecific e.g., bispecific, antibodies or fragments thereof of the present disclosure are described in PCT publication no. WO2014/145806 (for example, FIG. 3, FIG. 4 and FIG. 12 of WO2014/145806), PCT publication no. WO2014/1 10601, and PCT publication no. WO 2016/086186, WO 2016/086189, WO 2016/086196, WO 2016/182751 and WO 1996/027011. An example of a KIH variant comprises a first constant chain comprising a L368D and a K370S mutation, paired with a second constant chain comprising a S364K and E357Q mutation (EU numbering).

In an embodiment, a first CH3 domain is modified to display a hole like structure by introducing point mutations Y349C, T366S, L368A, Y407V and a second CH3 domain is modified to display a knob like structure by introducing point mutations S354C, T366W. The two modified CH3 regions then interact to form a bivalent bispecific antibody, i.e. a hetero-tetrameric protein, consisting of two different light and two different heavy chains.

In an embodiment, there is provided a first Fc polypeptide chain comprising an amino acid sequence as set forth in SEQ ID NO: 46, having knob variations (S354C, T366W (EU Numbering)), and a second Fc polypeptide chain comprising an amino acid sequence as set forth in SEQ ID NO: 47, having hole variations (Y349C, T366S, L368A, Y407V (EU Numbering)).

In any of the embodiments described herein, the CH3 domains may be additionally mutated to introduce a pair of cysteine residues. Without being bound by theory, it is believed that the introduction of a pair of cysteine residues capable of forming a disulfide bond provide stability to the heterodimerized antibody. In some embodiments, the first CH3 domain comprises a cysteine at position 354 (EU numbering) and the second CH3 domain that heterodimerizes with the first CH3 domain comprises a cysteine at position 349 (EU numbering).

### IgG Heterodimerization

In preferred embodiments, a multispecific, e.g., bispecific, antibody or fragment thereof is obtained by Fab arm exchange. The Fab arm exchange approach is described e.g. by Labrijn et al., Proc Natl Acad Sci USA. 2013 Mar. 26; 110(13):5145-50, and involves mixing of two separately expressed parental antibodies under controlled reducing conditions to separate the antibodies into half-antibodies and allow the recombining of half antibodies to form e.g., a bispecific antibody.

In some embodiments, heterodimerization of the polypeptide chains (e.g., of the half antibodies) of the multispecific, e.g., bispecific, antibody or fragment thereof is increased by introducing one or more FAE mutations in a CH3 domain. In an embodiment, the mutations comprise a K409R mutation introduced to one CH3 domain paired with F405L mutation in the second CH3 domain, according to the EU numbering scheme, or vice versa. In some embodiments, additional mutations may also, or alternatively, be introduced at positions selected from the group consisting of positions 366, 368, 370, 399, 405, 407, and 409 according to the EU numbering scheme.

The amino acid replacements described herein are introduced into the CH3 domains using techniques which are known in the art. Typically, the DNA encoding the heavy chain(s) is genetically engineered using standard techniques, e.g., described in Directed Mutagenesis: a Practical Approach (M J McPherson (ed.), 1991, IRL Press). Oligonucleotide-mediated mutagenesis is an exemplary method for preparing substitution variants of the DNA encoding the two hybrid heavy chains. This technique is known in the art as e.g., described by Adelman et al., (1983) DNA, 2:183. The IgG heterodimerization strategy is described in, for example, WO2008/119353, WO2011/131746, and WO2013/060867.

In some embodiments, the CH3 domains may be additionally mutated to introduce a pair of cysteine residues. In embodiments, the CH3 domain of the first antibody comprises a cysteine at position 354, and the CH3 domain of the second antibody that heterodimerizes with the CH3 domain of the first antibody comprises a cysteine at position 349 (EU numbering).

In some embodiments, there is provided a method for enhancing the formation of the multispecific (e.g., bispecific) antibody by mutating one or more residues in the hinge region with charged residues that favor heterodimer formation over homodimer formation. In some embodiments, multispecific (e.g., bispecific) antibody comprises a first part antibody having a mutation at position 221 and/or 228 (EU numbering). In some embodiments, the amino acid modification has a charged residue (e.g., Lys, Arg, His, Glu, and Asp) or polar residues (e.g., Ser and Thr). In some embodiments, the multispecific (e.g., bispecific) antibody comprises a first part antibody comprising Arg221 or Glu221 and Arg228 or Glu228 (EU numbering); and a second part antibody comprising Arg221 or Glu221 and Arg228 or Glu228 (EU numbering), wherein the amino acids at positions 221 and 228 of the first part antibody are not the same as the corresponding amino acids in the second part antibody. In some embodiments, the first part antibody comprises Arg221 or Glu221 and Arg228 or Glu228, and additionally (in the CH3 region) Arg409 or Glu368; and the part second antibody comprises Arg221 or Glu221 and Arg228 or Glu228, and additionally (in the CH3 region) Arg409 or Glu368, wherein the amino acids at positions 221 and 228 and 368 and 409 of the first part antibody are not the same as the corresponding amino acids in the second part antibody. In this embodiment, either the first antibody or second antibody may comprise a kappa light chain and the other may comprise a lambda light chain.

### Polar Bridge

In some embodiments, heterodimerization of the polypeptide chains (e.g., of the half antibodies) of the multispecific antibody (e.g. bispecific antibody) or fragments thereof is increased by introducing mutations based on the “polar-bridging” rational, which is to make residues at the binding interface of the two polypeptide chains to interact with residues of similar (or complimentary) physical property in the heterodimer configuration. In particular, these mutations are designed so that, in the heterodimer formation, polar residues interact with polar residues, while hydrophobic residues interact with hydrophobic residues. In contrast, in the homodimer formation, residues are mutated so that polar residues interact with hydrophobic residues. The favorable interactions in the heterodimer configuration and the unfavorable interactions in the homodimer configuration work together to make it more likely for CH3 domains to form heterodimers than to form homodimers. In an exemplary embodiment, the above mutations are generated at one or more positions selected from the group consisting of residues 364, 368, 399, 405, 409, and 411 of CH3 domain (EU numbering).

In an embodiment, one or more mutations is selected from a group consisting of: Ser364Leu, Thr366Val, Leu368Gln, Asp399Lys, Phe405Ser, Lys409Phe and Thr411 Lys (EU numbering) are introduced into one of the two CH3 domains. For example, Ser364Leu: original residue of serine at position 364 is replaced by leucine; Thr366Val: original residue of threonine at position 366 is replaced by valine; Leu368Gln: original residue of leucine at position 368 is replaced by glutamine; Asp399Lys: original residue aspartic acid at position 399 is replaced by lysine; Phe405Ser: original residue phenylalanine at position 405 is replaced by serine; Lys409Phe: original residue lysine at position 409 is replaced by phenylalanine; Thr411 Lys: original residue of threonine at position 411 is replaced by lysine.

In another embodiment, the other CH3 can be introduced with one or more mutations selected from a group consisting of: Tyr407Phe, Lys409Gln and Thr411Asp (for example, Tyr407Phe: original residue tyrosine at position 407 is replaced by phenylalanine; Lys409Glu: original residue lysine at position 409 is replaced by glutamic acid; Thr411Asp: original residue of threonine at position 411 is replaced by aspartic acid) (EU numbering).

In another embodiment, one CH3 domain has one or more mutations selected from a group consisting of: Ser364Leu, Thr366Val, Leu368Gln, Asp399Lys, Phe405Ser, Lys409Phe and Thr411 Lys, while the other CH3 domain has one or more mutations selected from a group consisting of: Tyr407Phe, Lys409Gln and Thr411Asp (EU numbering).

In another embodiment, the original residue of threonine at position 366 of one CH3 domain is replaced by valine, while the original residue of tyrosine at position 407 of the other CH3 domain is replaced by phenylalanine (EU numbering).

In another embodiment, the original residue of serine at position 364 of one CH3 domain is replaced by leucine, while the original residue of leucine at position 368 of the same CH3 domain is replaced by glutamine (EU numbering).

In another embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine (EU numbering).

In another embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid (EU numbering).

The polar bridge strategy is described in, for example, WO2006/106905, WO2009/089004 and Gunasekaran K et al., (2010) J Biol Chem., 285: 19637-19646. In any of the embodiments described herein, the CH3 domains may be additionally mutated to introduce a pair of cysteine residues. In some embodiments, the first CH3 domain comprises a cysteine at position 354 (EU numbering), and the second CH3 domain that heterodimerizes with the first CH3 domain comprises a cysteine at position 349 (EU numbering).

C. Antibodies with an Extended In Vivo Half-Life

The antibodies or fragments thereof described herein can be further modified to have an extended half-life in vivo. A variety of strategies known to the skilled person can be used to this end, e.g., chemical linkage to polyethylene glycol (PEG), reCODE PEG, antibody scaffold, polysialic acid (PSA), hydroxyethyl starch (HES), albumin-binding ligands, and carbohydrate shields; genetic fusion to proteins binding to serum proteins, such as albumin, IgG, FcRn, and transferring; coupling (genetically or chemically) to other binding moieties that bind to serum proteins such as nanobodies, Fabs, DARPins, avimers, affibodies, and anticalins; genetic fusion to rPEG, albumin, domain of albumin, albumin-binding proteins, and Fc, or by incorporation into nanocarriers or slow release formulations.

Antibodies or fragments thereof having an increased half-life in vivo can also be generated introducing one or more amino acid modifications (i.e., substitutions, insertions or deletions) into an IgG constant domain, or FcRn binding fragment thereof (preferably a Fc polypeptide or fragment thereof). See, e.g., International Publication No. WO 98/23289; International Publication No. WO 97/34631; and U.S. Pat. No. 6,277,375. Modifications to the Fc of the multispecific e.g., bispecific, antibodies or fragments thereof include the “LS” mutation (M428L, N434S, (EU numbering)) and, preferably, the “YTE” mutation (M252Y, S254T, T256E (EU Numbering)) for half-life extension.

Further, the antibodies or fragments thereof can be conjugated or fused to one or more human serum albumin (HSA) polypeptides, or a portion thereof. Albumin can be used to make the molecules more stable in vivo or have a longer half-life in vivo. Such techniques are known in the art, see, e.g., WO 1993/15199, WO 1993/15200, WO 2001/77137, and EP 413622. The use of N-terminal fragments of HSA for fusions to polypeptides has also been described (e.g. EP 399666). Accordingly, genetically or chemically fusing or conjugating the molecules to albumin can stabilize or extend the shelf-life, and/or retain the molecule's activity for extended periods of time in solution, in vitro and/or in vivo. Additional methods pertaining to HSA fusions can be found, for example, in WO 2001/077137 and WO 2003/06007. In some embodiments, the expression of the fusion protein is performed in mammalian cell lines, for example, CHO cell lines.

### D. Fc Silencing

In embodiments wherein one or more constant domains, e.g., heavy constant regions, are incorporated in the antibodies or fragments thereof described herein, it can be beneficial to include or more mutations to silence effector function, e.g., ADCC and/or CDC effector function within the Fc. Crosslinking of Fc via gamma receptors may induce antibody dependent cellular cytotoxicity (ADCC). Human Fc when complexed at the cell surface can also bind complement proteins and induce complement dependent cytotoxicity (CDC). Mutations to residues in Fc which reduce or abrogate these interactions may thus limit these effects and focus the impact of the antibodies or fragments thereof described herein upon target cells.

In some embodiments, one or more, e.g., all, of the heavy chain constant region domains of antibody or fragment thereof comprise the DAPA mutation (e.g., D265A and P329A in EU numbering). See e.g., Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001; 276(9):6591-604; U.S. Patent Publication US2015/0320880 A1.

In some embodiments, one or more, e.g., all, of the heavy chain constant region domains of the antibody or fragment thereof comprise an N279A mutation (according to EU numbering) (see, e.g., Tao M H, Morrison S L. Studies of aglycosylated chimeric mouse-human IgG. Role of carbohydrate in the structure and effector functions mediated by the human IgG constant region. J Immunol. 1989; 143(8):2595-601; Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001; 276(9):6591-604). Additional Fc mutations for providing silenced effector function are described in WO2014/145806. One example of a silent IgG1 antibody comprises a E233P, L234V, L235A, and S267K mutation, and a deletion of G236 (G236del) (EU numbering). Another example of a silent IgG1 antibody comprises a E233P, L234V, and L235A mutation, and a deletion of G236 (G236del) (EU numbering). Another example of a silent IgG1 antibody comprises a S267K mutation (EU numbering).

In some embodiments, one or more, e.g., all, of the heavy chain constant region domains of the antibody or fragment thereof comprise the LALA mutation (e.g., L234A and L235A in EU numbering). E.g., Hezareh M, Hessell A J, Jensen R C, van de Winkel J G J, Parren P W H I. Effector Function Activities of a Panel of Mutants of a Broadly Neutralizing Antibody against Human Immunodeficiency Virus Type 1. Journal of Virology. 2001; 75(24):12161-12168; Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fe gamma RIII, and FcRn and design of IgG1 variants with improved binding to the Fe gamma R. J Biol Chem. 2001; 276(9):6591-604.

In some embodiments, the antibody is a bispecific antibody comprising a heavy chain constant region domain comprising the “LALA” mutation (L234A and L235A in EU numbering) for Fc silencing, the “YTE” mutation (M252Y, S254T, T256E (EU Numbering)) for half-life extension and a Fab arm exchange “FAE” mutation (K409R or F405L (EU Numbering)) that facilitates proper chain pairing. In some embodiments, the antibody is a bispecific antibody comprising two Fc polypeptide chains, wherein the first Fc polypeptide chain comprises an amino acid sequence as set forth in SEQ ID NO: 44, comprising “LALA” mutation (L234A and L235A in EU numbering); “YTE” mutation (M252Y, S254T, T256E (EU Numbering)); and an FAE mutation (F405L (EU Numbering)), and the second Fc polypeptide chain comprises an amino acid sequence as set forth in SEQ ID NO: 45, comprising “LALA” mutation ((L234A and L235A in EU numbering); “YTE” mutation (M252Y, S254T, T256E (EU Numbering)); and an FAE mutation (K409R (EU Numbering)).

### Conjugates

The antibodies or fragments thereof described herein may be recombinantly fused or chemically conjugated (including both covalent and non-covalent conjugations) to a heterologous protein or polypeptide. Methods for fusing or conjugating proteins, polypeptides, or peptides to an antibody are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., (1991) Proc. Natl. Acad. Sci. USA 88:10535-10539; Zheng et al., (1995) J. Immunol. 154:5590-5600; and Vil et al., (1992) Proc. Natl. Acad. Sci. USA 89:11337-11341. Additional fusion proteins may be generated through the techniques of gene-shuffling, motif-shuffling, exon-shuffling, and/or codon-shuffling (collectively referred to as “DNA shuffling”). DNA shuffling may be employed to alter the activities of the antibodies or fragments thereof. See, e.g., U.S. Pat. Nos. 5,605,793, 5,811,238, 5,830,721, 5,834,252, and 5,837,458; Patten et al., (1997) Curr. Opinion Biotechnol. 8:724-33; Harayama (1998) Trends Biotechnol. 16(2):76-82; Hansson et al., (1999) J. Mal. Biol. 287: 265-76; and Lorenzo & Blasco (1998) Biotechniques, 24(2):308-313. The antibodies or fragments thereof may e.g., be altered by being subjected to random mutagenesis by error-prone PCR, random nucleotide insertion or other methods prior to recombination. A polynucleotide encoding a fragment of the present molecule may be recombined with one or more components, motifs, sections, parts, domains, fragments, etc. of one or more heterologous molecules.

Moreover, the antibodies or fragments thereof of the present invention can be fused to marker sequences, such as a peptide to facilitate purification. In some embodiments, the marker amino acid sequence is a hexa-histidine peptide, such as the tag provided in a pQE vector (QIAGEN, Inc., 9259 Eton Avenue, Chatsworth, CA, 91311), among others, many of which are commercially available. As described in Gentz et al., (1989) Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., (1984) Cell 37:767), and the “flag” tag.

In other embodiments, the antibodies of the present invention are conjugated to a diagnostic or detectable agent. Such molecules can be useful for monitoring or prognosing the onset, development, progression and/or severity of a disease or disorder as part of a clinical testing procedure, such as determining the efficacy of a particular therapy. Such diagnosis and detection can accomplished by coupling the molecules to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive isotopes, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, 47Sc, ¹⁸⁶Re, ¹⁸⁸Re, ¹⁴²Pr, ¹⁰⁵Rh, ⁹⁷Ru, ⁶⁸Ge ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; and positron emitting metals using various positron emission tomographies, and nonradioactive paramagnetic metal ions. Other examples of radioactive isotopes include iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵¹Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I) indium (¹¹¹In), technetium (⁹⁹Tc), phosphorus (³²P), carbon (¹⁴C), and tritium (³H), or one or more of the therapeutic isotopes listed above.

The present application further encompasses uses of the antibodies or fragments thereof conjugated to a therapeutic moiety. The molecules of the present disclosure or fragments thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, the antibodies or fragments thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, Pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine. Examples of suitable types of cytotoxins, linkers and methods for conjugating therapeutic agents to the molecules, can be found in Saito et al., (2003) Adv. Drug Del iv. Rev. 55: 199-215; Trail et al., (2003) Cancer Immunol. Immunother. 52: 328-337; Payne (2003) Cancer Cell 3: 207-212; Allen (2002) Nat. Rev. Cancer, 2:750-763; Pastan and Kreitman (2002) Curr. Opin. Investig. Drugs, 3: 1089-1091; Senter & Springer (2001) Adv. Drug Deliv. Rev. 53: 247-264. The antibodies or fragments thereof also can be conjugated to a radioactive isotope to generate cytotoxic radiopharmaceuticals, also referred to as radioimmunoconjugates. Examples of radioactive isotopes that can be conjugated to molecules for use diagnostically or therapeutically include, but are not limited to, iodine 131, indium 111, yttrium 90, and lutetium 177. Methods for preparing radioimmunconjugates are established in the art. See, e.g., Denardo et al., (1998) Clin Cancer Res. 4(10): 2483-90; Peterson et al., (1999) Bioconjug. Chem. 10(4):553-7; and Zimmerman et al., (1999) Nucl. Med. Biol. 26(8): 943-50.

Techniques for conjugating therapeutic moieties to antibodies are known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., (1982) Immunol. Rev. 62:119-58.

The antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Methods of Making the Antibodies of the Present Invention

### A. Preparing Polypeptide Chains

The antibodies or fragments thereof described herein can be produced by a variety of techniques, including conventional monoclonal antibody methodology e.g., the standard somatic cell hybridization technique of Kohler and Milstein, (1975) Nature 256: 495. Many techniques for producing monoclonal antibodies can be employed e.g., viral or oncogenic transformation of B lymphocytes, or production using recombinant host cells as described later herein.

An animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

In some embodiments, the antibodies or fragments thereof are humanized antibodies or fragments thereof.

Chimeric or humanized antibodies used in the present invention can e.g., be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567). To create a humanized antibody, the murine CDR regions can, for example, be inserted into a human framework and constant regions using methods known in the art. See e.g., U.S. Pat. No. 5,225,539 to Winter, and U.S. Pat. Nos. 5,530,101; 5,585,089; 5,693,762 and 6,180,370. Alternatively, point mutations to decrease immunogenicity to humans can be inserted in the variable (e.g., in the framework and/or CDR regions) and/or constant regions, following by humanized antibody expression using recombinant host cells. Alternatively, humanization may be achieved by a process of ‘veneering’, by reducing the immunogenicity of non-human Fv by replacing exposed residues in its framework regions that differ from those usually found in human antibodies. Because protein antigenicity may be correlated with surface accessibility, replacement of the surface residues can be sufficient to render a non-human (e.g., mouse) variable region invisible to the human immune system (see Mark G E et al., (1994) in Handbook of Experimental Pharmacology vol. 113: The pharmacology of monoclonal antibodies, Springer-Verlag, pp 105-134).

In some embodiments, the antibodies or fragments thereof are human monoclonal antibodies or fragments thereof. Human monoclonal antibodies can e.g., be generated using transgenic or transchromosomic mice carrying parts of the human immune system rather than the mouse system. These transgenic and transchromosomic mice are collectively referred to herein as “human Ig mice.” Human antibodies can e.g., be raised using a mouse that carries human immunoglobulin sequences on transgenes and transchromosomes such as a mouse that carries a human heavy chain transgene and a human light chain transchromosome. Such mice, referred to herein as “KM mice”, are described in detail in PCT Publication WO 02/43478.

Alternative transgenic animal systems expressing human immunoglobulin genes are available in the art and can be used to raise human antibodies used in the present invention. For example, an alternative transgenic system referred to as the Xenomouse (Abgenix, Inc.) can be used. Such mice are described in, e.g., U.S. Pat. Nos. 5,939,598; 6,075,181; 6,114,598; 6, 150,584 and 6,162,963. Human monoclonal antibodies can also be prepared using phage display or yeast display methods for screening libraries of human immunoglobulin genes. Phage display methods for isolating human antibodies are established in the art or described in the examples below. See for example: U.S. Pat. Nos. 5,223,409; 5,403,484; and 5,571,698; 5,427,908 and 5,580,717; 5,969,108 and 6,172,197; and 5,885,793; 6,521,404; 6,544,731; 6,555,313; 6,582,915 and 6,593,081. Human monoclonal antibodies can also be prepared using SCID mice into which human immune cells have been reconstituted such that a human antibody response can be generated upon immunization. Such mice are described in, for example, U.S. Pat. Nos. 5,476,996 and 5,698,767.

Methods of making multispecific e.g., bispecific, antibodies or fragments thereof are well known in the art. The multispecific antibodies or fragments thereof described herein (e.g., an IL-31/IL-13 bispecific antibody described herein) may be engineered from existing monospecific antibodies (e.g., monospecific IL-31 antibodies and IL-13 antibodies) e.g., made using the conventional monoclonal antibody technology described above.

The multispecific, e.g., bispecific, antibodies or fragments thereof can be based on any of the different formats described herein.

Where multispecific antibodies or fragments thereof of the present invention are prepared by cross-linking polypeptide chains, these functional linkages can be accomplished using methods known in the art. A variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohexane-1-carboxylate (sulfo-SMCC) (see e.g., Karpovsky et al., (1984) J. Exp. Med. 160:1686; Liu et al. (1985) Proc. Natl. Acad. Sci. USA 82:8648). Other methods include those described in Paulus (1985) Behring Ins. Mitt. No. 78:118-132; Brennan et al., (1985) Science 229:81-83), and Glennie et al., (1987) J. Immunol. 139: 2367-2375). Conjugating agents are SATA and sulfo-SMCC, both available from Pierce Chemical Co. (Rockford, IL).

In some embodiments, the heavy chains contain comprise an Fc polypeptide that is wild-type (i.e., not containing any mutations), for example described earlier herein. In some embodiments, the heavy chains comprise an Fc polypeptide comprising at least one mutation (e.g., “LALA”, “YTE”, or K409R/F405L (EU numbering) or any other mutation described herein).

If the antibodies or fragments thereof are produced recombinantly, the nucleic acid molecules encoding the antibodies or fragments thereof may be prepared first.

### B. Methods of Producing Antibodies of the Present Invention Recombinantly

The antibodies or fragments thereof of the invention may be generated de novo. The antibodies or fragments thereof, e.g., multispecific (such as bispecific) antibodies or fragments thereof described herein can, for example, be generated recombinantly by introducing DNA constructs encoding the desired molecules into expression vectors and expressing and assembling the desired molecules in host cells.

In a further aspect, there is provided an isolated nucleic acid molecule encoding an antibody or fragment thereof described herein. In some embodiments, the isolated nucleic acid molecule encodes a monospecific IL-31 antibody or fragment thereof described herein. In some embodiments, the isolated nucleic acid molecule encodes a multispecific e.g., bispecific antibody or fragment thereof described herein.

An isolated sequence encoding a first part antibody having a first specificity (e.g., an IL-31 binding antibody described herein) and an isolated sequence encoding a second part antibody having a second specificity (e.g., an IL-13 binding antibody described herein) may be disposed on separate polynucleotides, which is also referred to as a “set of nucleic acid molecules.” Therefore, in a further aspect, there is provided a set of nucleic acid molecules encoding a multispecific e.g., bispecific, antibody or fragment thereof described herein.

In an embodiment, the isolated nucleic acid molecule, or set of nucleic acid molecules, encodes an anti-IL-31/IL-13 bispecific antibody described herein.

In an embodiment, there is provided a set of isolated nucleic acid molecules encoding a multispecific e.g., bispecific, antibody or fragment thereof described herein, wherein the first part binding to IL-31 and the second part binding to IL-13 are encoded by a separate nucleic acid molecule.

In some embodiments, the isolated nucleic acid molecule(s) is/are complementary DNA (cDNA) or messenger RNA (mRNA) molecules.

In some embodiments, an isolated nucleic acid molecule encodes an anti-IL-31 antibody or fragment thereof comprising an amino acid sequence as set forth in any of SEQ ID NOs: 7, 8, 9, 10, 28, 29, 30, 31, 32, 33, 34, 35, 37, 38, 39, 104, 105, 107, 108, or 109, or comprising an amino acid sequence having at least 80%, at least 85%, at least 90%, at least 95%, at least 96%, at least 97%, at least 98%, or at least 99% identity with any of SEQ ID NOs: 7, 8, 9, 10, 28, 29, 30, 31, 32, 33, 34, 35, 37, 38, 39, 104, 105, 107, 108, or 109.

In some embodiments, the isolated nucleic acid molecule comprises a nucleic acid sequence as set forth in any of SEQ ID NOs: 119-122 or 127-141, or comprises a nucleic acid sequence having at least 80%, at least 85%, at least 90%, at least 95%, at least 96%, at least 97%, at least 98%, or at least 99% identity with any of SEQ ID NOs: 119-122 or 127-141.

In some embodiments, the isolated nucleic acid molecule additionally encodes an anti-IL-13 antibody comprising an amino acid sequence as set forth in any of SEQ ID NOs: 17-20 or 110, or comprising an amino acid sequence having at least 80%, at least 85%, at least 90%, at least 95%, at least 96%, at least 97%, at least 98%, or at least 99% identity with any of SEQ ID NOs: 17-20 or 110.

In some embodiments, the isolated nucleic acid molecule additionally comprises a nucleic acid sequence as set forth in any of SEQ ID NOs: 123-126 or 142, or comprises a nucleic acid sequence having at least 80%, at least 85%, at least 90%, at least 95%, at least 96%, at least 97%, at least 98%, or at least 99% identity with any of SEQ ID NOs: 123-126 or 142.

In some embodiments, the set of nucleic acid molecules comprises i) an isolated nucleic acid molecule encoding an anti-IL-31 antibody comprising an amino acid sequence as set forth in any of SEQ ID NOs: 7, 8, 9, 10, 28, 29, 30, 31, 32, 33, 34, 35, 37, 38, 39, 104, 105, 107, 108, or 109, or comprising an amino acid sequence having at least 80%, at least 85%, at least 90%, at least 95%, at least 96%, at least 97%, at least 98%, or at least 99% identity with any of SEQ ID NOs: 7, 8, 9, 10, 28, 29, 30, 31, 32, 33, 34, 35, 37, 38, 39, 104, 105, 107, 108, or 109. In an embodiment, the set of nucleic acid molecules additionally comprises ii) an isolated nucleic acid molecule encoding an anti-IL-13 antibody comprising an amino acid sequence as set forth in any of SEQ ID NOs: 17-20 or 110, or comprising an amino acid sequence having at least 80%, at least 85%, at least 90%, at least 95%, at least 96%, at least 97%, at least 98%, or at least 99% identity with any of SEQ ID NOs: 17-20 or 110.

In some embodiments, the set of nucleic acid molecules comprises i) an isolated nucleic acid molecule comprising a nucleic acid sequence as set forth in any of SEQ ID NOs: 119-122 or 127-141, or comprising a nucleic acid sequence having at least 80%, at least 85%, at least 90%, at least 95%, at least 96%, at least 97%, at least 98%, or at least 99% identity with any of SEQ ID NOs: 119-122 or 127-141. In some embodiments, the set of nucleic acid molecules additionally comprises ii) an isolated nucleic acid molecule comprising a nucleic acid sequence as set forth in any of SEQ ID NOs: 123-126 or 142, or comprising a nucleic acid sequence having at least 80%, at least 85%, at least 90%, at least 95%, at least 96%, at least 97%, at least 98%, or at least 99% identity with any of SEQ ID NOs: 123-126 or 142.

In some embodiments the isolated nucleic acid molecule encodes an IL-31 binding antibody comprising a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith); or a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), or a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith). In some embodiments, the isolated nucleic acid molecule encodes an IL-31 binding antibody comprising a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith).

In some embodiments, the isolated nucleic acid molecule encodes an IL-31 binding antibody comprising: i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith); or i) a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith). In some embodiments, the isolated nucleic acid molecule encodes an IL-31 binding antibody comprising i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith). In some embodiments, the isolated nucleic acid molecule encodes an IL-31 binding antibody comprising i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 10 (or an amino acid sequence having at least 80%, at least 85%, at least 90%, at least 95%, at least 96%, at least 97%, at least 98%, or at least 99% sequence identity therewith). In some embodiments, the isolated nucleic acid molecule encodes an IL-31 binding antibody comprising i) a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and ii) a heavy chain comprising or consisting of the amino acid sequence shown in SEQ ID NO: 109 (or an amino acid sequence having at least 80%, at least 85%, at least 90%, at least 95%, at least 96%, at least 97%, at least 98%, or at least 99% sequence identity therewith).

In some embodiments, the isolated nucleic acid molecule encoding the anti-IL-31 antibody comprises i) a nucleotide sequence as set forth in SEQ ID NO: 138 and a nucleotide sequence as set forth in SEQ ID NO: 121; or ii) a nucleotide sequence as set forth in SEQ ID NO: 139 and a nucleotide sequence as set forth in SEQ ID NO: 134; or iii) a nucleotide sequence as set forth in SEQ ID NO: 139 and a nucleotide sequence as set forth in SEQ ID NO: 135; or iv) a nucleotide sequence as set forth in SEQ ID NO: 139 and a nucleotide sequence as set forth in SEQ ID NO: 136; or v) a nucleotide sequence as set forth in SEQ ID NO: 139 and a nucleotide sequence as set forth in SEQ ID NO: 137; or vi) a nucleotide sequence as set forth in SEQ ID NO: 140 and a nucleotide sequence as set forth in SEQ ID NO: 134; or vii) a nucleotide sequence as set forth in SEQ ID NO: 140 and a nucleotide sequence as set forth in SEQ ID NO: 135; or viii) a nucleotide sequence as set forth in SEQ ID NO: 140 and a nucleotide sequence as set forth in SEQ ID NO: 136; or ix) a nucleotide sequence as set forth in SEQ ID NO: 140 and a nucleotide sequence as set forth in SEQ ID NO: 137; or x) a nucleotide sequence as set forth in SEQ ID NO: 141 and a nucleotide sequence as set forth in SEQ ID NO: 134; or xi) a nucleotide sequence as set forth in SEQ ID NO: 141 and a nucleotide sequence as set forth in SEQ ID NO: 135; or xii) a nucleotide sequence as set forth in SEQ ID NO: 141 and a nucleotide sequence as set forth in SEQ ID NO: 136; or xiii) a nucleotide sequence as set forth in SEQ ID NO: 141 and a nucleotide sequence as set forth in SEQ ID NO: 137. In some embodiments, the isolated nucleic acid molecule encoding the anti-IL-31 antibody comprises i) a nucleotide sequence as set forth in SEQ ID NO: 138 and a nucleotide sequence as set forth in SEQ ID NO: 121. In some embodiments, the isolated nucleic acid molecule encoding the anti-IL-31 antibody comprises a nucleotide sequence as set forth in SEQ ID NO: 122; and a nucleotide sequence as set forth in SEQ ID NO: 121.

In some embodiments wherein the antibody is an anti-IL-31/IL-13 multispecific, preferably bispecific, antibody there is provided a set of nucleic acid molecules, wherein the set of nucleic acid molecules comprises an isolated nucleic acid molecule that encodes an IL-31 binding part antibody as set forth above and an isolated nucleic acid molecule encoding an IL-13 binding part antibody as set forth above.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO:3;
iv) an LCDR1 comprising SEQ ID NO: 4;
v) an LCDR2 comprising SEQ ID NO: 5; and
vi) an LCDR3 comprising SEQ ID NO:6, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,

the encoded first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6, and
the encoded second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16, preferably wherein HCDR1, HCDR2, HCDR3, LCDR1, LCDR2, and LCDR3 are defined according to the Kabat numbering scheme.

In some embodiments,
the encoded first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 28 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 29 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 31 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 32 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 30 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a heavy chain variable region (VH) comprising or consisting of the amino acid sequence shown in SEQ ID NO: 7 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 8 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a heavy chain variable region (VH) comprising or consisting of the amino acid sequence shown in SEQ ID NO: 17 (or an amino acid sequence having at least 80%, at least 85%, at least 90%, at least 95%, at least 96%, at least 97%, at least 98%, or at least 99% sequence identity therewith) and a light chain variable region (VL) comprising or consisting of the amino acid sequence shown in SEQ ID NO: 18 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 37 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith); and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 38 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43.

In some embodiments,
the encoded first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 39 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 43 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 104 or 107 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 105 or 108 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 34 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 35 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 33 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 10 or 109 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 or 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises a light chain comprising or consisting of the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 10 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 20 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises or consisting of a light chain comprising the amino acid sequence shown in SEQ ID NO: 9 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 109 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 19 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 110 (or an amino acid sequence having at least 80%, at least 85%, at least 90%, at least 95%, at least 96%, at least 97%, at least 98%, or at least 99% sequence identity therewith).

In some embodiments,
the encoded first part that binds to IL-31 comprises or consisting of a light chain comprising the amino acid sequence shown in SEQ ID NO: 177 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 176 (or an amino acid sequence having at least 80%, at least 85%, at least 90%, at least 95%, at least 96%, at least 97%, at least 98%, or at least 99% sequence identity therewith), and;
the encoded second part that binds to IL-13 comprises a light chain comprising or consisting of the amino acid sequence shown in SEQ ID NO: 179 (or an amino acid sequence having at least 80%, at least 85%, at least 90%, at least 95%, at least 96%, at least 97%, at least 98%, or at least 99% sequence identity therewith) and a heavy chain comprising or consisting of the amino acid sequence shown in SEQ ID NO: 178 (or an amino acid sequence having at least 80%, at least 85%, at least 90%, at least 95%, at least 96%, at least 97%, at least 98%, or at least 99% sequence identity therewith).

In some embodiments, there is provided a set of isolated nucleic acid molecules comprising:
an isolated nucleic acid molecule comprising a nucleotide sequence as set forth in SEQ ID NO: 122; and a nucleotide sequence as set forth in SEQ ID NO: 121, and;
an isolated nucleic acid molecule comprising a nucleotide sequence as set forth in SEQ ID NO: 126; and a nucleotide sequence as set forth in SEQ ID NO: 125.

In some embodiments, the isolated nucleic acid molecule is a cDNA molecule. In some embodiments, the nucleic acid molecules comprised by the set of nucleic acid molecules are cDNA molecules.

In an embodiment, the antibodies or fragments described herein are encoded by a transgene whose sequence has been codon optimized for expression in a mammalian cell, for example a HEK cell or a CHO cell.

Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In a further aspect, there is provided an expression vector or set of expression vectors comprising the nucleic acid molecule or set of nucleic acid molecules described herein. In a further aspect, there is provided a set of expression vectors comprising a set of nucleic acid molecules encoding a multispecific, preferably bispecific, antibody comprising a first part binding to IL-31 and a second part binding to IL-13, preferably wherein the nucleic acid molecule encoding the first part that binds to IL-31 and the nucleic acid molecule encoding the second part that binds to IL-13 are comprised by different (separate) vectors. Preferably, the first part and second part specifically bind to, respectively, IL-31 and IL-13, preferably human IL-31 and human IL-13. Expression vectors can include, but are not limited to, a virus, plasmid, cosmid, lambda phage, or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (e.g., Rous Sarcoma Virus, MMTV, MOMLV) or SV40 virus. Another class of vectors utilizes RNA elements derived from RNA viruses such as Semliki Forest virus, Easter Equine Encephalitis virus and Flaviviruses. An example of a preferred expression vector is a plasmid.

In a further aspect, there is provided a host cell comprising the nucleic acid molecule, set of nucleic acid molecules, expression vector, or set of expression vectors described herein. In some embodiments, the host cell comprises a nucleic acid molecule encoding a first part antibody that binds to IL-31 of a multispecific, preferably bispecific, antibody described herein, or an expression vector comprising said nucleic acid molecule. In some embodiments, the host cell comprises a nucleic acid molecule encoding a second part antibody that binds to IL-13 of a multispecific, preferably bispecific, antibody described herein, or an expression vector comprising said nucleic acid molecule.

In a further aspect, there is provided a mixture of host cells comprising a first and second host cell, wherein:
a. the first host cell comprises a nucleic acid molecule encoding the first part that binds to IL-31 of the multispecific antibody, preferably bispecific antibody, or an expression vector comprising said nucleic acid molecule; and
b. the second host cell comprises a nucleic acid molecule encoding the second part that binds to IL-13 of the multispecific antibody, preferably bispecific antibody, or an expression vector comprising said nucleic acid molecule.

In a further aspect, there is provided a cell culture comprising a host cell or a mixture of host cells described herein. The host cell can be, but is not limited to, a prokaryotic cell (e.g., a bacterium, for example E. coli), a eukaryotic cell (e.g., a mammalian cell), an insect cell, or a human cell. The host cell may correspond to a cell line. Suitable eukaryotic cell lines include, but are not limited to, Vero cells, HeLa cells, COS cells, CHO cells, HEK cells, BHK cells, and MDCKII cells. Other examples of suitable eukaryotic cell lines include, but are not limited to, COS-1 (ATCC No. CRL 1650), COS-7 (ATCC No. CRL 1651), BHK570 (ATCC No. CRL 10314), DHFR-CHO (see Urlaub et al., 1986, Somatic Cell Mol. Genet. 12, 555-556), mouse Sertoli cells, monkey kidney cells, African green monkey kidney cells (ATCC No. CRL 1587), canine kidney cells (ATCC CCL34), human lung cells (ATCC CCL 75), Hep G2 and myeloma or lymphoma cells (e.g., NSO; see U.S. Pat. No. 5,807,715), Sp2/0, YO cells. Suitable insect cells, include, but are not limited to, Sf9 cells.

In some embodiments, the host cell is a HEK cell or a CHO cell, for example a HEK293 (e.g., a HEK293T) cell or a CHO-K1 cell.

The expression vectors and host cells can be used to manufacture the antibodies of the present invention.

In a further aspect, there is provided a method for manufacturing an anti-IL-31 antibody described herein, comprising culturing a host cell comprising an isolated nucleic acid molecule or set of isolated nucleic acid molecules (or expression vector or set of expression vectors) encoding the anti-IL-31 antibody under conditions sufficient to express it, and thereafter recovering, and optionally purifying, the anti-IL-31 antibody from the host cell culture. In some embodiments, the anti-IL-31 antibody is further formulated in a pharmaceutical composition, optionally in combination with one or more pharmaceutically acceptable excipients, diluents, or carriers. Further information on pharmaceutical compositions is provided later herein.

Multispecific, e.g., bispecific, antibodies may be produced via co-expression of the first part- and second-part described herein using a single host cell. Such methods may use multiple nucleic acid molecules or expression vectors in a single host cell. Alternatively, multispecific, e.g., bispecific, antibodies may be produced using multiple host cells, e.g., a first host cell may express the first part that binds to IL-31 and a second host cell may express the second part that binds to a target other than IL-31, preferably to IL-13. In such methods, each host cell may comprise an isolated nucleic acid molecule or expression vector encoding the first- or second-part, or may comprise a set of isolated nucleic acid molecules or expression vectors encoding the first- or second-part. The host cells may be cultured in the same or different cultures. Production of the multispecific, preferably bispecific, antibody will generally involve culturing the host cell(s) under conditions sufficient (conducive) to express the first- and second-part followed by recovery of the first- and second-part. Optionally, the first- and second-part may be purified after recovery using any of the methods available to the skilled person, examples of which are provided elsewhere herein and further demonstrated in the experimental section. Following their recovery, the first- and second-part can be incubated together under conditions for isomerization (dimerization) to occur. Exemplary such conditions are reducing conditions that allow inter-chain disulfide bond reduction in the hinge region, followed by oxidizing conditions to re-form the inter-chain disulfide bonds. Further information is provided later herein, and exemplified in the experimental section. Once the multispecific antibody is obtained, it may optionally be purified. In some embodiments, the multispecific antibody is further formulated in a pharmaceutical composition, optionally in combination with one or more pharmaceutically acceptable excipients, diluents, or carriers.

Thus, in a further aspect, there is provided a method for manufacturing a multispecific, preferably bispecific, antibody described herein, comprising culturing a host cell comprising an isolated nucleic acid molecule or set of isolated molecules (or expression vector or set of expression vectors) encoding the first part that binds to IL-31 and the second part that binds to a target other than IL-31, preferably to IL-13, under conditions sufficient to express them, and thereafter recovering, and optionally purifying, the multispecific antibody from the host cell culture.

In a further aspect, there is provided a method for manufacturing a multispecific, preferably bispecific, antibody described herein, comprising culturing a first host cell an isolated nucleic acid molecule or set of isolated nucleic acid molecules (or expression vector or set of expression vectors) encoding the first part that binds to IL-31, and a second host cell comprising an isolated nucleic acid molecule or set of isolated nucleic acid molecules (or expression vector or set of expression vectors) encoding the second part that binds to a target other than IL-31, preferably to IL-13, or a mixture of the first- and second-host cell, under conditions sufficient to express them, and thereafter recovering, and optionally purifying, the first and second part from the host cell culture. In some embodiments, the first host cell and second host cell are cultured in different host cell cultures. In some embodiments, the first host cell and the second host cell are cultured in the same host cell culture.

In a further aspect, there is provided a method for manufacturing a multispecific, preferably bispecific, antibody described herein, comprising culturing a first host cell comprising an isolated nucleic acid molecule or set of isolated nucleic acid molecules (or expression vector or set of expression vectors) encoding the first part that binds to IL-31, and a second host cell comprising an isolated nucleic acid molecule or set of isolated nucleic acid molecules (or expression vector or set of expression vectors) encoding the second part that binds to a target other than IL-31, preferably to IL-13, (in the same or different cultures) under conditions sufficient to express them, recovering, and optionally purifying, the first- and second-part from the host cell culture(s), incubating the first part- and second-part under conditions sufficient to allow multispecific antibody dimerization, and obtaining, and optionally purifying, the multispecific antibody.

In some embodiments, the multispecific antibody is further formulated in a pharmaceutical composition, optionally in combination with one or more pharmaceutically acceptable excipients, diluents, or carriers.

In some embodiments, there is provided a method of producing an anti-IL-31/IL-13 multispecific, preferably bispecific, antibody, comprising:
a) culturing a first host cell expressing the first part that binds to IL-31 in a culture under conditions sufficient to express the antibody, and a second host cell expressing the second part that binds to IL-13 in a culture under conditions sufficient to express the antibody, wherein the first and second host cell are cultured in the same or in different cultures;
b) recovering the first- and second-part from the host cell culture(s);
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part; and
e) obtaining the multispecific antibody; and optionally purifying the multispecific antibody.

In some embodiments, there is provided a method of producing a pharmaceutical composition comprising an anti-IL-31/IL-13 multispecific, preferably bispecific, antibody, comprising:
a) culturing a first host cell expressing the first part that binds to IL-31 in a culture under conditions sufficient to express the antibody, and a second host cell expressing the second part that binds to IL-13 in a culture under conditions sufficient to express the antibody, wherein the first and second host cell are cultured in the same or in different cultures;
b) recovering the first- and second-part from the host cell culture(s);
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part;
e) obtaining the multispecific antibody; and optionally purifying the multispecific antibody; and
f) formulating the multispecific antibody in pharmaceutical composition, optionally in combination with one or more pharmaceutically acceptable excipients, diluents, or carrier.

In some embodiments, the pharmaceutical composition is further introduced in a container (e.g., in a pre-filled syringe, a vial, an injection pen, an auto-injector, an IV drip and bag, an infusion pump, a patch, an infusion bag, and the like), followed by packaging the container in a pharmaceutical product, e.g., a kit as described later herein, optionally together with instructions for use.

In some embodiments of the methods described herein, recovering the first- and second-part of the multispecific antibody from the host cell culture(s) comprises centrifugation or depth filtration optionally followed by sterile filtration.

In some embodiments of the methods described herein, a purification step is performed prior to incubating the first- and second-part of the multispecific antibody in step c) and/or step d). Optionally, the purification step is performed utilizing affinity chromatography, for example protein A affinity chromatography.

In some embodiments of the methods described herein, the first- and second-part of the multispecific antibody are incubated in step c) in equimolar concentrations. In some embodiments, the first- and second-part of the multispecific antibody are incubated in step c) in a molar ratio in the range of from about 1:1.01 to about 1:2 (first-to second-part). In some embodiments, the first- and second-part of the multispecific antibody are incubated in step c) in a molar ratio in the range of from about 1:1.01 to about 1:2 (second-to first-part).

Preferably, the first and second part specifically bind to, respectively, IL-31 and IL-13, preferably to human IL-31 and human IL-13.

In a further aspect, a DNA sequence encoding the light chain of a first half IgG antibody, a DNA sequence encoding the heavy chain of the first IgG half antibody (e.g., a half IgG antibody comprising an IL-31-binding Fab and an Fc, for example a half IgG1 antibody), a DNA sequence encoding the light chain of a second half IgG antibody, and a DNA sequence encoding the heavy chain of the second half IgG antibody (e.g., a half IgG antibody comprising an IL-13-binding Fab and an Fc, for example a half IgG1 antibody), are placed in separate expression vectors. The expression vectors are then co-transfected into a host cell. The encoded heavy chains and light chains are expressed in the host cell and assemble into functional antibodies. Alternatively, the expression vectors may be transfected into different host cell populations, and the multispecific antibody assembled in solution following their production. The different host cell populations may be cultured in the same or different cultures. Alternatively, DNA sequences (e.g., a DNA sequence encoding a light chain of an anti-IL-31 antibody and a DNA sequence encoding a heavy chain of an anti-IL-31 antibody linked to an additional binding domain (e.g., an IL-13 scFv) are placed in one expression vector. The expression vector may then be transfected into a host cell. The encoded heavy chains and light chains are expressed in the host cell and assemble into functional antibodies.

The nucleic acid molecules described herein can e.g., be produced by de novo solid-phase DNA synthesis or by PCR mutagenesis of an existing sequence e.g., encoding heavy or light chains of the antibodies described herein. Direct chemical synthesis of nucleic acids can be accomplished by methods known in the art, such as the phosphotriester method of Narang et al., (1979) Meth. Enzymol. 68:90; the phosphodiester method of Brown et al., (1979) Meth. Enzymol. 68:109; the diethylphosphoramidite method of Beaucage et al., (1981) Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., (1991) Nucleic Acids Res. 19:967; and Eckert et al., (1991) PCR Methods and Applications 1:17. Further examples are provided in the experimental section herein.

Various expression vectors can be employed to express the polynucleotides encoding chains or binding domains of the antibodies described herein. Both viral-based and nonviral expression vectors can be used to produce the antibodies in host cell, e.g., a mammalian host cell (for example a HEK cell or a CHO cell). Nonviral vectors and systems include plasmids, episomal vectors, typically comprising an expression cassette for expressing a protein or RNA, and human artificial chromosomes (see, e.g., Harrington et al., (1997) Nat Genet 15:345). For example, nonviral vectors useful for expression of the polynucleotides and polypeptides in mammalian (e.g., human) cells include pThioHis A, B & C, pcDNA3.1/His, pEBVHis A, B & C, (Invitrogen, San Diego, CA), MPSV vectors, and numerous other vectors known in the art for expressing other proteins. Useful viral vectors include vectors based on retroviruses, adenoviruses, lentiviruses, adeno associated viruses, herpes viruses, vectors based on SV40, papilloma virus, HBP Epstein Barr virus, vaccinia virus vectors and Semliki Forest virus (SFV). See, Brent et al., (1995) supra; Smith, Annu. Rev. Microbiol. 49:807; and Rosenfeld et al., (1992) Cell 68:143.

The skilled person understands that the choice of expression vector depends on the intended host cells in which the vector is to be expressed. Typically, the nucleic acid molecules and expression vectors described herein comprise a promoter and other regulatory sequences (e.g., enhancers) that are operably linked to the polynucleotides encoding the antibodies or fragments thereof described herein. In some embodiments, an inducible promoter is employed to prevent expression of inserted sequences except under inducing conditions. Inducible promoters include, e.g., arabinose, lacZ, metallothionein-based promoter or a heat-shock protein promoter. Cultures of transformed host cells can be expanded under noninducing conditions without biasing the population for coding sequences whose expression products are better tolerated by the host cells. In addition to promoters, other regulatory elements may also be required or desired for efficient expression of the heavy chains and light chains of the antibodies or fragments thereof. These elements typically include an ATG initiation codon and adjacent ribosome binding site or other sequences. In addition, the efficiency of expression may be enhanced by the inclusion of enhancers appropriate to the cell system in use (see, e.g., Scharf et al., (1994) Results Probl. Cell Differ. 20:125; and Bittner et al., (1987) Meth. Enzymol., 153:516). For example, the SV40 enhancer or CMV enhancer may be used to increase expression in mammalian host cells. The nucleic acid molecules may also provide a secretion signal sequence positioned to form a fusion protein with polypeptides encoded by inserting the above-described sequences encoding the heavy chains and/or light chains or fragments thereof. More often, the inserted antibody encoding sequences are linked to a signal sequences before inclusion in an expression vector.

The host cells for harboring and expressing the present antibodies can be either prokaryotic or eukaryotic. E. coli is one prokaryotic host useful for cloning and expressing the polynucleotides described herein. Other microbial hosts suitable for use include bacilli, such as Bacillus subtilis, and other Enterobacteriaceae, such as Salmonella, Serratia, and various Pseudomonas species. In nucleic acid molecules and expression vectors intended for these prokaryotic hosts, additional expression control sequences compatible with the host cell (e.g., an origin of replication) can be included as appropriate. In addition, any number of a variety of known promoters will be present, such as the lactose promoter system, a tryptophan (trp) promoter system, a beta-lactamase promoter system, or a promoter system from phage lambda, and the like. The promoters typically control expression, optionally with an operator sequence, and include ribosome binding site sequences and the like, for initiating and completing transcription and translation. Other microbes, such as yeasts (e.g., Saccharomyces (such as Saccharomyces cerevisiae) or Pichia (such as Pichia pastoris)) or filamentous fungi (e.g., Aspergillus (such as Aspergillus niger)) can alternatively be employed to express the antibody of the invention. Insect cells (e.g., Spf9 cells), optionally in combination with baculovirus vectors, can also be used.

In some embodiments, mammalian host cells are used to express and produce the antibodies or fragments thereof of the present invention. Such cells can be e.g., a hybridoma cell line expressing endogenous immunoglobulin genes) or a mammalian cell line harboring an exogenous expression vector (e.g., HEK (e.g., HEK293T) or CHO (e.g., CHO-K1) cells, for example as exemplified in the experimental section). These include any normal mortal or normal or abnormal immortal animal or human cell. For example, a number of suitable host cell lines capable of secreting intact immunoglobulins have been developed including the CHO cell lines (e.g., CHO-K1), various Cos cell lines, HeLa cells, myeloma cell lines, transformed B-cells, HEK cells (e.g., HEK293T) and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., (1986) Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art, as well derivatives thereof.

Methods for introducing expression vectors containing the polynucleotide sequences of interest can vary depending on the type of cellular host. For example, calcium chloride transfection is commonly utilized for prokaryotic cells, whereas calcium phosphate treatment or electroporation may be used for other cellular hosts (see generally Sambrook, et al., Molecular Cloning: A Laboratory Manual (4^{th} Edition), 2012, Cold Spring Harbor Laboratory Press, USA). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (e.g., see Elliot and O'Hare, (1997) Cell 88:223), agent-enhanced uptake of DNA, and ex vivo transduction. For long-term, high-yield production of recombinant proteins, stable expression will often be desired. For example, cell lines which stably express antibody chains fragments thereof can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. Non-limiting examples of selectable markers that can be used are folic acid receptor (FolR) and dihydrofolate reductase (DHFR) genes. The folate receptor (FolR) mediates folate import, and dihydrofolate reductase (DHFR) converts folate into vital precursors for purine and methionine synthesis. Recombinant cells that have successfully integrated the expression vector into the genome and express FolR and DHFR have a clear growth advantage when low-folate culture medium is utilized. The selection pressure is further enhanced by supplementing the culture medium with MTX, a folate analogue that inhibits DHFR. The combination of FolR, DHFR, folate restriction and MTX provides a strong selective regime for enrichment of recombinant cells.

The nucleic acid molecules or expression vectors may include leaky stop codons, e.g., after the sequence encoding for a heavy chain of an antibody described herein. Inclusion of such codons, followed by a transmembrane section, can allow for a small fraction of antibody being anchored to the cell membrane instead of being secreted. Membrane-anchored antibodies can then be used as staining targets in combination with flow cytometry (e.g., using FACS) to select antibody-producing cells. An exemplary schematic overview of an expression vector using this setup is provided in FIG. 13.

The antibodies or fragments thereof described herein are typically recovered from a host cell culture medium as a secreted polypeptide, although they also may be recovered from host cell lysate when directly produced without being secreted. If the antibody or fragment thereof is membrane-bound, it can be released from the membrane using a suitable detergent solution (e.g., Triton-X 100). Lysis methods for prokaryotic and eukaryotic cells are well-known to the skilled person.

In some cases, the antibody is recovered and optionally purified from recombinant cell proteins or polypeptides to obtain preparations that are substantially homogeneous as to heteromultimer. As a first step, the culture medium or lysate is typically centrifuged to remove particulate cell debris. Alternatively, depth filtration followed by sterile filtration may be used. The produced antibodies or fragments thereof can be conveniently purified e.g., by hydroxylapatite chromatography, gel electrophoresis, dialysis, or affinity chromatography (e.g., on a CaptureSelect CH1-XL resin (Thermofisher Scientific, WA, MA, USA). Other techniques for protein purification such as fractionation on an ion-exchange column, ethanol precipitation, reverse phase HPLC, chromatography on silica, chromatography on heparin Sepharose, chromatography on an anion or cation exchange resin (such as a polyaspartic acid column), chromatofocusing, SDS-PAGE, protein A-affinity chromatography, and ammonium sulfate precipitation are also available. Purity may be e.g., determined using size exclusion chromatography, e.g., on commercial columns like the BEH200 SEC column (Waters #186005225). Purity may also be e.g., determined using CE-SDS capillary electrophoresis, e.g., on commercial systems like the Beckman Coulter PA 800 system (Beckman Coulter, CA, USA). Further exemplary assays for antibody isolation, purification and purity analysis are provided in the experimental section later herein.

### C. Heterodimerization Methods

Heterodimerization methods, such as Fab arm exchange and knobs into holes (K-i-H) technology, are well known in the art. Such methods typically use parental antibodies having mutations in the CH3 region of antibodies that favor, or “skew”, formation to heterodimerization over homodimer formation, as discussed elsewhere herein.

The Fab arm exchange approach is described e.g. by Labrijn et al., Proc Natl Acad Sci USA. 2013 Mar. 26; 110(13):5145-50 and WO2013/060867, and involves mixing of two separately expressed parental antibodies under controlled reducing conditions to separate the antibodies into half-antibodies (part-antibodies), allowing for the heterodimerization of half antibodies to form a multispecific, e.g., a bispecific antibody. The formation of bispecific antibodies can be assisted by subjecting the half-antibodies to conditions that allow oxidation of cysteines in the hinge region to form inter-chain disulfide bonds between the half-antibodies. Fab arm exchange protocols are also described in WO 08/119353, WO 2011/131746, and WO 2013/060867). Prior to Fab arm exchange, it is typically preferably to purify each parental antibody, e.g., using any of the purification methods described elsewhere herein.

Reducing conditions can e.g., be achieved by addition of a reducing agent. Non-limiting examples of suitable reducing agents include reduced glutathione (GSH), DTT (dithiothreitol), TCEP (tris(2-carboxyethyl)phosphine), 2-MEA (2-Mercaptoethylamine), L-cysteine, D-cysteine, and the like. The reducing agent can be removed after formation of the multispecific antibody, for example by buffer exchange, desalting chromatography, tangential-flow filtration, or normal flow filtration. Oxidizing conditions can e.g., be achieved by addition of an oxidizing agent. A non-limiting example of a suitable oxidizing agent is dehydroascorbic acid (dhAA). However, it is not necessary to add an oxidizing agent. For example, in some cases the reducing agent may be removed by e.g., buffer exchange, and the mixture comprising the antibodies may be incubated at e.g., 4° C. or other suitable temperature to allow for re-oxidation of inter-chain disulfide bonds. Oxidizing conditions may in some cases include the presence of oxygen, e.g., at least 0.01 mM oxygen, such as about 0.05 mM oxygen. Addition of oxygen can e.g., be performed by agitation, stirring, sparging with air, and the like. Oxidizing conditions may in some cases comprise adding a metal ion, e.g., copper, manganese, magnesium, iron, nickel, or cobalt, in a concentration of e.g., from about 0.1 to 100 pM. Additional product and process related impurities can be removed by e.g., ion-exchange chromatography (for example, by cation exchange chromatography (CEC) in bind-elute mode, followed by a multimodal anion exchange chromatography (MAC) in flow-through mode). The purified multispecific antibody can be concentrated and buffer exchanged to reach a desired concentration depending on the desired formulation and application. A further example of generation of a bispecific antibody according to the disclosure is provided in the experimental section.

Fab-arm exchange typically generates multispecific, e.g., bispecific, antibodies from parental antibodies (each one providing a “part” antibody as discussed elsewhere herein) and can be promoted by introducing e.g., F405L/K409R mutations pairs (EU numbering) as described earlier herein. For example, a first antibody having a first specificity (e.g., IL-31 binding specificity) may comprise a F405L mutation in the constant region and a second antibody having a second specificity (e.g., IL-13 binding specificity) may comprise a K409R in the constant region.

In an embodiment, a multispecific, e.g., bispecific, antibody according to the invention is generated by providing a first antibody having a first binding specificity, preferably having IL-31 binding specificity, and providing a second antibody having a second binding specificity which differs from said first binding specificity, preferably having IL-13 binding specificity, (herein termed “parental antibodies”), and incubating said first and second antibodies together under reducing conditions, followed by incubation under conditions that allow oxidation of cysteines in the hinge region to form inter-chain disulfide bonds between the heterodimeric multispecific antibody.

The reducing conditions selectively reduce the hinge disulfide bonds between the parental heavy chains, allowing the exchange e.g., of half-antibodies from parental homodimers to heterodimers. Heterodimerization of polypeptides comprising such mutations may be achieved e.g., under reducing conditions such as about 10-100 mM 2-MEA (e.g., 25, 50, or 100 mM 2-MEA) for about 1-10, e.g., 1.5-5, e.g., 5, hours at about 20-37° C., e.g., about 20° C. or 25° C. or 37° C. Non-limiting exemplary conditions are e.g., incubation for at least about 90 minutes at a temperature of at least about 20° C. in the presence of at least about 25 mM 2-mercaptoethylamine. In some cases, a metal chelating agent may be added, for example EDTA, EGTA, or citric acid.

In cases where an oxidizing agent is added, the amount required to achieve oxidizing conditions could vary depending on e.g., if the reducing agent is removed (e.g., by buffer exchange) prior to addition of the oxidizing agent. If the reducing agent is removed, then the amount can be lower. In the case of e.g., dehydroascorbic acid, the amount required can be typically in the range of 1-2 times the concentration of the reducing agent, but can be e.g., in the range of 0.01 mM to 1 mM in the absence of the reducing agent.

Another example of suitable FAE conditions are incubation with 2-MEA at a concentration of about 25 mM. The mixture of the first and second antibody can be incubated for about 90 min at about 37° C. or about 5 h at about 25° C. The 2-MEA can be removed by buffer-exchanging against PBS using e.g., desalting plates, desalting columns (5-kDA MWCO; such as provided by GE Healthcare) or diafiltration. Samples can be stored overnight, e.g., at about 4° C. to allow reoxidation of disulfide bonds to occur. Another example of suitable FAE conditions is provided in Labrijn et al, Proc Natl Acad Sci USA. 2013 Mar. 26; 110(13):5145-50.

The multispecific, e.g., bispecific, antibodies of the present invention can be made by producing a first antibody and a second antibody separately (e.g., in separate host cell cultures) and subjecting them to separate purifications prior to Fab-arm exchange. Alternatively, the first antibody and the second antibody can be produced together, e.g., by culturing a host cell expressing the first antibody and a host cell expressing the second antibody in the same culture, and co-purified e.g., on a single purification column, prior to Fab-arm exchange. Alternatively, in some embodiments wherein the host cell expressing the first antibody and the host cell expressing the second antibody are co-cultured, Fab-arm exchange made be performed prior to recovery and/or purification, e.g., in co-culture broth (without cell removal) or co-culture medium (following removal of the cells). Cells may be removed with any suitable method available to the skilled person, e.g., centrifugation, filtration, and the like. This can be advantageous in that the parental antibodies do not need to be separately recovered and/or purified, and antibody recovery and/or purification can take place after assembly of the multispecific antibody. Examples of performing Fab-arm exchange in a co-culture setting can e.g., be found in WO2015/171822.

In some embodiments, re-oxidation is performed on a purification column, e.g., as described in WO2024/209389.

In an aspect, there is provided a method for manufacturing (producing) a multispecific e.g., a bispecific heterodimeric antibody disclosed herein, said method comprising; a) a first step of culturing a first host cell expressing a first homodimeric IgG antibody that binds IL-31 (i.e., an IgG anti-IL-31 antibody, e.g., an IgG1 anti-IL-31 antibody); and culturing a second host cell expressing a second homodimeric IgG antibody having a second binding specificity which differs from said first binding specificity, preferably an IgG anti-IL-13 antibody (e.g., an IgG1 anti-IL-13 antibody, wherein the first and second host cell are cultured in the same or in different cultures; b) recovering the first and second homodimeric IgG antibody from the host cell culture(s); c) incubating together the IgG antibodies obtained in step b) under reducing conditions that allow inter-chain disulfide bond reduction in the hinge region; d) incubating together the IgG antibodies under conditions that allow oxidation of cysteines in the hinge region to form inter-chain disulfide bonds, e) obtaining the multispecific heterodimeric antibody; and optionally e) purifying the multispecific heterodimeric antibody. In some embodiments, a purification step is performed prior to recovering the first and second IgG antibodies in step b). In some embodiments, the first and second homodimeric IgG antibodies are incubated together in step c) and step d) in equimolar concentrations, or in a molar ratio from about 1:1.01 to about 1:2 (first-to-second or second-to-first antibody). In some embodiments, the multispecific antibody is formulated in a pharmaceutical composition, optionally in combination with one or more pharmaceutically acceptable excipients, diluents, or carriers.

In an aspect, there is provided a method of producing a multispecific, e.g., bispecific, heterodimeric antibody disclosed herein, comprising the steps of:
a) culturing a host cell engineered to express a first part that binds to IL-31, or that comprises a nucleic acid molecule encoding a first part that binds to IL-31, and a host cell engineered to express a second part that binds to IL-13, or that comprises a nucleic acid molecule encoding a second part that binds to IL-13, under conditions sufficient to produce the first part that binds to IL-31 and the second part that binds to IL-13;
b) recovering, and optionally purifying, the first part that binds to IL-31 and the second part that binds to IL-13 from the culture medium and/or cells;
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part; and
e) obtaining the multispecific antibody; and optionally purifying the multispecific antibody.

In some embodiments, the host cell engineered to express the first part that binds to IL-31, or that comprises a nucleic acid molecule encoding the first part that binds to IL-31, and the host cell expressing the second part that binds to IL-13, or that comprises a nucleic acid molecule encoding the second part that binds to IL-13, are cultured in the same or different cultures. In some embodiments, recovering the first- and second-part of the multispecific antibody from the host cell culture(s) comprises centrifugation or depth filtration optionally followed by sterile filtration. In some embodiments, a purification step is performed prior to incubating the first- and second-part of the multispecific antibody in step c) and/or step d). Optionally, the purification step is performed utilizing affinity chromatography, for example protein A affinity chromatography.

In an aspect, there is provided a method of producing a multispecific, e.g., bispecific, heterodimeric antibody disclosed herein, comprising the steps of:
a) culturing a host cell engineered to express a first part that binds to IL-31, or that comprises a nucleic acid molecule encoding the first part that binds to IL-31, and a host cell engineered to express a second part that binds to IL-13, or that comprises a nucleic acid molecule encoding the second part that binds to IL-13, under conditions sufficient to produce the first part that binds to IL-31 and the second part that binds to IL-13;
b) incubating together the first- and second-part, preferably under reducing conditions that allow inter-chain disulfide bond reduction in the hinge region;
c) incubating the first- and second-part under conditions that allow oxidation of cysteines in the hinge region to form inter-chain disulfide bonds between the first- and second-part; and
d) obtaining the multispecific antibody; and optionally purifying the multispecific antibody.

In some embodiments, the host cell engineered to express the first part that binds to IL-31, or that comprises a nucleic acid molecule encoding the first part that binds to IL-31, and the host cell engineered to express the second part that binds to IL-13, or that comprises a nucleic acid molecule encoding the second part that binds to IL-13, are cultured in the same culture. In some embodiments, the first- and second-part are incubated together in step b) without any prior recovery of the first- and second-part from the culture. In some embodiments, the first- and second-part are incubated together in the step b) without prior removal of the host cells from the culture. In some embodiments, the first- and second-part are incubated together after removal of the host cells from the culture. Optionally, the purification step comprises affinity chromatography, for example protein A affinity chromatography. In some embodiments, re-oxidation of the cysteines in the hinge region is performed during the protein A affinity chromatography step.

In some embodiments of the methods described herein, the first- and second-part of the multispecific antibody are incubated together in equimolar concentrations. In some embodiments, the first- and second-part of the multispecific antibody are incubated together in a molar ratio in the range of from about 1:1.01 to about 1:2 (first-to second-part). In some embodiments, the first- and second-part of the multispecific antibody are incubated together in a molar ratio in the range of from about 1:1.01 to about 1:2 (second-to first-part).

In some embodiments, the multispecific antibody is formulated in a pharmaceutical composition, optionally in combination with one or more pharmaceutically acceptable excipients, diluents, or carriers.

When using any of the heterodimerization methods described herein, preferably the first antibody e.g., IgG antibody, comprises an Fc region comprising an F405L mutation (EU numbering), and the second antibody e.g., IgG antibody, comprises an Fc region comprising a K409R mutation (EU numbering). The first antibody may be an anti-IL-31 antibody comprising an Fc region comprising a F405L mutation (EU numbering) and the second antibody may be an anti-IL-13 antibody comprising an Fc region comprising a K409R mutation (EU numbering). Alternatively, the first antibody may be an anti-IL-13 antibody comprising an Fc region comprising an F405L mutation and the second antibody may be an anti-IL-31 antibody comprising an Fc region comprising a K409R mutation.

Alternatively, knob-in-hole technology may be used, as described earlier herein. In some embodiments, when using the heterodimerization methods described herein, the first antibody, e.g., IgG antibody, comprises an Fc region comprising a knob mutation (e.g., S354C, T366W (EU Numbering)), and the second antibody, e.g., IgG antibody, comprises an Fc region comprising a hole mutation (e.g., Y349C, T366S, L368A, Y407V (EU Numbering)). The first antibody may be an IL-31 antibody comprising an Fc region comprising a knob mutation and the second antibody may be an IL-13 antibody comprising an Fc region comprising a hole mutation. Alternatively, the first antibody may be an IL-13 antibody comprising an Fc region comprising a knob mutation and the second antibody may be an IL-31 antibody comprising an Fc region comprising a hole mutation.

In some embodiments, there is provided a method for making the multispecific (e.g., bispecific) antibodies or fragments thereof of the invention by mutating one or more residues in the hinge region with charged residues that favor heterodimer formation over homodimer formation. In some embodiments, the mutation in the first antibody is at position 221 and/or 228 (EU numbering). In some embodiments, the amino acid modification(s) is a charged residue (e.g., Lys, Arg, His, Glu, and Asp) or polar residue (e.g., Ser and Thr). In some embodiments, the multispecific (e.g., bispecific) antibody comprises a first part antibody comprising Arg221 or Glu221 and Arg228 or Glu228 (EU numbering); and a second part antibody comprising Arg221 or Glu221 and Arg228 or Glu228 (EU numbering), wherein the amino acids at positions 221 and 228 of the first part antibody are not the same as the corresponding amino acids in the second part antibody. In some embodiments, the first part antibody comprises Arg221 or Glu221 and Arg228 or Glu228, and additionally (in the CH3 region) Arg409 or Glu368; and the second part antibody comprises Arg221 or Glu221 and Arg228 or Glu228, and additionally (in the CH3 region) Arg409 or Glu368, wherein the amino acids at positions 221 and 228 and 368 and 409 of the first part antibody are not the same as the corresponding amino acids in the second part antibody. In this embodiment, either the first antibody or second antibody may comprise a kappa light chain and the other may comprise a lambda light chain.

### Pharmaceutical Compositions

Provided herein are pharmaceutical compositions comprising an anti-IL-31 antibody e.g., a monospecific or multispecific e.g., bispecific, antibody as described herein, or a fragment thereof (e.g., a Fab or any other antigen-binding fragment described herein), optionally in combination with one or more pharmaceutically acceptable excipients, diluents or carriers. A composition, such as a pharmaceutical composition, may be formulated to be suitable for a particular mode of administration. Suitable administration modes are described later herein. The types of formulations, as well as formulation methods, that will be the most appropriate for a composition described herein for it to be suitable for a particular mode of administration are well within the capabilities of the skilled person and are described in standard handbooks, such as Remington: The Science and Practice of Pharmacy, 23rd edition, Elsevier (2020).

The phrase “pharmaceutically acceptable” means approved by a regulatory agency of a federal or a state government, or listed in the U.S. Pharmacopeia or other generally recognized pharmacopeia for use in animals, and more particularly, in humans. Pharmaceutically acceptable ingredients include pharmaceutically acceptable carriers, fillers, stabilizers, preservatives, solubilizers, vehicles, diluents and/or excipients. Such pharmaceutically acceptable carriers, fillers, stabilizers, preservatives, solubilizers, vehicles, diluents and/or excipients may for instance also be found in Remington: The Science and Practice of Pharmacy (supra).

Pharmaceutical compositions can be prepared in accordance with methods well known and routinely practiced in the art in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). Pharmaceutical compositions are preferably manufactured under GMP conditions.

The pharmaceutical compositions of the invention comprise a therapeutically effective dose or efficacious dose of the active ingredient (i.e., an antibody or fragment thereof described herein, e.g., a monospecific or multispecific, preferably bispecific, antibody).

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, preferably without being toxic to the patient and/or consistent with an acceptable level of side effects.

Selecting an administration regimen for a therapeutic depends on several factors, including the serum or tissue turnover rate of the therapeutic, the level of symptoms, the immunogenicity of the entity, and the accessibility of the target cells in the biological matrix.

Guidance in selecting appropriate doses of antibodies are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Determination of the appropriate dose is made by the clinician or veterinarian, e.g., using parameters or factors known or suspected in the art to affect treatment or predicted to affect treatment. Generally, the dose begins with an amount lower than that required to achieve the desired therapeutic effect and the dose is gradually increased until the desired effect is achieved, preferably without being toxic to the patient and/or consistent with an acceptable level of side effects. In general, the effective dose varies depending upon many different factors, including the activity of the particular antibody employed, rate of excretion, the route of administration, target site, physiological state of the patient, whether the patient is human or an animal, other medications administered, whether treatment is prophylactic or therapeutic, the age, sex, weight, condition, general health and prior medical history of the patient being treated and is affected by the half-life of the molecule dosed (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A pharmaceutical composition described herein can be administered e.g., systemically by methods known in the art. The route and/or mode of administration can vary depending upon the desired results. It is preferred that the composition is a solution or suspension for injection. The composition may e.g., be an aqueous solution for injection comprising one or more pharmaceutically acceptable excipients, diluents or carriers. Such antibody formulations may comprise one or more of an excipient to adjust tonicity and/or osmolality, a buffer and/or a surfactant. The composition should be sterile and fluid. Antibody formulations are known in the art and, for example, include lebrikizumab formulations, marketed as Ebglyss®, which comprises as excipients histidine, glacial acetic acid, sucrose, polysorbate 20 and water for injection.

An antibody (e.g., a monospecific or multispecific e.g., bispecific, antibody) or fragment thereof described herein can be administered via one or more routes of administration using one or more of a variety of methods known in the art. As will be appreciated by the skilled artisan, the route and/or mode of administration will vary depending upon the desired results. Administration will typically be via a syringe. Thus, the invention further provides a delivery device (e.g., a syringe) including a pharmaceutical composition of the invention (e.g., pre-filled syringe), and a kit comprising a syringe and a vial that includes a pharmaceutical composition of the invention.

In one aspect, the pharmaceutical composition is administered subcutaneously. In one aspect, the pharmaceutical composition is administered intravenously.

Subjects in need of treatment (e.g., patients) will receive an effective amount of the antibody or fragment thereof (e.g., the monospecific or multispecific e.g., bispecific, antibody) as the active ingredient (i.e., an amount that is sufficient to achieve or at least partially achieve the desired therapeutic effect). A dose is thus therapeutically effective if it can produce even an incremental change in the symptoms or conditions associated with the disease or disorder being treated. A therapeutically effective dose does not have to completely cure the disease or disorder or completely eliminate its symptoms. Preferably, the therapeutically effective dose can at least partially arrest the disease or disorder and its complications in a patient suffering from the disease or disorder.

Preferably, the therapeutically effective dose can at least partially ameliorate one or more symptoms of the disease or disorder. For example, a therapeutically effective dose can decrease one or more symptoms of the disease or disorder by at least 10%, at least 20%, at least 30%, at least 40%, or at least 50%, as measured using standard methods available to the clinician. Amounts effective for this use will depend upon the severity of the disorder being treated and the general state of the patient's own immune system. Suitably, a therapeutically effective dose achieves a therapeutically effective serum level, preferably maintained during the course of treatment. A dose is prophylactically effective if it can at least partially prevent the symptoms or conditions associated with a disease or disorder in a subject in danger of developing the disease or disorder.

An exemplary non-limiting dosage administered to a patient may be about 0.01 mg/kg to about 100 mg/kg of the patient's body weight, e.g., about 1 mg/kg to about 100 mg/kg of the patient's body weight.

When a series of doses are administered, these may, for example, be administered approximately daily, approximately every week, approximately every 2 weeks, approximately every 3 weeks, approximately every 4 weeks (monthly), approximately every 2 months, approximately every 3 months (quarterly), approximately every 6 months. The doses may, for example, be administered until disease progression or one or more symptoms of the disease are ameliorated, disease progression is halted and/or reversed, and the like, as determined by the physician. For example, from about two, three, four, up to about 17 or more doses may be administered.

In some embodiments, the antibodies or fragments thereof the present invention may be co-administered to a patient using a pharmaceutical composition comprising an antibody or fragment thereof in combination with one or more other therapies or therapeutic agents (e.g., a corticosteroid and/or other therapeutic agent). In some embodiments, the pharmaceutical composition further comprises one or more additional therapeutic agents.

Where necessary, the antibody or fragment thereof may be formulated in a pharmaceutical composition suitable for injection that includes a solubilizing agent and a local anaesthetic such as lidocaine to ease pain at the site of the injection.

The antibodies (e.g., the monospecific or multispecific e.g., bispecific, antibodies) or fragments thereof (e.g., Fabs) described herein can be administered via one or more routes of administration using one or more of a variety of methods known in the art. As will be appreciated by the skilled artisan, the route and/or mode of administration will vary depending upon the desired results. Selected routes of administration for the antibodies include intravenous, intramuscular, intradermal, intraperitoneal, subcutaneous, spinal or other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the present disclosure can be administered via a non-parenteral route, such as a topical, epidermal or mucosal route of administration, for example, intranasally, orally, vaginally, rectally, sublingually, or topically.

The antibodies (e.g., the monospecific or multispecific e.g., bispecific, antibodies) or fragments thereof (e.g., Fabs) described herein may be administered via any of the abovementioned routes using, e.g., an injection device, an injection pen, a vial and syringe, pre-filled syringe, autoinjector, an infusion pump, a patch pump, an infusion bag and needle, etc. If administered in a controlled release or sustained release system, a pump may be used to achieve controlled or sustained release (see e.g., Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neural. 25:351; Howard et al., 1989, J. Neurosurg. 7 1:105); U.S. Pat. Nos. 5,679,377; 5,916,597; 5,912,015; 5,989,463; 5,128,326; WO 1999/15154; and WO 1999/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising an antibody or fragment thereof (see, e.g., U.S. Pat. No. 4,526,938, WO 1991/05548, WO 1996/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760.

If the antibodies (e.g., the monospecific or multispecific e.g., bispecific, antibodies) or fragments thereof (e.g., Fabs) are administered topically, they can be formulated in the form of an ointment, cream, transdermal patch, lotion, gel, shampoo, spray, aerosol, solution, emulsion, or other forms known to one of skill in the art (see e.g., Remington: The Science and Practice of Pharmacy (supra)). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as Freon) or in a squeeze bottle. Moisturizers or humectants can also be added to pharmaceutical compositions and dosage forms if desired. Examples of such additional ingredients are known in the art.

If the antibodies (e.g., the monospecific or multispecific e.g., bispecific, antibodies) or fragments thereof are administered intranasally, they can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

The antibodies or fragments thereof of present disclosure can also be cyclically administered to a patient.

In certain embodiments, the antibodies (e.g., the monospecific or multispecific e.g., bispecific, antibodies) or fragments thereof of present disclosure can be formulated to ensure optimal distribution in vivo. For example, the blood-brain barrier (BBB) excludes many highly hydrophilic compounds. In cases wherein crossing the BBB is desirable, the antibodies or fragments thereof can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhancing targeted drug delivery (see, e.g., Ranade W (1989) J. Clin. Pharmacol. 29:685). Exemplary targeting moieties include folate or biotin (see, e.g., U.S. Pat. No. 5,416,016 to Low et al); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233: 134); p 120 (Schreier et al (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Therapeutic Uses The skilled person understands that aspects and embodiments described herein relating to the antibodies (e.g., the monospecific or multispecific e.g., bispecific, antibodies) or fragments thereof (e.g., Fabs or any other antigen-binding fragment described herein) of the invention, or to compositions, such as pharmaceutical compositions, comprising them, for use in therapeutic applications are also applicable to their use in therapeutic applications, to their use in the manufacture of medicaments, as well to therapeutic methods comprising their administration to a subject in need thereof. It is further understood that the aspects and embodiments referring to the therapeutic uses of the anti-IL-31 antibodies described herein are also applicable to their antigen-binding fragments. Preferably, the IL-31 is human IL-31. In embodiments wherein the anti-IL-31 antibody is multispecific, preferably bispecific, and further specifically binds to IL-13, the IL-13 is preferably human IL-13. In a further aspect, there is provided an antibody, multispecific antibody, preferably bispecific antibody, or a pharmaceutical composition thereof, as described herein, for use as a medicament. In a further aspect, there is provided the use of an antibody, multispecific antibody, preferably bispecific antibody, or a pharmaceutical composition thereof, as described herein as a medicament. In a further aspect, there is provided the use of an antibody, multispecific antibody, preferably bispecific antibody, or a pharmaceutical composition thereof, as described herein for the manufacture of a medicament. In a further aspect, there is provided a method of treatment comprising administering to a subject in need thereof a therapeutically effective amount of an antibody, multispecific antibody, preferably bispecific antibody, or a pharmaceutical composition thereof, as described herein.

The present invention provides a method of preventing and/or treating diseases or disorders described herein by administering to a subject in need thereof a therapeutically effective amount of the antibodies (e.g., the monospecific or multispecific e.g., bispecific, antibodies) described herein, or a pharmaceutical composition thereof.

In a further aspect, there is provided an antibody, multispecific antibody, preferably bispecific antibody, or a pharmaceutical composition thereof, as described herein for use in preventing and/or treating an IL-31-related (e.g., mediated) disease or disorder. In a further aspect, there is provided the use of an antibody, multispecific antibody, preferably bispecific antibody, or a pharmaceutical composition thereof, as described herein for the manufacture of a medicament for the treatment and/or prevention of an IL-31-related disease or disorder. In a further aspect, there is provided a method of preventing and/or treating an IL-31-related disease or disorder in a subject in need thereof, comprising administering to the subject a therapeutically effective amount of an antibody, multispecific antibody, preferably bispecific antibody, or a pharmaceutical composition thereof, as described herein. In some embodiments, a prophylactically effective amount is administered to the subject in need thereof. In some embodiments, the subject in need thereof is a mammal, preferably a human.

In some embodiments, the IL-31-related disease or disorder is an inflammatory or immune disease or disorder. In some embodiments, the inflammatory or immune disease or disorder is a skin disease or disorder.

The anti-IL-31 antibodies described herein (e.g., the monospecific or multispecific e.g., bispecific, antibodies) are useful for inhibiting IL-31 activity. Thus, in a further aspect, there is provided a method of inhibiting the activity of IL-31, the method comprising contacting an effective amount of an antibody, multispecific antibody, preferably bispecific antibody, or a pharmaceutical composition thereof, as described herein, with a plurality of cells. Preferably, the cells are mammalian cells, more preferably human cells. In some embodiments, the method is an ex vivo or an in vitro method. In some embodiments, the anti-IL-31 antibody is administered to a human subject.

In a further aspect, there is provided a multispecific, preferably bispecific, antibody or a pharmaceutical composition thereof, as described herein, wherein the multispecific antibody binds to IL-31 and IL-13, for use in preventing and/or treating an IL-31- and IL-13-related disease or disorder. In a further aspect, there is provided the use of a multispecific, preferably bispecific, antibody or a pharmaceutical composition thereof, as described herein, wherein the multispecific antibody binds to IL-31 and IL-13, for the manufacture of a medicament for the treatment and/or prevention of an IL-31- and IL-13-related disease or disorder. In a further aspect, there is provided a method of preventing and/or treating an IL-31- and IL-13-related disease or disorder in a subject in need thereof, comprising administering to the subject a therapeutically effective amount of a multispecific, preferably bispecific, antibody or a pharmaceutical composition thereof, as described herein, wherein the multispecific antibody binds to IL-31 and IL-13. In some embodiments, a prophylactically effective amount is administered to the subject in need thereof. In some embodiments, the subject in need thereof is a mammal, preferably a human.

In some embodiments, the IL-31- and IL-13-related disease or disorder is an inflammatory or immune disease or disorder. In some embodiments, the inflammatory or immune disease or disorder is a skin disease or disorder.

The anti-IL-31/IL-13 antibodies described herein are useful for simultaneously inhibiting IL-31 and IL-13 activity. Thus, in a further aspect, there is provided a method of inhibiting the activity of IL-31 and IL-13, the method comprising contacting an effective amount of a multispecific, preferably bispecific, antibody or a pharmaceutical composition thereof, as described herein, wherein the multispecific antibody binds to IL-31 and IL-13 with a plurality of cells. Preferably, the cells are mammalian cells, more preferably human cells. In some embodiments, the method is an ex vivo or an in vitro method. In some embodiments, the multispecific antibody is administered to a human subject.

In a further aspect, there is provided a multispecific, preferably bispecific, antibody or a pharmaceutical composition thereof, as described herein, wherein the multispecific antibody binds to IL-31 and IL-13, for use in preventing and/or treating an IL-13-related disease or disorder. In a further aspect, there is provided the use of a multispecific, preferably bispecific, antibody or a pharmaceutical composition thereof, as described herein, wherein the multispecific antibody binds to IL-31 and IL-13, for the manufacture of a medicament for the treatment and/or prevention of an IL-13-related disease or disorder. In a further aspect, there is provided a method of preventing and/or treating an IL-13-related disease or disorder in a subject in need thereof, comprising administering to the subject a therapeutically effective amount of a multispecific, preferably bispecific, antibody or a pharmaceutical composition thereof, as described herein, wherein the multispecific antibody binds to IL-31 and IL-13. In some embodiments, a prophylactically effective amount is administered to the subject in need thereof. In some embodiments, the subject in need thereof is a mammal, preferably a human.

In some embodiments, the IL-13-related disease or disorder is an inflammatory or immune disease or disorder. In some embodiments, the inflammatory or immune disease or disorder is a skin disease or disorder.

The anti-IL-31/IL-13 antibodies described herein are useful for inhibiting IL-13 activity. Thus, in a further aspect, there is provided a method of inhibiting the activity of IL-13, the method comprising contacting an effective amount of a multispecific, preferably bispecific, antibody or a pharmaceutical composition thereof, as described herein, wherein the multispecific antibody binds to IL-31 and IL-13 with a plurality of cells. Preferably, the cells are mammalian cells, more preferably human cells. In some embodiments, the method is an ex vivo or an in vitro method. In some embodiments, the multispecific antibody is administered to a human subject.

The phrases “IL-31-related disease or disorder”, “IL-13-related disease or disorder”, and “IL-31- and IL-13 related disease or disorder” are meant to encompass all diseases and medical conditions in which IL-31 and/or IL-13 (as applicable) are involved, whether directly or indirectly, in the disease or medical condition including the causation, development, progress, persistence or pathology of the disease or condition. Accordingly, the terms include diseases, disorders, and other medical conditions associated with or characterized by aberrant IL-31 and/or IL-13 levels (as applicable) and/or treatable by reducing or suppressing IL-31- and/or IL-13-induced activity in target cells or tissues (as applicable). The diseases or disorders or conditions to be treated may in some cases be caused e.g., by IL-31 and/or IL-13 dysregulation, for example aberrant signaling and/or expression of IL-31 and/or IL-13.

In some embodiments, the IL-31-related and/or IL-13 related disease is selected from the group consisting of allergic, inflammatory and autoimmune diseases, such as atopic dermatitis, acute allergic contact dermatitis, chronic spontaneous urticaria, bullous pemphigoid, alopecia areata, dermatomyositis, prurigo nodularis, psoriasis, asthma, allergic rhinitis, food hypersensitivity, urticaria, bullous skin diseases, erythema multiform, contact dermatitis, rheumatoid arthritis, juvenile chronic arthritis, inflammatory bowel diseases (e.g., ulcerative colitis, Crohn's disease), idiopathic interstitial pneumonia, idiopathic pulmonary fibrosis, hypersensitivity pneumonitis, chronic obstructive pulmonary disease, RSV infection, uvelitis, scleroderma, osteoporosis, Hodgkin's lymphoma, chronic sinusitis, systemic lupus erythematosus, systemic sclerosis, and Sjogren's syndrome, preferably is atopic dermatitis.

### Atopic Dermatitis

“Atopic Dermatitis”, “AD”, or “eczema”, as used herein, refers to an inflammatory disease characterized by chronic inflammation of the skin. Symptoms of AD include, but are not limited to, pruritus (itchy skin and/or an itch sensation), dry skin, itching, small, raised bumps which may leak fluid and crust over when scratched, thickened skin, cracked skin, scaly skin, raw skin, skin sensitivity, swollen skin, scaly and dry eczematous lesions, and interruption and/or loss of sleep. The term also includes, but is not limited to, AD caused by or associated with epidermal barrier dysfunction, allergy (e.g., skin allergy, allergy to certain foods, pollen, mold, dust mite, animals, etc.), radiation exposure, and/or asthma. AD most often begins before age 5 and may persist into adolescence and adulthood. In some patients, AD flares up periodically followed by periods of clearance that may last several years. Atopic dermatitis (AD) is estimated to affect up to 20% of children and 3% of adults worldwide (Sacotte et al., Clin. Dermatol. 36:595-605 (2018)). Approximately 40% of those affected have moderate-to-severe disease (Chiesa et al., J. Invest. Dermatol. 139:583-90 (2019)). Pruritus typically worsens at night resulting in sleep loss and a substantial negative impact on quality of life and mental health of the patient. (Bieber T., N Engl J Med 2008; 358: 1483-94).

Various immune-related genes are associated with the development of AD including the filaggrin (FLG) gene, IL-4, IL-13, IL-31, IL-33, signal transducer and activator of transcription (STAT) 6, thymic stromal lymphopoietin (TSLP) and its receptors (IL-7R and TSLPR), interferon regulatory factor 2, Toll-like receptor 2, high-affinity IgE receptor (FcεRI) α gene, vitamin D receptor and cytochrome P450 family 27 subfamily A member 1 (CYP27A1). Elevated levels of IL-31 or its receptor are found in the serum of adult and pediatric patients with atopic dermatitis, and serum IL-31 levels have been found to correlate with severity of disease, Ezzat, M., et al., J Eur Acad Dermatol Venereol. 2011; 25: 334-339. Serum levels of IL-13 have also been shown to correlate with disease severity. In recent years, several therapeutics have been approved for the treatment of moderate-to-severe AD including tralokinumab and lebrikizumab, antibodies targeting IL-13. More recently, dupilumab, a human monoclonal antibody of the IgG4 subclass that binds to the interleukin-4 receptor alpha (IL-4Ra) subunit and inhibits IL-4 and IL-13 signaling, has been approved by the United States Food and Drug Administration (FDA) and European Medicines Agency (EMA) for moderate to severe AD. Nemolizumab (an anti-IL-31R mAb targeting the IL-31 receptor a subunit (IL31Ra)) has also recently been approved in Japan.

In a further aspect, there is provided an antibody, multispecific antibody, preferably bispecific antibody, or a pharmaceutical composition thereof, as described herein for use in preventing and/or treating atopic dermatitis. In a further aspect, there is provided the use of an antibody, multispecific antibody, preferably bispecific antibody, or a pharmaceutical composition thereof, as described herein for treating and/or preventing atopic dermatitis. In a further aspect, there is provided the use of an antibody, multispecific antibody, preferably bispecific antibody, or a pharmaceutical composition thereof, as described herein for the manufacture of a medicament for the treatment and/or prevention of atopic dermatitis. In a further aspect, there is provided a method of preventing and/or treating atopic dermatitis in a subject in need thereof, comprising administering to the subject a therapeutically effective amount of an antibody, multispecific antibody, preferably bispecific antibody, or a pharmaceutical composition thereof, as described herein. In some embodiments, a prophylactically effective amount is administered. In some embodiments, the subject in need thereof is a mammal, preferably a human.

The antibodies, multispecific antibodies, and pharmaceutical compositions thereof described herein can be used in the treatment and/or prevention of a symptom associated with atopic dermatitis. Non-limiting examples of symptoms associated with atopic dermatitis that are treatable include, but are not limited to, pruritus; dry skin; itching; red to brownish-gray patches of skin; small, raised bumps which may leak fluid and crust over when scratched; thickened skin; cracked skin; scaly skin; raw skin; skin sensitivity; and swollen skin.

Thus, in a further aspect, there is provided an antibody, multispecific antibody, preferably bispecific antibody, or a pharmaceutical composition thereof, as described herein for use in preventing and/or treating pruritus, preferably pruritus associated with atopic dermatitis. In a further aspect, there is provided the use of an antibody, multispecific antibody, preferably bispecific antibody, or a pharmaceutical composition thereof, as described herein for treating and/or preventing pruritus, preferably pruritus associated with atopic dermatitis. In a further aspect, there is provided the use of an antibody, multispecific antibody, preferably bispecific antibody, or a pharmaceutical composition thereof, as described herein for the manufacture of a medicament for the treatment and/or prevention of pruritus, preferably pruritus associated with atopic dermatitis. In a further aspect, there is provided a method of preventing and/or treating pruritus, preferably pruritus associated with atopic dermatitis, in a subject in need thereof, comprising administering to the subject a therapeutically effective amount of an antibody, multispecific antibody, preferably bispecific antibody, or a pharmaceutical composition thereof, as described herein. In some embodiments, a prophylactically effective amount is administered. In some embodiments, the subject in need thereof is a mammal, preferably a human.

In some embodiments, the atopic dermatitis is moderate-to-severe. The term “moderate-to-severe” refers to AD characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have more than 10% or more that 20% of the body's skin area affected, or 10% or more of skin area affected in addition to involvement of the eyes, hands and body folds. Patients affected by moderate-to-severe AD also, in general, have (i) an Investigator's Global Assessment (IGA) score of 3 or 4, (ii) an Eczema Area and Severity Index (EASI) score of at least 10, preferably at least 12, and (iii) itch. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor or any other commonly used therapeutic agent known in the art.

Administration of a therapeutically effective amount may result in a decrease of one or more AD-associated biomarkers, for example one or more biomarkers selected from the group consisting of CCL17/TARC, IgE (e.g., serum IgE), CCL26/eotaxin-3, CCL22/MDC, hsCRP, CD40, IL-31, IL-13, IL-24, IL-22, IL-18 (e.g., serum IL-18, serum free IL-18 (bioactive)), and IL-18BP (e.g., serum IL-18BP), as compared to the level before treatment.

In therapeutics uses and methods which involve the administration of a multispecific, preferably bispecific, antibody, the antibody is preferably an anti-IL-31/IL-13 antibody as described herein.

Therapeutic uses and methods described herein may first comprise selecting a subject who exhibits at least one symptom associated with atopic dermatitis, for example any of the symptoms described above, followed by administration of a therapeutically effective amount of the antibody or pharmaceutical composition thereof to the subject. Alternatively or in addition, if a subject exhibits a level of an atopic dermatitis-associated biomarker which signifies a disease state, the individual is identified as a suitable patient for whom administration of the antibody or pharmaceutical composition thereof, of the invention would be beneficial.

Treatment efficacy can be measured by a decrease in disease severity e.g., symptoms, or by a decrease in an atopic dermatitis-associated biomarker and can e.g., be determined by comparing (i) the level of the biomarker measured in a subject at a defined time point after administration of the antibody or pharmaceutical composition thereof, to (ii) the level of the biomarker measured in the subject prior to the administration of the antibody or pharmaceutical composition thereof (i.e., to a “baseline measurement”).

In some embodiments, the methods herein may be used to treat atopic dermatitis in child subjects. For example, the present methods may be used to treat infant subjects who are at least 1 month, 2 months, 3 months, 4 months, 5 months, 6 months, 7 months, 8 months, 9 months, 10 months, 11 months, 12 months, or at least 24 months old. In other embodiments, the methods of the present disclosure may be used to treat children who are more than 3 years old, more than 4 years, 5 years, 6 years, 7 years, 8 years, 9 years, 10 years, 11 years, 12 years, 13 years, 14 years, or more than 15 years old. In some embodiments, the methods herein may be used to treat atopic dermatitis in adult subjects. By “adults,” it is meant that the subject is at least 16 years old, which includes, a subject whose age is, for example, 16 years, 17 years, 18 years, 19 years, 20 years, 25 years, 30 years, 40 years, 50 years, 60 years, 70 years, 80 years, 90 years, or greater. The methods for treating or preventing AD comprise treating subjects that have elevated levels of atopic dermatitis-associated markers.

### Combination Therapies

The antibodies (e.g., the monospecific or multispecific e.g., bispecific, antibodies) or fragments thereof (e.g., Fabs) of the invention, or compositions, such as pharmaceutical compositions, comprising them, can be used in combination with additional therapeutic agents and therapies for the treatment and/or prevention of diseases, disorders and conditions, such as the ones described earlier herein.

Administered “in combination”, in reference to an additional therapeutic agent, refers to two (or more) different treatments are delivered to the subject during the course of treatment. In some embodiments, the administration of one treatment is still occurring when the administration of the second begins, so that there is overlap in terms of administration. This is sometimes referred to herein as “simultaneous” or “concurrent” administration. In other embodiments, the administration of one treatment ends before the administration of the other treatment begins (“sequential” administration). In some embodiments of either case, the treatment is more effective because of the combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, administration is such that the reduction in a symptom, or other parameter related to the disorder is greater than what would be observed with one treatment delivered in the absence of the other. The effect of the two treatments can be partially additive, wholly additive, or greater than additive. The administration can be such that an effect of the first treatment delivered is still detectable when the second is administered.

The term “concurrently” is not limited to the administration of therapies (e.g., prophylactic or therapeutic agents) at exactly the same time, but rather it is meant that an antibody or fragments thereof of present disclosure (or pharmaceutical composition thereof) is administered to a subject in a sequence and within a time interval such that the molecules of the disclosure can act together with the additional therapeutic agent(s) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route, for example described earlier herein.

Additional therapeutic agents (e.g., additional prophylactic or therapeutic agents), which can be administered in combination with the molecules of the present application may be administered, for example, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the molecules or fragments thereof of the disclosure.

An antibody or fragment thereof of present disclosure and the additional therapeutic agent(s) can be administered simultaneously, in the same or in separate pharmaceutical compositions, or sequentially. For sequential administration, the antibody or fragment thereof can be administered first, and the additional agent can be administered second, or the order of administration can be reversed. The additional therapeutic agent(s) may be administered to a subject by the same or different routes of administration compared to the antibody or fragment thereof.

The multispecific antibody or fragment thereof and/or the additional therapeutic agent(s), procedures or modalities can be administered during periods of active disorder, or during a period of remission or less active disease. The antibody or fragment thereof can be administered before the other treatment, concurrently with the treatment, post-treatment, or during remission of the disorder.

The additional therapeutic agent(s) of the combination therapies of the present disclosure can also be cyclically administered. Combination cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

When administered in combination, the antibody or fragment thereof of present disclosure, and the additional therapeutic agent (e.g., second or third agent), or all, can be administered in an amount or dose that is higher, lower or the same than the amount or dosage of each agent used individually, e.g., as a monotherapy. In certain embodiments, the administered amount or dosage of the antibody or fragment thereof as described herein, of the additional agent (e.g., second or third agent), or of all compounds, is lower (e.g., at least 20%, at least 30%, at least 40%, or at least 50%) than the amount or dosage of each agent used individually, e.g., as a monotherapy. In other embodiments, the amount or dosage of the antibody or fragment thereof, of the additional agent (e.g., second or third agent), or of all compounds, that results in a desired effect (e.g., treatment of an immune disease or an inflammatory disease or condition, e.g., of AD) is lower (e.g., at least 20%, at least 30%, at least 40%, or at least 50% lower) than the amount or dosage of each agent used individually, e.g., as a monotherapy, required to achieve the same therapeutic effect.

Preferably, the additional therapeutic agent (e.g., second or third agent) is an AD agent, e.g., small molecule, biological therapy, or an agent employing AD modality, e.g., phototherapy, including topical therapy, systemic therapy, phototherapy, and combinations thereof. “AD agent” includes topical therapies in the form of creams, ointments, lotions, gels or sprays (e.g., low-medium potency corticosteroids [Group IV-VII according to WHO guidelines, see Bolognia J L, Jorizzo J L, Schaffer J V Glucocorticosteroids. Dermatology. 3rd ed. 2012. Ch 125, 2075-88; Ference J D, Last A R. Choosing topical corticosteroids. Am Fam Physician. 2009 Jan. 15; 79(2): 135-40]); over the counter (OTC) emollients, and medical devices or so-called barrier creams (such as atopiclair); and lubricants for the treatment of itch and/or pain, e.g. anti-itch lotions containing menthol, pramoxine or anti-histamines; local anesthetics, systemic agents (e.g., biological agents, e.g., IL-4R inhibitors, for example dupilumab; IL-13Ra1 inhibitors, for example ASLAN-004; IL-13Ra2 inhibitors; IL-13 inhibitors, for example lebrikizumab; IL-31 inhibitors, for example nemolizumab; TNF alpha inhibitors, for example adalimumab, infliximab, certolizumab and etanercept, alefacept; IL-la inhibitors, for example bermekimab (MABpI); IL-23 inhibitors, for example briakinumab, ustekinumab, guselkumab, risankizumab, tildrakizumab; IL-17 inhibitors, for example brodalumab, ixekizumab; CD11a inhibitors, for example efalizumab; IL-22 inhibitors, for example fezalimumab, IL-22 binding proteins; IL-5 inhibitors, for example mepolizumab, benralizumab; a synthetic form of IL-2, such as aldesleukin; recombinant IL-2 approaches targeting the interleukin-2 receptor complex, for example LY3471851; OSMR inhibitors, for example KPL-716; VAP-1 inhibitors; OX-40 inhibitors or OX40L inhibitors such as GBR830, KY1005; IgE inhibitors, for example omalizumab, ligelizumab; TSLP inhibitors, for example tezepelumab; IL-33 inhibitors, for example MED13506; IL-36 inhibitors, for example spesolimab, ANB019; B-cell modulating approaches, for example rituximab, ocrelizumab; non-biological immunomodulating treatments, e.g., cyclosporine and other calcineurin inhibitors, JAK inhibitors, for example tofacitinib, upadacitinib, abrocitinib, baricitinib; TYK2 inhibitors, for example deucravacitinib; methotrexate; PDE4 inhibitors, for example apremilast; Siglec inhibitors, for example AK-002; SiP agonists or antagonists, for example etrasimod or SCD-044; BTK inhibitors, for example TAS-5315, IRAK4 antagonists and CCR4-inhibiting approaches, for example RPT-193; systemic corticosteroids, cyclophosphamide, sulphasalazine, azathioprin, mycophenolate mofetil, dapson, hydroxychloroquine; retinoids (e.g., alitretinoin); leukotriene inhibitors or antileukotrienes, for example, montelukast, pranlukast or zafirlukast, as well as 5-LO inhibitors, for example zileuton, and LTA4H inhibitors, for example acebilustat, intralesional corticosteroid injections; phototherapy (e.g. UVB and UVA high dose). photochemotherapy (e.g. psoralen and UVA (PUVA)); topical calcineurin inhibitors (cyclosporine, tacrolimus, pimecrolimus) or topical PDE4 inhibitors, for example crisaborole, difamilast or roflumilast; topical JAK inhibitors, for example ruxolitinib, delgocitinib, or topical Vitamin D analogues and topical aryl hydrocarbon receptor (AhR) inhibitors, for example benvitimod/tapinarof; topical corticosteroids of high-ultrahigh potency (Group I, II, Ill as per WHO definition); anti-fungal drugs with known anti-inflammatory properties, e.g., griseofulvin, itraconazole, betamethasone, dexamethasone, INCB018424, triamcinolone, apremilast, turmeric past, glucosamine sulfate, triamcinolone acetonide, sesame oil, betamethasone dipropionate, clobetasol propionate, probiotics (e.g., Bifidobacterium animalis subst. lactis HN019, Lactobacilli reuteri), omega-3, prednisone, prednisolone, platelet rich plasma, orabase paste, lycopene, topical chamomile, green tea, CO2 laser treatment, allergen specific immunotherapies, polybiotics, photobiomodulation, metronidazole, doxycycline, minocycline, cedar honey, purslane, curcuminoids, alefacept, hexaminolevulinate, hydroxychloroquine, adcortyl, efalizumab, fluocinolone, co-enzyme Q10 mucoadhesive tablets, Chamaemelum nobile, sirolimus, tacrolimus, qingxuan decoction, NSAID topical rinse, NSAIDs, quercetin, NAVS naphthalan, valchlor, bupivacaine, oatmeal baths.

In some embodiments, the additional therapeutic agent is lebrikizumab (available as Ebglyss™). In some embodiments, the additional therapeutic agent is dupilumab (available as Dupixent®). In some embodiments, the additional therapeutic agent is nemolizumab (available as Mitchga®).

The antibodies and bispecific antibodies of the present invention may be used in combination with topical corticosteroids. In some embodiments, the additional therapeutic agent is an AD topical therapeutic agent, including but not limited to topical steroid, e.g., corticosteroid, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. crisaborole, adrenocorticotropic hormone analogs, dupilumab, etanercept, adalimumab, infliximab, omalizumab, secukinumab.

In a further aspect, there is provided an antibody, multispecific antibody, preferably bispecific antibody, or a pharmaceutical composition thereof, as described herein, for use in preventing and/or treating an inflammatory or immune condition, wherein the antibody, multispecific antibody, preferably bispecific antibody, or a pharmaceutical composition thereof is administered simultaneously or sequentially with an IL-13 antagonist to a subject in need thereof. In a further aspect, there is provided the use of an antibody, multispecific antibody, preferably bispecific antibody, or a pharmaceutical composition thereof, as described herein, in the manufacture of a medicament for preventing and/or treating an inflammatory or immune condition, wherein the antibody, multispecific antibody, preferably bispecific antibody, or a pharmaceutical composition thereof is administered simultaneously or sequentially with an IL-13 antagonist to a subject in need thereof. In a further aspect, there is provided a method of preventing and/or treating an inflammatory or immune condition in a subject in need thereof, the method comprising simultaneously or sequentially administering a therapeutically effective amount of the antibody, multispecific antibody, preferably bispecific antibody, or a pharmaceutical composition thereof, as described herein and an IL-13 antagonist to the subject. In preferred embodiments, the IL-13 antagonist is an anti-IL-13 antibody or fragment thereof. In some embodiments, the anti-IL-13 antibody is lebrikizumab. In some embodiments, the inflammatory or immune condition is atopic dermatitis. In some embodiments, the subject in need of treatment is a mammal, preferably a human.

### Diagnostic Methods

The antibodies (e.g., the monospecific or multispecific e.g., bispecific, antibodies) or fragments thereof (e.g., Fabs) of the invention, or compositions, such as pharmaceutical compositions, comprising them, can also be used in diagnostic applications, for example to detect the presence of IL-31 and/or IL-13 in a biological sample.

Therefore, in a further aspect of the invention, there is provided a method for detecting the presence and/or concentration of one or more proteins in a biological sample using one or more antibodies or fragments thereof or compositions thereof as described herein. In some embodiments, the method is an ex vivo or in vitro method.

In a further aspect, there is provided a method for detecting the presence and/or concentration of IL-31 protein in a biological sample, comprising the steps of:
(i) providing a biological sample from a subject,
(ii) reacting said biological sample with at least one anti-IL31 antibody or fragment thereof described herein, under conditions sufficient for binding IL-31 protein present in said biological sample to said at least one antibody through antigen-antibody interactions, thus forming an IL-31-antibody complex; and
(iii) detecting a signal proportional to the level of IL-31-antibody complex formed in step (ii),
wherein the intensity of the signal correlates with the concentration of IL-31 protein in the biological sample.

In a further aspect, there is provided a method for detecting the presence and/or concentration of IL-31 protein and/or IL-13 protein in a biological sample, comprising the steps of:
(i) providing a biological sample from a subject,
(ii) reacting said biological sample with at least one anti-IL-31/IL-13 multispecific antibody, preferably bispecific antibody, or fragment thereof described herein, under conditions sufficient for binding IL-31 protein and/or IL-13 protein present in said biological sample to said at least one antibody through antigen-antibody interactions, thus forming an IL-31- and/or IL-13-antibody complex; and
(iii) detecting a signal proportional to the level of IL-31- and/or IL-13-antibody complex formed in step (ii),
wherein the intensity of the signal correlates with the concentration of IL-31 protein and/or IL-13 protein in the biological sample.

The biological sample may take any suitable form, for example it may be a cell or collection of cells sample, a cell lysate, a protein or membrane extract of cells, a skin tissue sample, a connective tissue sample, an epithelial tissue sample, a body fluid sample, a blood sample, a saliva sample, and the like.

In some embodiments, the methods are ex vivo or in vitro methods. In some embodiments, the subject is a mammal, preferably a human. In preferred embodiments, the IL-31 and/or IL-13 are human.

Detecting a signal proportional to an antibody-protein complex (e.g., an antibody-IL-31 or an antibody-IL-13 complex) can be done using any standard method available to the skilled person. For example, the antibody may be labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. IL-31- and/or IL-13-antibody complex formation can, for example, be detected by measuring or visualizing either the molecule bound to IL-31 and/or IL-13 or the unbound molecule. Conventional detection assays can be used, e.g., an enzyme-linked immunosorbent assay (ELISA), a radioimmunoassay (RIA), or tissue immunohistochemistry.

The antibody or fragment thereof may be immobilized on an insoluble matrix. Immobilization entails separating the antibody or fragment thereof from any proteins that remain free in solution. This conventionally is accomplished by either insolubilizing the antibody or fragment thereof before the assay procedure, as by adsorption to a water-insoluble matrix or surface (Bennich et al, U.S. Pat. No. 3,720,760), or by covalent coupling (for example, using glutaraldehyde cross-linking), or by insolubilizing the antibody or fragment thereof after formation of a complex (e.g., with IL-31 and/or IL-13), e.g., by immunoprecipitation.

### Kits

The invention also provides kits comprising the antibodies (e.g., the monospecific or multispecific e.g., bispecific, antibodies) or fragments thereof of the invention (e.g., Fabs), or compositions, such as pharmaceutical compositions, comprising them. Such kits may be suitable for treating a patient, preferably a human, having a disease or disorder mediated by IL-31 and/or IL-31 and IL-13, e.g., an inflammatory disorder or condition, such as AD. Such kits comprise a therapeutically effective amount of an antibody or fragment thereof or of the composition. Therapeutically effective amounts may be comprised in the kits e.g., in a collection of single-use containers.

Additionally, the kits may comprise means for administering the antibody or fragment thereof and instructions for use. The phrase “means for administering” is used to indicate any available implement for systemically administering a drug to a patient, including, but not limited to, a container e.g., pre-filled syringe, a vial and syringe, an injection pen, an auto-injector, an IV drip and bag, an infusion pump, a patch, an infusion bag and needle, etc., which will depend on the administration route, examples of which are provided earlier herein. With such items, a patient may self-administer the drug (i.e., administer the drug without the assistance of a physician) or a medical practitioner may administer the drug. The instructions for use may provide the dose, route of administration, regimen, and total treatment duration for use with the comprised antibody, fragment thereof, or composition.

The kits may also contain additional therapeutic agents for treating a subject having a disease or disorder mediated by IL-31 and/or IL-13, for example the therapeutic or prophylactic agents described earlier herein. Such kits may e.g., comprise the additional therapeutic or prophylactic agent in the same pharmaceutical composition that comprises the antibody or fragment thereof, optionally at a fixed dose ration. Alternatively, such kits may comprise the additional therapeutic or prophylactic agent in a separate pharmaceutical composition to the one comprising the antibody or fragment thereof, for example in a different pre-filled syringe, vial and syringe, injection pen, auto-injector, IV drip and bag, infusion pump, a patch, infusion bag and needle, and the like (depending on the administration route).

The kits may also comprise instructions for use, for example instructions for administration of the antibody or fragment thereof or the composition to treat the patient.

### ADDITIONAL EMBODIMENTS

The following enumerated paragraphs represent additional embodiments of the invention.

Embodiment 1. An antibody that binds to IL-31, wherein the antibody comprises:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23;
an LCDR1 comprising SEQ ID NO: 4 or SEQ ID NO: 24;
an LCDR2 comprising SEQ ID NO: 5 or SEQ ID NO: 25; and
an LCDR3 comprising SEQ ID NO: 6 or SEQ ID NO: 26;
or
an HCDR1 comprising SEQ ID NO: 48;
an HCDR2 comprising SEQ ID NO: 49 or SEQ ID NO: 61;
an HCDR3 comprising SEQ ID NO: 50 or SEQ ID NO: 63;
an LCDR1 comprising SEQ ID NO: 51 or SEQ ID NO: 65;
an LCDR2 comprising the amino acid sequence YAK; and
an LCDR3 comprising SEQ ID NO: 53 or SEQ ID NO: 64;
or
an HCDR1 comprising SEQ ID NO: 66 or SEQ ID NO: 72;
an HCDR2 comprising SEQ ID NO: 67 or SEQ ID NO: 73;
an HCDR3 comprising SEQ ID NO: 68 or SEQ ID NO: 77;
an LCDR1 comprising SEQ ID NO: 69 or SEQ ID NO: 76;
an LCDR2 comprising the amino acid sequence YAK; and
an LCDR3 comprising SEQ ID NO: 71 or SEQ ID NO: 75;
or
an HCDR1 comprising SEQ ID NO: 78 or SEQ ID NO: 90;
an HCDR2 comprising SEQ ID NO: 79 or SEQ ID NO: 91;
an HCDR3 comprising SEQ ID NO: 80 or SEQ ID NO: 96;
an LCDR1 comprising SEQ ID NO: 81 or SEQ ID NO: 95;
an LCDR2 comprising SEQ ID NO: 82 or SEQ ID NO: 93; and
an LCDR3 comprising SEQ ID NO: 83 or SEQ ID NO: 94.

Embodiment 2. The antibody according to embodiment 1, wherein the antibody comprises:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23;
an LCDR1 comprising SEQ ID NO: 4 or SEQ ID NO: 24;
an LCDR2 comprising SEQ ID NO: 5 or SEQ ID NO: 25; and
an LCDR3 comprising SEQ ID NO: 6 or SEQ ID NO: 26,
preferably wherein the HCDR1, HCDR2, HCDR3, LCDR1, LCDR2, and LCDR3 are defined according to the Kabat numbering scheme.

Embodiment 3. The antibody according to embodiment 1 or 2, wherein the antibody comprises:
i) an HCDR1 comprising SEQ ID NO: 1
ii) an HCDR2 comprising SEQ ID NO: 21; and
iii) an HCDR3 comprising SEQ ID NO: 3;
or
i) an HCDR1 comprising SEQ ID NO: 1;
ii) an HCDR2 comprising SEQ ID NO: 21; and
iii) an HCDR3 comprising SEQ ID NO: 23;
or
i) an HCDR1 comprising SEQ ID NO: 1;
ii) an HCDR2 comprising SEQ ID NO: 2; and
iii) an HCDR3 comprising SEQ ID NO: 3,
preferably wherein the HCDR1, HCDR2, and HCDR3 are defined according to the Kabat numbering scheme.

Embodiment 4. The antibody according to any one of embodiments 1 to 3, wherein the antibody comprises:
i) an LCDR1 comprising SEQ ID NO: 4;
ii) an LCDR2 comprising SEQ ID NO: 25; and
iii) an LCDR3 comprising SEQ ID NO: 26;
or
i) an LCDR1 comprising SEQ ID NO: 4;
ii) an LCDR2 comprising SEQ ID NO: 5; and
iii) an LCDR3 comprising SEQ ID NO: 6;
or
i) an LCDR1 comprising SEQ ID NO:4;
ii) an LCDR2 comprising SEQ ID NO: 5; and
iii) an LCDR3 comprising SEQ ID NO:26;
or
i) an LCDR1 comprising SEQ ID NO: 24;
ii) an LCDR2 comprising SEQ ID NO: 5; and
iii) an LCDR3 comprising SEQ ID NO: 26,
preferably wherein the LCDR1, LCDR2, and LCDR3 are defined according to the Kabat numbering scheme.

Embodiment 5. The antibody according to any one of embodiments 1 to 4, wherein the antibody comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO:3;
iv) an LCDR1 comprising SEQ ID NO: 4;
v) an LCDR2 comprising SEQ ID NO: 5; and
vi) an LCDR3 comprising SEQ ID NO:6,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
preferably wherein the HCDR1, HCDR2, HCDR3, LCDR1, LCDR2, and LCDR3 are defined according to the Kabat numbering scheme.

Embodiment 6. The antibody according to any one of embodiments 1 to 5, wherein the antibody comprises a heavy chain variable region (VH) comprising the amino acid sequence shown in SEQ ID NO: 7 or SEQ ID NO: 28 or SEQ ID NO: 29.

Embodiment 7. The antibody according to any one of embodiments 1 to 6, wherein the antibody comprises a light chain variable region (VL) comprising the amino acid sequence shown in SEQ ID NO: 8 or SEQ ID NO: 30 or SEQ ID NO:31 or SEQ ID NO:32.

Embodiment 8. The antibody according to any one of embodiments 1 to 7, wherein the antibody comprises:
i) a heavy chain variable region (VH) comprising the amino acid sequence shown in SEQ ID NO: 28; and
ii) a light chain variable region (VL) comprising the amino acid sequence shown in SEQ ID NO: 31;
or
i) a heavy chain variable region (VH) comprising the amino acid sequence shown in SEQ ID NO: 28; and
ii) a light chain variable region (VL) comprising the amino acid sequence shown in SEQ ID NO: 32;
or
i) a heavy chain variable region (VH) comprising the amino acid sequence shown in SEQ ID NO: 28; and
ii) a light chain variable region (VL) comprising the amino acid sequence shown in SEQ ID NO: 30;
or
i) a heavy chain variable region (VH) comprising the amino acid sequence shown in SEQ ID NO: 28; and
ii) a light chain variable region (VL) comprising the amino acid sequence shown in SEQ ID NO: 8;
or
i) a heavy chain variable region (VH) comprising the amino acid sequence shown in SEQ ID NO: 29; and
ii) a light chain variable region (VL) comprising the amino acid sequence shown in SEQ ID NO: 31;
or
i) a heavy chain variable region (VH) comprising the amino acid sequence shown in SEQ ID NO: 29; and
ii) a light chain variable region (VL) comprising the amino acid sequence shown in SEQ ID NO: 32;
or
i) a heavy chain variable region (VH) comprising the amino acid sequence shown in SEQ ID NO: 29; and
ii) a light chain variable region (VL) comprising the amino acid sequence shown in SEQ ID NO: 30;
or
i) a heavy chain variable region (VH) comprising the amino acid sequence shown in SEQ ID NO: 29; and
ii) a light chain variable region (VL) comprising the amino acid sequence shown in SEQ ID NO: 8;
or
i) a heavy chain variable region (VH) comprising the amino acid sequence shown in SEQ ID NO: 7; and
ii) a light chain variable region (VL) comprising the amino acid sequence shown in SEQ ID NO: 31;
or
i) a heavy chain variable region (VH) comprising the amino acid sequence shown in SEQ ID NO: 7; and
ii) a light chain variable region (VL) comprising the amino acid sequence shown in SEQ ID NO: 32;
or
i) a heavy chain variable region (VH) comprising the amino acid sequence shown in SEQ ID NO: 7; and
ii) a light chain variable region (VL) comprising the amino acid sequence shown in SEQ ID NO: 30;
or
i) a heavy chain variable region (VH) comprising the amino acid sequence shown in SEQ ID NO: 7; and
ii) a light chain variable region (VL) comprising the amino acid sequence shown in SEQ ID NO: 8.

Embodiment 9. The antibody according to any one of embodiments 1 to 8, wherein the antibody comprises:
i) a light chain comprising the amino acid sequence shown in SEQ ID NO: 34; and
ii) a heavy chain comprising the amino acid sequence shown in SEQ ID NO: 37;
or
i) a light chain comprising the amino acid sequence shown in SEQ ID NO: 35; and
ii) a heavy chain comprising the amino acid sequence shown in SEQ ID NO: 37;
or
i) a light chain comprising the amino acid sequence shown in SEQ ID NO: 33; and
ii) a heavy chain comprising the amino acid sequence shown in SEQ ID NO: 37;
or
i) a light chain comprising the amino acid sequence shown in SEQ ID NO: 9; and
ii) a heavy chain comprising the amino acid sequence shown in SEQ ID NO: 37;
or
i) a light chain comprising the amino acid sequence shown in SEQ ID NO: 34; and
ii) a heavy chain comprising the amino acid sequence shown in SEQ ID NO: 38;
or
i) a light chain comprising the amino acid sequence shown in SEQ ID NO: 35; and
ii) a heavy chain comprising the amino acid sequence shown in SEQ ID NO: 38;
or
i) a light chain comprising the amino acid sequence shown in SEQ ID NO: 33; and
ii) a heavy chain comprising the amino acid sequence shown in SEQ ID NO: 38;
or
i) a light chain comprising the amino acid sequence shown in SEQ ID NO: 9; and
ii) a heavy chain comprising the amino acid sequence shown in SEQ ID NO: 38;
or
i) a light chain comprising the amino acid sequence shown in SEQ ID NO: 34; and
ii) a heavy chain comprising the amino acid sequence shown in SEQ ID NO: 39;
or
i) a light chain comprising the amino acid sequence shown in SEQ ID NO: 35; and
ii) a heavy chain comprising the amino acid sequence shown in SEQ ID NO: 39;
or
i) a light chain comprising the amino acid sequence shown in SEQ ID NO: 33; and
ii) a heavy chain comprising the amino acid sequence shown in SEQ ID NO: 39;
or
i) a light chain comprising the amino acid sequence shown in SEQ ID NO: 9; and
ii) a heavy chain comprising the amino acid sequence shown in SEQ ID NO: 39.

Embodiment 10. The antibody according to any one of embodiments 1 to 9, further comprising an Fc polypeptide comprising the amino acid sequence shown in SEQ ID NO: 42 or a variant thereof.

Embodiment 11. The antibody according to embodiment 10, wherein the Fc polypeptide comprises a variant of SEQ ID NO: 42 comprising one or more mutations selected from:
a) one or more silencing mutations, e.g., an L234A and an L235A mutation (“LALA”); and/or
b) one or more half-life extension mutations, e.g., an M252Y, an S254T, and a T256E mutation (“YTE”); and/or
c) one or more K-i-H mutations, e.g., an S354C mutation and/or a T366W mutation or an Y349C, a T366S, an L368A, and/or a Y407V mutation, or one or more FAE mutations e.g., an F405L mutation or a K409R mutation,
according to EU numbering.

Embodiment 12. The antibody according to embodiment 10 or 11, wherein the Fc polypeptide comprises a variant of SEQ ID NO: 42 comprising:
a) an L234A and an L235A mutation (“LALA”);
b) an M252Y, an S254T, and a T256E mutation (“YTE”); and
c) an F405L or a K409R mutation,
according to EU numbering.

Embodiment 13. The antibody according to any one of embodiments 10 to 12, wherein the Fc polypeptide comprises the amino acid sequence shown in SEQ ID NO: 44 or SEQ ID NO: 45.

Embodiment 14. An antibody that binds to IL-31, wherein the antibody comprises:
i) a light chain comprising the amino acid sequence shown in SEQ ID NO: 9; and
ii) a heavy chain comprising the amino acid sequence shown in SEQ ID NO: 10 or 109;
or
i) a light chain comprising the amino acid sequence shown in SEQ ID NO: 34; and
ii) a heavy chain comprising the amino acid sequence shown in SEQ ID NO: 104 or 107;
or
i) a light chain comprising the amino acid sequence shown in SEQ ID NO: 35; and
ii) a heavy chain comprising the amino acid sequence shown in SEQ ID NO: 104 or 107;
or
i) a light chain comprising the amino acid sequence shown in SEQ ID NO: 33; and
ii) a heavy chain comprising the amino acid sequence shown in SEQ ID NO: 104 or 107;
or
i) a light chain comprising the amino acid sequence shown in SEQ ID NO: 9; and
ii) a heavy chain comprising the amino acid sequence shown in SEQ ID NO: 104 or 107;
or
i) a light chain comprising the amino acid sequence shown in SEQ ID NO: 34; and
ii) a heavy chain comprising the amino acid sequence shown in SEQ ID NO: 105 or 108;
or
i) a light chain comprising the amino acid sequence shown in SEQ ID NO: 35; and
ii) a heavy chain comprising the amino acid sequence shown in SEQ ID NO: 105 or 108;
or
i) a light chain comprising the amino acid sequence shown in SEQ ID NO: 33; and
ii) a heavy chain comprising the amino acid sequence shown in SEQ ID NO: 105 or 108;
or
i) a light chain comprising the amino acid sequence shown in SEQ ID NO: 9; and
ii) a heavy chain comprising the amino acid sequence shown in SEQ ID NO: 105 or 108;
or
i) a light chain comprising the amino acid sequence shown in SEQ ID NO: 34; and
ii) a heavy chain comprising the amino acid sequence shown in SEQ ID NO: 10 or 109;
or
i) a light chain comprising the amino acid sequence shown in SEQ ID NO: 35; and
ii) a heavy chain comprising the amino acid sequence shown in SEQ ID NO: 10 or 109;
or
i) a light chain comprising the amino acid sequence shown in SEQ ID NO: 33; and
ii) a heavy chain comprising the amino acid sequence shown in SEQ ID NO: 10 or 109.

Embodiment 15. An antibody that binds to IL-31, comprising:
(1) a heavy chain variable region comprising a variant of SEQ ID NO: 40, wherein the variant comprises relative to SEQ ID NO: 40 a substitution of the leucine (L) at position 29 (VH L29) by a different amino acid, wherein the glutamine (Q) at the first position of SEQ ID NO: 40 is denoted as position 1 (VH Q1); and
(2) a light chain variable region comprising SEQ ID NO: 8 or SEQ ID NO: 30 or SEQ ID NO:31 or SEQ ID NO:32.

Embodiment 16. The antibody according to embodiment 15, wherein the substitution of the leucine (L) at position 29 (VH L29) is a substitution by an arginine (R).

Embodiment 17. An antibody that binds to IL-31, comprising:
(1) a heavy chain variable region comprising a variant of SEQ ID NO: 40, wherein the variant comprises relative to SEQ ID NO: 40 a substitution of the glycine (G) at position 54 (VH G54), a substitution of the aspartic acid (D) at position 55 (VH D55), and a substitution of the glutamine (Q) at position 65 (VH Q65) by a different amino acid, wherein the glutamine (Q) at the first position of SEQ ID NO: 40 is denoted as position 1 (VH Q1); and
(2) a light chain variable region comprising SEQ ID NO: 8 or SEQ ID NO: 30 or SEQ ID NO:31 or SEQ ID NO:32.

Embodiment 18. The antibody according to embodiment 17, wherein the substitution of the glycine (G) at position 54 (VH G54) is a substitution by an alanine (A), the substitution of the aspartic acid (D) at position 55 (VH D55) is a substitution by a glutamic acid (E), and the substitution of the glutamine (Q) at position 65 (VH Q65) is a substitution by a lysine (K).

Embodiment 19. An antibody that binds to IL-31, comprising:
(1) a heavy chain variable region comprising a variant of SEQ ID NO: 40, wherein the variant comprises relative to SEQ ID NO: 40 a substitution of the aspartic acid (D) at position 100 (VH D100), a substitution of the tyrosine (Y) at position 102 (VH Y102), and a substitution of the aspartic acid (D) at position 110 (VH D 110) by a different amino acid, wherein the glutamine (Q) at the first position of SEQ ID NO: 40 is denoted as position 1 (VH Q1); and
(2) a light chain variable region comprising SEQ ID NO: 8 or SEQ ID NO: 30 or SEQ ID NO:31 or SEQ ID NO:32.

Embodiment 20. The antibody according to embodiment 19, wherein the substitution of the aspartic acid (D) at position 100 (VH D100) is a substitution by a glutamic acid (E), the substitution of the tyrosine (Y) at position 102 (VH Y102) is a substitution by a proline (P), and the substitution of the aspartic acid (D) at position 110 (VH D110) is a substitution by a histidine (H).

Embodiment 21. The antibody according to any one of embodiments 15 to 20, wherein the variant of SEQ ID NO: 40 further comprises relative to SEQ ID NO: 40 a substitution of the alanine (A) at position 16 (VH A16), a substitution of the leucine (L) at position 70 (VH L70), and a substitution of the leucine (L) at position 117 (VH L117) by a different amino acid, wherein the glutamine (Q) at the first position of SEQ ID NO: 40 is denoted as position 1 (VH Q1).

Embodiment 22. The antibody according to embodiment 21, wherein the substitution of the alanine (A) at position 16 (VH A16) is a substitution by a serine (S), the substitution of the leucine (L) at position 70 (VH L70) is a substitution by an isoleucine (I), and the substitution of the leucine (L) at position 117 (VH L117) is a substitution by a threonine (T).

Embodiment 23. An antibody that binds to IL-31, comprising:
(1) a heavy chain variable region comprising the amino acid sequence shown in SEQ ID NO: 7, SEQ ID NO: 28, or SEQ ID NO: 29; and
(2) a light chain variable region comprising a variant of SEQ ID NO: 41, wherein the variant comprises relative to SEQ ID NO: 41 a substitution of the glycine (G) at position 27 (VL G27) and a substitution of the histidine (H) at position 30 (VL H30) by a different amino acid, wherein the aspartic acid (D) at the first position of SEQ ID NO: 41 is denoted as position 1 (VL D1).

Embodiment 24. The antibody according to embodiment 23, wherein the substitution of the glycine (G) at position 27 (VL G27) is a substitution by an arginine (R) and the substitution of the histidine (H) at position 30 (VL H30) is a substitution by a glycine (G).

Embodiment 25. An antibody that binds to IL-31, comprising:
(1) a heavy chain variable region comprising the amino acid sequence shown in SEQ ID NO: 7, SEQ ID NO: 28, or SEQ ID NO: 29; and
(2) a light chain variable region comprising a variant of SEQ ID NO: 41, wherein the variant comprises relative to SEQ ID NO: 41 a substitution of the asparagine (N) at position 50 (VL N50), a substitution of the leucine (L) at position 54 (VL L54), and a substitution of the aspartic acid (D) at position 56 (VL D56) by a different amino acid, wherein the aspartic acid (D) at the first position of SEQ ID NO: 41 is denoted as position 1 (VL D1).

Embodiment 26. The antibody according to embodiment 25, wherein the substitution of the asparagine (N) at position 50 (VL N50) is a substitution by a tyrosine (Y), the substitution of the leucine (L) at position 54 (VL L54) is a substitution by a serine (S), and the substitution of the aspartic acid (D) at position 56 (VL D56) is a substitution by a serine (S).

Embodiment 27. The antibody according to any one of embodiments 23 to 26, wherein the variant of SEQ ID NO: 41 further comprises relative to SEQ ID NO: 40 a substitution of the aspartic acid (D) at position 1 (VL D1), a substitution of the glutamic acid (E) at position 68 (VL E68), a substitution of the glutamine (Q) at position 70 (VL Q70), a substitution of the serine (S) at position 72 (VL S72), and a substitution of the glutamine (Q) at position 100 (VL Q100), wherein the aspartic acid (D) at the first position of SEQ ID NO: 41 is denoted as position 1 (VL D1).

Embodiment 28. The antibody according to embodiment 27, wherein the substitution of the aspartic acid (D) at position 1 (VL D1) is a substitution by an alanine (A), the substitution of the glutamic acid (E) at position 68 (VL E68) is a substitution by a glycine (G), the substitution of the glutamine (Q) at position 70 (VL Q70) is a substitution by an aspartic acid (D), the substitution of the serine (S) at position 72 (VL S72) is a substitution by a threonine (T), and the substitution of the glutamine (Q) at position 100 (VL Q100) is a substitution by a glycine (G).

Embodiment 29. An antibody that binds to IL-31, comprising:
(1) a heavy chain variable region comprising a variant of SEQ ID NO: 40, wherein the variant comprises relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1), and;
(2) a light chain variable region comprising a variant of SEQ ID NO: 41, wherein the variant comprises relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

Embodiment 30. The antibody according to any one of embodiments 1 to 29, wherein the antibody is monospecific for IL-31.

Embodiment 31. The antibody according to any one of embodiments 1 to 30, wherein the antibody inhibits IL-31 activity, preferably wherein the antibody inhibits IL-31 activity with an IC50 of less than 30 pM, less than 25 pM, less than 15 pM or less than about 15 pM, preferably as measured by an IL-31 dependent Baf3 cell proliferation assay.

Embodiment 32. The antibody according to any one of embodiments 1 to 31, wherein the antibody binds IL-31 with a dissociation constant (KD) of less than 30 pM, less than 25 pM, less than 20 pM, less than 15 pM, less than 10 pM, less than 5 pM or less than about 5 pM as measured using surface plasmon resonance.

Embodiment 33. The antibody according to any one of embodiments 1 to 32, wherein the antibody comprises a Fab domain having a melting temperature (Tm) of at least 70° C., such as 70° C., 71° C., 72° C., 73° C., 74° C., 75° C., 76° C., 77° C., preferably at least 75° C., more preferably 76° C. or about 76° C., as measured by DSF.

Embodiment 34. The antibody according to any one of embodiments 1 to 33, wherein the antibody is monovalent with respect to binding IL-31.

Embodiment 35. The antibody according to any one of embodiments 1 to 33, wherein the antibody is multivalent, preferably bivalent, with respect to binding IL-31.

Embodiment 36. The antibody according to any one of embodiments 1 to 35, wherein the antibody is a humanized, preferably monoclonal, antibody.

Embodiment 37. The antibody according to any one of embodiments 1 to 36, wherein the antibody is an IgG type, for example an IgG1 type.

Embodiment 38. The antibody according to any one of embodiments 1 to 37, wherein the IL-31 is human.

Embodiment 39. A multispecific antibody, wherein the antibody comprises:
(i) a first part that binds to IL-31 comprising:
   an HCDR1 comprising SEQ ID NO: 1;
   an HCDR2 comprising SEQ ID NO: 2 or SEQ ID NO: 21;
   an HCDR3 comprising SEQ ID NO: 3 or SEQ ID NO: 23; and,
   an LCDR1 comprising SEQ ID NO: 4 or SEQ ID NO: 24;
   an LCDR2 comprising SEQ ID NO: 5 or SEQ ID NO: 25, and;
   an LCDR3 comprising SEQ ID NO: 6 or SEQ ID NO: 26, and
(ii) a second part that binds to a target different from IL-31,
preferably wherein the HCDR1, HCDR2, HCDR3, LCDR1, LCDR2, and LCDR3 are defined according to the Kabat numbering scheme.

Embodiment 40. The multispecific antibody according to embodiment 39, wherein the second part binds to IL-13.

Embodiment 41. The multispecific antibody according to embodiment 39 or 40, wherein the antibody is bispecific.

Embodiment 42. The multispecific antibody according to any one of embodiments 39 to 41, wherein the first part that binds to IL-31 comprises:
i) an HCDR1 comprising SEQ ID NO: 1
ii) an HCDR2 comprising SEQ ID NO: 21, and;
iii) an HCDR3 comprising SEQ ID NO: 3;
or
i) an HCDR1 comprising SEQ ID NO: 1;
ii) an HCDR2 comprising SEQ ID NO: 21, and;
iii) an HCDR3 comprising SEQ ID NO: 23;
or
i) an HCDR1 comprising SEQ ID NO: 1;
ii) an HCDR2 comprising SEQ ID NO: 2, and;
iii) an HCDR3 comprising SEQ ID NO: 3,
preferably wherein the HCDR1, HCDR2, and HCDR3 are defined according to the Kabat numbering scheme.

Embodiment 43. The multispecific antibody according any one of embodiments 39 to 42, wherein the first part that binds to IL-31 comprises:
i) an LCDR1 comprising SEQ ID NO: 4;
ii) an LCDR2 comprising SEQ ID NO: 25; and
iii) an LCDR3 comprising SEQ ID NO: 26 or
i) an LCDR1 comprising SEQ ID NO: 4;
ii) an LCDR2 comprising SEQ ID NO: 5; and
iii) an LCDR3 comprising SEQ ID NO: 6 or
i) an LCDR1 comprising SEQ ID NO: 4;
ii) an LCDR2 comprising SEQ ID NO: 5; and
iii) an LCDR3 comprising SEQ ID NO: 26 or
i) an LCDR1 comprising SEQ ID NO: 24;
ii) an LCDR2 comprising SEQ ID NO: 5; and
iii) an LCDR3 comprising SEQ ID NO: 26,
preferably wherein the LCDR1, LCDR2, and LCDR3 are defined according to the Kabat numbering scheme.

Embodiment 44. The multispecific antibody according to any one of embodiments 39 to 43, wherein the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO:3;
iv) an LCDR1 comprising SEQ ID NO: 4;
v) an LCDR2 comprising SEQ ID NO: 5; and
vi) an LCDR3 comprising SEQ ID NO:6,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26, or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
wherein the HCDR1, HCDR2, HCDR3, LCDR1, LCDR2, and LCDR3 are defined according to the Kabat numbering scheme.

Embodiment 45. The multispecific antibody according to any one of embodiments 39 to 44, wherein the first part that binds to IL-31 comprises a heavy chain variable region (VH) comprising the amino acid sequence shown in SEQ ID NO: 7 or SEQ ID NO: 28 or SEQ ID NO: 29.

Embodiment 46. The multispecific antibody according to any one of embodiments 39 to 45, wherein the first part that binds to IL-31 comprises a light chain variable region (VL) comprising the amino acid sequence shown in SEQ ID NO: 8 or SEQ ID NO: 30 or SEQ ID NO:31 or SEQ ID NO:32.

Embodiment 47. The multispecific antibody according to any one of embodiments 39 to 46, wherein the first part that binds to IL-31 comprises:
i) a heavy chain variable region (VH) comprising the amino acid sequence shown in SEQ ID NO: 28; and
ii) a light chain variable region (VL) comprising the amino acid sequence shown in SEQ ID NO: 31;
or
i) a heavy chain variable region (VH) comprising the amino acid sequence shown in SEQ ID NO: 28; and
ii) a light chain variable region (VL) comprising the amino acid sequence shown in SEQ ID NO: 32;
or
i) a heavy chain variable region (VH) comprising the amino acid sequence shown in SEQ ID NO: 28; and
ii) a light chain variable region (VL) comprising the amino acid sequence shown in SEQ ID NO: 30;
or
i) a heavy chain variable region (VH) comprising the amino acid sequence shown in SEQ ID NO: 28; and
ii) a light chain variable region (VL) comprising the amino acid sequence shown in SEQ ID NO: 8;
or
i) a heavy chain variable region (VH) comprising the amino acid sequence shown in SEQ ID NO: 29; and
ii) a light chain variable region (VL) comprising the amino acid sequence shown in SEQ ID NO: 31;
or
i) a heavy chain variable region (VH) comprising the amino acid sequence shown in SEQ ID NO: 29; and
ii) a light chain variable region (VL) comprising the amino acid sequence shown in SEQ ID NO: 32;
or
i) a heavy chain variable region (VH) comprising the amino acid sequence shown in SEQ ID NO: 29; and
ii) a light chain variable region (VL) comprising the amino acid sequence shown in SEQ ID NO: 30;
or
i) a heavy chain variable region (VH) comprising the amino acid sequence shown in SEQ ID NO: 29; and
ii) a light chain variable region (VL) comprising the amino acid sequence shown in SEQ ID NO: 8;
or
i) a heavy chain variable region (VH) comprising the amino acid sequence shown in SEQ ID NO: 7; and
ii) a light chain variable region (VL) comprising the amino acid sequence shown in SEQ ID NO: 31;
or
i) a heavy chain variable region (VH) comprising the amino acid sequence shown in SEQ ID NO: 7; and
ii) a light chain variable region (VL) comprising the amino acid sequence shown in SEQ ID NO: 32;
or
i) a heavy chain variable region (VH) comprising the amino acid sequence shown in SEQ ID NO: 7; and
ii) a light chain variable region (VL) comprising the amino acid sequence shown in SEQ ID NO: 30;
or
i) a heavy chain variable region (VH) comprising the amino acid sequence shown in SEQ ID NO: 7; and
ii) a light chain variable region (VL) comprising the amino acid sequence shown in SEQ ID NO: 8.

Embodiment 48. The multispecific antibody according to any one of embodiments 39 to 47, wherein the first part that binds to IL-31 comprises:
i) a light chain comprising the amino acid sequence shown in SEQ ID NO: 34; and
ii) a heavy chain comprising the amino acid sequence shown in SEQ ID NO: 37;
or
i) a light chain comprising the amino acid sequence shown in SEQ ID NO: 35; and
ii) a heavy chain comprising the amino acid sequence shown in SEQ ID NO: 37;
or
i) a light chain comprising the amino acid sequence shown in SEQ ID NO: 33; and
ii) a heavy chain comprising the amino acid sequence shown in SEQ ID NO: 37;
or
i) a light chain comprising the amino acid sequence shown in SEQ ID NO: 9; and
ii) a heavy chain comprising the amino acid sequence shown in SEQ ID NO: 37;
or
i) a light chain comprising the amino acid sequence shown in SEQ ID NO: 34; and
ii) a heavy chain comprising the amino acid sequence shown in SEQ ID NO: 38;
or
i) a light chain comprising the amino acid sequence shown in SEQ ID NO: 35; and
ii) a heavy chain comprising the amino acid sequence shown in SEQ ID NO: 38;
or
i) a light chain comprising the amino acid sequence shown in SEQ ID NO: 33; and
ii) a heavy chain comprising the amino acid sequence shown in SEQ ID NO: 38;
or
i) a light chain comprising the amino acid sequence shown in SEQ ID NO: 9; and
ii) a heavy chain comprising the amino acid sequence shown in SEQ ID NO: 38;
or
i) a light chain comprising the amino acid sequence shown in SEQ ID NO: 34; and
ii) a heavy chain comprising the amino acid sequence shown in SEQ ID NO: 39;
or
i) a light chain comprising the amino acid sequence shown in SEQ ID NO: 35; and
ii) a heavy chain comprising the amino acid sequence shown in SEQ ID NO: 39;
or
i) a light chain comprising the amino acid sequence shown in SEQ ID NO: 33; and
ii) a heavy chain comprising the amino acid sequence shown in SEQ ID NO: 39;
or
i) a light chain comprising the amino acid sequence shown in SEQ ID NO: 9; and
ii) a heavy chain comprising the amino acid sequence shown in SEQ ID NO: 39.

Embodiment 49. The multispecific antibody according to any one of embodiments 40 to 48, wherein the second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16,
preferably wherein the HCDR1, HCDR2, HCDR3, LCDR1, LCDR2, and LCDR3 are defined according to the Kabat numbering scheme.

Embodiment 50. The multispecific antibody according to any one of embodiments 40 to 49, wherein:
the first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
and
the second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16,
preferably wherein the HCDR1, HCDR2, HCDR3, LCDR1, LCDR2, and LCDR3 are defined according to the Kabat numbering scheme.

Embodiment 51. The multispecific antibody according to any one of embodiments 40 to 50, wherein the second part that binds to IL-13 comprises a heavy chain variable region (VH) comprising the amino acid sequence shown in SEQ ID NO: 17.

Embodiment 52. The multispecific antibody according to any one of embodiments 40 to 51, wherein the second part that binds to IL-13 comprises a light chain variable region (VL) comprising the amino acid sequence shown in SEQ ID NO: 18.

Embodiment 53. The multispecific antibody according to any one of embodiments 40 to 52, wherein:
the first part that binds to IL-31 comprises a heavy chain variable region (VH) comprising the amino acid sequence shown in SEQ ID NO: 7 and a light chain variable region (VL) comprising the amino acid sequence shown in SEQ ID NO: 8; and
the second part that binds to IL-13 comprises a heavy chain variable region (VH) comprising the amino acid sequence shown in SEQ ID NO: 17 and a light chain variable region (VL) comprising the amino acid sequence shown in SEQ ID NO: 18.

Embodiment 54. The multispecific antibody according to any one of embodiments 40 to 53, wherein the second part that binds to IL-13 comprises a light chain comprising an amino acid sequence shown in SEQ ID NO: 19.

Embodiment 55. The multispecific antibody according to any one of embodiments 40 to 54, wherein the second part that binds to IL-13 comprises a heavy chain comprising an amino acid sequence shown in SEQ ID NO: 43.

Embodiment 56. The multispecific antibody according to any one of embodiments 40 to 55, wherein:
the first part that binds to IL-31 comprises a light chain comprising the amino acid sequence shown in SEQ ID NO: 9 and a heavy chain comprising the amino acid sequence shown in SEQ ID NO: 39; and
the second part that binds to IL-13 comprises a light chain comprising the amino acid sequence shown in SEQ ID NO: 19 and a heavy chain comprising the amino acid sequence shown in SEQ ID NO: 43.

Embodiment 57. The multispecific antibody according to any one of embodiments 40 to 56, wherein
the first part that binds to IL-31 comprises a first Fc polypeptide, and
the second part that binds to IL-13 comprises a second Fc polypeptide, wherein the first and/or second Fc polypeptide comprises the amino acid sequence shown in SEQ ID NO: 42 or a variant thereof.

Embodiment 58. The multispecific antibody according to embodiment 57 wherein the first and/or second Fc polypeptide comprises a variant of SEQ ID NO: 42 comprising one or more mutations selected from:
a. one or more silencing mutations e.g., an L234A and an L235A mutation (“LALA”); and/or
b. one or more half-life extension mutations e.g., an M252Y, an S254T and a T256E mutation “YTE”; and/or
c. one or more K-i-H mutations e.g., an S354C and/or a T366W mutation or an Y349C, a T366S, an L368A, and/or Y407V mutation, or FAE mutations e.g., an F405L mutation or a K409R mutation,
according to EU numbering.

Embodiment 59. The multispecific antibody according to embodiment 57 or 58, wherein:
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) an F405L mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an K409R mutation;
or
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) an K409R mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an F405L mutation,
according to EU numbering.

Embodiment 60. The multispecific antibody according to any one of embodiments 57 to 59, wherein:
i) the first part that binds to IL-31 comprises a first Fc polypeptide comprising the amino acid sequence shown in SEQ ID NO: 44, and the second part that binds to IL-13 comprises a second Fc polypeptide comprising the amino acid sequence shown in SEQ ID NO: 45; or
ii) the first part that binds to IL-31 comprises a first Fc polypeptide comprising the amino acid sequence shown in SEQ ID NO: 45, and the second part that binds to IL-13 comprises a second Fc polypeptide comprising the amino acid sequence shown in SEQ ID NO: 44.

Embodiment 61. A multispecific, preferably bispecific, antibody comprising a first part that binds to IL-31 and a second part that binds to IL-13, wherein the first part that binds to IL-31 comprises:
i) a light chain comprising the amino acid sequence shown in SEQ ID NO: 9; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 34; and
ii) a heavy chain comprising the amino acid sequence shown in SEQ ID NO: 104 or 107;
and the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 35; and
ii) a heavy chain comprising the amino acid sequence shown in SEQ ID NO: 104 or 107;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 33; and
ii) a heavy chain comprising the amino acid sequence shown in SEQ ID NO: 104 or 107;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 9; and
ii) a heavy chain comprising the amino acid sequence shown in SEQ ID NO: 104 or 107;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 34; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 35; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 33; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 9; and
ii) a heavy chain comprising the amino acid sequence shown in SEQ ID NO: 105 or 108;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 34; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 35; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110;
or
wherein the first part that binds to IL-31 comprises:
i) a light chain comprising the amino acid sequence shown in SEQ ID NO: 33; and
ii) a heavy chain comprising the amino acid sequence shown in SEQ ID NO: 10 or 109;
and wherein the second part that binds to IL-13 comprises:
iii) a light chain comprising the amino acid sequence shown in SEQ ID NO: 19, and
iv) a heavy chain comprising the amino acid sequence shown in SEQ ID NO: 20 or 110.

Embodiment 62. A multispecific, preferably bispecific, antibody according to any one of embodiments 40 to 61, wherein the first part that binds to IL-31 comprises:
i) a light chain comprising or consisting of the amino acid sequence shown in SEQ ID NO: 9; and
ii) a heavy chain comprising or consisting of the amino acid sequence shown in SEQ ID NO: 10;
and wherein the second part that binds to IL-13 comprises:
iii) a light chain comprising or consisting of the amino acid sequence shown in SEQ ID NO: 19; and
iv) a heavy chain comprising or consisting of the amino acid sequence shown in SEQ ID NO: 20.

Embodiment 63. A multispecific, preferably bispecific, antibody according to any one of embodiments 40 to 61, wherein the first part that binds to IL-31 comprises:
i) a light chain comprising or consisting of the amino acid sequence shown in SEQ ID NO: 9; and
ii) a heavy chain comprising or consisting of the amino acid sequence shown in SEQ ID NO: 109;
and wherein the second part that binds to IL-13 comprises:
iii) a light chain comprising or consisting of the amino acid sequence shown in SEQ ID NO: 19; and
iv) a heavy chain comprising or consisting of the amino acid sequence shown in SEQ ID NO: 110.

Embodiment 64. The multispecific antibody according to any one of embodiments 40 to 63, wherein the antibody is monovalent with respect to binding IL-31 and monovalent with respect to binding IL-13.

Embodiment 65. The multispecific antibody according to any one of embodiments 40 to 64, wherein the light chain of the first part that binds to IL-31 is a kappa light chain and wherein the light chain of the second part that binds to IL-13 is a kappa light chain.

Embodiment 66. The multispecific antibody according to any one of embodiments 40 to 65, wherein the antibody has an IgG, preferably an IgG1 isotype.

Embodiment 67. The multispecific antibody according to any one of embodiments 40 to 66, wherein the antibody is bivalent, and wherein the antibody is monovalent with respect to binding IL-31 and monovalent with respect to binding IL-13.

Embodiment 68. The multispecific antibody according to any one of embodiments 40 to 67, wherein the antibody is humanized.

Embodiment 69. The multispecific antibody according to any one of embodiments 40 to 68, wherein the first part that binds to IL-31 comprises a Fab and wherein the second part that binds to IL-13 comprises a Fab.

Embodiment 70. The multispecific antibody according to any one of embodiments 40 to 69, wherein the first part that binds to IL-31 is monoclonal and wherein the second part that binds to IL-13 is monoclonal.

Embodiment 71. The multispecific antibody according to any one of embodiments 40 to 70, wherein the IL-31 and IL-13 are human.

Embodiment 72. The multispecific antibody according to any one of embodiments 40 to 71, wherein the antibody binds human IL-31 with a dissociation constant (KD) of less than 30 pM, less than 25 pM, less than 20 pM, less than 15 pM, less than 10 pM, or less than about 10 pM as measured using surface plasmon resonance.

Embodiment 73. The multispecific antibody according to any one of embodiments 40 to 72, wherein the antibody binds human IL-13 with a dissociation constant (KD) of less than 30 pM, less than 25 pM, less than 20 pM, less than 15 pM, less than 10 pM, less than 5 pM, less than 1 pM or less than about 1 pM, as measured using surface plasmon resonance.

Embodiment 74. The multispecific antibody according to any one of embodiments 40 to 73, wherein the antibody inhibits human IL-31 activity, preferably wherein the antibody inhibits human IL-31 activity with an IC50 of less than 30 pM, such as less than 25 pM, or less than 20 pM or less than about 20 pM, as measured by an IL-31 dependent Baf3 cell proliferation assay.

Embodiment 75. The multispecific antibody according to any one of embodiments 40 to 74, wherein the antibody inhibits human IL-13 activity, preferably wherein the antibody inhibits human IL-13 activity with an IC50 of less than 50 pM, less than 40 pM, less than 30 pM, less than 20 pM, less than 15 pM or less than about 15 pM, as measured in a HEK-Blue cell reporter assay.

Embodiment 76. An ex vivo method for detecting the presence and/or concentration of IL-31 protein in a biological sample, comprising the steps of:
(i) providing a biological sample from a subject,
(ii) reacting said biological sample with at least one antibody of any one of embodiments 1 to 38, under conditions sufficient for binding IL-31 protein present in said biological sample to said at least one antibody through antigen-antibody interactions, thus forming an IL-31-antibody complex; and
(iii) detecting a signal proportional to the level of IL-31-antibody complex formed in step (ii), wherein the intensity of the signal correlates with the concentration of IL-31 protein in the biological sample.

Embodiment 77. A pharmaceutical composition comprising the antibody of any one of embodiments 1 to 38, or the multispecific antibody of any one of embodiments 39 to 75, optionally in combination with one or more pharmaceutically acceptable excipients, diluents, or carriers.

Embodiment 78. An antibody according to any one of embodiments 1 to 38, a multispecific antibody according to any one of embodiments 39 to 75, or a pharmaceutical composition according to embodiment 77 for use as a medicament.

Embodiment 79. Use of the antibody of any one of embodiments 1 to 38, the multispecific antibody of any one of embodiments 39 to 75, or the pharmaceutical composition of embodiment 77 as a medicament.

Embodiment 80. An antibody according to any one of embodiments 1 to 38, a multispecific antibody according to any one of embodiments 39 to 75, or a pharmaceutical composition according to embodiment 77, for use in preventing and/or treating an IL-31-related disease or disorder.

Embodiment 81. Use of the antibody according of one of embodiments 1 to 38, the multispecific antibody of any one of embodiments 39 or 75, or the pharmaceutical composition of embodiment 77, for the manufacture of a medicament for the treatment and/or prevention of an IL-31-related disease or disorder.

Embodiment 82. A method of preventing and/or treating an IL-31-related disease or disorder in a subject in need thereof, comprising administering to the subject a therapeutically effective amount of the antibody of any one of embodiments 1 to 38, the multispecific antibody of any one of embodiments 39 to 75, or the pharmaceutical composition of embodiment 77.

Embodiment 83. The antibody, multispecific antibody, or pharmaceutical composition for use of embodiment 80, the use of embodiment 81, or the method of embodiment 82, wherein the IL-31-related disease or disorder is an inflammatory or immune disease or disorder.

Embodiment 84. The antibody, multispecific antibody, or pharmaceutical composition for use of embodiment 83, the use of embodiment 83, or the method of embodiment 83, wherein the inflammatory or immune disease or disorder is a skin disease or disorder.

Embodiment 85. The antibody, multispecific antibody, or pharmaceutical composition for use according to any one of embodiments 80, 83, or 84, the use according to any one of embodiments 81, 83, or 84, or the method according to any one of embodiments 82 to 84, wherein the IL-31 is human.

Embodiment 86. A method of inhibiting the activity of IL-31, the method comprising contacting an effective amount of the antibody of any one of embodiments 1 to 38, the multispecific antibody of any one of embodiments 39 to 75, or the pharmaceutical composition of embodiment 77, with a plurality of mammalian, preferably human cells.

Embodiment 87. An ex vivo method for detecting the presence and/or concentration of IL-31 protein and/or IL-13 protein in a biological sample, comprising the steps of:
(i) providing a biological sample from a subject,
(ii) reacting said biological sample with at least one multispecific antibody, preferably bispecific antibody, according to any one of embodiments 40 to 75, under conditions sufficient for binding IL-31 protein and/or IL-13 protein present in said biological sample to said at least one antibody through antigen-antibody interactions, thus forming an IL-31- and/or IL-13-antibody complex; and
(iii) detecting a signal proportional to the level of IL-31- and/or IL-13-antibody complex formed in step (ii),
wherein the intensity of the signal correlates with the concentration of IL-31 protein and/or IL-13 protein in the biological sample.

Embodiment 88. A multispecific antibody, preferably bispecific antibody, according to any one of embodiments 40 to 75, or a pharmaceutical composition according to embodiment 77, for use in preventing and/or treating an IL-31- and IL-13-related disease or disorder.

Embodiment 89. Use of the multispecific antibody, preferably bispecific antibody, of any one of embodiments 40 to 75, or the pharmaceutical composition of embodiment 77, for the manufacture of a medicament for the treatment or prevention of an IL-31 and IL-13-related disease or disorder.

Embodiment 90. A method of preventing and/or treating an IL-31- and IL-13-related disease or disorder in a subject in need thereof, comprising administering to the subject a therapeutically effective amount of the multispecific antibody, preferably a bispecific antibody, of any one of embodiments 40 to 75, or the pharmaceutical composition of embodiment 77.

Embodiment 91. The multispecific antibody, preferably bispecific antibody, or pharmaceutical composition for use of embodiment 88, the use of embodiment 89, or the method of embodiment 90, wherein the IL-31- and IL-13-related disease or disorder is an inflammatory or immune disease or disorder.

Embodiment 92. The multispecific antibody, preferably bispecific antibody, or pharmaceutical composition for use of embodiment 91, the use of embodiment 91, or the method of embodiment 91, wherein the inflammatory or immune disease or disorder is a skin disease or disorder.

Embodiment 93. The multispecific antibody, preferably bispecific antibody, or pharmaceutical composition for use according to any one of embodiments 88, 91 or 92, the use according to any one of embodiments 89, 91, or 92, or the method according to any one of embodiments 90 to 92, wherein the IL-31 and IL-13 are human.

Embodiment 94. A method of simultaneously inhibiting the activity of IL-31 and IL-13, the method comprising contacting an effective amount of the multispecific antibody, preferably bispecific antibody, of any one of embodiments 40 to 75 with a plurality of mammalian, preferably human cells.

Embodiment 95. The method of embodiment 94, wherein the method comprises administering the antibody to a subject, preferably a human subject.

Embodiment 96. The method of embodiment 94, wherein the method is an ex vivo or an in vitro method.

Embodiment 97. An isolated nucleic acid molecule or set of isolated nucleic acid molecules encoding the antibody of any one of embodiments 1 to 38 or the multispecific antibody of any one of embodiments 39 to 75.

Embodiment 98. A set of isolated nucleic acid molecules encoding a multispecific antibody, preferably bispecific antibody, of any one of embodiments 40 to 75, wherein the first part binding to IL-31 and the second part binding to IL-13 are encoded by a separate nucleic acid molecule.

Embodiment 99. An expression vector or set of expression vectors comprising the isolated nucleic acid molecule or set of isolated nucleic acid molecules of embodiment 97 or 98.

Embodiment 100. A set of expression vectors comprising the set of isolated nucleic acid molecules of embodiment 99, wherein the nucleic acid molecule encoding the first part that binds to IL-31 and the nucleic acid molecule encoding the second part that binds to IL-13 are comprised by different vectors.

Embodiment 101. A host cell comprising the nucleic acid molecule or set of nucleic acid molecules of embodiment 97 or 98 or the expression vector or set of expression vectors of embodiment 99 or 100.

Embodiment 102. A host cell comprising a nucleic acid molecule encoding the first part that binds to IL-31 of the multispecific antibody, preferably bispecific antibody, of any one of embodiments 40 to 75, or an expression vector comprising said nucleic acid molecule.

Embodiment 103. A host cell comprising a nucleic acid molecule encoding the second part that binds to IL-13 of the multispecific antibody, preferably bispecific antibody, of any one of embodiments 40 to 75, or an expression vector comprising said nucleic acid molecule.

Embodiment 104. A host cell according to any one of embodiments 101 to 103, wherein the host cell is a HEK cell or a CHO cell.

Embodiment 105. A mixture of host cells comprising a first and second host cell, wherein:
a. the first host cell comprises a nucleic acid molecule encoding the first part that binds to IL-31 of the multispecific antibody, preferably bispecific antibody, of any one of embodiments 40 to 75, or an expression vector comprising said nucleic acid molecule; and
b. the second host cell comprises a nucleic acid molecule encoding the second part that binds to IL-13 of the multispecific antibody, preferably bispecific antibody, of any one of embodiments 40 to 75, or an expression vector comprising said nucleic acid molecule.

Embodiment 106. A cell culture comprising the host cell of any one of embodiments 101 to 104, or the mixture of host cells of embodiment 105.

Embodiment 107. An antibody according to any one of embodiments 1 to 38, a multispecific antibody, preferably a bispecific antibody, according to any one of embodiments 39 to 75, or a pharmaceutical composition according to embodiment 77, for use in treating and/or preventing atopic dermatitis.

Embodiment 108. Use of the antibody of any one of embodiments 1 to 38, the multispecific antibody, preferably bispecific antibody, of any one of embodiments 39 to 75, or the pharmaceutical composition of embodiment 77, for treating and/or preventing atopic dermatitis.

Embodiment 109. Use of the antibody of any one of embodiments 1 to 38, the multispecific antibody, preferably bispecific antibody, of any one of embodiments 39 to 75, or the pharmaceutical composition of embodiment 77, for the manufacture of a medicament for treating and/or preventing atopic dermatitis.

Embodiment 110. A method of treating and/or preventing atopic dermatitis in a subject in need thereof, comprising administering to the subject a therapeutically effective amount of the antibody of any one of embodiments 1 to 38, the multispecific antibody, preferably bispecific antibody, of any one of embodiments 39 to 75, or the pharmaceutical composition of embodiment 77.

Embodiment 111. An antibody according to any one of embodiments 1 to 38, a multispecific antibody, preferably a bispecific antibody, according to any one of embodiments 39 to 75, or a pharmaceutical composition according to embodiment 77, for use in preventing and/or treating pruritus, preferably pruritus associated with atopic dermatitis.

Embodiment 112. Use of the antibody of any one of embodiments 1 to 38, the multispecific antibody, preferably bispecific antibody, of any one of embodiments 39 to 75, or the pharmaceutical composition of embodiment 77, in the prevention and/or treatment of pruritus, preferably pruritus associated with atopic dermatitis.

Embodiment 113. Use of the antibody of any one of embodiments 1 to 38, the multispecific antibody, preferably bispecific antibody, of any one of embodiments 39 to 75, or the pharmaceutical composition of embodiment 77, for the manufacture of a medicament for the treatment and/or prevention of pruritus, preferably pruritus associated with atopic dermatitis.

Embodiment 114. A method of preventing and/or treating pruritus in a subject in need thereof, preferably pruritus associated with atopic dermatitis, comprising administering to the subject a therapeutically effective amount of the antibody of any one of embodiments 1 to 38, the multispecific antibody, preferably bispecific antibody, of any one of embodiments 39 to 75, or the pharmaceutical composition of embodiment 77.

Embodiment 115. The antibody, multispecific antibody, or pharmaceutical composition for use of embodiment 107 or 111, the use of any one of embodiments 108-109 or 112-113, or the method of embodiment 110 or 114, wherein the atopic dermatitis is moderate to severe atopic dermatitis.

Embodiment 116. A method of preventing and/or treating an inflammatory or immune condition in a subject in need thereof, the method comprising simultaneously or sequentially administering a therapeutically effective amount of the antibody of any one of embodiments 1 to 38 and an IL-13 antagonist to the subject.

Embodiment 117. The method of embodiment 116, wherein the IL-13 antagonist is an antibody.

Embodiment 118. The method of embodiments 116 or 117, wherein the inflammatory or immune condition is atopic dermatitis.

Embodiment 119. The method of any one of embodiments 110 or 114 to 118, wherein the subject is a human.

Embodiment 120. A method of producing the antibody of any one of c embodiments 1 to 38, or the multispecific, preferably bispecific, antibody of any one of embodiments 39 to 75, comprising culturing the host cell of embodiment 101 or 104 under conditions sufficient to express the antibody, and thereafter recovering and optionally purifying the antibody from the host cell culture.

Embodiment 121. A method of producing the multispecific, preferably bispecific, antibody of any one of embodiments 40 to 75, comprising culturing the host cell of embodiment 102 and the host cell of embodiment 103, or the mixture of host cells of embodiment 105, under conditions sufficient to express the first part that binds to IL-31 and the second part that binds to IL-13, and thereafter recovering and optionally purifying the first and second part from the host cell culture.

Embodiment 122. The method according to embodiment 121, wherein the host cell of embodiment 102 and the host cell of embodiment 103 are cultured in different host cell cultures.

Embodiment 123. The method according to embodiment 121, wherein the host cell of embodiment 102 and the host cell of embodiment 103 are cultured in the same host cell culture.

Embodiment 124. A method of producing the multispecific antibody, preferably bispecific antibody, of any one of embodiments 40 to 75, said method comprising:
a) culturing a first host cell expressing the first part that binds to IL-31 in a culture under conditions sufficient to express the antibody, and a second host cell expressing the second part that binds to IL-13 in a culture under conditions sufficient to express the antibody,
wherein the first and second host cell are cultured in the same or in different cultures;
b) recovering the first- and second-part from the host cell culture(s);
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part; and
e) obtaining the multispecific antibody; and optionally purifying the multispecific antibody.

Embodiment 125. A method of producing a pharmaceutical composition comprising the multispecific antibody, preferably bispecific antibody, of any one of embodiments 40 to 75, said method comprising:
a) culturing a first host cell expressing the first part that binds to IL-31 in a culture under conditions sufficient to express the antibody, and a second host cell expressing the second part that binds to IL-13 in a culture under conditions sufficient to express the antibody, wherein the first and second host cell are cultured in the same or in different cultures;
b) recovering the first- and second-part from the host cell culture(s);
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part;
e) obtaining the multispecific antibody; and optionally purifying the multispecific antibody; and
f) formulating the multispecific antibody in a pharmaceutical composition, optionally in combination with one or more pharmaceutically acceptable excipients, diluents, or carriers.

Embodiment 126. The method according to embodiment 124 or 125, wherein recovering the first- and second-part from the host cell culture(s) comprises centrifugation or depth filtration optionally followed by sterile filtration.

Embodiment 127. The method according to any one of embodiments 124 to 126, wherein a purification step is performed prior to incubating the first- and second-part in step c) and/or step d).

Embodiment 128. The method according to embodiment 127, wherein the purification step is performed utilizing affinity chromatography, for example protein A affinity chromatography.

Embodiment 129. The method according to any one of embodiments 124 to 128, wherein the first- and second-part are incubated in step c) in equimolar concentrations, in a molar ratio of first-to-second part from about 1:1.01 to about 1:2, or in a molar ratio of second-to-first part from about 1:1.01 to about 1:2.

Embodiment 130. A method of producing a multispecific antibody, preferably bispecific antibody, of any one of embodiments 40 to 75, said method comprising:
a) culturing a first host cell expressing the first part that binds to IL-31 in a culture under conditions sufficient to express the antibody, and a second host cell expressing the second part that binds to IL-13 in a culture under conditions sufficient to express the antibody, wherein the first and second host cell are cultured in the same culture;
b) incubating together the first- and second-part, preferably under reducing conditions that allow inter-chain disulfide bond reduction in the hinge region;
c) incubating the first- and second-part under conditions that allow oxidation of cysteines in the hinge region to form inter-chain disulfide bonds between the first- and second-part; and
d) obtaining the multispecific antibody; and optionally purifying the multispecific antibody,
wherein incubating together the first- and second-part in step b) is done without any prior recovery of the first- and second-art from the culture.

Embodiment 131. A method of producing a pharmaceutical composition comprising the multispecific antibody, preferably bispecific antibody, of any one of embodiments 40 to 75, said method comprising:
a) producing the multispecific antibody according to the method of embodiment 130;
b) formulating the multispecific antibody in a pharmaceutical composition, optionally in combination with one or more pharmaceutically acceptable excipients, diluents, or carriers.

Embodiment 132. An antibody that competes for binding to an IL-31 epitope with an antibody comprising: an HCDR1 comprising SEQ ID NO: 1; an HCDR2 comprising SEQ ID NO: 2 or SEQ ID NO: 21; an HCDR3 comprising SEQ ID NO: 3 or SEQ ID NO: 23; an LCDR1 comprising SEQ ID NO: 4 or SEQ ID NO: 24; an LCDR2 comprising SEQ ID NO: 5 or SEQ ID NO: 25, and an LCDR3 comprising SEQ ID NO: 6 or SEQ ID NO: 26, preferably wherein the HCDR1, HCDR2, HCDR3, LCDR1, LCDR2, and LCDR3 are defined according to the Kabat numbering scheme.

Embodiment 133. An antibody according to embodiment 132 that competes for binding to an IL-31 epitope with an antibody comprising:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26,
or
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
preferably wherein the HCDR1, HCDR2, HCDR3, LCDR1, LCDR2, and LCDR3 are defined according to the Kabat numbering scheme.

Embodiment 134. An antibody that binds to a human IL-31 epitope, wherein the epitope consists of amino acid residues S35, D37, V38, K40, I41, E44, N81, P86, R89, A90, K93, R96, K101, D105, E106, I108, E109, H110, D112, K113, L114, I115, F116, Q117, D118, A119, P120, E121, and T122 of human IL-31, preferably wherein human IL-31 is represented by SEQ ID NO: 143 or SEQ ID NO: 173.

Embodiment 135. The antibody according to embodiment 134, wherein the antibody comprises a heavy chain variable region (VH) comprising at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29 and a light chain variable region (VL) comprising at least 80%, at least 85%, at least 90%, at least 95%, at least 96%, at least 97%, at least 98%, or at least 99% sequence identity with SEQ ID NO: 8 or SEQ ID NO: 30 or SEQ ID NO: 31 or SEQ ID NO: 32.

Embodiment 136. The antibody according to embodiment 134 or 135, wherein the antibody comprises a heavy chain variable region (VH) comprising at least 80%, at least 85%, at least 90%, at least 95%, at least 96%, at least 97%, at least 98%, or at least 99% sequence identity with SEQ ID NO: 7 and a light chain variable region (VL) comprising at least 80%, at least 85%, at least 90%, at least 95%, at least 96%, at least 97%, at least 98%, or at least 99% sequence identity with SEQ ID NO: 8.

Embodiment 137. The antibody according to embodiment 135 or 136, wherein the heavy chain variable region comprises relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1).

Embodiment 138. The antibody according to any one of embodiments 134 to 137, wherein the light chain variable region comprises relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

Embodiment 139. A multispecific, preferably bispecific, antibody comprising:
a first part that binds to a human IL-31 epitope, wherein the epitope consists of amino acid residues S35, D37, V38, K40, I41, E44, N81, P86, R89, A90, K93, R96, K101, D105, E106, I108, E109, H110, D112, K113, L114, I115, F116, Q117, D118, A119, P120, E121, and T122 of human IL-31, preferably wherein human IL-31 is represented by SEQ ID NO: 143 or SEQ ID NO: 173, and;
a second part that binds to IL-13 comprising a heavy chain variable region (VH) comprising the amino acid sequence shown in SEQ ID NO: 17 and a light chain variable region (VL) comprising the amino acid sequence shown in SEQ ID NO: 18.

Embodiment 140. The multispecific antibody according to embodiment 139, wherein the first part that binds to the human IL-31 epitope comprises a heavy chain variable region (VH) comprising at least 80%, at least 85%, at least 90%, at least 95%, at least 96%, at least 97%, at least 98%, or at least 99% sequence identity with SEQ ID NO: 7 or SEQ ID NO: 28 or SEQ ID NO: 29 and a light chain variable region (VL) comprising at least 80%, at least 85%, at least 90%, at least 95%, at least 96%, at least 97%, at least 98%, or at least 99% sequence identity with SEQ ID NO: 8 or SEQ ID NO: 30 or SEQ ID NO:31 or SEQ ID NO:32.

Embodiment 141. The multispecific antibody according to embodiment 139 or 140, wherein the first part that binds to the human IL-31 epitope comprises a heavy chain variable region (VH) comprising at least 80%, at least 85%, at least 90%, at least 95%, at least 96%, at least 97%, at least 98%, or at least 99% sequence identity with SEQ ID NO: 7 and a light chain variable region (VL) comprising at least 80%, at least 85%, at least 90%, at least 95%, at least 96%, at least 97%, at least 98%, or at least 99% sequence identity with SEQ ID NO: 8.

Embodiment 142. The multispecific antibody according to embodiment 140 or 141, wherein the first part that binds to the human IL-31 epitope comprises a heavy chain variable region comprising relative to SEQ ID NO: 40 a substitution of the leucine (L) at position 29 (VH L29) by a different amino acid, preferably by an arginine (R), a substitution of the glycine at position 54 (VH G54) by a different amino acid, preferably by an alanine (A), and a substitution of the aspartic acid (D) at position 55 (VH D55) by a different amino acid, preferably by a glutamic acid (E), wherein the glutamine (Q) at the first position of SEQ ID NO: 40 is denoted as position 1 (VH Q1).

Embodiment 143. The multispecific antibody according to any one of embodiments 139 to 141, wherein the first part that binds to the human IL-31 epitope comprises a light chain variable region comprising relative to SEQ ID NO: 41 a substitution of the histidine at position 30 (VL H30) by a different amino acid, preferably by a glycine (G) and a substitution of the asparagine (N) at position 50 (VL N50) by a different amino acid, preferably by a tyrosine (Y), wherein the aspartic acid (D) at the first position of SEQ ID NO: 41 is denoted as position 1 (VL D1).

Embodiment 144. The antibody of any one of embodiments 134 to 138, or the multispecific antibody of any one of embodiments 138 to 141, wherein the antibody or the multispecific antibody is humanized.

Embodiment 145. The antibody of any one of embodiments 134 to 138 or 144, or the multispecific antibody of any one of embodiments 138 to 142, wherein the antibody or the multispecific antibody is an IgG type, for example an IgG1 type.

Embodiment 146. The multispecific antibody of any one of embodiments 139 to 145, wherein the multispecific antibody binds human IL-31 with a dissociation constant (KD) of less than 30 pM, less than 25 pM, less than 20 pM, less than 15 pM, less than 10 pM or less than about 10 pM, as measured using surface plasmon resonance.

Embodiment 147. The multispecific antibody of any one of embodiments 139 to 145, wherein the multispecific antibody inhibits human IL-31 activity with an IC50 of less than 30 pM, less than 25 pM, or less than 20 pM or less than about 20 pM, as measured by an IL-31 dependent Baf3 cell proliferation assay.

Embodiment 148. The multispecific antibody according to any one of embodiments 139 to 147, wherein the multispecific antibody binds human IL-13 with a dissociation constant (KD) of less than 30 pM, less than 25 pM, less than 20 pM, less than 15 pM, less than 10 pM, less than 5 pM, less than 1 pM or less than about 1 pM, as measured using surface plasmon resonance. 149. The multispecific antibody of any one of embodiments 139 to 148, wherein the multispecific antibody inhibits human IL-13 activity with an IC50 of less than 50 pM, less than 40 pM, less than 30 pM, less than 20 pM, less than 15 pM or less than about 15 pM, as measured in a HEK-Blue cell reporter assay.

### GENERAL

The contents of any patents, patent applications, and references cited throughout this specification are hereby incorporated by reference in their entireties. Unless otherwise required by context, singular terms used herein shall include pluralities and plural terms shall include the singular.

Various embodiments of the invention are described herein. It will be recognized that features specified in each embodiment may be combined with other specified features to provide further embodiments of the present invention. When an embodiment is described as being “according to” or “of” a previous embodiment, the previous embodiment includes sub-embodiments thereof.

The present disclosure includes a sequence listing. Certain sequences represented by specific SEQ ID NOs are further included in the detailed description. In cases where a discrepancy arises between a sequence represented by a SEQ ID NO in the detailed description and a sequence represented by the same SEQ ID NO as disclosed in the sequence listing, the detailed description prevails.

The following examples are provided to further illustrate the disclosure but should not be considered to limit its scope in any way. Other variants of the disclosure will be readily apparent to one of ordinary skill in the art and are encompassed by the description and the appended claims.

### Examples

### Example 1. Humanization of Monoclonal Antibodies

The process of humanization is well described in the art (Jones, et al 1986, Queen, et al 1989, Riechmann, et al 1988, Verhoeyen, Milstein and Winter 1988). The term humanization is described as the transfer of the antigen-binding site of a non-human antibody, e.g., a murine derived antibody, to a human acceptor framework, e.g., a human germline sequence (Retter, et al 2005). The main rationale for humanizing an antibody is to minimize the risk of developing an immunogenic response towards the antibody when the antibody is administered as a therapeutic in humans (Rebello, et al 1999).

The antigen-binding site comprises the complementary determining regions (CDRs) (Chothia and Lesk 1987, Kabat, et al 1991) and positions in the framework region of the variable domains (VL and VH) that directly or indirectly affect binding. Framework residues that may directly affect binding can, for example, be found in the so called “outer” loop region located between CDR2 and CDR3. Residues that indirectly affect binding are for example found at so called Vernier Zones (Foote and Winter 1992). They are thought to support CDR conformation. Those positions outside the CDRs are considered when choosing a suitable acceptor framework to minimize the number of deviations of the final humanized antibody to the human germline acceptor sequence in the framework regions.

The humanized anti-IL31 Fab used in the present disclosure is TPP-13348 (SEQ ID NO: 155 (HC) and 156 (LC)), with the VH and VL regions being represented by SEQ ID NO: 159 and 160 respectively.

Human IGHV1-69*06 germline sequence (IMGT) was used as template for humanization of VH domain. Human IGHJ6*01 germline sequence (IMGT) was used as template for humanization of the heavy chain J domain.

Human IGKV1-27*01 germline sequence (IMGT) was used as template for humanization of VL domain. Human IGKJ4*01 germline sequence (IMGT) was used as template for humanization of the light chain J domain.

The humanized anti-IL31 Fab used as reference (TPP-20012; SEQ ID NO 153 (HC) and 154 (LC)) is derived from the clone 292.12.3.1 described in patent application WO2008/028192, with the VH and VL regions being represented by SEQ ID NO: 40 and SEQ ID NO: 41 respectively.

SEQ ID NO: 159 (TPP-13348) differs from SEQ ID NO: 40 (TPP-20012) in that it comprises a serine at position 16 (A16S substitution), a lysine in position 65 (Q65K substitution), an isoleucine at position 70 (L70I substitution), and a threonine at position 117 (L117T substitution), when the positions are numbered starting from the glutamine (Q) at position 1 of SEQ ID NO: 40 or SEQ ID NO: 159.

SEQ ID NO: 160 (TPP-13348) differs from SEQ ID NO: 41 (TPP-20012) in that it comprises an aspartic acid at position 70 (Q70D substitution), a threonine at position 72 (S72T substitution), and a glycine at position 100 (Q100G substitution), when the positions are numbered starting from the aspartic acid (D) at position 1 of SEQ ID NO: 41 or SEQ ID NO: 160.

### Example 2. Sequence Optimization

Certain amino acid sequence motifs are known to undergo post-translational modification (PTM) such as glycosylation (i.e. N×S/T, × any but P), oxidation of free cysteines, deamidation (e.g. NG) or isomerization (e.g. DG). If present in the CDR regions, those motifs are ideally removed by site-directed mutagenesis in order to increase product homogeneity.

Two PTM sites were removed from the humanized Fab TPP-13348. DG isomerization site in H-CDR3 was removed by substituting VH Asp100 with a Glu residue (when the positions are numbered starting from the glutamine (Q) at position 1 of the VH as discussed above). DG isomerization site in L-CDR2 was removed by substituting VL Asp56 with a Glu residue (when the positions are numbered starting from the aspartic acid (D) at position 1 of the VL as discussed above). The resulting Fab TPP-13357 (SEQ ID NO: 147 (HC) and 148 (LC)) was used for crystallization in complex with human IL-31 (see Example 3).

### Example 3. Structure of the Humanized Anti-IL-31 Fab in Complex with Human IL-31 for Structure-Guided Antibody Engineering

### (i) Material and Methods

Standard molecular biological protocols were used to obtain the Fab TPP-13357. In brief, the Fab was cloned and expressed in Human Embryonic Kidney cells (HE293-T ATCC11268). The recombinant protein was purified by affinity chromatography on a CaptureSelect CH1-XL column (Thermofisher, Waltham, MA, US) and polished by size exclusion on a HiLoad 16/600 Superdex 200 resin (Cytiva, Marlborough, MA, US).

Standard molecular biological protocols were also used to obtain the human IL-31 protein. In brief, the full-length, untagged human IL-31 C133S (variant of SEQ ID NO: 173 comprising an C133S substitution; aa 24-164 of human IL-31) was cloned and expressed in E. coli BL21 (DE3) cells. IL-31 C133S was refolded from inclusion bodies and purified. LC-MS analysis showed the expected mass (15819.8 Da).

The Fab fragment complex with human IL-31 was then prepared using standard methodology. In brief, a 1.8-fold excess of human IL-31 was added to the Fab. The mixture was concentrated by ultrafiltration and purified by SEC on a Superdex 75 10/30 column (Cytiva) equilibrated with 50 mM Tris pH 7.5, 75 mM NaCl. Pooled fractions used for crystallization were concentrated by ultrafiltration to 27 mg/ml.

Standard crystallization protocols were followed. In brief, the Fab complex with IL-31 C133S at 27 mg/ml in 50 mM Tris pH 7.5, 75 mM NaCl was crystallized using the sitting drop vapour diffusion technique by mixing 0.241 of protein stock with 0.241 of crystallization buffer (0.2M sodium iodure, 20% PEG 3350, 0.1M Bis-Tris Propane pH 8.5) and equilibrating against the same buffer from the reservoir in an Innovadyne SD2 96-well plate at 20° C. Crystals used for X-ray diffraction appeared after 2 days and grew to full size within one week.

X-ray data collection and processing was carried out using standard protocols. Briefly, crystals were cryo-protected with a 1:1 mix of the reservoir solution with a solution consisting of 30% PEG 3,350 and 30% glycerol and then directly flash-cooled into liquid nitrogen. X-ray data were collected at the European Synchrotron Radiation Facility, beamline ID30B, using X-ray at wavelength of 0.97625 Å. A total of 2700 images with 0.10° oscillation each were recorded at a crystal-to-detector distance of 540 mm using a Pilatus 6M pixel detector and processed with autoPROC program version 1.1.7 (20210716). Isotropic analysis using the Pearson correlation coefficient CC_{1/2} statistics led to a resolution cut-off of 2.796 Å.

The crystal structure of human IL-31 C133S in complex with the humanized antibody Fab was determined by molecular replacement with Phaser using model derived from anti-human NKP46 NKP46-1 (PDB entry 6IAP) Fv and an AlphaFold predicted model of human IL-31 (34-164) as search models. Iterative model building and refinement were carried out using programs Coot 0.9, autoBUSTER (2.11.7) and PHENIX (1.19.2). The final structure model of the Fab in complex with IL-31 C133S was refined to R-value of 21.9% and free R-value of 24.4%, both of which indicate a good quality of the structure model.

### (ii) Results

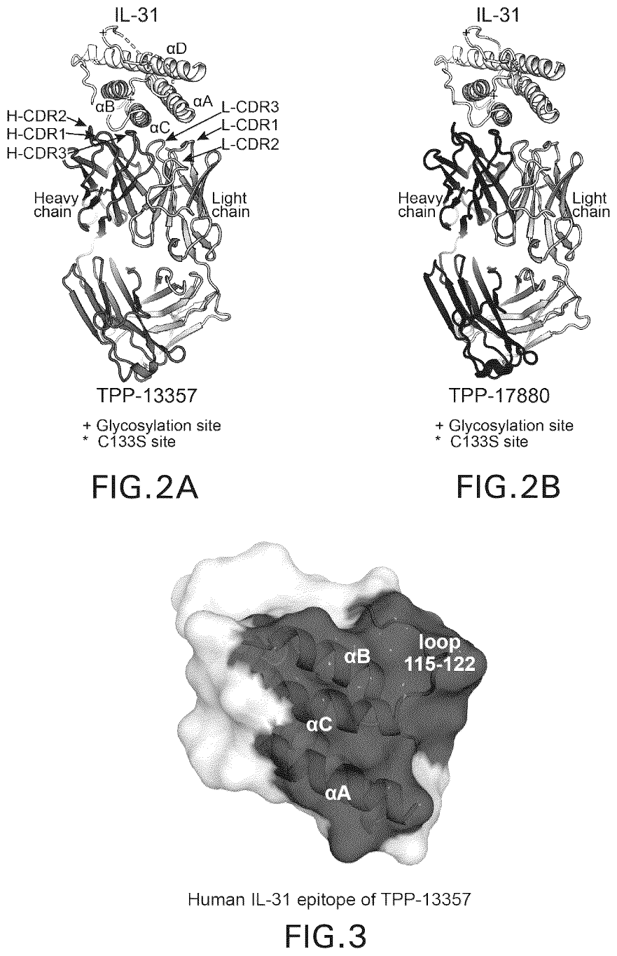

The crystal structure determined here provides the first experimental structure of IL-31 as well as its complexing with an antibody Fab. The structure shows that human IL-31 consists of four α-helices, namely αA, αB, αC, and αD (FIG. 2A-2B), which is a characteristic of four-helical cytokine family that also includes IL-2, IL-13, IL-23, and etc. Helices αA, αB, αC, and an extended loop region 115-122 between αC and αD are bound by the antibody TPP-13357 (FIG. 3). αB and the loop region between αC and αD plays a central role in the Fab binding, which constitute of the majority of key epitope residues. Helices αA and αB make additional contributions to the epitope. C133 S and the two potential N-lined glycosylation sites are distant from the Fab binding interface. We determined that the human IL-31 epitope to which TPP-13357 binds consists of residues D37, K40, I41, E44, N81, P86, R89, K93, R96, K101, D105, E106, I108, E109, H110, D112, K113, L114, I115, F116, Q117, D118, A119, P120, E121, T122 (FIG. 4A), which totals about 14% surface area of IL-31 (952 out of 6993 A2, FIG. 3). All epitope residues are highly conserved in cynomolgus IL-31 (FIG. 4A; SEQ ID NO: 144, 146). Among these epitope residues, D37, R89, K93, R96, D105, E109, H110, K113, F116, Q117, A119, and E121 are key residues recognized by TPP-13357.

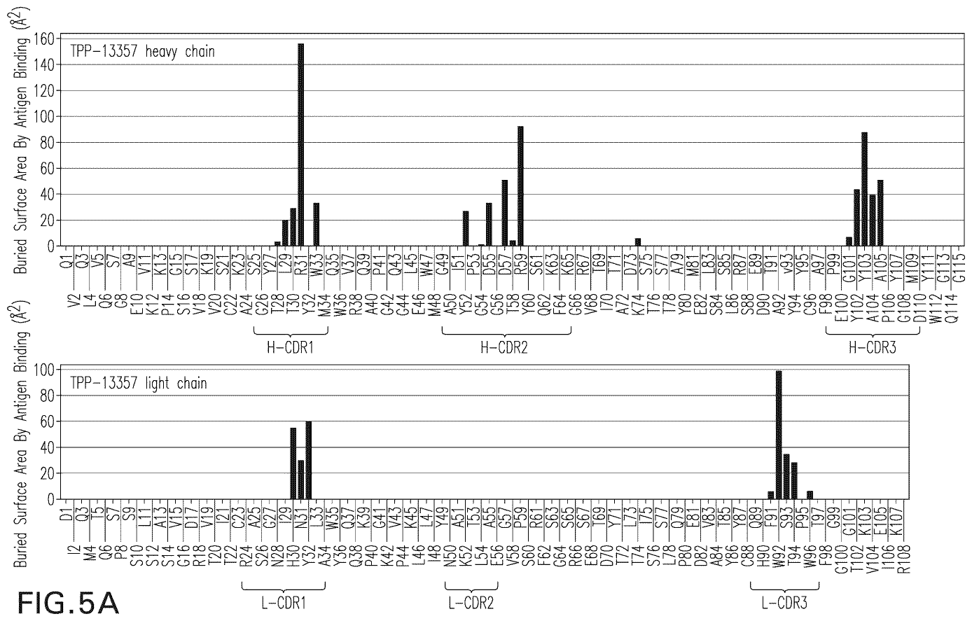

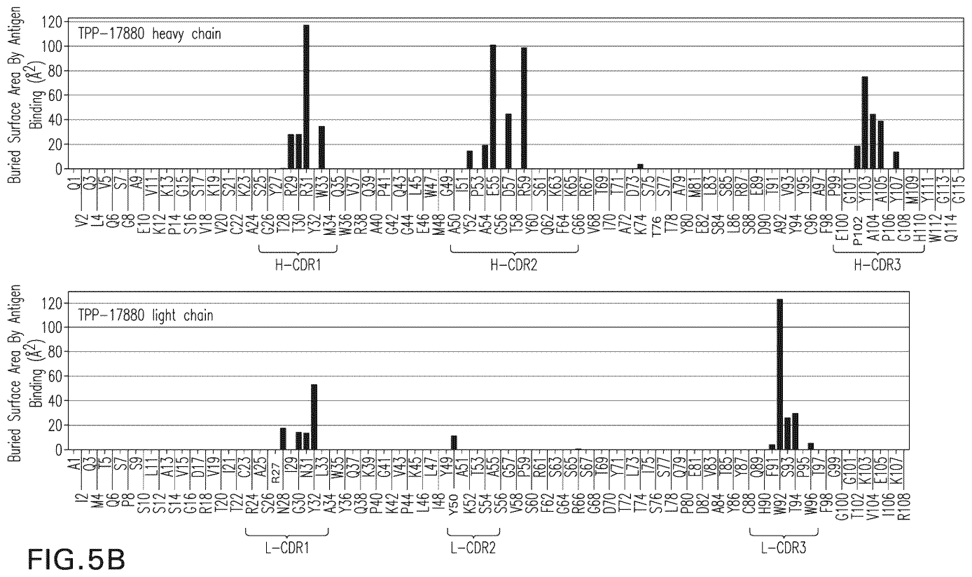

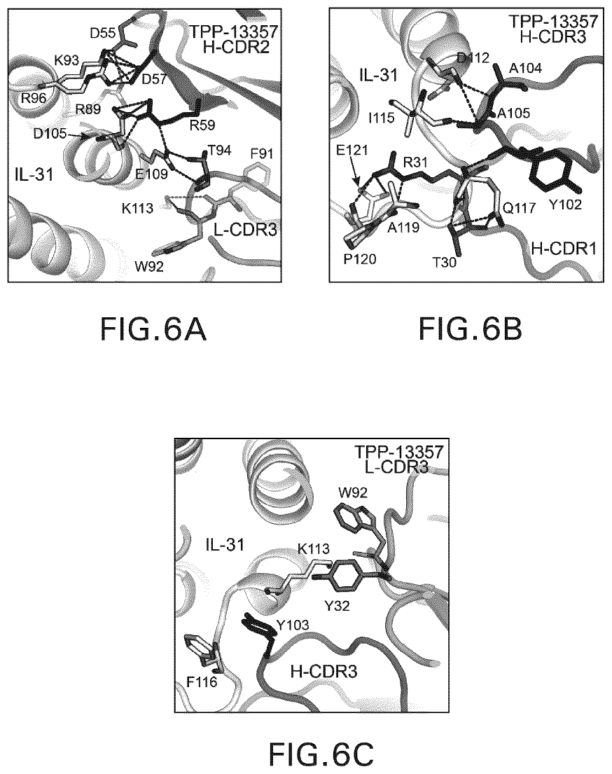

All CDRs except L-CDR2 in TPP-13357 contribute to the IL-31 binding (FIG. 5A-5B). The antibody heavy chain forms extensive polar interactions with IL-31 (FIG. 6A-C). D55, D57, and R59 from TPP-13357 H-CDR2 form 11 salt bridges with IL-31 R89, K93, R96, D105, and D109 (FIG. 6A). H-CDR1 T30 and R31 hydrogen bond to IL-31 Q117, A119, and E121 (FIG. 6B). The backbone of H-CDR3 Y102, A104, and A105 also hydrogen bonds to the backbone of IL-31 D112, 1115, and Q117. Meanwhile, L-CDR3 also contributes to some of the key interactions with IL-31 (FIG. 6A). L-CDR3 F91, W92, and T94 of the antibody hydrogen bond to IL-31 D109 and K113. In addition to these polar interactions, non-polar interactions also contribute to the binding to IL-31 (FIG. 6C). L-CDR1 Y32 and L-CDR3 W92 interact with the hydrophobic portion of the IL-31 K113 sidechain. H-CDR3 Y103 interacts with IL-31 F116 via π-π interaction.

### Example 4. Generation of Affinity Matured Antibody Variants

Actual affinity maturation of the initial anti-IL31 Fab (TPP-13357) was carried out in three steps: (i) library generation, (ii) library screening, and (iii) candidate characterization.

The protein engineering work (i.e. affinity maturation) was carried out in the Fab fragment format for ease of handling. Candidates were formatted back to full IgG after engineering.

### (i) Library Generation

The DNA sequence encoding the variable domain of the light chain was mutated to create a library of gene variants. Two different approaches (A and B) were used for library generation, providing two separate sets of libraries.

### A) Random Mutation by Error Prone PCR:

Two libraries were generated, one for random mutagenesis of the VH domain and another for random mutagenesis of the VL domain. The DNA region encoding the variable domain of interest was randomly mutated using error prone PCR. In more detail, this region was amplified using the polymerase Mutazyme II, which introduced mutations at a high frequency (for more detail, see the guidance supplied with the GeneMorph II random mutagenesis kit, supplied by Stratagene #200550). However, any suitable random mutation technique or strategy could be used.

The pool of PCR fragment was amplified using Phusion® High-Fidelity DNA Polymerase (New England Biolabs, Ipswich, MA, USA) using forward and reverse primers to add sequences at the 5′ and 3′ of the library that are homologous to the yeast surface display vector, allowing efficient homologous recombination in yeast cells. The final pool of PCR fragment variants was then transformed into S. cerevisiae along with the digested yeast display vector for yeast display. The S. cerevisiae strain had a BJ5465 (ATCC 208289) background and was tryptophan auxotrophic to enable plasmid selection. Library transformation was carried out as published (Benatuil et al., Protein Eng. Des. Sei. 23, 155-159 (2010)). This resulted in a library of yeast display vector variants comprising a variety of randomly mutagenized variable domain sequences.

### B) Mutation by Rational Design:

Under this approach, specific amino acid residues were targeted based on the epitope and paratope information derived from the crystal structure described above and based on results from random mutagenesis approach.

Two libraries were generated, one for the mutagenesis of amino acids residues of the VH domain and another for the mutagenesis of amino acids residues of the VL domain.

These libraries were synthesized by Twist Biosciences (CA, USA). The quality of these libraries was checked by high throughput sequencing (Twist Biosciences).

DNA fragments with the desired mutations, positions and frequency are described below. The synthesized VL library had a theoretical diversity of 98 304 variants and the VH library of 62 208 variants. The libraries were PCR amplified using Q5 Hot Start High-Fidelity DNAPolymerase (New England Biolabs) using forward and reverse primers to add sequences at the 5′ and 3′ of the fragments that are homologous to the yeast surface display vector, allowing efficient homologous recombination in yeast cells.

VH positions are numbered starting from the glutamine (Q) at position 1 of the VH of TPP-13357, and VL positions are numbered starting from the aspartic acid (D) at position 1 of the VL of TPP-13357 as discussed above.

TABLE 2
Design of VL library:

| L-chain | Amino- | | |
|---|---|---|---|
| Region | acid | Variants | Rationale |
| N- | Asp1 | Ala | Improved electrostatic complementarity with |
| terminus | | | antigen |
| L-CDR1 | Gly27 | Arg | Engage electrostatic interactions with Glu44 |
| | | | and/or Glu106 of IL-31 - (also found by error- |
| | | | prone PCR) |
| L-CDR1 | Asn28 | Asp | Target Lys40 of IL-31 |
| L-CDR1 | His 30 | Ser; | Improved electrostatic complementarity, |
| | | Gly; Ala | engage interaction with Lys40 of IL-31 |
| L-CDR2 | Asn50 | Arg; | Engage electrostatic interaction with Asp37 |
| | | Ala; Tyr | (Tyr: found by error-prone PCR) |
| L-CDR2 | Lys52 | Asp; | Catch Arg33/Arg30 of IL-31 (disordered in |
| | | Glu; Ser | X-ray); Ser is the germline residue |
| L-CDR2 | Thr53 | Asp; | Catch Arg33/Arg30 of IL-31 (disordered in |
| | | Glu | X-ray) |
| L-CDR2 | Leu54 | Ser | Germlining supported by structure (Ser: |
| | | | found by error-prone PCR) |
| L-CDR2 | Ala55 | Gln | Germlining supported by structure |
| L-CDR2 | Glu56 | Ser | Germlining supported by structure |
| L-CDR3 | Glu68 | Gly | Germlining supported by structure |
| L-CDR3 | Ser93 | Lys; | Engage electrostatic or polar interactions |
| | | Thr; Gln | with Glu44 and/or Glu106, or with Glu109 (Thr) |

TABLE 3
Design of VH library:

| H-chain | Amino- | | |
|---|---|---|---|
| Region | acid | Variants | Rationale |
| H-CDR1 | Leu29 | Arg; Ser | Ser: found by error-prone PCR; Arg: |
| | | | additional mutation introduced in library |
| | | | synthesis |
| H-CDR1 | Trp33 | His | Additional mutation introduced in library |
| | | | synthesis |
| H-CDR2 | Gly54 | Ala | Rigidification/stabilization of H-CDR2 (also |
| | | | found by error-prone PCR) |
| H-CDR2 | Asp55 | Glu | Electrostatic interactions with Lys93 and/or |
| | | | Arg89, PTM removal |
| H-CDR2 | Gly56 | Ala | PTM removal, not supported by structure |
| H-CDR2 | Lys65 | Glu, Gln | Improved electrostatic complementarity with |
| | | | IL-31 (Lys101) |
| H-CDR3 | Glu100 | Gln | Negatively charged cluster at V_{H}-V_{L} |
| | | | interface |
| H-CDR3 | Gly101 | Ala, Ser | Stabilization/rigidification of H-CDR3 |
| H-CDR3 | Tyr102 | His, | Exposed aromatic residue not involved in |
| | | Pro, Gln, | antigen binding; improved phys-chem |
| | | Arg, Ser | properties, improved stability of H-CDR3 |
| H-CDR3 | Ala104 | Asn, Ser | Engage H-bonded interactions with Ile115 O |
| | | | and/or Asp112 Oδ1 |
| H-CDR3 | Ala105 | Ser | Engage H-bonded interactions with Lys113 |
| | | | Nζ |
| H-CDR3 | Asp110 | His | found by error-prone PCR |

### (ii) Library Screening

Once libraries were generated, they were screened in order to select those which had superior characteristics to the parental sequence, for example higher affinity for IL-31. Fab yeast display was used herein because it provides high precision in retrieving the desired population when combined with flow cytometry.

Yeast display selections were performed as described in Chao G. et al, Nat Protocols 1: 755-768 (2006). Briefly, cells were induced in selective media containing 2% galactose overnight at 22° C. Induced cells were washed twice with cold washing buffer (PBS pH 7.4, 0.5% BSA) and incubated at room temperature with the biotinylated human IL-31 antigen diluted in PBS.

Standard molecular biological protocols were used to produce the recombinant human IL-31 protein. In brief, the human IL-31 sequence (Uniprot Q6EBC2; aa24-164) with N-terminal APP6- and Avi- tags was cloned and expressed in Human Embryonic Kidney cells (HE293-T ATCC11268). The recombinant protein was purified by affinity chromatography on an anti-APP Sepharose resin (internal) and polished by size exclusion on a Hi Load Superdex 200 resin (Cytiva). The purified protein was biotinylated using the BirAbiotin-protein ligase kit from Avidity following supplier's instructions.

Equilibrium selection was performed by incubating the Fab-displaying yeast cells with decreasing concentrations of biotinylated antigen as the selections round progress. After the biotinylated antigen incubation step the cells were washed and stained promptly with anti-kappa LC labeled with PE (phycoerythrin; Southern Biotech/2060-09; labels cells displaying Fab) and streptavidin labeled with APC (Invitrogen/5868; labels cells bound to biotinylated antigen) and then cells binding the antigen were sorted on a FACS Aria (BD Biosciences, NJ, US). Selected yeast cells were sorted into yeast selective medium, grown and induced, then stained again for consecutive rounds of selection.

(iii) Candidate Characterization

The hits identified during the screening were produced on a larger scale for further physicochemical characterization and to confirm high affinity binding to IL-31, and/or other advantageous properties in additional assays. These are described below in more detail.

### (iv) Results: Screening and Initial Characterization of Candidates Following Affinity Maturation

### 1) Random Mutagenesis Approach (Method A):

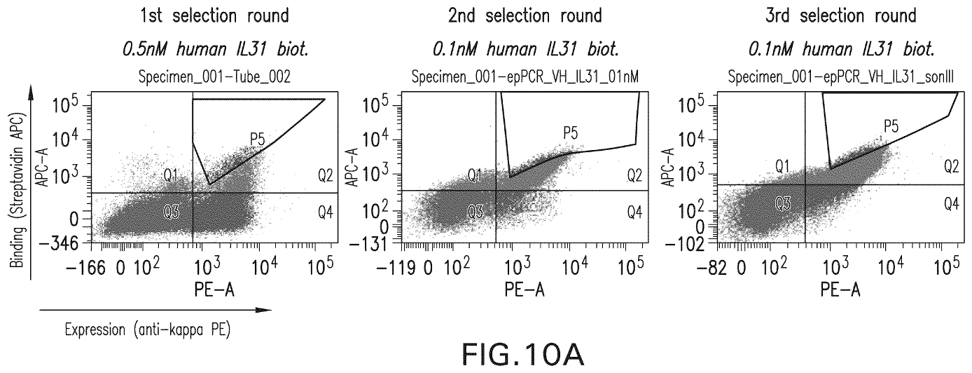

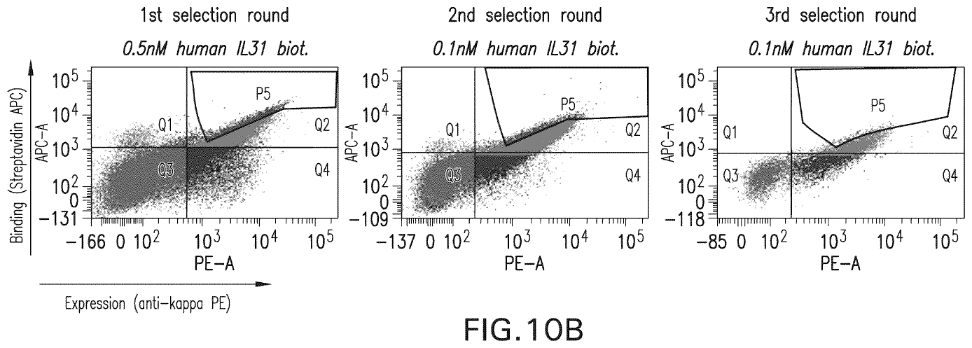

The mutation rate after the error-prone PCR library generation was found to peak at 2 to 3 mutations per gene. The present disclosure performed 3 rounds of selection using fluorescence activated cell sorting (FACS) to enable precise sorting of the cells of interest. An assessment of the population obtained for both libraries after each round of selection showed a slight improvement of affinity to human IL-31 (FIGS. 10A and 10B).

1.A) VL library

To evaluate the variants enriched by this selection, 94 clones from the third round of selection were randomly picked for monoclonal binding verification as yeast display by flow cytometry. Cells were washed twice with cold washing buffer (PBS pH 7.4 0.5% BSA) and incubated at room temperature with 0.1 nM of biotinylated antigen. After the biotinylated antigen incubation step the cells were washed and stained with the anti-kappa LC labeled with PE and streptavidin labeled with APC and then cells were analyzed by flow cytometry. The 13 clones showing the highest binding signal were submitted for Sanger sequencing. Thirteen clones were sequenced, and 8 unique sequences were identified. The most frequent mutation was G27R in L-CDR1, three clones presented this single point mutation. Two clones presented mutation G27R in L-CDR1 plus the mutation L54M in L-CDR2. One clone presented mutation G27R in L-CDR1 plus the mutation L54S in L-CDR2. Two clones presented mutation G27R in L-CDR1 plus the mutation S9Y in FW-1. Two clones presented mutation G27R in L-CDR1 plus the mutation S76P in FW-3. One clone presented mutation N50Y in L-CDR1. One clone presented mutation L46Q in FW-2. One clone presented mutation L46Q in FW-2 plus the mutation Q38L in FW-2.

1.B) VH library

To evaluate the variants enriched by this selection, 95 clones from the third round of selection were randomly picked for monoclonal binding verification as yeast display by flow cytometry. Cells were washed twice with cold washing buffer (PBS pH 7.4 0.5% BSA) and incubated at room temperature with 0.5 nM of biotinylated antigen. After the biotinylated antigen incubation step the cells are washed and stained with the anti-kappa LC labeled with PE and streptavidin labeled with APC and then cells were analyzed by flow cytometry. The 20 clones showing the highest binding signal were submitted for Sanger sequencing. Twenty clones were sequenced, and 11 unique sequences were identified. The most frequent mutation was Y107N in H-CDR3, five clones presented this single point mutation. Two clones presented mutation Y107N in H-CDR3 plus the mutation R87T in FW-3. One clone presented mutation Y107N in H-CDR3 plus the mutation S25Y in FW-1. One clone presented mutation Y107N in H-CDR3 plus the mutation the mutation P14L in FW-1. The second most frequent mutation was A101S in H-CDR3, three clones presented this single point mutation. One clone presented mutation A101S in H-CDR3 plus the mutation S77F in FW-3. One clone presented mutation A101S in H-CDR3 plus the mutation A9D in FW-1. The third most frequent mutation was L29S in H-CDR1, two clones presented this single point mutation. Two clones presented mutation L29S in H-CDR1 plus the mutation G54A in H-CDR2. One clone presented the mutation L29K in H-CDR1. One clone presented the mutation A101G in H-CDR3. One clone presented the mutation A101G in H-CDR3 plus the mutation D110H in H-CDR3. One clone presented the mutation K23M in FW-1.

### 2) Rational Mutagenesis Approach (Method B):

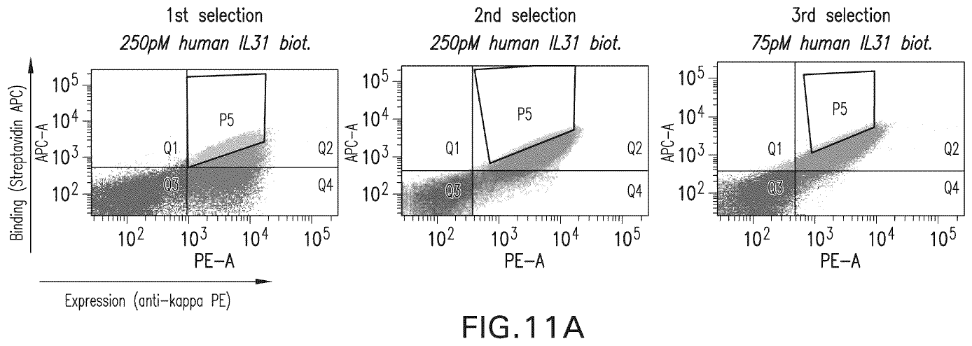

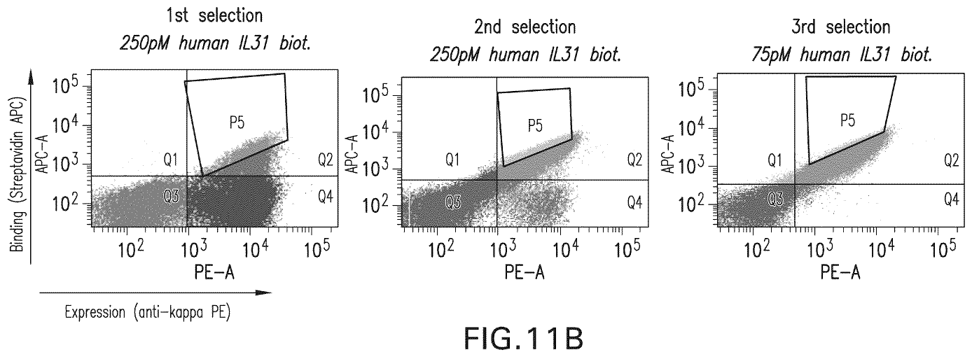

For both VL and VH libraries, a snapshot of the amino acid distribution at the randomized positions was generated by sequencing of 24 randomly picked members. 3 rounds of selection using fluorescence activated cell sorting (FACS) to enable precise sorting of the cells of interest were performed. The first 2 rounds were performed by incubating the Fab-displaying yeast cells with the biotinylated antigen at 0.25 nM and the third round at 0.075 nM. An assessment of the population obtained for both libraries after each round of selection showed a significant improvement of affinity (FIGS. 1A and 11B).

### 2.A) SG VL Library

To evaluate the variants enriched by this selection, 24 clones from the third round of selection were randomly picked and subjected to yeast colony PCR and Saenger sequencing. The amino acid distribution at the randomized positions is described in Table 4:

TABLE 4
SG VL library: Amino acid distribution at the
randomized positions pre- and pos-selection.

| L-chain | Amino- | Distribution from | Distribution from the |
|---|---|---|---|
| Region | acid | Naïve library (%) | output of round 3 (%) |
| N- | Asp1 | Ala: 46 | Ala: 82 |
| terminus | | Asp: 54 | Asp: 18 |
| L-CDR1 | Gly27 | Gly: 42 | Gly: 17 |
| | | Arg: 58 | Arg: 83 |
| L-CDR1 | Asn28 | Asn: 71 | Asn: 75 |
| | | Asp: 29 | Asp: 25 |
| L-CDR1 | His 30 | His: 4 | His: 9 |
| | | Ser: 33 | Ser: 4 |
| | | Gly: 21 | Gly: 60 |
| | | Ala: 38 | Ala: 27 |
| L-CDR2 | Asn50 | Asn: 9 | Asn: 0 |
| | | Arg: 26 | Arg: 8 |
| | | Ala: 30 | Ala: 4 |
| | | Tyr: 35 | Tyr: 88 |
| L-CDR2 | Lys52 | Lys: 43 | Lys: 50 |
| | | Asp: 13 | Asp: 0 |
| | | Glu: 13 | Glu: 5 |
| | | Ser: 30 | Ser: 45 |
| L-CDR2 | Thr53 | Thr: 35 | Thr: 63 |
| | | Asp: 22 | Asp: 4 |
| | | Glu: 43 | Glu: 33 |
| L-CDR2 | Leu 54 | Leu: 30 | Leu: 25 |
| | | Ser: 70 | Ser: 75 |
| L-CDR2 | Ala55 | Ala: 52 | Ala: 83 |
| | | Gln: 48 | Gln: 17 |
| L-CDR2 | Glu56 | Glu: 57 | Glu: 26 |
| | | Ser: 43 | Ser: 74 |
| L-CDR3 | Glu68 | Glu: 26 | Glu: 41 |
| | | Gly: 74 | Gly: 59 |
| L-CDR3 | Ser93 | Ser: 26 | Ser: 13 |
| | | Lys: 13 | Lys: 52 |
| | | Thr: 22 | Thr: 35 |
| | | Gln: 39 | Gln: 0 |

Having thus identified the substitutions enriched in the population of binders after three rounds of selection, four VL sequences were designed, including different combinations of these substitutions: SEQ ID NO: 31; SEQ ID NO: 32; SEQ ID NO: 30; SEQ LD NO: 8.

### 2.B) SG VH Library

To evaluate the variants enriched by this selection, 24 clones from the third round of selection were randomly picked and subjected to yeast colony PCR and Saenger sequencing. The amino acid distribution at the randomized position is described in Table 5:

TABLE 5
SG VL library: Amino acid distribution at the
randomized positions pre- and post-selection.

| H-chain | Amino- | Distribution from | Distribution from the |
|---|---|---|---|
| Region | acid | Naïve library (%) | output of round 3 (%) |
| H-CDR1 | Leu29 | Leu: 50 | Leu: 0 |
| | | Arg: 42 | Arg: 87 |
| | | Ser: 8 | Ser: 13 |
| H-CDR1 | Trp33 | Trp: 50 | Trp: 100 |
| | | His: 50 | His: 0 |
| H-CDR2 | Gly54 | Gly: 48 | Gly: 50 |
| | | Ala: 52 | Ala: 50 |
| H-CDR2 | Asp55 | Asp: 61 | Asp: 37 |
| | | Glu: 39 | Glu: 63 |
| H-CDR2 | Gly56 | Gly: 35 | Gly: 96 |
| | | Ala: 65 | Ala: 4 |
| H-CDR2 | Lys65 | Lys: 13 | Lys: 69 |
| | | Glu: 35 | Glu: 22 |
| | | Gln: 52 | Gln: 13 |
| H-CDR3 | Glu100 | Glu: 61 | Glu: 75 |
| | | Gln: 39 | Gln: 25 |
| H-CDR3 | Gly101 | Gly: 39 | Gly: 43 |
| | | Ala: 30 | Ala: 9 |
| | | Ser: 30 | Ser: 48 |
| H-CDR3 | Tyr102 | Tyr: 9 | Tyr: 4 |
| | | His: 9 | His: 13 |
| | | Pro: 35 | Pro: 38 |
| | | Gln: 13 | Gln: 25 |
| | | Arg: 13 | Arg: 21 |
| | | Ser: 22 | Ser: 22 |
| H-CDR3 | Ala104 | Ala: 43 | Ala: 96 |
| | | Asn: 35 | Asn: 0 |
| | | Ser: 22 | Ser: 4 |
| H-CDR3 | Ala105 | Ala: 61 | Ala: 100 |
| | | Ser: 39 | Ser: 0 |
| H-CDR3 | Asp110 | Asp: 30 | Asp: 0 |
| | | His: 70 | His: 100 |

Having thus identified the substitutions enriched in the population of binders after three rounds of selection, three VH sequences were designed, including different combinations of these substitutions: SEQ ID NO: 28; SEQ I3 NO: 29; SEQ ID NO: 7.

Twelve Fab candidates w ere finally cloned and expressed in Human Embryonic Kidney cells (HEK293-T ATCC11268):

TABLE 6
Affinity matured Fab variants produced in HEK expression system

| Fab ID | Fab HC sequence | Fab LC sequence |
|---|---|---|
| TPP-17867 | SEQ ID NO: 37 | SEQ ID NO: 34 |
| TPP-17868 | SEQ ID NO: 37 | SEQ ID NO: 35 |
| TPP-17869 | SEQ ID NO: 37 | SEQ ID NO: 33 |
| TPP-17871 | SEQ ID NO: 37 | SEQ ID NO: 9 |
| TPP-17872 | SEQ ID NO: 38 | SEQ ID NO: 34 |
| TPP-17873 | SEQ ID NO: 38 | SEQ ID NO: 35 |
| TPP-17874 | SEQ ID NO: 38 | SEQ ID NO: 33 |
| TPP-17875 | SEQ ID NO: 38 | SEQ ID NO: 9 |
| TPP-17876 | SEQ ID NO: 39 | SEQ ID NO: 34 |
| TPP-17877 | SEQ ID NO: 39 | SEQ ID NO: 35 |
| TPP-17878 | SEQ ID NO: 39 | SEQ ID NO: 33 |
| TPP-17880 | SEQ ID NO: 39 | SEQ ID NO: 9 |

The recombinant Fabs were purified by affinity chromatography on a CaptureSelect CH1 XL resin (Thermofisher). Size-exclusion chromatography-multi angle light scattering methodology (SEC-MALS), well known to a person skilled in the art, was used to monitor the aggregation levels in the samples. The percentages of monomer measured by SEC-MALS are reported in Table 7 below.

TABLE 7
SEC-MALS analysis of affinity matured Fab variants.

| | Monomer |
|---|---|
| Fab Id | (%) |
| TPP-17867 | 97.1 |
| TPP-17868 | 95.7 |
| TPP-17869 | 99.3 |
| TPP-17871 | 96.4 |
| TPP-17872 | 95.7 |
| TPP-17873 | 95.6 |
| TPP-17874 | 97.4 |
| TPP-17875 | 99.6 |
| TPP-17876 | 98.5 |
| TPP-17877 | 97.8 |
| TPP-17878 | 98.6 |
| TPP-17880 | 99.1 |

### Example 5. Stability Measurement of Anti-IL31 Affinity Matured Fab Variants by DSF

Fab variants were assessed for thermal stability by differential scanning fluorimetry (DSF) using standard techniques. Briefly, 13 μg of each construct was add to 1× Sypro Orange (Thermo-Fisher) in 50 μl total volume in 96-well PCR plate. Using a Bio-Rad CFX96 RT-PCR system equipped with C1000 Thermal Cycler, the temperature was increased from 20° C. to 95° C. at 0.5° C./minute and the fluorescence monitored. The manufacturer-supplied software was used to determine Tm on-set and Tm.

Results of DSF measurements for the constructs are shown in Table 8 below.

TABLE 8
DSF analysis of affinity matured Fab variants.

| | Tm on-set | |
|---|---|---|
| Fab Id | (° C.) | Tm (° C.) |
| TPP-17867 | 65 | 75 |
| TPP-17868 | 67 | 74 |
| TPP-17869 | 68 | 74 |
| TPP-17871 | 65 | 75 |
| TPP-17872 | 68 | 75 |
| TPP-17873 | 66 | 75 |
| TPP-17874 | 68 | 75 |
| TPP-17875 | 68 | 74 |
| TPP-17876 | 68 | 76 |
| TPP-17877 | 68 | 77 |
| TPP-17878 | 64 | 76 |
| TPP-17880 | 68 | 76 |

### Example 6. Affinity Measurements by Surface Plasmon Resonance (Biacore™)

Determination of kinetic binding parameters was performed by surface plasmon resonance measurements using the optical biosensor Biacore™ T200 or T100 (Cytiva). This technology allows for label-free determination of the microscopic rate constants for binding (Ka) and dissociation (KD) of a ligand to a receptor. It is therefore especially suited for characterizing the antibody-antigen interactions. Kinetic analysis was performed with alternating cycles of analyte injections and surface regeneration known as multi-cycle kinetics (MCK). Each injection of an analyte was done in a separate sequence, and a single SPR curve was generated per analyte concentration.

### Materials and Reagents

The list of anti-IL-31 Fab variants and recombinant human IL-31 used to determine binding kinetics by SPR are listed in Table 9 and Table 10.

TABLE 9
Anti-IL-31Fab variants.

| Format | Comment | Fab ID |
|---|---|---|
| Fab | Humanized variant | TPP-20012 |
| Fab | Affinity matured variant | TPP-17867 |
| Fab | Affinity matured variant | TPP-17868 |
| Fab | Affinity matured variant | TPP-17869 |
| Fab | Affinity matured variant | TPP-17871 |
| Fab | Affinity matured variant | TPP-17872 |
| Fab | Affinity matured variant | TPP-17873 |
| Fab | Affinity matured variant | TPP-17874 |
| Fab | Affinity matured variant | TPP-17875 |
| Fab | Affinity matured variant | TPP-17876 |
| Fab | Affinity matured variant | TPP-17877 |
| Fab | Affinity matured variant | TPP-17878 |
| Fab | Affinity matured variant | TPP-17880 |

TABLE 10
Target protein

| | |
|---|---|
| Antigen | MW |

| | | |
|---|---|---|
| (Da) | Buffer | Comment |

| | | | |
|---|---|---|---|
| hIL31bt | 18420 | dPBS pH | APP6-Avi-hsIL31 (aa24- |

| | |
|---|---|
| 7.2-4 | 164)_biotinylated as |

described in Example 4

Commercially available assay reagents and consumables are listed in Table 11.

TABLE 11
Reagents

| Reagent | Catalog No. | Vendor |
|---|---|---|
| Series S Sensor Chip SA | BR-1005-31 | Cytiva |
| NaCl, 1 Kg | 27810. 295 | VWR |
| NaOH, 10M in H₂O solution, 100 mL | 72068-100 ml | Merck |
| | | (Sigma) |
| Isopropanol, anhydrous 99.5% | 278475 | Merck |
| | | (Sigma) |
| Glycine 2.0, 100 mL (10 mM Glycine HCl pH 2.0) | BR-1003-55 | Cytiva |
| HBS-EP + Buffer 20x (0.2M HEPES, 3M Sodium | H8022 | Teknova |
| Chloride, 60 mM EDTA, 1.0% Polysorbate 20, pH 7.6) | | |

### Protein SA Sensor Chip Surface Activation

Series S Sensor Chip SA is designed to bind biotinylated molecules for interaction analysis in Biacore™ systems. The surface consists of a carboxymethylated dextran matrix pre-immobilized with streptavidin. The sensor surface was conditioned on both the active and the reference surface with three consecutive one-minute injections of 1 M NaCl in 50 mM NaOH before ligand was immobilized followed by a wash using 50% isopropanol in 1 M NaCl and 50 mM NaOH after each ligand injection.

### Human IL-31-Biotin Capture

hIL31bt immobilization on the SA sensor chip was accomplished using a wizard procedure for SA-biotin capture applying hIL31bt at 1 μg/mL in HBS-EP+ buffer, aiming for immobilization levels of about 70 RU for flow cell 2 and 160 RU for flow cell 3. Flow cell 1 underwent blank immobilization. The resulting average ligand capture levels across two chips were 95 RU for Fc2 and 195 RU for Fc3.

### Binding Kinetics Experiment (Multi Cycle Kinetics)

Multicycle kinetics (MCK) experiments with all Fab candidates were performed on Biacore T200 at 25° C. Triplicate measurements of each antibody were performed by applying a 1:2 dilution series of the antibodies in HBS-EP+ buffer in range of 32 to 0.25 nM for Fabs 11810, 23599, 23600 and 27375 and 4 to 0.125 nM for the remaining Fabs/IgG samples. Running buffer was HBS-EP+ at a flow rate of 40 L/min, analyte contact time was set to 240 s and dissociation time to 900 s. After each analysis cycle, the sensor chip surface was regenerated using one 30 s shot of 10 mM Glycine pH 2.0 at a flow rate of 40 μL/min.

Data were evaluated using the Biacore T200 evaluation software. Raw data were double referenced, i.e. the response of the measuring flow cell was corrected for the response of the reference flow cell, and in a second step the response of a blank injection was subtracted. Outlier sensorgrams were removed, if necessary. The sensorgrams were fitted applying a 1:1 binding model to calculate kinetic rate constants and the equilibrium dissociation constant. Both Rmax and RI were fitted locally. Data were processed individually for each run. The generated values were used to calculate average values and standard deviations of the respective kinetic constants from n=3 experiments each.

### Results

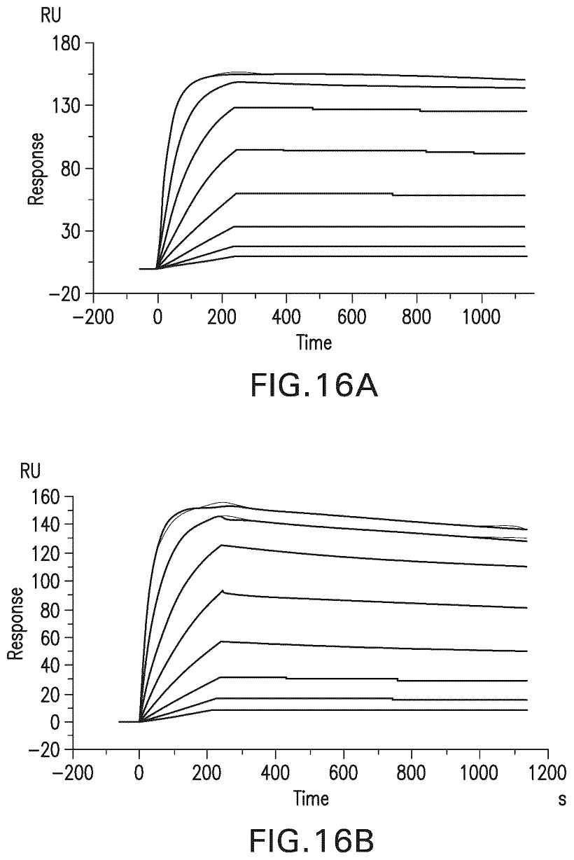

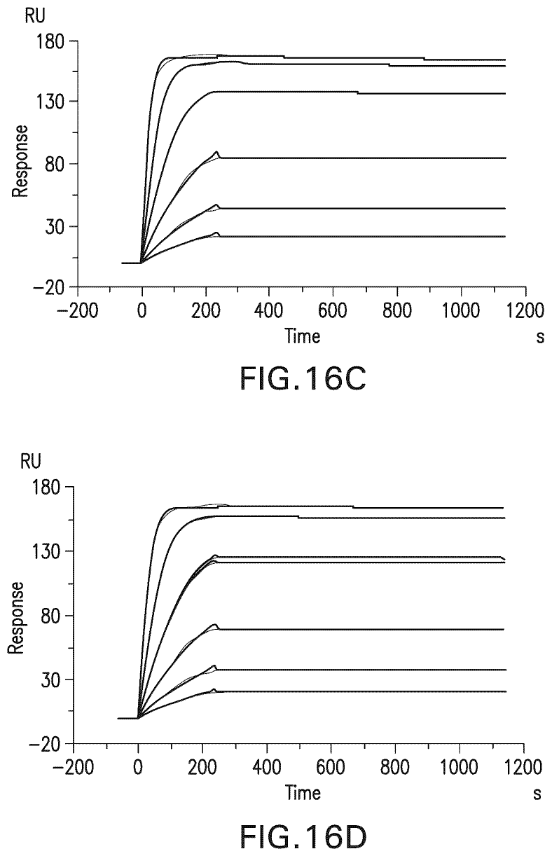

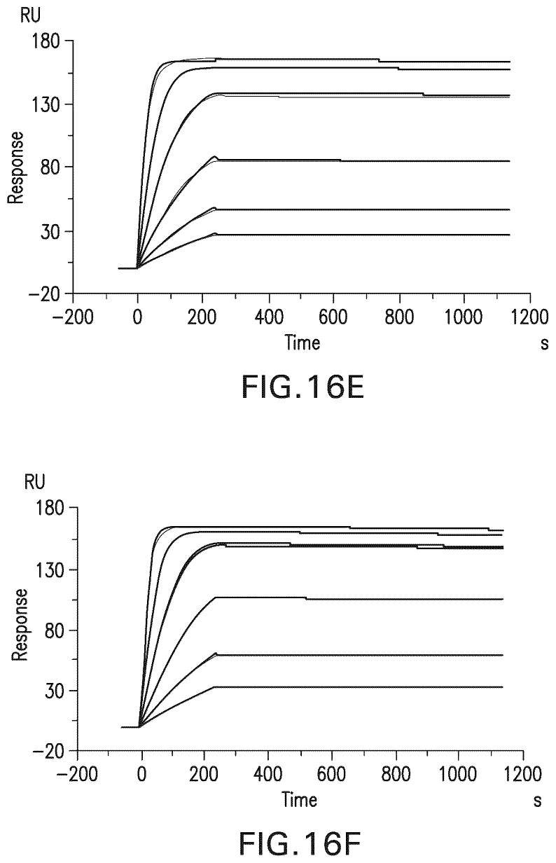

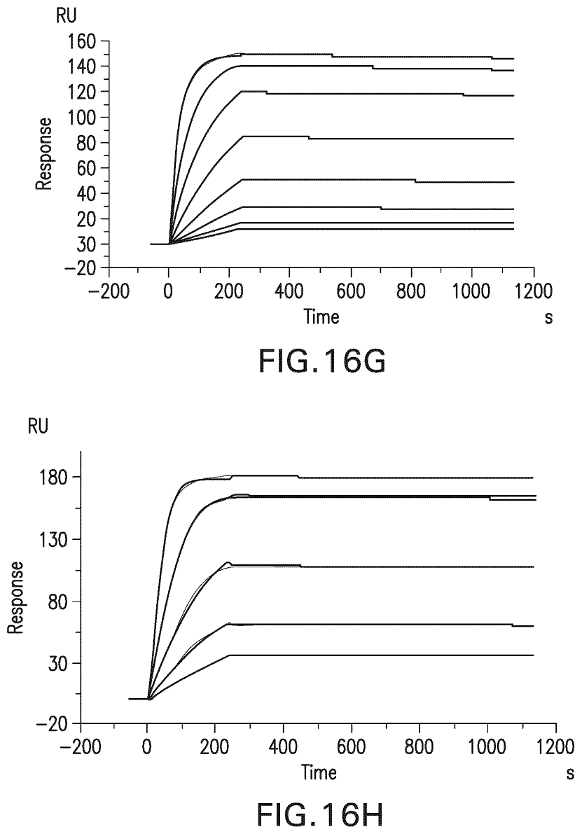

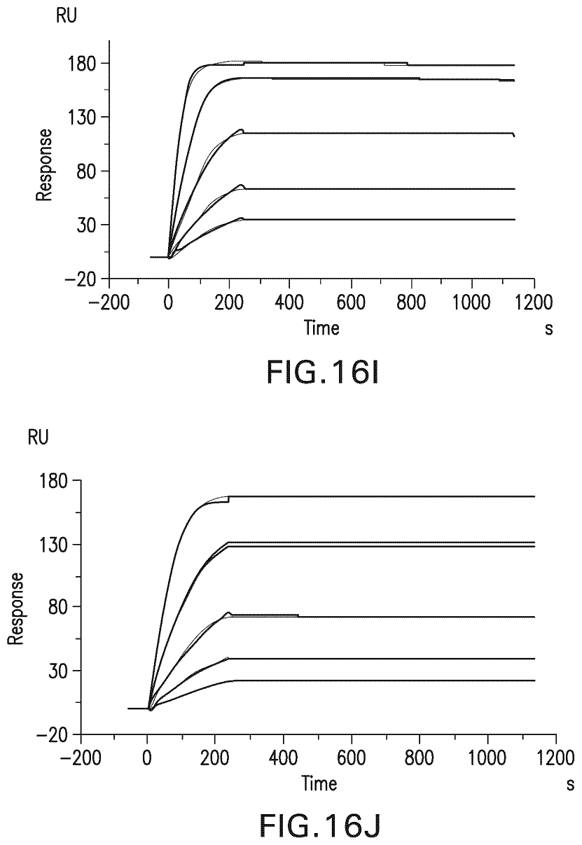

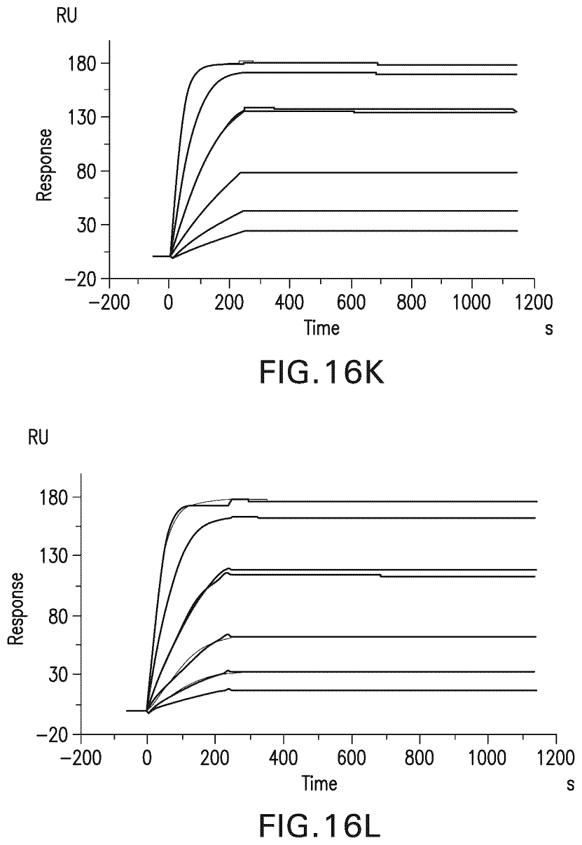

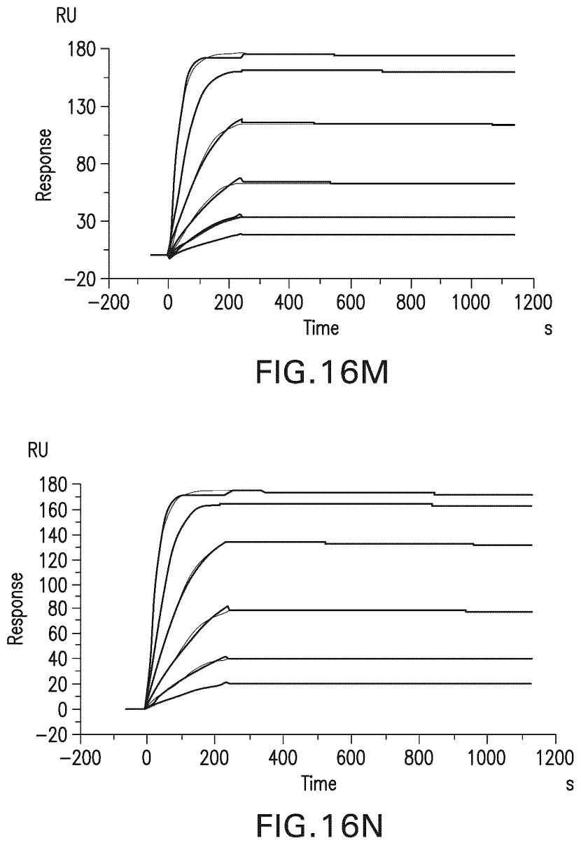

The kinetics constants of the parental and affinity optimized Fabs are summarized in Table 12. Examples of human IL-31 SPR binding sensorgrams are presented in FIG. 16a-o.

All sensorgrams were evaluated applying the fitting model for 1:1 binding interaction. One sensorgram per antibody/antigen interaction is shown in FIG. 16; a) TPP-13357, b) TPP-13348, c) TPP-17877, d) TPP-17876, e) TPP-17878, f) TPP-17880, g) TPP-20012, h) TPP-17867, i) TPP-17868, j) TPP-17869, k) TPP-17871, l) TPP-17872, m) TPP-17873, n) TPP-17874, o) TPP-17875.

TABLE 12
Affinities to human IL-31 measured by Biacore

| | | | Human IL-31 binding kinetics and | | |
|---|---|---|---|---|---|
| | | | dissociation constant | | |
| | | | | | KD |
| Fab ID | Format | Comment | ka (1/Ms) | kd (1/s) | (pM) |
| TPP- | Fab | Humanized variant | 8.64E+05 ± | 2.88E−05 ± | 33.50 ± |
| 20012 | | from patent | 0.47E+05 | 0.56E−05 | 7.68 |
| | | WO2009071696 (clone | | | |
| | | 33) | | | |
| TPP- | Fab | Humanized in this | 9.53E+05 ± | 1.32E−04 ± | 138.65 ± |
| 13348 | | work | 0.47E+05 | 0.03E−04 | 9.20 |
| TPP- | Fab | Humanized in this | 1.10E+06 ± | 3.92E−05 ± | 36.08 ± |
| 13357 | | work + 2 PTM sites | 0.16E+06 | 0.33E−05 | 4.55 |
| | | removed | | | |
| TPP- | Fab | Affinity matured | 1.91E+07 ± | 2.41E−05 ± | 1.58 ± |
| 17867 | | variant | 0.92E+07 | 1.28E−05 | 1.05 |
| TPP- | Fab | Affinity matured | 2.43E+07 ± | 2.68E−05 ± | 1.46 ± |
| 17868 | | variant | 1.03E+07 | 1.24E−05 | 1.28 |
| TPP- | Fab | Affinity matured | 1.61E+07 ± | 2.37E−05 ± | 1.62 ± |
| 17869 | | variant | 0.32E+07 | 1.43E−05 | 1.28 |
| TPP- | Fab | Affinity matured | 1.33E+07 ± | 3.13E−05 ± | 1.46 ± |
| 17871 | | variant | 0.12E+07 | 1.94E−05 | 0.50 |
| TPP- | Fab | Affinity matured | 1.37E+07 ± | 2.17E−05 ± | 1.67 ± |
| 17872 | | variant | 0.18E+07 | 1.10E−05 | 1.09 |
| TPP- | Fab | Affinity matured | 1.42E+07 ± | 2.79E−05 ± | 2.13 ± |
| 17873 | | variant | 0.28E+07 | 1.16E−05 | 1.31 |
| TPP- | Fab | Affinity matured | 1.40E+07 ± | 2.47E−05 ± | 1.96 ± |
| 17874 | | variant | 0.29E+07 | 1.18E−05 | 1.36 |
| TPP- | Fab | Affinity matured | 1.40E+07 ± | 3.02E−05 ± | 2.32 ± |
| 17875 | | variant | 0.33E+07 | 1.18E−05 | 1.38 |
| TPP- | Fab | Affinity matured | 2.00E+07 ± | 2.23E−05 ± | 1.11 ± |
| 17876 | | variant | 0.36E+07 | 0.93E−05 | 0.41 |
| TPP- | Fab | Affinity matured | 2.02E+07 ± | 2.63E−05 ± | 1.33 ± |
| 17877 | | variant | 0.18E+07 | 0.70E−05 | 0.43 |
| TPP- | Fab | Affinity matured | 1.83E+07 ± | 2.26E−05 ± | 1.24 ± |
| 17878 | | variant | 0.15E+07 | 1.50E−05 | 0.76 |
| TPP- | Fab | Affinity matured | 1.84E+07 ± | 3.09E−05 ± | 1.80 ± |
| 17880 | | variant | 0.34E+07 | 1.26E−05 | 1.04 |

### Example 7. Inhibition of IL-31 in BaF3 Cell Proliferation Assay

### (1) Materials and Methods

Reagents and consumables for the determination of the inhibition of IL-31 dependent proliferation of human IL-31Ra/OSMRb-transfected BaF3 cells were as follows:
IL-3 growth dependent mouse BaF3 cells
Recombinant mouse IL-3 (Peprotech #200-03)
hIL-31Ra-expressing plasmid (Uniprot: Q8NI17)
pCMV3 (hygromycin selection cassette) for hOSMRb (SinoBiological #HG11226-UT)
Recombinant human IL-31 (CD33-APP6-Avi-hsIL31 (aa 24-164); see Example 4)
Recombinant cynomolgus monkey IL-31 (CD33-APP6-Avi-cynoIL31 (aa 24-163, produced using the same procedure as for human IL-31)
RPMI1640+GlutaMAX™-I (Gibco by Life Technologies #61870-010), 10% FCS, 0.3 ng/ml hIL-31
Fetal Bovine Serum GOLD EU approved Heat inactivated (PAA #A15-152)
Neon™ Transfectionssystem 100 μl-Kit (Thermofisher #MPK10025)
Geneticin™ Selective Antibiotic (G418 Sulfate) (50 mg/mL) (Thermofisher #10131035)
Hygromycin B (50 mg/ml) (Thermofisher #10687010)
384-well black walled cell culture plate, clear flat bottom (Corning #CLS3764)
Dulbecco's Phosphate Buffered Saline, DPBS, 1× without calcium and without magnesium (Gibco #14190-094)
CellTiter-Blue® Cell Viability Assay (Promega #G8081)
Biotinylated-hu-IL31 (see Example 4)
anti-human OSMR antibody (eBioscience, #12-1303-42)
Goat Anti-Human IgG, F(ab′)₂ fragment specific secondary antibody (Jackson ImmunoResearch, #109-606-097)
   Generation of hIL-31 Growth Dependent BaF3 Cell Line

The human IL-31 growth dependent mouse BaF3 cell line was generated by overexpressing both human IL-31Ra (Uniprot Ref Q8NI17) and human OSMRb (Gene ID: 9180). Each plasmid was transfected into mouse BaF3 cells using nucleofection and stable pools were generated by antibiotic selection (hygromycin and G418).

Stable pools of cells expressing both hIL-31Ra and hOSMRb (referred to as IL-31 dependent BaF3 cells) were further selected for functional hIL-31Ra receptor expressing cells by replacing msIL-3 with human IL-31 in the growth medium. Different human IL-31-dependent BaF3 cell pools were selected using different concentrations of human IL-31. Cells selected for growth with 0.3 ng/ml hIL-31 (hIL-31 dependent BaF3 cells) were used for subsequent experiments. Cell pools expressing one receptor only (referred to as hIL-31Ra-BAaF3 cells and hOSMRb BaF3 cells) were generated by selection of antibiotic resistant stable pools and sorting for high receptor expression.

hIL-31Ra and hOSMR Surface Expression by BaF3 Transfectants

Receptor expression/sorting of BaF3 cells for hIL-31Ra cell surface expression was performed by flow cytometry after surface staining using nemolizumab and the Alexa Fluor® 647 AffiniPure™ F(ab′)₂ Fragment Goat Anti-Human IgG, F(ab′)₂ fragment specific secondary antibody (Jackson ImmunoResearch, #109-606-097). To analyze for hOSMRb cell surface expression and for sorting of hOSMRb-expressing BaF3 cells, cells were stained with a PE-labelled anti-human OSMR antibody (eBioscience, #12-1303-42) and analyzed by flow cytometry.

### IL-31 Concentration Response of IL-31 Dependent BaF3 Cell Line

To determine the EC50 values for hIL-31 and cynomolgus IL-31 dependent proliferation, hIL-31 selected BaF3 cells (selected using 0.3 ng/ml of human IL-31) were incubated with increasing concentrations of IL-31 for 48 hrs at 37° C., and cell growth was determined by measuring the fluorescent signals (540Ex/590Em) 8 hrs after adding CellTiter-Blue reagent according to the manufacturer's protocol. EC50 values were calculated using GraphPad Prism 9.5.1 (GraphPad, LLC) using 4-parameter fitting with variable slope.

Inhibition of hIL-31 induced proliferation of IL-31 dependent BaF3 cells IL-31 dependent BaF3 cells were incubated with 20 pM of human or cynomolgus monkey IL-31 premixed with increasing concentrations of the corresponding antibody (concentration range 2 nM to 0.244 pM final concentration). 3000 cells/well were seeded in 24 μl medium in a Corning® 384 well microplate, low flange, tissue Culture (TC)-treated surface, black polystyrene, flat bottom plates (#CLS3764). 6 μl of IL-31/AB mix was added/well and cells were incubated for 48 hrs at 37° C. 8 hrs after adding cell Titer-Blue reagent, cell growth was determined by measuring the fluorescent signals (540Ex/590Em) on a PHERAstar FSX multiplate reader (BMG Labtech, DE).

### (2) Results

### EC50 Values for Human and Cynomolgus Monkey IL-31 in the BAF3 Cell Proliferation Assay

BaF3 cells dependent on 0.3 ng/ml hu IL-31 (IL-31 dependent BaF3 cells) stably expressing both human IL-31Ra and human OSMRb showed similar IL-31 concentration-dependent proliferative responses to both human and cynomolgus monkey IL-31 with EC50 values of 4.4+/−4.6 pM for human IL-31 (n=12) and 3.8+/−3.8 pM for cynomolgus IL-31 (n=8), respectively (Table 13; for a representative graph see FIG. 12).

TABLE 13
Potency of human and cynomolgus IL-31 in BaF3 cell growth assay

| | | | | | | | | | | | | | std |
|---|---|---|---|---|---|---|---|---|---|---|---|---|---|
| EC50 [pM] | | | | | | | | | | | | average | ev. |
| hIL-31 | 61 | 17.3 | 1.7 | 1.2 | 3.9 | 7.2 | 5.6 | 2.2 | 0.7 | 1.2 | 3.0 | 4.4 | 4.6 |
| cIL-31 | 12.5 | 2.0 | 4.4 | 4.5 | 2.0 | 0.75 | 0.9 | 3.3 | | | | 3.8 | 3.8 |

Inhibition of Human IL-31 Induced hIL-31 BaF3 Cell Proliferation

Since the BaF3 cells dependent on 0.3 ng/ml hu IL-31 (IL-31-dependent BaF3 cells) responded equally well to both human and cynomolgus monkey IL-31, this cell pool was used to determine inhibitory potencies of human and cynomolgus monkey IL-31-dependent cell growth by anti-IL-31 Fabs.

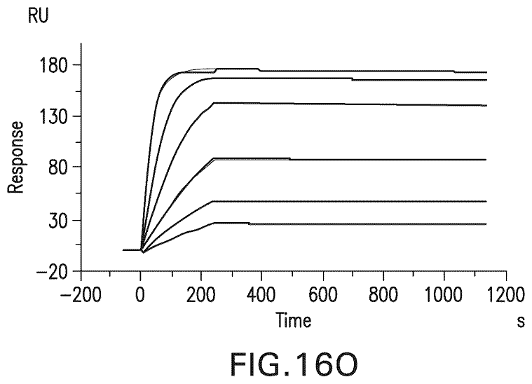

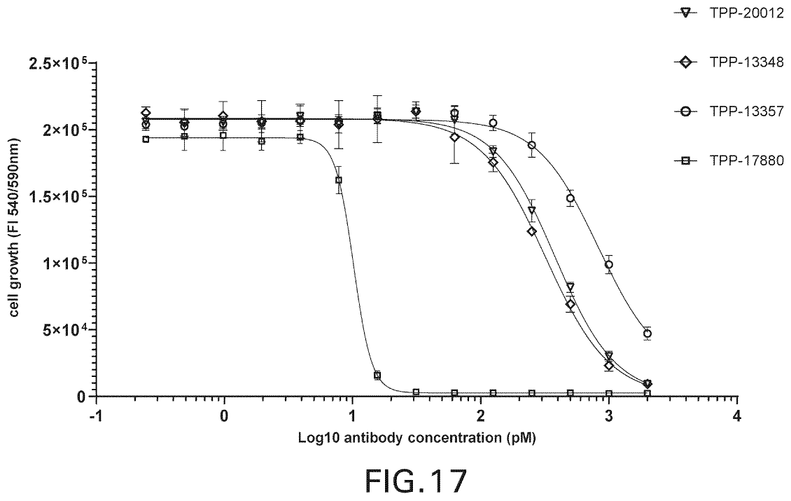

The affinity matured Fab TPP-17880 inhibited cytokine induced cell-growth with an IC50 value of 10.2+/−0.4 pM for human IL-31 (n=3) (FIG. 17; Table 14). This potency shows a significant improvement over the non-affinity matured anti-IL31 Fabs and TPP-20012.

TABLE 14
IC50 values [pM] of anti-IL-31 Fabs for inhibition of the proliferation
of IL-31 dependent Baf3 cells in response to human IL-31 (n = 3)

| Exp. | TPP- | TPP- | TPP- | TPP- |
|---|---|---|---|---|
| Nr. | 20012 | 13348 | 13357 | 17880 |
| CB42 | 181.7 | 156.6 | 477.8 | 9.8 |
| CB45 | 408.3 | 288.5 | 816.9 | 10.5 |
| CB48 | 380.8 | 320 | 825.6 | 10.2 |
| Average | 323.6 | 255 | 706.8 | 10.2 |
| stdev | 123.7 | 86.7 | 198.3 | 0.4 |

### Example 8. Structure Determination and Analysis of the Affinity Matured Candidate TPP-17880 Fab in Complex with Human IL-31

### (i) Material and Methods

The production of untagged human IL-31 C133S (SEQ ID NO: 173) and antibody Fab TPP-17880 was done using the same protocols as mentioned in Example 3. To obtain the Fab-antigen complex, excess amount of human IL-31 was mixed with Fab TPP-17880. The mixture was purified by SEC on a Superdex 75 10/300 column (Cytiva) equilibrated with 20 mM HEPES pH 7.5, 150 mM NaCl. Pooled fractions used for crystallization were concentrated by ultrafiltration to 12.6 mg/ml. The complex crystallization protocol is also the same as used in Example 3 except using 0.2 M Ammonium Iodide, 20% w/v Polyethylene glycol 3350, pH 6.2 as the crystallization buffer. Crystals used for X-ray diffraction appeared after 6 days and grew to full size within two weeks.

X-ray data collection and processing was carried out using standard protocols. Briefly, crystals were cryo-protected with a 1:1 mix of the reservoir solution with a solution consisting of 15% ethylene glycol and then directly flash-cooled into liquid nitrogen. X-ray data were collected at the Stanford Synchrotron Radiation Lightsource (SSRL), beamline BL14-1, using X-ray at wavelength of 1.03317 Å. A total of 220 images with 1° oscillation each were recorded at a crystal-to-detector distance of 450 mm using a Pilatus 6M pixel detector and processed with program HKL3000. The final diffraction data were processed to the resolution of 3.5 Å with CC_{1/2} of 0.129.

The crystal structure of human IL-31 C133S in complex with Fab TPP-17880 was determined by molecular replacement with Phaser using models derived from the complex structure of parental Fab as search models. Iterative model building and refinement were carried out in Coot 0.9 and PHENIX (1.19.2). The final structure model of the Fab in complex with IL-31 C133S was refined to R-value of 22.5% and free R-value of 29.6%, both of which indicate correct determination of the complex structure.

### (ii) Results

The overall structure of Fab TPP-17880 in complex with IL-31 is almost identical to that of its parental TPP-13357 (FIG. 2B). Structural alignment of the two antibody-antigen complexes shows root mean square deviation (RMSD) of 1.035 Å, indicating highly similarity in between. The human IL-31 epitope of TPP-17880 is almost identical to that of TPP-13357 (FIG. 4B), which consists of residues S35, D37, V38, K40, I41, E44, N81, P86, R89, A90, K93, R96, K101, D105, E106, I108, E109, H110, D112, K113, L114, I115, F116, Q117, D118, A119, P120, E121, and T122 (FIG. 4B). Human IL-31 D37, K40, E44, R89, K93, R96, E109, K113, Q117, and A119 are key epitope residues recognized by TPP-17880.

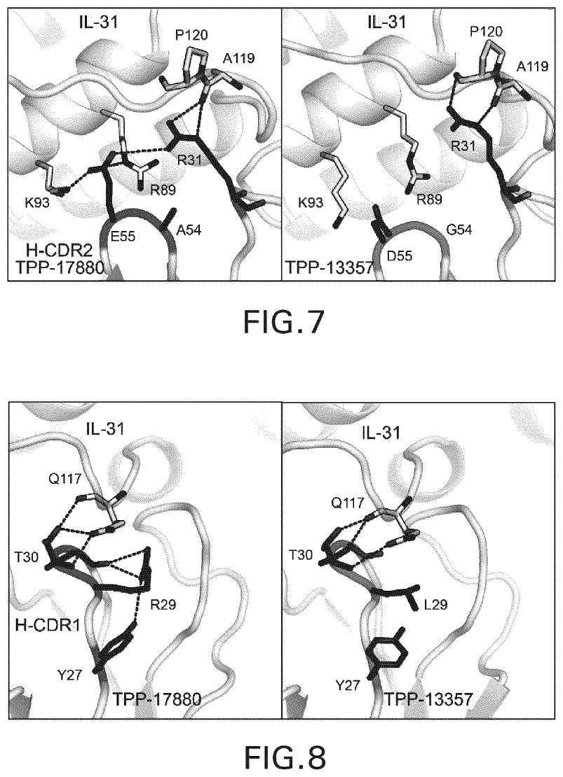

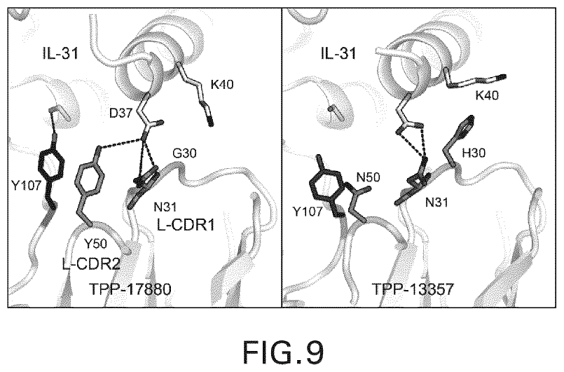

Key interactions between human IL-31 and TPP-13357 are largely kept in TPP-17880 (FIG. 5B). However, the mutated residues in TPP-17880 show various impacts on the binding to IL-31 compared to TPP-13357. G54A and D55E mutations in H-CDR2 lead to a different conformation of the CDR loop that forms additional hydrogen bonds (shown as dashed lines) to IL-31 K93 and R89 (FIG. 7). R29 in TPP-17880 H-CDR1 eliminates the unfavorable contact between TPP-13357 L29 and IL-31 Q117 (FIG. 8). G30 in TPP-17880 L-CDR1 allows the placement of IL-31 K40 sidechain while H30 in TPP-13357 pushes IL-31 K40 away from the antibody-antigen interface (FIG. 9). Meanwhile, N50Y mutation in L-CDR2 forms an additional hydrogen bond to IL-31 D37 as expected from the structure-guided design. N50Y also leads to conformational change of Y107 in TPP-17880 H-CDR3 that forms an additional hydrogen bond to the backbone of IL-31 K113. In short, mutations in TPP-17880 either form additional bonding to IL-31 or eliminate unfavorable contacts that seen in TPP-13357. All CDRs in TPP-17880 contribute to the IL-31 binding (FIG. 5B). The structural analysis of both TPP-13357 and TPP-17880 shows a successful engineering of anti-IL-31 antibody towards high affinity binding to IL-31 as well as higher thermal stability of the antibody, which are key to the design of IL-13/IL-31 bispecific antibodies.

### Example 9. Generation of IL-31/IL-13 Bispecific Antibodies in CHO Cell Line

### (1) Vector Design

Two vectors, vector A and vector B, were generated according to the following setup:

Vector A was designed for expression of antibody mAb1 (anti-IL13 IgG1; SEQ ID NO: 19, SEQ ID NO: 20; variable regions represented by SEQ ID NO: 17, SEQ ID NO: 18). The constant region of the heavy chain comprised a K409R (EU numbering) mutation to facilitate Fab arm exchange chain pairing.

Vector B was designed for expression of antibody mAb2 (anti IL-31 IgG1; SEQ ID NO: 9, SEQ ID NO: 10; variable regions represented by SEQ ID NO: 7, SEQ ID NO: 8). The constant region of the heavy chain comprised a F405L (EU numbering) mutation to facilitate Fab arm exchange chain pairing.

Both mAb1 and mAb2 additionally comprised the following mutations in the constant region of the heavy chain: L234A/L235A (EU numbering) (so-called “LALA” mutations), for partial silencing of Fc effector functions; and M252Y/S254T/T256E (EU numbering; so-called “YTE” mutations) for half-life extension.

Both mAb1 and mAb2 have a variable light region of the kappa type.

Vector A and B both comprised folic acid receptor (FolR) and dihydrofolate reductase (DHFR) genes to facilitate selection of recombinant cells. In vector A and B, the heavy chain ends with a so-called leaky stop codon, followed by a transmembrane section, which results in a small fraction of antibody being anchored to the cell membrane instead of being secreted. Membrane-anchored antibody can be used to stain antibody-producing cells for cytometry (see below). A schematic overview of the vector set-up of both vectors A and B is seen in FIG. 13.

### (2) Host Cell Line and Transfection

A parental CHO cell line was used as host cell line for the production of the mAb1 and mAb2 expressing cell lines. The host cell line was derived from the CHO-K1 cell line, well known to a person skilled in the art, in a way described e.g. in the patent applications WO2015/092737 and WO2015/092735. CHO cells were cultivated in shake flasks in a non-humidified shaker cabinet at 150 rpm, 10% CO2 at 36.5° C. Cell viabilities and growth rates were monitored by means of an automated cell counter (ViCell, Beckman Coulter). Cells were passaged 2-3 times per week into fresh medium and were maintained in logarithmic growth phase. Separate CHO cell lines were generated for expression of mAb1 and mAb2, respectively.

Expression vectors A and B were each transfected into CHO host cells by electroporation, using the Amaxa Nucleofection system (Lonza, Germany), according to the manufacturer's instructions. Prior to transfection, expression vectors were linearized using the restriction enzyme SwaI. The transfection reaction was performed in chemically defined cultivation medium. Immediately after transfection, cells were transferred into a shake flask containing chemically defined cultivation medium.

### (3) Cell Selection and Sorting

Selection for recombinant cells was based on folate deprivation. Folate is an essential vitamin for purine and methionine synthesis that needs to be taken up by mammalian cells from the culture medium. Two enzymes encoded by the vector facilitate folate metabolism: folate receptor (FolR) mediates folate import, and dihydrofolate reductase (DHFR) converts folate into vital precursors for purine and methionine synthesis. Recombinant cells that have successfully integrated the vector into the genome and express FolR and DHFR have a clear growth advantage at the low folate levels present in the culture medium. The selection pressure is further enhanced by supplementing the culture medium with methotrexate (MTX), a folate analogue that inhibits DHFR. The combination of FolR, DHFR, folate restriction and MTX provides a strong selective regime for enrichment of recombinant cells.

After transfection, cell pools were first maintained in low folate medium without MTX for two days and were then cultivated in medium containing 10 nM MTX for about three weeks. After an initial crisis with low cell viability, cells recovered to a viability above 95%.

To obtain clonal cell lines, single cell cloning by cytometry was performed, using the CHO cell pools producing mAb1 or mAb2, respectively. Single cell cloning by cytometry is described e.g. in the patent application WO2010/022961. To facilitate the selection of high-producer clones, cells were stained with a fluorescently labelled antibody directed against the Fc part of mAb1 or mAb2, a process well known to a person skilled in the art, for example as described in WO2010/022961.

Single cell cloning was performed with a Sony Cell Sorter instrument equipped with a 96-well plate holder, using a 100 μm disposable sorting chip. To assure that only single cells are sorted, settings were adjusted to single cell mode, 3-droplet sorting. With these settings, cell-containing droplets are only sorted if the previous and subsequent droplets are both empty. Cell concentration and flow rate were optimized at the expense of yield to increase the probability that each droplet contains not more than one single cell. Multiple gates were set to select single live cells with high fluorescence. Each cell was seeded into a separate well of a 96-well plate and a high-resolution microscopy image of each well was acquired to document monoclonality and verify the single cell cloning procedure.

### (4) Cell Expansion

After single cell cloning, the clones were expanded in chemically defined cultivation medium in 96-well plates, 24-well plates, and shake flasks, well known to a person skilled in the art. Clones were characterized with regards to productivity and bioprocess suitability, genetic integrity, and cell line stability using standard methods. Based on clone characterization, final clones were selected for production of mAb1 or mAb2, respectively.

### (5) Upstream Processing

The anti-IL13 and anti-IL31 parental antibodies were produced individually in stable CHO pools. The vials were thawed, expanded, and transferred in chemically defined medium to initiate the expression in either shake flasks or bioreactors. These expression cultures were subsequently cultivated and fed for a period of 13-14 days. At the end of the cultivation process cells were separated from the culture supernatant either by centrifugation or depth filtration followed by sterile filtration.

### (6) Downstream Processing

The anti-IL13 and anti-IL31 parental antibodies were individually captured utilizing protein A affinity chromatography. The antibodies were eluted under acidic conditions followed by protein adjustment to pH 7.0.

Both parental antibodies were mixed equimolar and the Fab-arm exchange (described e.g. by Labrijn et al, Proc Natl Acad Sci USA. 2013 Mar. 26; 110(13):5145-50) was initiated by the addition of a reducing agent. After incubation the reducing agent was removed by either desalting chromatography or tangential-flow filtration. Removal of product and process related impurities was achieved by cation exchange chromatography (CEC) in bind-elute mode, followed by a multimodal anion exchange chromatography (MAC) in flow-through mode. Finally, the anti-IL13/IL31 bispecific antibody was concentrated, and buffer exchanged to reach the desired concentration and formulation.

### (7) Analytical Characterization and Purity Assessment

### (a) Size Exclusion

Purity assessment and relative quantification of their high- and low molecular weight species of bbmAb samples were assessed by size exclusion chromatography on BEH200 SEC column (Waters #186005225, 1.7 μm, 4.6 mm×150 mm), pore size 200A and a Waters ACQUITY UHPLC system. Mobile phase was 50 mM sodium phosphate solution, 400 mM sodium perchlorate pH 6.0, flow rate 0.4 mL/min, and column temperature 40±3° C. UV was recorded at 210 nm. Data acquisition and peak integration was performed using Chromeleon™ 7.3 (Thermo Scientific). The final purified bbmAb showed a purity by 99.7% and 0.3% high molecular weight species.

### (b) Capillary Electrophoresis CE-SDS

Purity assessment and relative quantification of their low-molecular weight species of bbmAb samples were assessed by capillary electrophoresis CE-SDS on Beckman Coulter PA 800 system equipped with bare fused silica capillary (50 μm, 375 OD, 67 cm, Beckman). For non-reducing CE-SDS, bbmAb was mixed with sample buffer (0.1 sodium phosphate/1% SDS, pH 6.6) and then mixed with iodacetamide solution. For reducing CE-SDS, the bbmAb was mixed with 0.1 M Tris/1% SDS sample buffer, pH 8.0 and reduced with 5% (v/v) mercaptoethanol. Both samples underwent a heat denaturation step at 70° C. for 10 minutes. Samples were subjected to the capillary with a total length of 30 cm filled with Beckman SDS MW sieving gel buffer and separated from negative to positive polarity at 15 kV and 25° C. capillary temperature. Detection by UV was at 214 nm. Electropherograms were processed and integrated using Chromeleon™ 7.3 (Thermo Scientific). In reduced CE-SDS 0.7% low molecular weight (LMW) and a bbmAb purity of 98.5% were detected and in non-reducing mode 2.7% (LMW) and a bbmAb purity of 97.4% were detected.

### (c) Capillary Zone Electrophoresis CZE

Charged variants and their relative quantification were assessed by capillary zone electrophoresis CZE on Beckman Coulter PA 800 system equipped with 2140 nm UV detector. bbmAb was separated on fused silica capillary (50 μm ID) of a total length of 40 cm with an applied voltage of 20 kV at 25° C. and positive polarity and 400 mM 6-aminocaproic acid/acetic acid, pH 5.7 with 2 mM TETA and 0.03% Tween 20. Electropherograms were processed and integrated using Chromeleon™ 7.3 (Thermo Scientific). 13.6% acidic variants and 19.6 basic variants were measured in the final purified bbmAb.

### (d) LC-MS Characterization

In depth characterization confirming identity and integrity and relative quantification of mis-paired species and post-translational modifications were assessed by intact (deglyco) LC-MS, reduced LC-MS, intact/reduced FabALACTICA LC-MS and peptide mapping LC-MS.

Intact deglycosilated bbmAb: Purified bbmAb was diluted in 50 mM Tris-HCL pH 7.5 to 1 mg/mL and deglycosilated for 2 h at 37° C. using 2 μL PNGaseF enzyme (Promega). The reaction was stopped by adding trifluoracetic acid to 2%.

Reduced bbmAb: 44 pg bbmAb at 1 mg/mL were denatured and reduced by mixing with 1 μL 1 M DTT, 5 μL 1 m Tris pH 7.5 and 50 μL 8 M Guanidine HCl with subsequent incubation at 1 h at 37° C.

Reduced deglycosilated bbmAb: Purified bbmAb was diluted in 50 mM Tris-HCL pH 7.5 to 1 mg/mL and deglycosilated for 2 h at 37° C. using 2 μL PNGaseF enzyme (Promega). After deglycosilation the bbmAb was denaturated and reduced by mixing with 1 μL 1 M DTT and 50 μL 8 M Guanidine HCl with subsequent incubation at 1 h at 37° C. The reaction was stopped by adding trifluoracetic acid to 2%.

FabALACTICA bbmAb: 100 pg bbmAb were mixed with cleavage buffer (150 mM Na-phosphate buffer pH 7.0) and digested with 80 U FabALACTICA (Genovis) and 2 L PNGaseF overnight at 37° C.

LysC/Trp Peptide mapping: 200 pg bbmAb were denatured using 50 μL denaturing buffer solution (6 M guanidine hydrochloride, 20 mM His/His-HCl, pH 6.0 and reduced by adding 1.5 μL 1M DTT with subsequent incubation at 37° C. for 1 hour. The alkylation was performed by adding 3 μL 1 M iodacetamide followed by incubation at room temperature in the dark. The reaction was quenched with 1 μL 1 M DTT. Following denaturing/reduction 750 μL 50 mM Tris-HCl, pH 8.0 were added to the sample with subsequent digestion 4 L of a 1 mg/mL endoproteinase Lys-C solution and incubated at 37° C. for 2 h. The reaction was stopped by adding 5 μL of 1M CaCl₂) followed by addition of 4 μL of a 1 mg/mL Trypsin solution with incubation at 37° C. for 18 h. The reaction is quenched by adding 5 μl TFA.

LC-MS measurements: Protein samples were subjected to LC-MS system using Waters UPLC H-Class equipped with a Acquity BioResolve, 2.1×150 mm, 2.7 μm, 450 Å and a Xevo G2 TOF mass spectrometer (Waters). Eluents were A: 0.1% TFA in water and B: 0.09 & TFA in acetonitrile. The column was set to 80° C. and the flow rate to 0.3 mL/min. Proteins were eluted using 10 min gradient from 30% to 50% for reduced analysis and a 5.4 min gradient for intact analysis. MS settings: ESI (+) TOF mode, Resolution mode, Mass Range 400-4000 Da, Scan Time 1 s, capillary voltage 3 kV, sampling cone 25 V-40 V. System was calibrated using NaCl solution.

Peptide digests were analyzed by RP-LC-MS on H-Class coupled to a Xevo G2 TOF mass spectrometer (Waters) using an CSH130 C18 2.1 mm×150, 1.7 um). Eluents were A: 0.1% TFA in water and B: 0.09 & TFA in acetonitrile. Column temperature was 40° C. Peptide were eluted from the column with following gradient: 0-5 min 0% B, 5-10 min 0-2%, 10-40 min 2-20%, 40-120 min 20-40% B, 120-135 min 40-70%. UV chromatograms were recorded at 214 nm and MS data acquisition was performed in positive ES (+) resolution mode as an MSE experiment using low energy (4 eV) and high energy fragmentation (30-55 V). Lock maa was Leucin Enkephalin (Waters). Data Processing and evaluation was performed on MassLynx 4.2 or UNIFI 1.6 software (Waters). MAxEntm algorithm was used for deconvolution of protein mass spectra. Theoretical mass calculations were performed with GPMAW 9.2 software (Light house data).

On intact (deglyco) level the detected mass matches the expected computed mass within the mass deviation of the instrumentation (A5 Da). The major species in the sample was identified as bbmAb (2pQ, OK). No mis-paired species were detected above 1% as quantified by MS signal. On reduced level and after Fabalactica digestions the identity and integrity was confirmed on chain level respectively subunit level (see below). No LC swapped species (L1H2:H1L2, L2H1:H2L1) were detected above 3% based on MS signal. Glycosylation profiles show commonly observed species for bbmAb expressed in CHO. High sequence coverage of 92% was observed after LysC/Trypsin digestion. Low levels of modification e.g. non-formed N-terminal pQ (≤7% perHC), 2% C-terminal (Lysine variants), C-terminal proline amide variant (4%), isomerization (5%) and oxidation (≤10%) in purified material.

TABLE 15
Assignment of measured bbmAb masses by RP-LC-MS

| | | | | Mass |
|---|---|---|---|---|
| | | Observed | Theoretical | deviation |
| Analysis | Identity | mass (Da) | mass (Da) | (Δ Da) |
| Intact | L1H1:L2H2 | 145676 | 145672 | 4 |
| deglycosilated | bbmAb 2pQ, | | | |
| | 0K, | | | |
| | deglycosylated | | | |
| Reduced | L1 | 23832.4 | 23833.2 | −0.8 |
| deglyco | | | | |
| | H1 | 49458.3 | 49459.2 | 0.9 |
| | L2 | 23496.5 | 23497.0 | −0.5 |
| | H2 | 48913.8 | 48914.8 | 1 |
| Fabalactica | Fc dimer | 50120.3 | 50120.0 | 0.3 |
| | H1:H2, 0K | | | |
| | Fab | 47629.4 | 47629.8 | −0.4 |
| | L1:H1/2, pQ | | | |
| | Fab | 47899.5 | 47900.1 | −0.6 |
| | L2:H2/2, pQ | | | |

### Example 10. Affinities to Recombinant Human and Cynomolgus IL-13 and IL-31 Measured by SET

### (1) SET Method

The Solution Equilibrium Titration (SET) assay (Friguet et al., J Immunol Methods; 77(2): 305-319, 1985) allows the determination of antibody-antigen interaction affinities for tight binders. This technique does not require immobilization or labeling of either interaction partner. A constant concentration of antibody (at or below the expected KD) is co-incubated with serial antigen dilutions until equilibrium is reached. The fraction of unbound antibody is determined by transferring the antigen-antibody mixtures on antigen-coated plates for a brief incubation. The free antibody consequently binds to the coated antigen and is, after a washing step to remove antibody-antigen complexes, detected with an electrochemiluminescence-labeled detection antibody. The resulting signal is plotted versus the antigen concentration. The KD is determined by non-linear curve fitting with a 1:2 binding model for bivalent monospecific antibodies, and with a 1:1 binding model for monovalent bispecific antibodies. To measure KD values down to the low picomolar level, a sensitive read-out technology such as electrochemiluminescence (ECL) is required (Haenel et al., Anal Biochem; 339(1):182-184, 2005).

### (2) Reagents and Consumables for SET

Blocking buffer consisted of PBS containing 5% BSA, and sample buffer of PBS containing 0.5% BSA and 0.02% Tween-20. The wash buffer contained TBS plus 0.05% Tween 20 and PBS served as coating buffer. Read buffer was prepared by diluting 4×MSD read buffer T 1:4 with water. All buffers were prepared using deionized water. Ordering information of commercially available reagents and consumables is listed below.

TABLE 16
Reagents

| Reagent | Vendor | Order number |
|---|---|---|
| BSA | Sigma-Aldrich | A9647 |
| MSD Streptavidin Multi-Array ® | MSD | L21SA-5 |
| 384-Well Plate | | |
| MSD read buffer T (4x) | MSD | R92TC-1 |
| with surfactant | | |
| PBS 10x | Teknova | P0195 |
| Polypropylene 384 MWP | Greiner | 781280 |
| Sulfo-tag labeled anti-hu IgG | MSD | R32AJ |
| TBS 10x | Teknova | T1680 |
| Tween-20 | Fluka | 93773 |

TABLE 17
Antigens

| | | Source #Cat-No. or | |
|---|---|---|---|
| ID | Full name | Database-ID | Buffer |
| hsIL-13 | Recombinant | PeproTech, #200-13- | Sodium Phosphate |
| | Human IL-13 | 10UG | pH 7.0 |
| | (115aa) | PPB-33019 | |
| cyIL- | M-cyIL13(40- | As described in Example | 50 mM CHES, 50 |
| 13 | 146) -Avi-H | 3 | mM NaCl, pH 8.5 |
| hsIL-13 | Biotinylated | AcroBiosystems, # IL3- | PBS pH 7.4 |
| biotin | Human IL-13 | H82E5, 25 μg, lot BV3403- | |
| | Protein, | 9B4F1-ZV | |
| | His, Avitag ™ | | |
| hsIL-31 | APP6-Avi- | As described in Example | PBS pH 7.4 |
| | hsIL31 (aa24-164) | 3 | |
| cyIL- | APP6-Avi- | As described in Example | dPBS pH 7.2-7.4 |
| 31 | cyIL31(24-163) | 3 | |
| hsIL- | APP6-Avi- | As described in Example | PBS pH 7.4 |
| 31biotin | hsIL31 (aa24-163)_{—} | 3 | |
| | bio | | |

The bispecific anti-IL-31/IL-13 antibody generated in Example 9 (SEQ ID NO: 9, SEQ ID NO: 10; SEQ ID NO: 19, SEQ ID NO: 20) was characterized.

### (3) Instrument and Software for SET

All SET assays were measured on a Sector Imager 6000 (Meso Scale Discovery, Gaithersburg, USA) controlled by Methodical Mind Plate Reader V.1.0.37 (Methodical Mind, LLC. Rockville, MD, US). Data were processed using Xlfit (IDBS) version 5.5.0.5 as an add-in of Microsoft Office Excel.

### (4) SET Assay Procedure

Twenty-two serial 2-fold dilutions of the antigens (creating ranges of 10 nM to 4.8 fM for human and cynomolgus monkey IL-13, and 1 nM to 477 fM for human and cynomolgus monkey IL-31) were prepared in sample buffer and a constant concentration of anti-IL-31/IL-13 bispecific antibody was added (for human and cynomolgus monkey IL-13 and cynomolgus IL-31, 2 pM; for human IL-31, 1 or 2 pM). A volume of 60 μl/well of each antigen-antibody mix was distributed in duplicates to a 384-well polypropylene microtiter plate (MTP). Sample buffer served as negative control and a sample containing only antibody as positive control (maximal electrochemiluminescence signal without antigen, Bmax). The plate was sealed and incubated overnight (at least 16 h) at room temperature (RT) on a shaker.

IL-13 and IL-31 readouts: An MSD Streptavidin Multi-Array® 384-Well Plate was coated with 30 μl/well biotinylated human IL-13 or human IL-31 (0.1 or 0.2 μg/ml in PBS) and incubated overnight at 4° C. in a fridge w/o shaking. After washing with wash buffer, a blocking step with 50 μl/well blocking buffer was applied for at least 1 h at RT with shaking.

After further washing, a volume of 30 μl/well of the equilibrated antigen/antibody mix was transferred from the polypropylene MTP to the coated MSD plate and incubated for 20 min at RT with shaking. After an additional wash step, 30 μl Sulfo-tag-labeled anti-IgG detection antibody (0.36 μg/ml) diluted in sample buffer were added to each well and incubated for 30 min at RT on a shaker. The MSD plate was washed, and 35 μl/well MSD read buffer were added followed by an incubation for 5 min at RT. ECL signals were generated and measured by the MSD Sector Imager 6000. The SET Data were exported to XLfit. Average ECL-signals were calculated from duplicate measurements within each assay. Data were baseline adjusted by subtracting the lowest value from all data points and plotted against the corresponding antigen concentration to generate titration curves. KD values were determined by fitting the plot according to the following models:
1:1 binding model for Fabs or bispecific mAb (anti-IL31/IL-13 bispecific antibody)

$$y = {B}_{max} - ( \frac{{B}_{max}}{2 [ Fab ]} ( [ Fab ] + x + {K}_{p} - \sqrt{{( [ Fab ] + x + {K}_{p} )}^{2} - 4 x [ Fab ]} ) ) ,$$

wherein:
y: blank subtracted ECL signal
Bmax: maximal ECL signal at zero antigen concentration
[Fab]: applied Fab or bispecific antibody concentration (as applicable)
KD: Dissociation equilibrium constant
x: applied antigen concentration

### (5) Results

Using the SET method, we observed that the anti-IL-31/IL-13 bispecific antibody binds with high affinity to recombinant human as well as cynomolgus monkey IL-13 and IL-31. The individual binding affinities for human and cynomolgus monkey IL-13 and IL-31 were in the sub-pM range and reported each with a KD<1 pM which is at the lower limit of detection for this method. Affinities on human and cynomolgus antigens match each other confirming full cross-reactivity.

TABLE 18
Affinities

| Affinity | IL-13 | IL-31 |
|---|---|---|
| (mean ± SD) | K_{D} [pM] | K_{D} [pM] |
| human (n = 2) | <1 | <1 |
| cynomolgus (n = 2) | <1 | <1 |

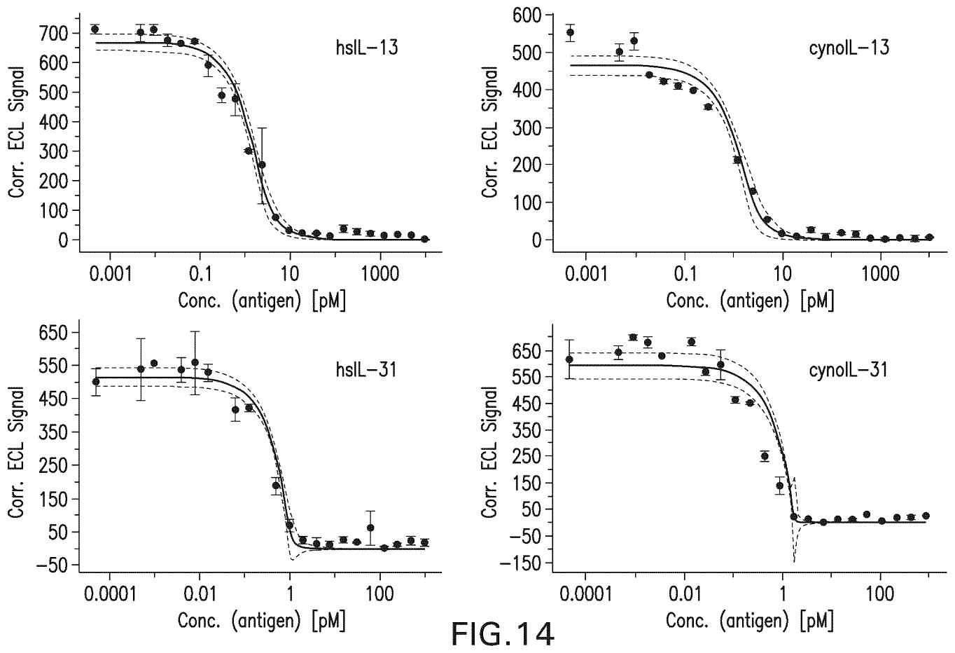

SET assay 1:1 binding model fits are shown in FIG. 14.

### Example 11. Affinities to Recombinant Human and Cynomolgus IL-31 and IL-13 Measured by Surface Plasmon Resonance (Biacore™)

To confirm the high binding affinity results of the anti-IL-31/IL-13 antibody to IL-31 and IL-13, we used SPR (Biacore) in addition to SET. Biacore assays for affinity to IL-31 were performed as described in Example 6. Kinetics constants of anti-IL-31/IL-13 bispecific antibody generated in Example 9 (SEQ ID NO: 9, SEQ ID NO: 10; SEQ ID NO: 19, SEQ ID NO: 20) with human IL-31 were determined.

TABLE 19
IL-31 binding kinetics

Human IL-31 binding kinetics and
dissociation constant

| | | | KD |
|---|---|---|---|
| Antibody | ka (1/Ms) | kd (1/s) | (pM) |
| Anti-IL-31/IL-13 | 5.99E+06 ± | 2.35E−05 ± | 4.29 ± |
| bispecific | 2.27E+06 | 0.59E−05 | 1.97 |

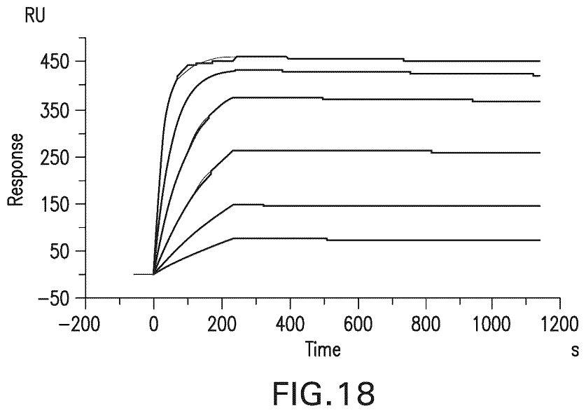

The human IL-31 SPR binding sensorgram is presented in FIG. 18.

The Biacore assay method for measuring affinity to IL-13 differed from the method described for affinity to IL-31 in the following points:

The surface plasmon resonance experiments were carried on a Biacore 8k equipped with Protein A Sensor Chip Series S. The IgG mAbs were diluted in HBS-EP+ buffer at the final concentration of 5 μg/ml and captured on the sensor chip. The flow cell 1 was left blank to serve as a reference surface. The kinetic binding data were collected by subsequent injections of 1:2 dilution series of the IL-13 proteins over the flow cell 2 at a flow rate of 50 μl/min and at a temperature of 25° C. The anti-IL13 mAb/IL13 complexes were allowed to associate for 240 s and then dissociate for 1200 s. Kinetic analysis was performed with alternating cycles of analyte injections and surface regeneration known as multi-cycle kinetics (MCK). Each injection of an analyte was done in a separate sequence, and a single SPR curve were generated per analyte concentration. The surfaces were regenerated with two subsequent injections of 10 mM glycine-HCl at pH 1.5 for 30 s at a flow rate of 50 μl/min. Data were collected at a rate of 10 Hz.

TABLE 20
Antigens

| | | Source #Cat-No. or | |
|---|---|---|---|
| ID | Full name | Database-ID | Buffer |
| hsIL-13 | Recombinant | PeproTech, #200-13- | Sodium Phosphate |
| | Human IL-13 | 10UG | pH 7.0 |
| | (115aa) | | |
| cyIL- | Recombinant | Sino Biological | PBS, pH 7.4. |
| 13 | Cyno IL-13 | Cat#11057-CNAH | |

TABLE 21
Results

| | IL-13 binding kinetics and dissociation constant | | |
|---|---|---|---|
| ID | ka (1/Ms) | kd (1/s) | KD (pM) |
| hsIL-13 | 1.9E+06 ± 0.6E+06 | 4.6E−08 ± 2.4E−08^{§} | 0.03 ± 0.002^{§} |
| cyIL-13 | 1.95 + 06 ± 0.03 + 06 | 4.1 − 05 ± 1.8 − 05 | 20.9 ± 9.1 |

^{§}Kinetic constant kd was outside the limits that can be measured by the Biacore 8k instrument. Note that the detection limit is 1 × 10⁻⁶. Kinetic constants could not be uniquely determined.

### Example 12. Inhibition of IL-31 in BaF3 Cell Proliferation Assay and Inhibition of IL-31 Binding to BaF3 Cells by Anti-IL-31/IL-13 Bispecific Antibody

The materials and methods used for inhibiting IL-31 in the IL-31 dependent BaF3 cell proliferation assay are detailed in Example 7.

### Results

Since the huIL-31RA/OSMR transgenic BaF3 cell pool (selected by using 16.3 pM of human IL-31 in culture) responded equally well to both human and cynomolgus monkey IL-31, this cell pool was used to determine inhibitory potencies of human and cynomolgus monkey IL-31-dependent cell growth by the anti-IL-31/IL-13 bispecific antibody generated in Example 9 (SEQ ID NO: 9, SEQ ID NO: 10; SEQ ID NO: 19, SEQ ID NO: 20) and various other anti-IL-31 antibodies.

The anti-IL-31/IL-13 bispecific inhibited cytokine induced cell-growth with an IC50 values of 10.8+/−5.0 pM for human IL-31 (n=9) and 10.7+/−4.4 pM for cynomolgus monkey IL-31 (n=5).

TABLE 22
IC50 values of anti-IL-31/IL-13 bispecific antibody inhibiting cell proliferation
of IL-31 growth dependent BAF3 cells in response to human IL-31 (n = 9):

| Exp. ID | CB29 | CB31 | CB32 | CB42 | CB45 | CB48 | CB56 | CB57 | CB58 | Avg | SD |
|---|---|---|---|---|---|---|---|---|---|---|---|
| IC₅₀ | 8.1 | 13 | 9.5 | 9.1 | 9.1 | 8.7 | 23.0 | 11.1 | 5.6 | 10.8 | 5.0 |
| [pM] | | | | | | | | | | | |
| IC₉₀ | 15.2 | 23.2 | 15.3 | 13.7 | 14.5 | 13.1 | 33.0 | 16.9 | 10.0 | 17.2 | 6.9 |
| [pM] | | | | | | | | | | | |

TABLE 23
IC50 values of anti-IL-31/IL-13 bispecific antibody inhibiting
cell proliferation of IL-31 dependent BAF3 cells in
response to cynomolgus monkey IL-31 (n = 6)

| Exp. ID | CB31 | CB32 | CB34 | CB56 | CB57 | CB58 | Avg | SD |
|---|---|---|---|---|---|---|---|---|
| IC₅₀ | 19.3 | 8.8 | 8.8 | 10.5 | 9.3 | 7.1 | 10.7 | 4.4 |
| [pM] | | | | | | | | |
| IC₉₀ | 25.3 | 12.9 | 11.9 | 13.9 | 12.1 | 11.2 | 14.5 | 5.3 |
| [pM] | | | | | | | | |

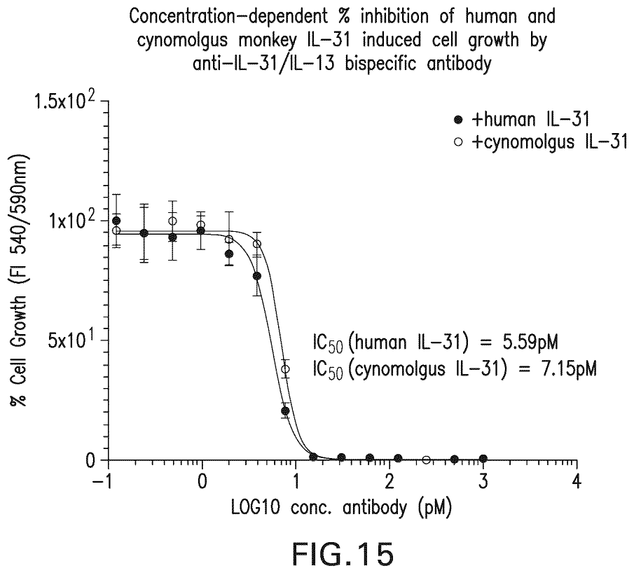

Concentration-dependent 00 inhibition of human and cynomolgus monkey IL-31 induced cell growth by the anti-IL-31/IL-13 bispecific is shown in FIG. 15.

### Example 13. Inhibition of IL-13 in HEK-Blue Reporter Assay by Anti-IL-31/IL-13 Bispecific Antibody

To assess the inhibitory potency of the anti-IL-31/IL-13 antibody generated in Example 9 (SEQ ID NO: 9, SEQ ID NO: 10; SEQ ID NO: 19, SEQ ID NO: 20) on human and cynomolgus IL-13, HEK-Blue reporter cells were stimulated with recombinant or native (contained in supernatants of MyLa cells) human IL-13 as well as recombinant cynomolgus IL-13.

HEK-Blue (Human embryonic kidney) reporter cell lines were stimulated with either 1 ng/ml (80 pM)/0.25 ng/mL (20 pM) of recombinant human IL-13, 0.04 ng/mL (3.2 pM) native human IL-13 or 10 ng/mL (690 pM) recombinant cyno IL-13 for 24 hours in the presence of increasing concentrations of bispecific antibody and inhibition of the secretion of secreted alkaline phosphatase (SEAP) was determined using the QUANTI-Blue™ method. hIL-31 dependent BaF3 cells were stimulated with 0.37 ng/ml (20 pM) IL-31 for 48 hours in the presence of increasing concentrations of bispecific antibody and inhibition of the proliferation was determined by CellTiterBlue assay. The EC ranges observed in the different experiments are also indicated.

Concentration-dependent inhibition with IC50 and IC90 values in the single to double digit pM ranges were measured:

TABLE 24
IC50 and IC90 values

| | Cells/Readout | | | | | |
|---|---|---|---|---|---|---|
| | | | | | IL-31: BaF3/ | |
| | IL-13: HEK-Blue/SEAP | | | | proliferation | |
| | Cytokine/concentration | | | | | |
| | Rec. hu IL-13 | Rec. hu IL-13 | native | | | |
| | High conc. | Low conc. | hu IL-13 | Rec. cyno | Rec. hu IL-31 | |
| | (80 pM of | (20 pM of | (3.2 pM of | IL-13 (690 pM | (20 pM of | Rec. cyno IL-31 |
| | hu IL-13; | hu IL-13; | hu IL-13; | of cyno IL-13; | hu IL-31; | (20 pM cy IL-31; |
| | observed | observed | observed | observed | observed | observed |
| | EC values: | EC values: | EC values: | EC values: | EC values: | EC values: |
| | EC₅₁ to EC₈₇) | EC₃₉ to EC₅₂) | EC₆₂ to EC₆₇) | EC₄₂ to EC₅₃) | EC₇₈ to EC₉₈) | EC₈₄ to EC₉₄) |
| IC₅₀ | 27.8 ± | 8.2 ± | 10.3 = | 389.3 ± | 10.8 = | 10.7 = |
| [pM] ± SD | 8.6 (n = 9) | 3.2 (n = 5) | 4.5 (n = 3) | 102.6 (n = 3) | 5.0 (n = 9) | 4.4 (n = 6) |
| IC₉₀ | 75.9 ± | 21.6 ± | 42.2 ± | 731.5 ± | 17.2 ± | 14.5 ± |
| [pM] ± SD | 29.9 (n = 9) | 4.8 (n = 5) | 8.6 (n = 3) | 244.9 (n = 3) | 6.9 (n = 9) | 5.3 (n = 6) |

### Example 14. Comparison Between Anti-IL-31/IL-13 Bispecific Antibody and IL-31 or IL-13 Monospecific Antibodies in Inhibition of IL-31 or IL-13

In this Example, the IL-31 or IL-13 inhibition shown by the bispecific IL-31/IL-13 bispecific antibody generated in Example 9 (SEQ ID NO: 9, SEQ ID NO: 10; SEQ ID NO: 19, SEQ ID NO: 20) was compared against anti-IL-31 or anti-IL-13 monospecific Fabs.

In the case of IL-31 inhibition, the anti-IL-31/IL-13 bispecific antibody was compared against an anti-IL-31 Fab of the humanized reference comprising the VH and VL regions represented by SEQ ID NO: 40 and SEQ ID NO: 41 respectively. The materials and methods used for the IL-31 inhibition assay (IL-31 dependent BaF3 cell proliferation assay) are detailed in Example 7.

The anti-IL-31/IL-13 antibody showed a far lower IL-31 IC50 value in this assay (three experiments) compared to the anti-IL-31 Fab.

TABLE 25
IC50 values

| IC₅₀ values | | | | |
|---|---|---|---|---|
| (pM) in IL-31 | Experiment | Experiment | Experiment | Mean A + |
| inhibition assay | A | B | C | B + C |
| anti-IL-31/IL-13 antibody | 9.1 | 9.1 | 8.7 | 9.0 |
| anti-IL-31 Fab | 181.7 | 408.3 | 380.8 | 323.6 |

In the case of IL-13 inhibition, the anti-IL-31/IL-13 bispecific antibody was compared against an anti-IL-13 Fab comprising the same IL-13-binding VH and VL regions as the bispecific. The materials and methods used for the IL-13 inhibition assay (HEK-Blue reporter assay) are detailed in Example 13. The anti-IL-31/IL-13 antibody showed a similar IL-13 IC50 value in this assay (four experiments) compared to the anti-IL-13 Fab.

TABLE 26
IC50 values

| IC₅₀ values (pM) | | | | | |
|---|---|---|---|---|---|
| in IL-13 inhibition | | | | | Mean A + |
| assay | Exp A | Exp B | Exp C | Exp D | B + C + D |
| anti-IL-31/IL-13 | 2.2 | 1.5 | 3.8 | 3.3 | 2.7 |
| antibody | | | | | |
| anti-IL-13 Fab | 3.8 | 1.8 | 2.2 | 3.1 | 2.7 |

### Example 15. Comparison Between Anti-IL-31/IL-13 Bispecific Antibody and Available Molecules in Inhibition of IL-31 in BaF3 Cell Proliferation Assay

In this Example, the IL-31 inhibition shown by the bispecific anti-IL-31/IL-13 antibody generated in Example 9 (SEQ ID NO: 9, SEQ ID NO: 10; SEQ ID NO: 19, SEQ ID NO: 20) was compared against an anti-IL-31/IL-4R bispecific antibody described in WO2022/136669 (SEQ ID NO: 174; SEQ ID NO: 175). The materials and methods used for inhibiting IL-31 in the IL-31 dependent BaF3 cell proliferation assay are detailed in Example 7.

The anti-IL-31/IL-13 antibody showed a far lower IC50 value in this assay (two experiments) compared to the anti-IL-31/IL-4R antibody.

TABLE 27
IC50 values

| IC₅₀ values (pM) in IL-31 | Experiment | Experiment | Mean A + |
|---|---|---|---|
| inhibition assay | A | B | B |
| anti-IL-31/IL-13 antibody | 9.1 | 9.1 | 9.1 |
| anti-IL-31/IL4R antibody | 253.4 | 664.7 | 459.1 |

### Example 16. Assessment of an Anti-IL-31/IL-13 Bispecific Antibody with Modified Fc Mutations for Fab Arm Exchange

The production of an anti-IL-31/IL-13 bispecific antibody, differing from the one described in Example 9 solely in the mutations introduced in the constant domains of the heavy chains to facilitate Fab arm exchange and correct chain pairing (SEQ ID NOs: 176-179), was assessed in this Example.

For the anti-IL-13 monoclonal parental antibody (mAbA), the constant region of the heavy chain comprised a T366W mutation (EU numbering). For the anti-IL-31 monoclonal antibody (mAbB), the constant region of the heavy chain comprised an T366S, an L368A and a Y407V mutation (EU numbering). Additional mutations in the constant region of the heavy chain for silencing of Fc effector function and for half-life extension were identical to the anti-IL-31/IL-13 bispecific antibody described in Example 9.

In this example, the parental monospecific antibodies of both anti-IL-31/IL-13 bispecifics were produced following the same methodology described in Example 9. Following production, protein A affinity chromatography capture, and elution, the parental antibodies were characterized using size exclusion chromatography and capillary electrophoresis CE-SDS (performed as described in Example 9).

The characterization of the parental antibodies revealed a notably higher heterogeneity in mAbA and mAbB compared to mAb1 and mAb2. Both mAbA and mAbB antibodies exhibited over 70% low molecular weight (LMW) species, indicating substantial molecular variability.

TABLE 28
HMW and LMW values

| | | HMW | LMW | LMW |
|---|---|---|---|---|
| | | (%, by | (%, by | (%, by HT- |
| Candidates | | SEC) | SEC) | CE-SDS) |
| anti-IL-31/IL-13 | mAbA | 2.6 | 75 | 81 |
| bispecific with | (anti-IL13) | | | |
| modified Fc | mAbB | 2.9 | 70.6 | 84.3 |
| (SEQ ID NOs: | (anti-IL31) | | | |
| 176-179) | | | | |
| anti-IL-31/IL-13 | mAb1 | 3.5 | <LOQ | 4.4 |
| bispecific from | (anti-IL13) | | | |
| Example 9 | mAb2 | 2.3 | 5.4 | 6.1 |
| (SEQ ID NOs: 9, | (anti-IL31) | | | |
| 10, 19, 20) | | | | |

TABLE 29
Additional non-limiting Examples of
sequences pertaining to the disclosure.

| SEQ ID | |
|---|---|
| NO | Sequence |
| SEQ ID | QVQLVQSGAEVKKPGASVKVSCKASGYTLTRYWMQWVRQAPGQ |
| NO: 40 | GLEWMGAIYPGDGDTRYSQKFQGRVTLTADKSTSTAYMELSSLRSED |
| | TAVYYCAFPDGYYAAPYGMDYWGQGTLVTVSS |
| SEQ ID | DIQMTQSPSSLSASVGDRVTITCRASGNIHNYLAWYQQKPGKVPK |
| NO: 41 | LLIYNAKTLADGVPSRFSGSRSETQYSLTISSLQPEDVATYYCQHFWST |
| | PWTFGQGTKVEIK |
| SEQ ID | QVQLVQSGAEVKKPGSSVKVSCKASGYTRTRYWMQWVRQAPGQ |
| NO: 112 | GLEWMGAIYPGEGDTRYSQKFKGRVTITADKSTSTAYMELSSLRSEDT |
| | AVYYCAFPEGPYAAPYGMHYWGQGTTVTVSSASTKGPSVFPLAPSSK |
| | STSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCP |
| | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVYTLPPCREEMTKNQVSLWCLVKGF |
| | YPSDIAVEWESNGQPENNYKTTPPVLDSDGSFFLYSKLTVDKSRWQQ |
| | GNVFSCSVMHEALHNHYTQKSLSLSPGK |
| SEQ ID | QVQLVQSGAEVKKPGSSVKVSCKASGYTRTRYWMQWVRQAPGQ |
| NO: 113 | GLEWMGAIYPGEGDTRYSQKFKGRVTITADKSTSTAYMELSSLRSEDT |
| | AVYYCAFPEGQYAAPYGMHYWGQGTTVTVSSASTKGPSVFPLAPSSK |
| | STSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCP |
| | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVYTLPPCREEMTKNQVSLWCLVKGF |
| | YPSDIAVEWESNGQPENNYKTTPPVLDSDGSFFLYSKLTVDKSRWQQ |
| | GNVFSCSVMHEALHNHYTQKSLSLSPGK |
| SEQ ID | QVQLVQSGAEVKKPGSSVKVSCKASGYTRTRYWMQWVRQAPGQ |
| NO: 114 | GLEWMGAIYPAEGDTRYSQKFKGRVTITADKSTSTAYMELSSLRSEDT |
| | AVYYCAFPEGPYAAPYGMHYWGQGTTVTVSSASTKGPSVFPLAPSSK |
| | STSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCP |
| | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVYTLPPCREEMTKNQVSLWCLVKGF |
| | YPSDIAVEWESNGQPENNYKTTPPVLDSDGSFFLYSKLTVDKSRWQQ |
| | GNVFSCSVMHEALHNHYTQKSLSLSPGK |
| SEQ ID | QVQLVQSGAEVKKPGSSVKVSCKASGYTRTRYWMQWVRQAPGQ |
| NO: 116 | GLEWMGAIYPGEGDTRYSQKFKGRVTITADKSTSTAYMELSSLRSEDT |
| | AVYYCAFPEGPYAAPYGMHYWGQGTTVTVSSASTKGPSVFPLAPSSK |
| | STSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCP |
| | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVCTLPPSREEMTKNQVSLSCAVKGF |
| | YPSDIAVEWESNGQPENNYKTTPPVLDSDGSFFLVSKLTVDKSRWQQ |
| | GNVFSCSVMHEALHNHYTQKSLSLSPGK |
| SEQ ID | QVQLVQSGAEVKKPGSSVKVSCKASGYTRTRYWMQWVRQAPGQ |
| NO: 117 | GLEWMGAIYPGEGDTRYSQKFKGRVTITADKSTSTAYMELSSLRSEDT |
| | AVYYCAFPEGQYAAPYGMHYWGQGTTVTVSSASTKGPSVFPLAPSSK |
| | STSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCP |
| | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVCTLPPSREEMTKNQVSLSCAVKGF |
| | YPSDIAVEWESNGQPENNYKTTPPVLDSDGSFFLVSKLTVDKSRWQQ |
| | GNVFSCSVMHEALHNHYTQKSLSLSPGK |
| SEQ ID | QVQLVQSGAEVKKPGSSVKVSCKASGYTRTRYWMQWVRQAPGQ |
| NO: 118 | GLEWMGAIYPAEGDTRYSQKFKGRVTITADKSTSTAYMELSSLRSEDT |
| | AVYYCAFPEGPYAAPYGMHYWGQGTTVTVSSASTKGPSVFPLAPSSK |
| | STSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCP |
| | APELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVCTLPPSREEMTKNQVSLSCAVKGF |
| | YPSDIAVEWESNGQPENNYKTTPPVLDSDGSFFLVSKLTVDKSRWQQ |
| | GNVFSCSVMHEALHNHYTQKSLSLSPGK |
| SEQ ID | caggttcagttggtgcagtccggcgctgaagtgaagaaacctggctcctctgtgaaggtgtcctgcaagg |
| NO: 119 | cctctggctacacccggaccagatattggatgcagtgggtccgacaggctcctggacaaggattggagtggat |
| | gggcgctatctaccctgctgagggcgacacccggtactcccagaaattcaagggcagagtgaccatcaccgc |
| | cgacaagtctacctccaccgcctacatggaactgtccagcctgagatccgaggacaccgccgtgtactactgtg |
| | cctttcctgagggaccttacgccgctccttacggcatgcattattggggccagggcaccaccgtgaccgtgtcct |
| | ct |
| SEQ ID | gccatccagatgacccagtctccatcctctctgtctgcctctgtgggcgacagagtgaccatcacctgtcg |
| NO: 120 | ggcttcccggaacatcggcaactacctggcctggtatcagcagaaacctggcaaggtgcccaagctgctgatc |
| | tactacgccaagacctctgccagcggcgtgccaagcagattttctggaagcagatccggcaccgactacaccc |
| | tgaccatttctagcctgcagcctgaggacgtggccacctactactgtcagcacttctggtctaccccttggaccttt |
| | ggcggaggcaccaaggtggaaatcaag |
| SEQ ID | gccatccagatgacccagtctccatcctctctgtctgcctctgtgggcgacagagtgaccatcacctgtcg |
| NO: 121 | ggcttcccggaacatcggcaactacctggcctggtatcagcagaaacctggcaaggtgcccaagctgctgatc |
| | tactacgccaagacctctgccagcggcgtgccaagcagattttctggaagcagatccggcaccgactacaccc |
| | tgaccatttctagcctgcagcctgaggacgtggccacctactactgtcagcacttctggtctaccccttggaccttt |
| | ggcggaggcaccaaggtggaaatcaagcgtacggtggccgctccttccgtgttcatcttcccaccatctgacga |
| | gcagctgaagtctggcaccgcttctgtcgtgtgcctgctgaacaacttctaccctcgggaagccaaggtgcagt |
| | ggaaggtggacaatgccctgcagtccggcaactcccaagagtctgtgaccgagcaggactccaaggactcta |
| | cctacagcctgtcctccacactgaccctgtctaaggccgactacgagaagcacaaggtgtacgcctgtgaagtg |
| | acccaccagggactgtctagccccgtgaccaagtctttcaacagaggcgagtgc |
| SEQ ID | caggttcagttggtgcagtccggcgctgaagtgaagaaacctggctcctctgtgaaggtgtcctgcaagg |
| NO: 122 | cctctggctacacccggaccagatattggatgcagtgggtccgacaggctcctggacaaggattggagtggat |
| | gggcgctatctaccctgctgagggcgacacccggtactcccagaaattcaagggcagagtgaccatcaccgc |
| | cgacaagtctacctccaccgcctacatggaactgtccagcctgagatccgaggacaccgccgtgtactactgtg |
| | cctttcctgagggaccttacgccgctccttacggcatgcattattggggccagggcaccaccgtgaccgtgtcct |
| | ctgcttctacaaagggcccctctgtgttccctctggctcctagctctaagtccacctctggtggaaccgctgctctg |
| | ggctgtctggtcaaggattacttccctgagccagtgacagtgtcctggaactctggtgctctgacctccggcgtg |
| | cacacatttccagctgtgctgcagtcctccggcctgtactctctgtcctctgtcgtgacagtgccttccagctctctg |
| | ggcacccagacctacatctgcaacgtgaaccacaagccttccaataccaaggtggacaagagagtggaaccc |
| | aagtcctgcgacaagacccacacgtgccctccatgtcctgctccagaagctgctggcggaccctccgttttcct |
| | gtttccacctaagcctaaggacaccctgtacatcacccgcgagcctgaagtgacctgtgtggtggtggatgtgtc |
| | ccacgaggaccccgaagtgaagtttaattggtacgtggacggcgtggaagtgcacaacgccaagaccaagcc |
| | tagagaggaacagtacaactccacctacagagtggtgtccgtgctgaccgtgctgcaccaggattggctgaac |
| | ggcaaagagtacaagtgcaaggtgtccaacaaggcactgcccgctcctatcgaaaagaccatctccaaggcta |
| | agggccagcctcgggaacctcaggtttacacactgcctccaagccgggaagagatgaccaagaatcaggtgt |
| | ccctgacctgcctcgtgaagggcttctacccttccgatatcgccgtggaatgggagtctaacggccagccagag |
| | aacaactacaagaccacacctcctgtgctggactccgacggctctttcctgctgtactccaagctgacagtggac |
| | aagtccagatggcagcagggcaacgtgttctcctgctctgtgatgcacgaggccctgcacaaccactacaccc |
| | agaagtccctgtctctgtcccctggcaaa |
| SEQ ID | caagtcactctgcgcgaatcaggaccggccctggtcaagcccacccagaccctgaccctgacttgtactg |
| NO: 123 | tgtcgggtttctccctgtccgcgtatagcgtgaactggattagacagcctcccggaaaggccctggagtggcttg |
| | cgatgatctggggggacgggaagatcgtgtacaacagcgccctcaagagccgcctgaccatcagcaaggac |
| | acctctaagaatcaggtcgtgctgactatgaccaacatggatcccgtggacaccgccacctactactgtgctgg |
| | ggacggctactatccctacgccatggacaactggggacagggttccctggttactgtgtcctcc |
| SEQ ID | gacattgtgatgacccagtccccggactccctctcggtgtccctgggagaacgcgcgactatcaactgtc |
| NO: 124 | gggcttccaagtccgtggactcctacggaaactcgttcatgcattggtaccagcagaaacctggacagccgcc |
| | aaagctgctcatctacctggcatccaacctggagagcggagtgccggatagattcagcggatcgggttcaggc |
| | accgacttcaccctgaccatctcatcactccaagcggaggatgtggccgtgtactactgccaacagaacaacga |
| | ggatcccaggaccttcgggggtggcactaaggtcgagatcaag |
| SEQ ID | gacattgtgatgacccagtccccggactccctctcggtgtccctgggagaacgcgcgactatcaactgtc |
| NO: 125 | gggcttccaagtccgtggactcctacggaaactcgttcatgcattggtaccagcagaaacctggacagccgcc |
| | aaagctgctcatctacctggcatccaacctggagagcggagtgccggatagattcagcggatcgggttcaggc |
| | accgacttcaccctgaccatctcatcactccaagcggaggatgtggccgtgtactactgccaacagaacaacga |
| | ggatcccaggaccttcgggggtggcactaaggtcgagatcaagcgtacggtggccgcacctagcgtgttcatt |
| | ttcccgccctccgatgaacagctgaagtcgggcactgccagcgtggtctgcctgctgaacaacttttaccctcgg |
| | gaagccaaggtccagtggaaagtggacaatgccctgcaatccgggaactcccaggagagcgtgaccgaaca |
| | ggactctaaggacagcacctactcgctctcctcaacccttacactgtccaaggccgactatgagaagcacaagg |
| | tctacgcctgcgaagtgacccaccaaggattgtcgtcccccgtgaccaagagcttcaatcggggcgaatgt |
| SEQ ID | caagtcactctgcgcgaatcaggaccggccctggtcaagcccacccagaccctgaccctgacttgtactg |
| NO: 126 | tgtcgggtttctccctgtccgcgtatagcgtgaactggattagacagcctcccggaaaggccctggagtggcttg |
| | cgatgatctggggggacgggaagatcgtgtacaacagcgccctcaagagccgcctgaccatcagcaaggac |
| | acctctaagaatcaggtcgtgctgactatgaccaacatggatcccgtggacaccgccacctactactgtgctgg |
| | ggacggctactatccctacgccatggacaactggggacagggttccctggttactgtgtcctccgcttcgacca |
| | agggcccgtcggtgtttccactcgccccgagctccaagagcacttcaggcggtactgcagccttgggatgcct |
| | cgtgaaggactacttccctgagccggtcaccgtgtcctggaacagcggcgccctgacttccggggtgcacacc |
| | ttcccggccgtgctgcaatcctccggcctgtactcactgtcctcggtcgtgaccgtgccgagcagcagcctggg |
| | cacccagacctacatctgcaacgtgaaccacaagccctccaacaccaaggtcgataagagggtggaacccaa |
| | gtcctgcgacaagacccacacgtgcccaccttgtcctgcgcctgaagcggctggaggaccatccgtgttcctgt |
| | tcccgcctaaaccaaaggacacgctctacattactcgcgaaccggaagtcacttgcgtggtggtggatgtgtcg |
| | cacgaggatcccgaggtcaagtttaattggtacgtggatggcgtggaagtgcacaacgccaagactaagccgc |
| | gggaggaacagtacaactccacctaccgggtggtgtcggtgctgaccgtgctgcatcaggactggctcaacg |
| | gaaaggagtacaagtgcaaagtgtccaacaaggcacttccagccccgattgagaaaaccatctcaaaggcca |
| | agggacagccgagagagccccaggtctacacactccctccttcgcgggaggagatgaccaagaaccaagtg |
| | tcacttacttgcctggtcaagggcttctacccgtccgatatcgccgtggagtgggaatccaacggacagccgga |
| | aaacaactacaagaccacccctcccgtactggacagcgacggatcgttcttcctgtactcccggctcactgtgg |
| | acaagtcacggtggcagcagggaaacgtgttctcctgctctgtcatgcatgaggcgctgcataatcactataccc |
| | aaaagtccctgtccctgagtcccgggaaa |
| SEQ ID | caggtgcagctggttcagtctggcgccgaagtgaagaaacccggcagcagcgtgaaggtgtcctgtaaa |
| NO: 127 | gccagcggctacacccggaccagatactggatgcagtgggttcgacaggcccctggacaaggacttgagtgg |
| | atgggagccatctatcctggcgagggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggaccttacgccgctccttacggcatgcattattggggccagggcaccaccgtgaccgt |
| | ttcttct |
| SEQ ID | caggtgcagctggttcagtctggcgccgaagtgaagaaacccggcagcagcgtgaaggtgtcctgtaaa |
| NO: 128 | gccagcggctacacccggaccagatactggatgcagtgggttcgacaggcccctggacaaggacttgagtgg |
| | atgggagccatctatcctggcgagggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggacagtacgccgctccttacggcatgcattattggggccagggcaccaccgtgaccg |
| | tttcttct |
| SEQ ID | caggtgcagctggttcagtctggcgccgaagtgaagaaacccggcagcagcgtgaaggtgtcctgtaaa |
| NO: 129 | gccagcggctacacccggaccagatactggatgcagtgggttcgacaggcccctggacaaggacttgagtgg |
| | atgggagccatctatcctgccgagggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggaccttacgccgctccttacggcatgcattattggggccagggcaccaccgtgaccgt |
| | ttcttct |
| SEQ ID | gccatccagatgacacagagccctagcagcctgtctgcctctgtgggcgatagagtgaccatcacctgtc |
| NO: 130 | gggccagccggaacatcggaaactatctggcctggtatcagcagaaacccggcaaggtgcccaagctgctga |
| | tctactatgccaagacaagcgccagcggcgtgccaagcagattttctggcagcagaagcggcaccgactaca |
| | ccctgaccatttctagcctgcagcctgaggacgtggccacctactactgccagcacttctggaaaaccccttgga |
| | cctttggcggaggcaccaaggtggaaatcaag |
| SEQ ID | gccatccagatgacacagagccctagcagcctgtctgcctctgtgggcgatagagtgaccatcacctgtc |
| NO: 131 | gggccagccggaacatccacaactatctggcctggtatcagcagaaacccggcaaggtgcccaagctgctga |
| | tctactatgccaagacaagcgccagcggcgtgccaagcagattttctggcagcagaagcggcaccgactaca |
| | ccctgaccatttctagcctgcagcctgaggacgtggccacctactactgccagcacttctggaaaaccccttgga |
| | cctttggcggaggcaccaaggtggaaatcaag |
| SEQ ID | gccatccagatgacacagagccctagcagcctgtctgcctctgtgggcgatagagtgaccatcacctgtc |
| NO: 132 | gggccagccggaacatcggaaactatctggcctggtatcagcagaaacccggcaaggtgcccaagctgctga |
| | tctactatgccaagacatctgccgagggcgtgccaagcagattttctggcagcagaagcggcaccgactacac |
| | cctgaccatttctagcctgcagcctgaggacgtggccacctactactgccagcacttctggaaaaccccttggac |
| | ctttggcggaggcaccaaggtggaaatcaag |
| SEQ ID | gccatccagatgacacagagccctagcagcctgtctgcctctgtgggcgatagagtgaccatcacctgtc |
| NO: 133 | gggccagccggaacatcggaaactatctggcctggtatcagcagaaacccggcaaggtgcccaagctgctga |
| | tctactatgccaagacaagcgccagcggcgtgccaagcagattttctggcagcagaagcggcaccgactaca |
| | ccctgaccatttctagcctgcagcctgaggacgtggccacctactactgtcagcacttttggagcaccccttgga |
| | cctttggcggaggcaccaaggtggaaatcaag |
| SEQ ID | gccatccagatgacacagagccctagcagcctgtctgcctctgtgggcgatagagtgaccatcacctgtc |
| NO: 134 | gggccagccggaacatcggaaactatctggcctggtatcagcagaaacccggcaaggtgcccaagctgctga |
| | tctactatgccaagacaagcgccagcggcgtgccaagcagattttctggcagcagaagcggcaccgactaca |
| | ccctgaccatttctagcctgcagcctgaggacgtggccacctactactgccagcacttctggaaaaccccttgga |
| | cctttggcggaggcaccaaggtggaaatcaagcgtacggtggccgctcccagcgtgttcatcttcccccccag |
| | cgacgagcagctgaagagcggcaccgccagcgtggtgtgcctgctgaacaacttctacccccgggaggcca |
| | aggtgcagtggaaggtggacaacgccctgcagagcggcaacagccaggagagcgtcaccgagcaggaca |
| | gcaaggactccacctacagcctgagcagcaccctgaccctgagcaaggccgactacgagaagcataaggtgt |
| | acgcctgcgaggtgacccaccagggcctgtccagccccgtgaccaagagcttcaacaggggcgagtgc |
| SEQ ID | gccatccagatgacacagagccctagcagcctgtctgcctctgtgggcgatagagtgaccatcacctgtc |
| NO: 135 | gggccagccggaacatccacaactatctggcctggtatcagcagaaacccggcaaggtgcccaagctgctga |
| | tctactatgccaagacaagcgccagcggcgtgccaagcagattttctggcagcagaagcggcaccgactaca |
| | ccctgaccatttctagcctgcagcctgaggacgtggccacctactactgccagcacttctggaaaaccccttgga |
| | cctttggcggaggcaccaaggtggaaatcaagcgtacggtggccgctcccagcgtgttcatcttcccccccag |
| | cgacgagcagctgaagagcggcaccgccagcgtggtgtgcctgctgaacaacttctacccccgggaggcca |
| | aggtgcagtggaaggtggacaacgccctgcagagcggcaacagccaggagagcgtcaccgagcaggaca |
| | gcaaggactccacctacagcctgagcagcaccctgaccctgagcaaggccgactacgagaagcataaggtgt |
| | acgcctgcgaggtgacccaccagggcctgtccagccccgtgaccaagagcttcaacaggggcgagtgc |
| SEQ ID | gccatccagatgacacagagccctagcagcctgtctgcctctgtgggcgatagagtgaccatcacctgtc |
| NO: 136 | gggccagccggaacatcggaaactatctggcctggtatcagcagaaacccggcaaggtgcccaagctgctga |
| | tctactatgccaagacatctgccgagggcgtgccaagcagattttctggcagcagaagcggcaccgactacac |
| | cctgaccatttctagcctgcagcctgaggacgtggccacctactactgccagcacttctggaaaaccccttggac |
| | ctttggcggaggcaccaaggtggaaatcaagcgtacggtggccgctcccagcgtgttcatcttcccccccagc |
| | gacgagcagctgaagagcggcaccgccagcgtggtgtgcctgctgaacaacttctacccccgggaggccaa |
| | ggtgcagtggaaggtggacaacgccctgcagagcggcaacagccaggagagcgtcaccgagcaggacag |
| | caaggactccacctacagcctgagcagcaccctgaccctgagcaaggccgactacgagaagcataaggtgta |
| | cgcctgcgaggtgacccaccagggcctgtccagccccgtgaccaagagcttcaacaggggcgagtgc |
| SEQ ID | gccatccagatgacacagagccctagcagcctgtctgcctctgtgggcgatagagtgaccatcacctgtc |
| NO: 137 | gggccagccggaacatcggaaactatctggcctggtatcagcagaaacccggcaaggtgcccaagctgctga |
| | tctactatgccaagacaagcgccagcggcgtgccaagcagattttctggcagcagaagcggcaccgactaca |
| | ccctgaccatttctagcctgcagcctgaggacgtggccacctactactgtcagcacttttggagcaccccttgga |
| | cctttggcggaggcaccaaggtggaaatcaagcgtacggtggccgctcccagcgtgttcatcttcccccccag |
| | cgacgagcagctgaagagcggcaccgccagcgtggtgtgcctgctgaacaacttctacccccgggaggcca |
| | aggtgcagtggaaggtggacaacgccctgcagagcggcaacagccaggagagcgtcaccgagcaggaca |
| | gcaaggactccacctacagcctgagcagcaccctgaccctgagcaaggccgactacgagaagcataaggtgt |
| | acgcctgcgaggtgacccaccagggcctgtccagccccgtgaccaagagcttcaacaggggcgagtgc |
| SEQ ID | caggttcagttggtgcagtccggcgctgaagtgaagaaacctggctcctctgtgaaggtgtcctgcaagg |
| NO: 138 | cctctggctacacccggaccagatattggatgcagtgggtccgacaggctcctggacaaggattggagtggat |
| | gggcgctatctaccctgctgagggcgacacccggtactcccagaaattcaagggcagagtgaccatcaccgc |
| | cgacaagtctacctccaccgcctacatggaactgtccagcctgagatccgaggacaccgccgtgtactactgtg |
| | cctttcctgagggaccttacgccgctccttacggcatgcattattggggccagggcaccaccgtgaccgtgtcct |
| | ctgcttctacaaagggcccctctgtgttccctctggctcctagctctaagtccacctctggtggaaccgctgctctg |
| | ggctgtctggtcaaggattacttccctgagccagtgacagtgtcctggaactctggtgctctgacctccggcgtg |
| | cacacatttccagctgtgctgcagtcctccggcctgtactctctgtcctctgtcgtgacagtgccttccagctctctg |
| | ggcacccagacctacatctgcaacgtgaaccacaagccttccaataccaaggtggacaagagagtggaaccc |
| | aagtcctgc |
| SEQ ID | caggtgcagctggttcagtctggcgccgaagtgaagaaacccggcagcagcgtgaaggtgtcctgtaaa |
| NO: 139 | gccagcggctacacccggaccagatactggatgcagtgggttcgacaggcccctggacaaggacttgagtgg |
| | atgggagccatctatcctggcgagggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggaccttacgccgctccttacggcatgcattattggggccagggcaccaccgtgaccgt |
| | ttcttctgctagcaccaagggccccagcgtgttccccctggcccccagcagcaagagcaccagcggcggcac |
| | agccgccctgggctgcctggtgaaggactacttccccgagcccgtgaccgtgtcctggaacagcggagccct |
| | gacctccggcgtgcacaccttccccgccgtgctgcagagcagcggcctgtacagcctgtccagcgtggtgac |
| | agtgcccagcagcagcctgggcacccagacctacatctgcaacgtgaaccacaagcccagcaacaccaagg |
| | tggacaagagagtggagcccaagagctgc |
| SEQ ID | caggtgcagctggttcagtctggcgccgaagtgaagaaacccggcagcagcgtgaaggtgtcctgtaaa |
| NO: 140 | gccagcggctacacccggaccagatactggatgcagtgggttcgacaggcccctggacaaggacttgagtgg |
| | atgggagccatctatcctggcgagggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggacagtacgccgctccttacggcatgcattattggggccagggcaccaccgtgaccg |
| | tttcttctgctagcaccaagggccccagcgtgttccccctggcccccagcagcaagagcaccagcggcggca |
| | cagccgccctgggctgcctggtgaaggactacttccccgagcccgtgaccgtgtcctggaacagcggagccc |
| | tgacctccggcgtgcacaccttccccgccgtgctgcagagcagcggcctgtacagcctgtccagcgtggtgac |
| | agtgcccagcagcagcctgggcacccagacctacatctgcaacgtgaaccacaagcccagcaacaccaagg |
| | tggacaagagagtggagcccaagagctgc |
| SEQ ID | caggtgcagctggttcagtctggcgccgaagtgaagaaacccggcagcagcgtgaaggtgtcctgtaaa |
| NO: 141 | gccagcggctacacccggaccagatactggatgcagtgggttcgacaggcccctggacaaggacttgagtgg |
| | atgggagccatctatcctgccgagggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggaccttacgccgctccttacggcatgcattattggggccagggcaccaccgtgaccgt |
| | ttcttctgctagcaccaagggccccagcgtgttccccctggcccccagcagcaagagcaccagcggcggcac |
| | agccgccctgggctgcctggtgaaggactacttccccgagcccgtgaccgtgtcctggaacagcggagccct |
| | gacctccggcgtgcacaccttccccgccgtgctgcagagcagcggcctgtacagcctgtccagcgtggtgac |
| | agtgcccagcagcagcctgggcacccagacctacatctgcaacgtgaaccacaagcccagcaacaccaagg |
| | tggacaagagagtggagcccaagagctgc |
| SEQ ID | caagtcactctgcgcgaatcaggaccggccctggtcaagcccacccagaccctgaccctgacttgtactg |
| NO: 142 | tgtcgggtttctccctgtccgcgtatagcgtgaactggattagacagcctcccggaaaggccctggagtggcttg |
| | cgatgatctggggggacgggaagatcgtgtacaacagcgccctcaagagccgcctgaccatcagcaaggac |
| | acctctaagaatcaggtcgtgctgactatgaccaacatggatcccgtggacaccgccacctactactgtgctgg |
| | ggacggctactatccctacgccatggacaactggggacagggttccctggttactgtgtcctccgcttcgacca |
| | agggcccgtcggtgtttccactcgccccgagctccaagagcacttcaggcggtactgcagccttgggatgcct |
| | cgtgaaggactacttccctgagccggtcaccgtgtcctggaacagcggcgccctgacttccggggtgcacacc |
| | ttcccggccgtgctgcaatcctccggcctgtactcactgtcctcggtcgtgaccgtgccgagcagcagcctggg |
| | cacccagacctacatctgcaacgtgaaccacaagccctccaacaccaaggtcgataagagggtggaacccaa |
| | gtcctgc |
| SEQ ID | MASHSGPSTSVLFLFCCLGGWLASHTLPVRLLRPSDDVQKIVEELQ |
| NO: 143 | SLSKMLLKDVEEEKGVLVSQNYTLPCLSPDAQPPNNIHSPAIRAYLKTI |
| | RQLDNKSVIDEIIEHLDKLIFQDAPETNISVPTDTHECKRFILTISQQFSE |
| | CMDLALKSLTSGAQQATT |
| SEQ ID | MASHSGPATSVLFLLCCLGGWLTSHTLPVHFLQPSDIQKIVEELQS |
| NO: 144 | LSKMLLKDVKEDKGVLVSQNYTLPCLTPDAQPPNIIHSPAIRAYLKTIR |
| | QLDNKSVIDEIIEHLDKLIFQDAPETNISVPTDTHECKRFILTISQQFSEC |
| | MDLALKSLTSGAQQATT |
| SEQ ID | PSDDVQKIVEELQSLSKMLLKDVEEEKGVLVSQNYTLPCLSPDAQ |
| NO: 145 | PPNNIHSPAIRAYLKTIRQLDNKSVIDEIIEHLDKLIFQDAPETNISVPTD |
| | THESKRFILTISQQFSECMDLALKSLTSG |
| SEQ ID | SDIQKIVEELQSLSKMLLKDVKEDKGVLVSQNYTLPCLTPDAQPPN |
| NO: 146 | IIHSPAIRAYLKTIRQLDNKSVIDEIIEHLDKLIFQDAPETNISVPTDTHES |
| | KRFILTISQQFSECMDLALKSLTSG |
| SEQ ID | QVQLVQSGAEVKKPGSSVKVSCKASGYTLTRYWMQWVRQAPGQ |
| NO: 147 | GLEWMGAIYPGDGDTRYSQKFKGRVTITADKSTSTAYMELSSLRSED |
| | TAVYYCAFPEGYYAAPYGMDYWGQGTTVTVSSASTKGPSVFPLAPSS |
| | KSTSGGTAALGCLVKDYFPEPVTVSWNSGALTSGVHTFPAVLQSSGL |
| | YSLSSVVTVPSSSLGTQTYICNVNHKPSNTKVDKRVEPKSC |
| SEQ ID | DIQMTQSPSSLSASVGDRVTITCRASGNIHNYLAWYQQKPGKVPK |
| NO: 148 | LLIYNAKTLAEGVPSRFSGSRSETDYTLTISSLQPEDVATYYCQHFWST |
| | PWTFGGGTKVEIKRTVAAPSVFIFPPSDEQLKSGTASVVCLLNNFYPRE |
| | AKVQWKVDNALQSGNSQESVTEQDSKDSTYSLSSTLTLSKADYEKHK |
| | VYACEVTHQGLSSPVTKSFNRGEC |
| SEQ ID | QVQLVQSGAEVKKPGSSVKVSCKASGYTLTRYWMQWVRQAPGQ |
| NO: 149 | GLEWMGAIYPGDGDTRYSQKFKGRVTITADKSTSTAYMELSSLRSED |
| | TAVYYCAFPEGYYAAPYGMDYWGQG |
| SEQ ID | DIQMTQSPSSLSASVGDRVTITCRASGNIHNYLAWYQQKPGKVPK |
| NO: 150 | LLIYNAKTLAEGVPSRFSGSRSETDYTLTISSLQPEDVATYYCQHFWST |
| | PWTFGGGTKVEIKR |
| SEQ ID | QVQLVQSGAEVKKPGSSVKVSCKASGYTRTRYWMQWVRQAPGQ |
| NO: 151 | GLEWMGAIYPAEGDTRYSQKFKGRVTITADKSTSTAYMELSSLRSEDT |
| | AVYYCAFPEGPYAAPYGMHYWGQG |
| SEQ ID | AIQMTQSPSSLSASVGDRVTITCRASRNIGNYLAWYQQKPGKVPK |
| NO: 152 | LLIYYAKTSASGVPSRFSGSRSGTDYTLTISSLQPEDVATYYCQHFWST |
| | PWTFGGGTKVEIKR |
| SEQ ID | QVQLVQSGAEVKKPGASVKVSCKASGYTLTRYWMQWVRQAPGQ |
| NO: 153 | GLEWMGAIYPGDGDTRYSQKFQGRVTLTADKSTSTAYMELSSLRSED |
| | TAVYYCAFPDGYYAAPYGMDYWGQGTLVTVSSASTKGPSVFPLAPSS |
| | KSTSGGTAALGCLVKDYFPEPVTVSWNSGALTSGVHTFPAVLQSSGL |
| | YSLSSVVTVPSSSLGTQTYICNVNHKPSNTKVDKRVEPKSC |
| SEQ ID | DIQMTQSPSSLSASVGDRVTITCRASGNIHNYLAWYQQKPGKVPK |
| NO: 154 | LLIYNAKTLADGVPSRFSGSRSETQYSLTISSLQPEDVATYYCQHFWST |
| | PWTFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKHK |
| | VYACEVTHQGLSSPVTKSFNRGEC |
| SEQ ID | QVQLVQSGAEVKKPGSSVKVSCKASGYTLTRYWMQWVRQAPGQ |
| NO: 155 | GLEWMGAIYPGDGDTRYSQKFKGRVTITADKSTSTAYMELSSLRSED |
| | TAVYYCAFPDGYYAAPYGMDYWGQGTTVTVSSASTKGPSVFPLAPSS |
| | KSTSGGTAALGCLVKDYFPEPVTVSWNSGALTSGVHTFPAVLQSSGL |
| | YSLSSVVTVPSSSLGTQTYICNVNHKPSNTKVDKRVEPKSC |
| SEQ ID | DIQMTQSPSSLSASVGDRVTITCRASGNIHNYLAWYQQKPGKVPK |
| NO: 156 | LLIYNAKTLADGVPSRFSGSRSETDYTLTISSLQPEDVATYYCQHFWST |
| | PWTFGGGTKVEIKRTVAAPSVFIFPPSDEQLKSGTASVVCLLNNFYPRE |
| | AKVQWKVDNALQSGNSQESVTEQDSKDSTYSLSSTLTLSKADYEKHK |
| | VYACEVTHQGLSSPVTKSFNRGEC |
| SEQ ID | QVQLVQSGAEVKKPGSSVKVSCKASGYTLTRYWMQWVRQAPGQ |
| NO: 157 | GLEWMGAIYPGDGDTRYSQKFKGRVTITADKSTSTAYMELSSLRSED |
| | TAVYYCAFPEGYYAAPYGMDYWGQGTTVTVSS |
| SEQ ID | DIQMTQSPSSLSASVGDRVTITCRASGNIHNYLAWYQQKPGKVPK |
| NO: 158 | LLIYNAKTLAEGVPSRFSGSRSETDYTLTISSLQPEDVATYYCQHFWST |
| | PWTFGGGTKVEIK |
| SEQ ID | QVQLVQSGAEVKKPGSSVKVSCKASGYTLTRYWMQWVRQAPGQ |
| NO: 159 | GLEWMGAIYPGDGDTRYSQKFKGRVTITADKSTSTAYMELSSLRSED |
| | TAVYYCAFPDGYYAAPYGMDYWGQGTTVTVSS |
| SEQ ID | DIQMTQSPSSLSASVGDRVTITCRASGNIHNYLAWYQQKPGKVPK |
| NO: 160 | LLIYNAKTLADGVPSRFSGSRSETDYTLTISSLQPEDVATYYCQHFWST |
| | PWTFGGGTKVEIK |
| SEQ ID | caggtgcagctggttcagtctggcgccgaagtgaagaaacccggcagcagcgtgaaggtgtcctgtaaa |
| NO: 161 | gccagcggctacaccctgaccagatactggatgcagtgggttcgacaggcccctggacaaggacttgagtgg |
| | atgggagctatctaccctggcgacggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgacggctactacgccgctccttacggcatggattattggggccagggcaccaccgtgaccgt |
| | ttcttct |
| SEQ ID | gacatccagatgacacagagccctagcagcctgtctgcctctgtgggcgatagagtgaccatcacatgca |
| NO: 162 | gagccagcggcaacatccacaactacctggcctggtatcagcagaaacccggcaaggtgcccaagctgctga |
| | tctacaatgccaagacactggccgatggcgtgccaagcagattttccggcagcagaagcgagacagactaca |
| | ccctgaccatcagcagcctgcagcctgaggatgtggccacctactactgccagcacttttggagcaccccttgg |
| | acctttggcggaggcaccaaggtggaaatcaag |
| SEQ ID | caggtgcagctggttcagtctggcgccgaagtgaagaaacccggcagcagcgtgaaggtgtcctgtaaa |
| NO: 163 | gccagcggctacaccctgaccagatactggatgcagtgggttcgacaggcccctggacaaggacttgagtgg |
| | atgggagctatctaccctggcgacggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggctactacgccgctccttacggcatggattattggggccagggcaccaccgtgaccgt |
| | ttcttct |
| SEQ ID | gacatccagatgacacagagccctagcagcctgtctgcctctgtgggcgatagagtgaccatcacatgca |
| NO: 164 | gagccagcggcaacatccacaactacctggcctggtatcagcagaaacccggcaaggtgcccaagctgctga |
| | tctacaatgccaagacactggccgagggcgtgccaagcagattttctggcagcagaagcgagacagactaca |
| | ccctgaccatcagcagcctgcagcctgaggatgtggccacctactactgccagcacttttggagcaccccttgg |
| | acctttggcggaggcaccaaggtggaaatcaag |
| SEQ ID | caggtgcagctggttcagtctggcgccgaagttaagaaaccaggcgcctctgtgaaggtgtcctgcaag |
| NO: 165 | gcctctggctacaccctgaccagatactggatgcagtgggttcgacaggcccctggacaaggacttgagtgga |
| | tgggagctatctaccctggcgacggcgatacccggtacagccagaaatttcagggcagagtgaccctgaccg |
| | ccgacaagagcacaagcacagcctacatggaactgagcagcctgagaagcgaggacaccgccgtgtactac |
| | tgtgcctttcctgacggctactacgccgctccttacggcatggattattggggccagggcaccctggtcaccgttt |
| | cttct |
| SEQ ID | gacatccagatgacacagagccctagcagcctgtctgcctctgtgggcgatagagtgaccatcacatgca |
| NO: 166 | gagccagcggcaacatccacaactacctggcctggtatcagcagaaacccggcaaggtgcccaagctgctga |
| | tctacaatgccaagacactggccgatggcgtgccaagcagattttccggcagcagaagcgagacacagtaca |
| | gcctgacaatcagcagcctgcagcctgaggatgtggccacctactactgccagcacttttggagcaccccttgg |
| | acctttggccagggcaccaaggtggaaatcaag |
| SEQ ID | caggtgcagctggttcagtctggcgccgaagtgaagaaacccggcagcagcgtgaaggtgtcctgtaaa |
| NO: 167 | gccagcggctacaccctgaccagatactggatgcagtgggttcgacaggcccctggacaaggacttgagtgg |
| | atgggagctatctaccctggcgacggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgacggctactacgccgctccttacggcatggattattggggccagggcaccaccgtgaccgt |
| | ttcttctgctagcaccaagggccccagcgtgttccccctggcccccagcagcaagagcaccagcggcggcac |
| | agccgccctgggctgcctggtgaaggactacttccccgagcccgtgaccgtgtcctggaacagcggagccct |
| | gacctccggcgtgcacaccttccccgccgtgctgcagagcagcggcctgtacagcctgtccagcgtggtgac |
| | agtgcccagcagcagcctgggcacccagacctacatctgcaacgtgaaccacaagcccagcaacaccaagg |
| | tggacaagagagtggagcccaagagctgc |
| SEQ ID | gacatccagatgacacagagccctagcagcctgtctgcctctgtgggcgatagagtgaccatcacatgca |
| NO: 168 | gagccagcggcaacatccacaactacctggcctggtatcagcagaaacccggcaaggtgcccaagctgctga |
| | tctacaatgccaagacactggccgatggcgtgccaagcagattttccggcagcagaagcgagacagactaca |
| | ccctgaccatcagcagcctgcagcctgaggatgtggccacctactactgccagcacttttggagcaccccttgg |
| | acctttggcggaggcaccaaggtggaaatcaagcgtacggtggccgctcccagcgtgttcatcttccccccca |
| | gcgacgagcagctgaagagcggcaccgccagcgtggtgtgcctgctgaacaacttctacccccgggaggcc |
| | aaggtgcagtggaaggtggacaacgccctgcagagcggcaacagccaggagagcgtcaccgagcaggac |
| | agcaaggactccacctacagcctgagcagcaccctgaccctgagcaaggccgactacgagaagcataaggt |
| | gtacgcctgcgaggtgacccaccagggcctgtccagccccgtgaccaagagcttcaacaggggcgagtgc |
| SEQ ID | caggtgcagctggttcagtctggcgccgaagtgaagaaacccggcagcagcgtgaaggtgtcctgtaaa |
| NO: 169 | gccagcggctacaccctgaccagatactggatgcagtgggttcgacaggcccctggacaaggacttgagtgg |
| | atgggagctatctaccctggcgacggcgatacccggtacagccagaaattcaagggcagagtgaccatcacc |
| | gccgacaagagcacaagcaccgcctacatggaactgagcagcctgagaagcgaggacaccgccgtgtacta |
| | ctgtgcctttcctgagggctactacgccgctccttacggcatggattattggggccagggcaccaccgtgaccgt |
| | ttcttctgctagcaccaagggccccagcgtgttccccctggcccccagcagcaagagcaccagcggcggcac |
| | agccgccctgggctgcctggtgaaggactacttccccgagcccgtgaccgtgtcctggaacagcggagccct |
| | gacctccggcgtgcacaccttccccgccgtgctgcagagcagcggcctgtacagcctgtccagcgtggtgac |
| | agtgcccagcagcagcctgggcacccagacctacatctgcaacgtgaaccacaagcccagcaacaccaagg |
| | tggacaagagagtggagcccaagagctgc |
| SEQ ID | gacatccagatgacacagagccctagcagcctgtctgcctctgtgggcgatagagtgaccatcacatgca |
| NO: 170 | gagccagcggcaacatccacaactacctggcctggtatcagcagaaacccggcaaggtgcccaagctgctga |
| | tctacaatgccaagacactggccgagggcgtgccaagcagattttctggcagcagaagcgagacagactaca |
| | ccctgaccatcagcagcctgcagcctgaggatgtggccacctactactgccagcacttttggagcaccccttgg |
| | acctttggcggaggcaccaaggtggaaatcaagcgtacggtggccgctcccagcgtgttcatcttccccccca |
| | gcgacgagcagctgaagagcggcaccgccagcgtggtgtgcctgctgaacaacttctacccccgggaggcc |
| | aaggtgcagtggaaggtggacaacgccctgcagagcggcaacagccaggagagcgtcaccgagcaggac |
| | agcaaggactccacctacagcctgagcagcaccctgaccctgagcaaggccgactacgagaagcataaggt |
| | gtacgcctgcgaggtgacccaccagggcctgtccagccccgtgaccaagagcttcaacaggggcgagtgc |
| SEQ ID | caggtgcagctggttcagtctggcgccgaagttaagaaaccaggcgcctctgtgaaggtgtcctgcaag |
| NO: 171 | gcctctggctacaccctgaccagatactggatgcagtgggttcgacaggcccctggacaaggacttgagtgga |
| | tgggagctatctaccctggcgacggcgatacccggtacagccagaaatttcagggcagagtgaccctgaccg |
| | ccgacaagagcacaagcacagcctacatggaactgagcagcctgagaagcgaggacaccgccgtgtactac |
| | tgtgcctttcctgacggctactacgccgctccttacggcatggattattggggccagggcaccctggtcaccgttt |
| | cttctgctagcaccaagggccccagcgtgttccccctggcccccagcagcaagagcaccagcggcggcaca |
| | gccgccctgggctgcctggtgaaggactacttccccgagcccgtgaccgtgtcctggaacagcggagccctg |
| | acctccggcgtgcacaccttccccgccgtgctgcagagcagcggcctgtacagcctgtccagcgtggtgaca |
| | gtgcccagcagcagcctgggcacccagacctacatctgcaacgtgaaccacaagcccagcaacaccaaggt |
| | ggacaagagagtggagcccaagagctgc |
| SEQ ID | Gacatccagatgacacagagccctagcagcctgtctgcctctgtgggcgatagagtgaccatcacatgc |
| NO: 172 | agagccagcggcaacatccacaactacctggcctggtatcagcagaaacccggcaaggtgcccaagctgctg |
| | atctacaatgccaagacactggccgatggcgtgccaagcagattttccggcagcagaagcgagacacagtac |
| | agcctgacaatcagcagcctgcagcctgaggatgtggccacctactactgccagcacttttggagcaccccttg |
| | gacctttggccagggcaccaaggtggaaatcaagcgtacggtggccgctcccagcgtgttcatcttccccccc |
| | agcgacgagcagctgaagagcggcaccgccagcgtggtgtgcctgctgaacaacttctacccccgggaggc |
| | caaggtgcagtggaaggtggacaacgccctgcagagcggcaacagccaggagagcgtcaccgagcagga |
| | cagcaaggactccacctacagcctgagcagcaccctgaccctgagcaaggccgactacgagaagcataagg |
| | tgtacgcctgcgaggtgacccaccagggcctgtccagccccgtgaccaagagcttcaacaggggcgagtgc |
| SEQ ID | SHTLPVRLLRPSDDVQKIVEELQSLSKMLLKDVEEEKGVLVSQNY |
| NO: 173 | TLPCLSPDAQPPNNIHSPAIRAYLKTIRQLDNKSVIDEIIEHLDKLIFQDA |
| | PETNISVPTDTHESKRFILTISQQFSECMDLALKSLTSGAQQATT |
| SEQ ID | EVQLVESGGGLVQPGGSLRLSCAASGFSSSSAHYMCWVRQAPGK |
| NO: 174 | GLEWIGCIYAGSRGSTYYASWAKGRFTISRDNSKNTVYLQMNSLRAE |
| | DTAVYYCARAAFVNRGVSWIWPYYFSLWGQGTLVTVSSASTKGPSVF |
| | PLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPP |
| | CPPCPAPEFLGGPSVFLFPPKPKDTLMISRTPEVTCVVVDVSQEDPEVQ |
| | FNWYVDGVEVHNAKTKPREEQFNSTYRVVSVLTVLHQDWLNGKEY |
| | KCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSR |
| | WQEGNVFSCSVMHEALHNHYTQKSLSLSLGGGGGSGGGGSAFQLTQ |
| | SPSSLSASVGDRVTITCQASQSIDSWLAWYQQKPGKPPKLLIYQASKL |
| | ASGVPSRFSGSGSGTDYTLTISSLQPEDFATYYCQTYYGGGHIGWGFG |
| | TGTKVTVLGGGGGSGGGGSGGGGSGGGGSQSQLVESGGGRVQPGGS |
| | LRLSCAASGFSFSANYWICWVRQAPGKGLEWIVCIYIGSRADTYYAP |
| | WAKGRFTISKDNSKNTVYLQMNSLRAEDTATYFCARFSSGIYDLDRFF |
| | LWGQGTQVTVSS |
| SEQ ID | DIQMTQSPSSLSARVGDRVTITCQASEDIESYLAWYQQKPGKAPKL |
| NO: 175 | LIYRASTLAYGVPSRFSGSGSGRDFTLTISSLQPEDFATYYCLGSSYSTG |
| | SNIFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKHK |
| | VYACEVTHQGLSSPVTKSFNRGEC |

1. A multispecific antibody, wherein the antibody comprises:
(i) a first part that binds to IL-31 comprising:
an HCDR1 comprising SEQ ID NO: 1;
an HCDR2 comprising SEQ ID NO: 2 or SEQ ID NO: 21;
an HCDR3 comprising SEQ ID NO: 3 or SEQ ID NO: 23; and,
an LCDR1 comprising SEQ ID NO: 4 or SEQ ID NO: 24;
an LCDR2 comprising SEQ ID NO: 5 or SEQ ID NO: 25, and;
an LCDR3 comprising SEQ ID NO: 6 or SEQ ID NO: 26, and
(ii) a second part that binds to IL-13.

2. (canceled)

3. The multispecific antibody according to claim 1, wherein the antibody is bispecific.

4. The multispecific antibody according to claim 1, wherein the first part that binds to IL-31 comprises:
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or;
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or;
i) an HCDR1 comprising SEQ ID NO: 1;
ii) an HCDR2 comprising SEQ ID NO: 21;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or;
i) an HCDR1 comprising SEQ ID NO: 1;
ii) an HCDR2 comprising SEQ ID NO: 21;
iii) an HCDR3 comprising SEQ ID NO:3;
iv) an LCDR1 comprising SEQ ID NO: 4;
v) an LCDR2 comprising SEQ ID NO: 5; and
vi) an LCDR3 comprising SEQ ID NO:6,
or;
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 26,
or;
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 24;
v) an LCDR2 comprising SEQ ID NO: 5; and
vi) an LCDR3 comprising SEQ ID NO: 26,
or;
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 25; and
vi) an LCDR3 comprising SEQ ID NO: 26,
or;
i) an HCDR1 comprising SEQ ID NO: 1;
ii) an HCDR2 comprising SEQ ID NO: 21;
iii) an HCDR3 comprising SEQ ID NO: 23;
iv) an LCDR1 comprising SEQ ID NO: 4;
v) an LCDR2 comprising SEQ ID NO: 5; and
vi) an LCDR3 comprising SEQ ID NO: 6,
or;
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 26,
or;
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 24;
v) an LCDR2 comprising SEQ ID NO: 5; and
vi) an LCDR3 comprising SEQ ID NO: 26,
or;
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 25; and
vi) an LCDR3 comprising SEQ ID NO: 26,
or;
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6.

5. The multispecific antibody according to claim 1, wherein the first part that binds to IL-31 comprises:
i) a heavy chain variable region (VH) comprising the amino acid sequence shown in SEQ ID NO: 28; and
ii) a light chain variable region (VL) comprising the amino acid sequence shown in SEQ ID NO: 31;
or
i) a heavy chain variable region (VH) comprising the amino acid sequence shown in SEQ ID NO: 28; and
ii) a light chain variable region (VL) comprising the amino acid sequence shown in SEQ ID NO: 32;
or
i) a heavy chain variable region (VH) comprising the amino acid sequence shown in SEQ ID NO: 28; and
ii) a light chain variable region (VL) comprising the amino acid sequence shown in SEQ ID NO: 30;
or
i) a heavy chain variable region (VH) comprising the amino acid sequence shown in SEQ ID NO: 28; and
ii) a light chain variable region (VL) comprising the amino acid sequence shown in SEQ ID NO: 8;
or
i) a heavy chain variable region (VH) comprising the amino acid sequence shown in SEQ ID NO: 29; and
ii) a light chain variable region (VL) comprising the amino acid sequence shown in SEQ ID NO: 31;
or
i) a heavy chain variable region (VH) comprising the amino acid sequence shown in SEQ ID NO: 29; and
ii) a light chain variable region (VL) comprising the amino acid sequence shown in SEQ ID NO: 32;
or
i) a heavy chain variable region (VH) comprising the amino acid sequence shown in SEQ ID NO: 29; and
ii) a light chain variable region (VL) comprising the amino acid sequence shown in SEQ ID NO: 30;
or
i) a heavy chain variable region (VH) comprising the amino acid sequence shown in SEQ ID NO: 29; and
ii) a light chain variable region (VL) comprising the amino acid sequence shown in SEQ ID NO: 8;
or
i) a heavy chain variable region (VH) comprising the amino acid sequence shown in SEQ ID NO: 7; and
ii) a light chain variable region (VL) comprising the amino acid sequence shown in SEQ ID NO: 31;
or
i) a heavy chain variable region (VH) comprising the amino acid sequence shown in SEQ ID NO: 7; and
ii) a light chain variable region (VL) comprising the amino acid sequence shown in SEQ ID NO: 32;
or
i) a heavy chain variable region (VH) comprising the amino acid sequence shown in SEQ ID NO: 7; and
ii) a light chain variable region (VL) comprising the amino acid sequence shown in SEQ ID NO: 30;
or
i) a heavy chain variable region (VH) comprising the amino acid sequence shown in SEQ ID NO: 7; and
ii) a light chain variable region (VL) comprising the amino acid sequence shown in SEQ ID NO: 8.

6. (canceled)

7. The multispecific antibody according to claim 1, wherein:
the first part that binds to IL-31 comprises:
i) an HCDR1 comprising SEQ ID NO: 1;
ii) an HCDR2 comprising SEQ ID NO: 2;
iii) an HCDR3 comprising SEQ ID NO: 3;
iv) an LCDR1 comprising SEQ ID NO: 4;
v) an LCDR2 comprising SEQ ID NO: 5; and
vi) an LCDR3 comprising SEQ ID NO: 6,
and
the second part that binds to IL-13 comprises:
i) an HCDR1 comprising SEQ ID NO: 11;
ii) an HCDR2 comprising SEQ ID NO: 12;
iii) an HCDR3 comprising SEQ ID NO: 13;
iv) an LCDR1 comprising SEQ ID NO: 14;
v) an LCDR2 comprising SEQ ID NO: 15; and
vi) an LCDR3 comprising SEQ ID NO: 16,
preferably wherein the HCDR1, HCDR2, HCDR3, LCDR1, LCDR2, and LCDR3 are defined according to the Kabat numbering scheme.

8. The multispecific antibody according to claim 1, wherein:
the first part that binds to IL-31 comprises a heavy chain variable region (VH) comprising the amino acid sequence shown in SEQ ID NO: 7 and a light chain variable region (VL) comprising the amino acid sequence shown in SEQ ID NO: 8; and
the second part that binds to IL-13 comprises a heavy chain variable region (VH) comprising the amino acid sequence shown in SEQ ID NO: 17 and a light chain variable region (VL) comprising the amino acid sequence shown in SEQ ID NO: 18.

9. The multispecific antibody according to claim 1, wherein
the first part that binds to IL-31 comprises a first Fc polypeptide, and
the second part that binds to IL-13 comprises a second Fc polypeptide, wherein the first and/or second Fc polypeptide comprises the amino acid sequence shown in SEQ ID NO: 42 or a variant thereof.

10. The multispecific antibody according to claim 9 wherein the first and/or second Fc polypeptide comprises a variant of SEQ ID NO: 42 comprising one or more mutations selected from:
a. one or more silencing mutations e.g., an L234A and an L235A mutation (“LALA”); and/or
b. one or more half-life extension mutations e.g., an M252Y, an S254T and a T256E mutation “YTE”; and/or
c. one or more K-i-H mutations e.g., an S354C and/or a T366W mutation or an Y349C, a T366S, an L368A, and/or Y407V mutation, or FAE mutations e.g., an F405L mutation or a K409R mutation,
according to EU numbering.

11. The multispecific antibody according to claim 9, wherein:
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) an F405L mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an K409R mutation;
or
the first part that binds to IL-31 comprises a first Fc polypeptide comprising a variant of SEQ ID NO: 42 comprising:
a) an L234A and an L235A mutation (“LALA”);
b) an M252Y, an S254T, and a T256E mutation (“YTE”); and
c) an K409R mutation, and
the second part that binds to IL-13 comprises a second Fc polypeptide comprising a variant of SEQ ID NO: 42 comprising:
a) an L234A and an L235A mutation (“LALA”);
b) an M252Y, an S254T, and a T256E mutation (“YTE”); and
c) an F405L mutation,
according to EU numbering.

12. The multispecific antibody according to claim 9, wherein:
i) the first part that binds to IL-31 comprises a first Fc polypeptide comprising the amino acid sequence shown in SEQ ID NO: 44, and the second part that binds to IL-13 comprises a second Fc polypeptide comprising the amino acid sequence shown in SEQ ID NO: 45; or
ii) the first part that binds to IL-31 comprises a first Fc polypeptide comprising the amino acid sequence shown in SEQ ID NO: 45, and the second part that binds to IL-13 comprises a second Fc polypeptide comprising the amino acid sequence shown in SEQ ID NO: 44.

13. (canceled)

14. A multispecific antibody according to claim 2, wherein the first part that binds to IL-31 comprises:
i) a light chain comprising or consisting of the amino acid sequence shown in SEQ ID NO: 9; and
ii) a heavy chain comprising or consisting of the amino acid sequence shown in SEQ ID NO: 10;
and wherein the second part that binds to IL-13 comprises:
iii) a light chain comprising or consisting of the amino acid sequence shown in SEQ ID NO: 19; and
iv) a heavy chain comprising or consisting of the amino acid sequence shown in SEQ ID NO: 20.

15. A multispecific antibody according to claim 1, wherein the first part that binds to IL-31 comprises:
i) a light chain comprising or consisting of the amino acid sequence shown in SEQ ID NO: 9; and
ii) a heavy chain comprising or consisting of the amino acid sequence shown in SEQ ID NO: 109;
and wherein the second part that binds to IL-13 comprises:
iii) a light chain comprising or consisting of the amino acid sequence shown in SEQ ID NO: 19; and
iv) a heavy chain comprising or consisting of the amino acid sequence shown in SEQ ID NO: 110.

16. The multispecific antibody according to claim 1, wherein the antibody is monovalent with respect to binding IL-31 and monovalent with respect to binding IL-13.

17. (canceled)

18. The multispecific antibody according to claim 1, wherein the antibody has an IgG isotype.

19. The multispecific antibody according to claim 1, wherein the antibody is isolated.

20. The multispecific antibody according to claim 1, wherein the IL-31 and IL-13 are human.

21. A pharmaceutical composition comprising the multispecific antibody of claim 1, optionally in combination with one or more pharmaceutically acceptable excipients, diluents, or carriers.

22. (canceled)

23. (canceled)

24. (canceled)

25. (canceled)

26. A method of preventing and/or treating an IL-31- and IL-13-related disease or disorder in a subject in need thereof, comprising administering to the subject a therapeutically effective amount of the multispecific antibody of claim 1.

27. The method of claim 26, wherein the IL-31- and IL-13-related disease or disorder is an inflammatory or immune disease or disorder.

28. The method of claim 27, wherein the inflammatory or immune disease or disorder is a skin disease or disorder.

29. (canceled)

30. An isolated nucleic acid molecule or set of isolated nucleic acid molecules encoding the multispecific antibody of claim 1.

31. (canceled)

32. (canceled)

33. A host cell comprising a nucleic acid molecule encoding the first part that binds to IL-31 of the multispecific antibody claim 1, or an expression vector comprising said nucleic acid molecule.

34. A host cell comprising a nucleic acid molecule encoding the second part that binds to IL-13 of the multispecific antibody claim 1, or an expression vector comprising said nucleic acid molecule.

35. (canceled)

36. (canceled)

37. A method of treating and/or preventing atopic dermatitis, e.g., moderate to severe atopic dermatitis, in a subject in need thereof, comprising administering to the subject a therapeutically effective amount of a multispecific antibody claim 1.

38. (canceled)

39. A method of producing the multispecific antibody of claim 1, said method comprising:
a) culturing a first host cell expressing the first part that binds to IL-31 in a culture under conditions sufficient to express the antibody, and a second host cell expressing the second part that binds to IL-13 in a culture under conditions sufficient to express the antibody,
wherein the first and second host cell are cultured in the same or in different cultures;
b) recovering the first- and second-part from the host cell culture(s);
c) incubating together the first- and second-part recovered in step b), preferably under reducing conditions that allow inter-chain disulfide bond reduction in the hinge region;
d) incubating the first- and second-part under conditions that allow oxidation of cysteines in the hinge region to form inter-chain disulfide bonds between the first- and second-part;
e) obtaining the multispecific antibody; and optionally purifying the multispecific antibody.

40. (canceled)

41. A method of producing the multispecific antibody of claim 1, said method comprising:
a) culturing a first host cell expressing the first part that binds to IL-31 in a culture under conditions sufficient to express the antibody, and a second host cell expressing the second part that binds to IL-13 in a culture under conditions sufficient to express the antibody,
wherein the first and second host cell are cultured in the same culture;
b) incubating together the first- and second-part, preferably under reducing conditions that allow inter-chain disulfide bond reduction in the hinge region;
c) incubating the first- and second-part under conditions that allow oxidation of cysteines in the hinge region to form inter-chain disulfide bonds between the first- and second-part;
d) obtaining the multispecific antibody; and optionally purifying the multispecific antibody,
wherein incubating together the first- and second-part in step b) is done without any prior recovery of the first- and second-art from the culture.

42. A method of producing a pharmaceutical composition comprising the multispecific antibody claim 1, said method comprising:
a) producing the multispecific antibody according to the method of claim 41;
b) formulating the multispecific antibody in a pharmaceutical composition, optionally in combination with one or more pharmaceutically acceptable excipients, diluents, or carriers.


SEQUENCE LISTING (179 sequences)

SEQ ID NO: 1 - protein, 5 aa, Synthetic construct
  source 1..5: note = HCDR1
RYWMQ

SEQ ID NO: 2 - protein, 17 aa, Synthetic construct
  source 1..17: note = HCDR2
AIYPAEGDTRYSQKFKG

SEQ ID NO: 3 - protein, 13 aa, Synthetic construct
  source 1..13: note = HCDR3
PEGPYAAPYGMHY

SEQ ID NO: 4 - protein, 11 aa, Synthetic construct
  source 1..11: note = LCDR1
RASRNIGNYLA

SEQ ID NO: 5 - protein, 7 aa, Synthetic construct
  source 1..7: note = LCDR2
YAKTSAS

SEQ ID NO: 6 - protein, 9 aa, Synthetic construct
  source 1..9: note = LCDR3
QHFWSTPWT

SEQ ID NO: 7 - protein, 122 aa, Synthetic construct
  source 1..122: note = VH
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQGTTVTVSS

SEQ ID NO: 8 - protein, 107 aa, Synthetic construct
  source 1..107: note = VL
AIQMTQSPSSLSASVGDRVTITCRASRNIGNYLAWYQQKPGKVPKLLIYYAKTSASGVPSRFSGSRSGTDYTLTISSLQPEDVATYYCQHFWSTPWTFGGGTKVEIK

SEQ ID NO: 9 - protein, 214 aa, Synthetic construct
  source 1..214: note = Light Chain
AIQMTQSPSSLSASVGDRVTITCRASRNIGNYLAWYQQKPGKVPKLLIYYAKTSASGVPSRFSGSRSGTDYTLTISSLQPEDVATYYCQHFWSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Synthetic construct
  source 1..452: note = IL-31 antibody Heavy Chain (LALA; YTE ; F405L)
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 11 - protein, 5 aa, Synthetic construct
  source 1..5: note = HCDR1
AYSVN

SEQ ID NO: 12 - protein, 16 aa, Synthetic construct
  source 1..16: note = HCDR2
MIWGDGKIVYNSALKS

SEQ ID NO: 13 - protein, 10 aa, Synthetic construct
  source 1..10: note = HCDR3
DGYYPYAMDN

SEQ ID NO: 14 - protein, 15 aa, Synthetic construct
  source 1..15: note = LCDR1
RASKSVDSYGNSFMH

SEQ ID NO: 15 - protein, 7 aa, Synthetic construct
  source 1..7: note = LCDR2
LASNLES

SEQ ID NO: 16 - protein, 9 aa, Synthetic construct
  source 1..9: note = LCDR3
QQNNEDPRT

SEQ ID NO: 17 - protein, 118 aa, Synthetic construct [sequence omitted]

SEQ ID NO: 18 - protein, 111 aa, Synthetic construct [sequence omitted]

SEQ ID NO: 19 - protein, 218 aa, Synthetic construct
  source 1..218: note = Light Chain
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 448 aa, Synthetic construct
  source 1..448: note = IL-13 antibody Heavy Chain (LALA; YTE ; K409R)
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 21 - protein, 17 aa, Synthetic construct
  source 1..17: note = HCDR2
AIYPGEGDTRYSQKFKG

SEQ ID NO: 22 - skipped

SEQ ID NO: 23 - protein, 13 aa, Synthetic construct
  source 1..13: note = HCDR3
PEGQYAAPYGMHY

SEQ ID NO: 24 - protein, 11 aa, Synthetic construct
  source 1..11: note = LCDR1
RASRNIHNYLA

SEQ ID NO: 25 - protein, 7 aa, Synthetic construct
  source 1..7: note = LCDR2
YAKTSAE

SEQ ID NO: 26 - protein, 9 aa, Synthetic construct
  source 1..9: note = LCDR3
QHFWKTPWT

SEQ ID NO: 27 - skipped

SEQ ID NO: 28 - protein, 122 aa, Synthetic construct
  source 1..122: note = VH
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGPYAAPYGMHYWGQGTTVTVSS

SEQ ID NO: 29 - protein, 122 aa, Synthetic construct
  source 1..122: note = VH
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGQYAAPYGMHYWGQGTTVTVSS

SEQ ID NO: 30 - protein, 107 aa, Synthetic construct
  source 1..107: note = VL
AIQMTQSPSSLSASVGDRVTITCRASRNIGNYLAWYQQKPGKVPKLLIYYAKTSAEGVPSRFSGSRSGTDYTLTISSLQPEDVATYYCQHFWKTPWTFGGGTKVEIK

SEQ ID NO: 31 - protein, 107 aa, Synthetic construct
  source 1..107: note = VL
AIQMTQSPSSLSASVGDRVTITCRASRNIGNYLAWYQQKPGKVPKLLIYYAKTSASGVPSRFSGSRSGTDYTLTISSLQPEDVATYYCQHFWKTPWTFGGGTKVEIK

SEQ ID NO: 32 - protein, 107 aa, Synthetic construct
  source 1..107: note = VL
AIQMTQSPSSLSASVGDRVTITCRASRNIHNYLAWYQQKPGKVPKLLIYYAKTSASGVPSRFSGSRSGTDYTLTISSLQPEDVATYYCQHFWKTPWTFGGGTKVEIK

SEQ ID NO: 33 - protein, 214 aa, Synthetic construct
  source 1..214: note = Light Chain
AIQMTQSPSSLSASVGDRVTITCRASRNIGNYLAWYQQKPGKVPKLLIYYAKTSAEGVPSRFSGSRSGTDYTLTISSLQPEDVATYYCQHFWKTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, Synthetic construct
  source 1..214: note = Light Chain
AIQMTQSPSSLSASVGDRVTITCRASRNIGNYLAWYQQKPGKVPKLLIYYAKTSASGVPSRFSGSRSGTDYTLTISSLQPEDVATYYCQHFWKTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 214 aa, Synthetic construct
  source 1..214: note = Light Chain
AIQMTQSPSSLSASVGDRVTITCRASRNIHNYLAWYQQKPGKVPKLLIYYAKTSASGVPSRFSGSRSGTDYTLTISSLQPEDVATYYCQHFWKTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - skipped

SEQ ID NO: 37 - protein, 225 aa, Synthetic construct
  source 1..225: note = Fd
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 38 - protein, 225 aa, Synthetic construct
  source 1..225: note = Fd
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGQYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 39 - protein, 225 aa, Synthetic construct
  source 1..225: note = Fd
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 40 - protein, 122 aa, Synthetic construct
  source 1..122: note = TPP-20012 VH
QVQLVQSGAEVKKPGASVKVSCKASGYTLTRYWMQWVRQAPGQGLEWMGAIYPGDGDTRYSQKFQGRVTLTADKSTSTAYMELSSLRSEDTAVYYCAFPDGYYAAPYGMDYWGQGTLVTVSS

SEQ ID NO: 41 - protein, 107 aa, Synthetic construct
  source 1..107: note = TPP-20012 VL
DIQMTQSPSSLSASVGDRVTITCRASGNIHNYLAWYQQKPGKVPKLLIYNAKTLADGVPSRFSGSRSETQYSLTISSLQPEDVATYYCQHFWSTPWTFGQGTKVEIK

SEQ ID NO: 42 - protein, 227 aa, Synthetic construct
  source 1..227: note = Fc (wild-type)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 221 aa, Synthetic construct
  source 1..221: note = Fd
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 44 - protein, 227 aa, Synthetic construct
  source 1..227: note = Fc (LALA; YTE; F405L)
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 45 - protein, 227 aa, Synthetic construct
  source 1..227: note = Fc (LALA; YTE ; K409R)
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 46 - protein, 227 aa, Synthetic construct
  source 1..227: note = Fc with Knob mutations (S354C, T366W)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 227 aa, Synthetic construct
  source 1..227: note = Fc with Hole mutations (Y349C, T366S, L368A, Y407V)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 7 aa, Synthetic construct
  source 1..7: note = HCDR1
GYTRTRY

SEQ ID NO: 49 - protein, 6 aa, Synthetic construct
  source 1..6: note = HCDR2
YPAEGD

SEQ ID NO: 50 - protein, 13 aa, Synthetic construct
  source 1..13: note = HCDR3
PEGPYAAPYGMHY

SEQ ID NO: 51 - protein, 7 aa, Synthetic construct
  source 1..7: note = LCDR1
SRNIGNY

SEQ ID NO: 52 - skipped

SEQ ID NO: 53 - protein, 6 aa, Synthetic construct
  source 1..6: note = LCDR3
FWSTPW

SEQ ID NO: 54 - protein, 7 aa, Synthetic construct
  source 1..7: note = HCDR1
GFSLSAY

SEQ ID NO: 55 - protein, 5 aa, Synthetic construct
  source 1..5: note = HCDR2
WGDGK

SEQ ID NO: 56 - protein, 10 aa, Synthetic construct
  source 1..10: note = HCDR3
DGYYPYAMDN

SEQ ID NO: 57 - protein, 11 aa, Synthetic construct
  source 1..11: note = LCDR1
SKSVDSYGNSF

SEQ ID NO: 58 - skipped

SEQ ID NO: 59 - protein, 6 aa, Synthetic construct
  source 1..6: note = LCDR3
NNEDPR

SEQ ID NO: 60 - skipped

SEQ ID NO: 61 - protein, 6 aa, Synthetic construct
  source 1..6: note = HCDR2
YPGEGD

SEQ ID NO: 62 - skipped

SEQ ID NO: 63 - protein, 13 aa, Synthetic construct
  source 1..13: note = HCDR3
PEGQYAAPYGMHY

SEQ ID NO: 64 - protein, 6 aa, Synthetic construct
  source 1..6: note = LCDR3
FWKTPW

SEQ ID NO: 65 - protein, 7 aa, Synthetic construct
  source 1..7: note = LCDR1
SRNIHNY

SEQ ID NO: 66 - protein, 8 aa, Synthetic construct
  source 1..8: note = HCDR1
GYTRTRYW

SEQ ID NO: 67 - protein, 8 aa, Synthetic construct
  source 1..8: note = HCDR2
IYPAEGDT

SEQ ID NO: 68 - protein, 15 aa, Synthetic construct
  source 1..15: note = HCDR3
AFPEGPYAAPYGMHY

SEQ ID NO: 69 - protein, 6 aa, Synthetic construct
  source 1..6: note = LCDR1
RNIGNY

SEQ ID NO: 70 - skipped

SEQ ID NO: 71 - protein, 9 aa, Synthetic construct
  source 1..9: note = LCDR3
QHFWSTPWT

SEQ ID NO: 72 - skipped

SEQ ID NO: 73 - protein, 8 aa, Synthetic construct
  source 1..8: note = HCDR2
IYPGEGDT

SEQ ID NO: 74 - skipped

SEQ ID NO: 75 - protein, 9 aa, Synthetic construct
  source 1..9: note = LCDR3
QHFWKTPWT

SEQ ID NO: 76 - protein, 6 aa, Synthetic construct
  source 1..6: note = LCDR1
RNIHNY

SEQ ID NO: 77 - protein, 15 aa, Synthetic construct
  source 1..15: note = HCDR3
AFPEGQYAAPYGMHY

SEQ ID NO: 78 - protein, 10 aa, Synthetic construct
  source 1..10: note = HCDR1
GYTRTRYWMQ

SEQ ID NO: 79 - protein, 17 aa, Synthetic construct
  source 1..17: note = HCDR2
AIYPAEGDTRYSQKFKG

SEQ ID NO: 80 - protein, 13 aa, Synthetic construct
  source 1..13: note = HCDR3
PEGPYAAPYGMHY

SEQ ID NO: 81 - protein, 11 aa, Synthetic construct
  source 1..11: note = LCDR1
RASRNIGNYLA

SEQ ID NO: 82 - protein, 7 aa, Synthetic construct
  source 1..7: note = LCDR2
YAKTSAS

SEQ ID NO: 83 - protein, 9 aa, Synthetic construct
  source 1..9: note = LCDR3
QHFWSTPWT

SEQ ID NO: 84 - protein, 10 aa, Synthetic construct
  source 1..10: note = HCDR1
GFSLSAYSVN

SEQ ID NO: 85 - protein, 16 aa, Synthetic construct
  source 1..16: note = HCDR2
MIWGDGKIVYNSALKS

SEQ ID NO: 86 - protein, 10 aa, Synthetic construct
  source 1..10: note = HCDR3
DGYYPYAMDN

SEQ ID NO: 87 - protein, 15 aa, Synthetic construct
  source 1..15: note = LCDR1
RASKSVDSYGNSFMH

SEQ ID NO: 88 - protein, 7 aa, Synthetic construct
  source 1..7: note = LCDR2
LASNLES

SEQ ID NO: 89 - protein, 9 aa, Synthetic construct
  source 1..9: note = LCDR3
QQNNEDPRT

SEQ ID NO: 90 - skipped

SEQ ID NO: 91 - protein, 17 aa, Synthetic construct
  source 1..17: note = HCDR2
AIYPGEGDTRYSQKFKG

SEQ ID NO: 92 - skipped

SEQ ID NO: 93 - protein, 7 aa, Synthetic construct
  source 1..7: note = LCDR2
YAKTSAE

SEQ ID NO: 94 - protein, 9 aa, Synthetic construct
  source 1..9: note = LCDR3
QHFWKTPWT

SEQ ID NO: 95 - protein, 11 aa, Synthetic construct
  source 1..11: note = LCDR1
RASRNIHNYLA

SEQ ID NO: 96 - protein, 13 aa, Synthetic construct
  source 1..13: note = HCDR3
PEGQYAAPYGMHY

SEQ ID NO: 97 - protein, 8 aa, Synthetic construct
  source 1..8: note = HCDR1
GFSLSAYS

SEQ ID NO: 98 - protein, 7 aa, Synthetic construct
  source 1..7: note = HCDR2
IWGDGKI

SEQ ID NO: 99 - protein, 12 aa, Synthetic construct
  source 1..12: note = HCDR3
AGDGYYPYAMDN

SEQ ID NO: 100 - protein, 10 aa, Synthetic construct
  source 1..10: note = LCDR1
KSVDSYGNSF

SEQ ID NO: 101 - skipped

SEQ ID NO: 102 - protein, 9 aa, Synthetic construct
  source 1..9: note = LCDR3
QQNNEDPRT

SEQ ID NO: 103 - skipped

SEQ ID NO: 104 - protein, 452 aa, Synthetic construct
  source 1..452: note = IL-31 antibody Heavy chain (LALA; YTE ; F405L)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 105 - protein, 452 aa, Synthetic construct
  source 1..452: note = IL-31 antibody Heavy chain (LALA; YTE ; F405L)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGQYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 106 - skipped

SEQ ID NO: 107 - protein, 452 aa, Synthetic construct
  source 1..452: note = IL-31 antibody Heavy Chain (LALA; YTE ; K409R)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 108 - protein, 452 aa, Synthetic construct
  source 1..452: note = IL-31 antibody Heavy Chain (LALA; YTE ; K409R)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGQYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 109 - protein, 452 aa, Synthetic construct
  source 1..452: note = IL-31 antibody Heavy Chain (LALA; YTE ; K409R)
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 110 - protein, 448 aa, Synthetic construct
  source 1..448: note = IL-13 antibody Heavy Chain (LALA; YTE ; F405L)
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 111 - skipped

SEQ ID NO: 112 - protein, 452 aa, Synthetic construct
  source 1..452: note = Heavy Chain with Knob mutations (S354C, T366W)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 452 aa, Synthetic construct
  source 1..452: note = Heavy Chain with Knob mutations (S354C, T366W)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGQYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 452 aa, Synthetic construct
  source 1..452: note = heavy chain with Knob mutations (S354C, T366W)
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - skipped

SEQ ID NO: 116 - protein, 452 aa, Synthetic construct
  source 1..452: note = Heavy Chain with Hole mutations (Y349C, T366S, L368A, Y407V)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 117 - protein, 452 aa, Synthetic construct
  source 1..452: note = Heavy Chain with Hole mutations (Y349C, T366S, L368A, Y407V)
QVQLVQSGAEVKKPGSSVKVSCKASGYTRTRYWMQWVRQAPGQGLEWMGAIYPGEGDTRYSQKFKGRVTITADKSTSTAYMELSSLRSEDTAVYYCAFPEGQYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 118 - protein, 452 aa, Synthetic construct
  source 1..452: note = Heavy Chain with Hole mutations (Y349C, T366S, L368A, Y407V)
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 119 - DNA, 366 bp, Synthetic construct
  source 1..366: mol_type = other DNA; note = IL-31 VH DNA
caggttcagttggtgcagtccggcgctgaagtgaagaaacctggctcctctgtgaaggtgtcctgcaaggcctctggctacacccggaccagatattggatgcagtgggtccgacaggctcctggacaaggattggagtggatgggcgctatctaccctgctgagggcgacacccggtactcccagaaattcaagggcagagtgaccatcaccgccgacaagtctacctccaccgcctacatggaactgtccagcctgagatccgaggacaccgccgtgtactactgtgcctttcctgagggaccttacgccgctccttacggcatgcattattggggccagggcaccaccgtgaccgtgtcctct

SEQ ID NO: 120 - DNA, 321 bp, Synthetic construct
  source 1..321: mol_type = other DNA; note = IL-31 VL DNA
gccatccagatgacccagtctccatcctctctgtctgcctctgtgggcgacagagtgaccatcacctgtcgggcttcccggaacatcggcaactacctggcctggtatcagcagaaacctggcaaggtgcccaagctgctgatctactacgccaagacctctgccagcggcgtgccaagcagattttctggaagcagatccggcaccgactacaccctgaccatttctagcctgcagcctgaggacgtggccacctactactgtcagcacttctggtctaccccttggacctttggcggaggcaccaaggtggaaatcaag

SEQ ID NO: 121 - DNA, 642 bp, Synthetic construct
  source 1..642: mol_type = other DNA; note = IL-31 Light Chain DNA
gccatccagatgacccagtctccatcctctctgtctgcctctgtgggcgacagagtgaccatcacctgtcgggcttcccggaacatcggcaactacctggcctggtatcagcagaaacctggcaaggtgcccaagctgctgatctactacgccaagacctctgccagcggcgtgccaagcagattttctggaagcagatccggcaccgactacaccctgaccatttctagcctgcagcctgaggacgtggccacctactactgtcagcacttctggtctaccccttggacctttggcggaggcaccaaggtggaaatcaagcgtacggtggccgctccttccgtgttcatcttcccaccatctgacgagcagctgaagtctggcaccgcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaacagaggcgagtgc

SEQ ID NO: 122 - DNA, 1356 bp, Synthetic construct
  source 1..1356: mol_type = other DNA; note = IL-31 Heavy Chain (LALA; YTE ; F405L) DNA
caggttcagttggtgcagtccggcgctgaagtgaagaaacctggctcctctgtgaaggtgtcctgcaaggcctctggctacacccggaccagatattggatgcagtgggtccgacaggctcctggacaaggattggagtggatgggcgctatctaccctgctgagggcgacacccggtactcccagaaattcaagggcagagtgaccatcaccgccgacaagtctacctccaccgcctacatggaactgtccagcctgagatccgaggacaccgccgtgtactactgtgcctttcctgagggaccttacgccgctccttacggcatgcattattggggccagggcaccaccgtgaccgtgtcctctgcttctacaaagggcccctctgtgttccctctggctcctagctctaagtccacctctggtggaaccgctgctctgggctgtctggtcaaggattacttccctgagccagtgacagtgtcctggaactctggtgctctgacctccggcgtgcacacatttccagctgtgctgcagtcctccggcctgtactctctgtcctctgtcgtgacagtgccttccagctctctgggcacccagacctacatctgcaacgtgaaccacaagccttccaataccaaggtggacaagagagtggaacccaagtcctgcgacaagacccacacgtgccctccatgtcctgctccagaagctgctggcggaccctccgttttcctgtttccacctaagcctaaggacaccctgtacatcacccgcgagcctgaagtgacctgtgtggtggtggatgtgtcccacgaggaccccgaagtgaagtttaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacacactgcctccaagccgggaagagatgaccaagaatcaggtgtccctgacctgcctcgtgaagggcttctacccttccgatatcgccgtggaatgggagtctaacggccagccagagaacaactacaagaccacacctcctgtgctggactccgacggctctttcctgctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 123 - DNA, 354 bp, Synthetic construct
  source 1..354: mol_type = other DNA; note = IL-13 VH DNA
caagtcactctgcgcgaatcaggaccggccctggtcaagcccacccagaccctgaccctgacttgtactgtgtcgggtttctccctgtccgcgtatagcgtgaactggattagacagcctcccggaaaggccctggagtggcttgcgatgatctggggggacgggaagatcgtgtacaacagcgccctcaagagccgcctgaccatcagcaaggacacctctaagaatcaggtcgtgctgactatgaccaacatggatcccgtggacaccgccacctactactgtgctggggacggctactatccctacgccatggacaactggggacagggttccctggttactgtgtcctcc

SEQ ID NO: 124 - DNA, 333 bp, Synthetic construct
  source 1..333: mol_type = other DNA; note = IL-13 VL DNA
gacattgtgatgacccagtccccggactccctctcggtgtccctgggagaacgcgcgactatcaactgtcgggcttccaagtccgtggactcctacggaaactcgttcatgcattggtaccagcagaaacctggacagccgccaaagctgctcatctacctggcatccaacctggagagcggagtgccggatagattcagcggatcgggttcaggcaccgacttcaccctgaccatctcatcactccaagcggaggatgtggccgtgtactactgccaacagaacaacgaggatcccaggaccttcgggggtggcactaaggtcgagatcaag

SEQ ID NO: 125 - DNA, 654 bp, Synthetic construct
  source 1..654: mol_type = other DNA; note = IL-13 Light Chain DNA
gacattgtgatgacccagtccccggactccctctcggtgtccctgggagaacgcgcgactatcaactgtcgggcttccaagtccgtggactcctacggaaactcgttcatgcattggtaccagcagaaacctggacagccgccaaagctgctcatctacctggcatccaacctggagagcggagtgccggatagattcagcggatcgggttcaggcaccgacttcaccctgaccatctcatcactccaagcggaggatgtggccgtgtactactgccaacagaacaacgaggatcccaggaccttcgggggtggcactaaggtcgagatcaagcgtacggtggccgcacctagcgtgttcattttcccgccctccgatgaacagctgaagtcgggcactgccagcgtggtctgcctgctgaacaacttttaccctcgggaagccaaggtccagtggaaagtggacaatgccctgcaatccgggaactcccaggagagcgtgaccgaacaggactctaaggacagcacctactcgctctcctcaacccttacactgtccaaggccgactatgagaagcacaaggtctacgcctgcgaagtgacccaccaaggattgtcgtcccccgtgaccaagagcttcaatcggggcgaatgt

SEQ ID NO: 126 - DNA, 1344 bp, Synthetic construct
  source 1..1344: mol_type = other DNA; note = IL-13 Heavy Chain (LALA; YTE ; K409R) DNA
caagtcactctgcgcgaatcaggaccggccctggtcaagcccacccagaccctgaccctgacttgtactgtgtcgggtttctccctgtccgcgtatagcgtgaactggattagacagcctcccggaaaggccctggagtggcttgcgatgatctggggggacgggaagatcgtgtacaacagcgccctcaagagccgcctgaccatcagcaaggacacctctaagaatcaggtcgtgctgactatgaccaacatggatcccgtggacaccgccacctactactgtgctggggacggctactatccctacgccatggacaactggggacagggttccctggttactgtgtcctccgcttcgaccaagggcccgtcggtgtttccactcgccccgagctccaagagcacttcaggcggtactgcagccttgggatgcctcgtgaaggactacttccctgagccggtcaccgtgtcctggaacagcggcgccctgacttccggggtgcacaccttcccggccgtgctgcaatcctccggcctgtactcactgtcctcggtcgtgaccgtgccgagcagcagcctgggcacccagacctacatctgcaacgtgaaccacaagccctccaacaccaaggtcgataagagggtggaacccaagtcctgcgacaagacccacacgtgcccaccttgtcctgcgcctgaagcggctggaggaccatccgtgttcctgttcccgcctaaaccaaaggacacgctctacattactcgcgaaccggaagtcacttgcgtggtggtggatgtgtcgcacgaggatcccgaggtcaagtttaattggtacgtggatggcgtggaagtgcacaacgccaagactaagccgcgggaggaacagtacaactccacctaccgggtggtgtcggtgctgaccgtgctgcatcaggactggctcaacggaaaggagtacaagtgcaaagtgtccaacaaggcacttccagccccgattgagaaaaccatctcaaaggccaagggacagccgagagagccccaggtctacacactccctccttcgcgggaggagatgaccaagaaccaagtgtcacttacttgcctggtcaagggcttctacccgtccgatatcgccgtggagtgggaatccaacggacagccggaaaacaactacaagaccacccctcccgtactggacagcgacggatcgttcttcctgtactcccggctcactgtggacaagtcacggtggcagcagggaaacgtgttctcctgctctgtcatgcatgaggcgctgcataatcactatacccaaaagtccctgtccctgagtcccgggaaa

SEQ ID NO: 127 - DNA, 366 bp, Synthetic construct
  source 1..366: mol_type = other DNA; note = IL-31 VH DNA
caggtgcagctggttcagtctggcgccgaagtgaagaaacccggcagcagcgtgaaggtgtcctgtaaagccagcggctacacccggaccagatactggatgcagtgggttcgacaggcccctggacaaggacttgagtggatgggagccatctatcctggcgagggcgatacccggtacagccagaaattcaagggcagagtgaccatcaccgccgacaagagcacaagcaccgcctacatggaactgagcagcctgagaagcgaggacaccgccgtgtactactgtgcctttcctgagggaccttacgccgctccttacggcatgcattattggggccagggcaccaccgtgaccgtttcttct

SEQ ID NO: 128 - DNA, 366 bp, Synthetic construct
  source 1..366: mol_type = other DNA; note = IL-31 VH DNA
caggtgcagctggttcagtctggcgccgaagtgaagaaacccggcagcagcgtgaaggtgtcctgtaaagccagcggctacacccggaccagatactggatgcagtgggttcgacaggcccctggacaaggacttgagtggatgggagccatctatcctggcgagggcgatacccggtacagccagaaattcaagggcagagtgaccatcaccgccgacaagagcacaagcaccgcctacatggaactgagcagcctgagaagcgaggacaccgccgtgtactactgtgcctttcctgagggacagtacgccgctccttacggcatgcattattggggccagggcaccaccgtgaccgtttcttct

SEQ ID NO: 129 - DNA, 366 bp, Synthetic construct
  source 1..366: mol_type = other DNA; note = IL-31 VH DNA
caggtgcagctggttcagtctggcgccgaagtgaagaaacccggcagcagcgtgaaggtgtcctgtaaagccagcggctacacccggaccagatactggatgcagtgggttcgacaggcccctggacaaggacttgagtggatgggagccatctatcctgccgagggcgatacccggtacagccagaaattcaagggcagagtgaccatcaccgccgacaagagcacaagcaccgcctacatggaactgagcagcctgagaagcgaggacaccgccgtgtactactgtgcctttcctgagggaccttacgccgctccttacggcatgcattattggggccagggcaccaccgtgaccgtttcttct

SEQ ID NO: 130 - DNA, 321 bp, Synthetic construct
  source 1..321: mol_type = other DNA; note = IL-31 VL DNA
gccatccagatgacacagagccctagcagcctgtctgcctctgtgggcgatagagtgaccatcacctgtcgggccagccggaacatcggaaactatctggcctggtatcagcagaaacccggcaaggtgcccaagctgctgatctactatgccaagacaagcgccagcggcgtgccaagcagattttctggcagcagaagcggcaccgactacaccctgaccatttctagcctgcagcctgaggacgtggccacctactactgccagcacttctggaaaaccccttggacctttggcggaggcaccaaggtggaaatcaag

SEQ ID NO: 131 - DNA, 321 bp, Synthetic construct
  source 1..321: mol_type = other DNA; note = IL-31 VL DNA
gccatccagatgacacagagccctagcagcctgtctgcctctgtgggcgatagagtgaccatcacctgtcgggccagccggaacatccacaactatctggcctggtatcagcagaaacccggcaaggtgcccaagctgctgatctactatgccaagacaagcgccagcggcgtgccaagcagattttctggcagcagaagcggcaccgactacaccctgaccatttctagcctgcagcctgaggacgtggccacctactactgccagcacttctggaaaaccccttggacctttggcggaggcaccaaggtggaaatcaag

SEQ ID NO: 132 - DNA, 321 bp, Synthetic construct
  source 1..321: mol_type = other DNA; note = IL-31 VL DNA
gccatccagatgacacagagccctagcagcctgtctgcctctgtgggcgatagagtgaccatcacctgtcgggccagccggaacatcggaaactatctggcctggtatcagcagaaacccggcaaggtgcccaagctgctgatctactatgccaagacatctgccgagggcgtgccaagcagattttctggcagcagaagcggcaccgactacaccctgaccatttctagcctgcagcctgaggacgtggccacctactactgccagcacttctggaaaaccccttggacctttggcggaggcaccaaggtggaaatcaag

SEQ ID NO: 133 - DNA, 321 bp, Synthetic construct
  source 1..321: mol_type = other DNA; note = IL-31 VL DNA
gccatccagatgacacagagccctagcagcctgtctgcctctgtgggcgatagagtgaccatcacctgtcgggccagccggaacatcggaaactatctggcctggtatcagcagaaacccggcaaggtgcccaagctgctgatctactatgccaagacaagcgccagcggcgtgccaagcagattttctggcagcagaagcggcaccgactacaccctgaccatttctagcctgcagcctgaggacgtggccacctactactgtcagcacttttggagcaccccttggacctttggcggaggcaccaaggtggaaatcaag

SEQ ID NO: 134 - DNA, 642 bp, Synthetic construct
  source 1..642: mol_type = other DNA; note = IL-31 Light Chain
gccatccagatgacacagagccctagcagcctgtctgcctctgtgggcgatagagtgaccatcacctgtcgggccagccggaacatcggaaactatctggcctggtatcagcagaaacccggcaaggtgcccaagctgctgatctactatgccaagacaagcgccagcggcgtgccaagcagattttctggcagcagaagcggcaccgactacaccctgaccatttctagcctgcagcctgaggacgtggccacctactactgccagcacttctggaaaaccccttggacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 135 - DNA, 642 bp, Synthetic construct
  source 1..642: mol_type = other DNA; note = IL-31 Light Chain
gccatccagatgacacagagccctagcagcctgtctgcctctgtgggcgatagagtgaccatcacctgtcgggccagccggaacatccacaactatctggcctggtatcagcagaaacccggcaaggtgcccaagctgctgatctactatgccaagacaagcgccagcggcgtgccaagcagattttctggcagcagaagcggcaccgactacaccctgaccatttctagcctgcagcctgaggacgtggccacctactactgccagcacttctggaaaaccccttggacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 136 - DNA, 642 bp, Synthetic construct
  source 1..642: mol_type = other DNA; note = IL-31 Light Chain
gccatccagatgacacagagccctagcagcctgtctgcctctgtgggcgatagagtgaccatcacctgtcgggccagccggaacatcggaaactatctggcctggtatcagcagaaacccggcaaggtgcccaagctgctgatctactatgccaagacatctgccgagggcgtgccaagcagattttctggcagcagaagcggcaccgactacaccctgaccatttctagcctgcagcctgaggacgtggccacctactactgccagcacttctggaaaaccccttggacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 137 - DNA, 642 bp, Synthetic construct
  source 1..642: mol_type = other DNA; note = IL-31 Light Chain
gccatccagatgacacagagccctagcagcctgtctgcctctgtgggcgatagagtgaccatcacctgtcgggccagccggaacatcggaaactatctggcctggtatcagcagaaacccggcaaggtgcccaagctgctgatctactatgccaagacaagcgccagcggcgtgccaagcagattttctggcagcagaagcggcaccgactacaccctgaccatttctagcctgcagcctgaggacgtggccacctactactgtcagcacttttggagcaccccttggacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 138 - DNA, 675 bp, Synthetic construct
  source 1..675: mol_type = other DNA; note = IL-31 Fd
caggttcagttggtgcagtccggcgctgaagtgaagaaacctggctcctctgtgaaggtgtcctgcaaggcctctggctacacccggaccagatattggatgcagtgggtccgacaggctcctggacaaggattggagtggatgggcgctatctaccctgctgagggcgacacccggtactcccagaaattcaagggcagagtgaccatcaccgccgacaagtctacctccaccgcctacatggaactgtccagcctgagatccgaggacaccgccgtgtactactgtgcctttcctgagggaccttacgccgctccttacggcatgcattattggggccagggcaccaccgtgaccgtgtcctctgcttctacaaagggcccctctgtgttccctctggctcctagctctaagtccacctctggtggaaccgctgctctgggctgtctggtcaaggattacttccctgagccagtgacagtgtcctggaactctggtgctctgacctccggcgtgcacacatttccagctgtgctgcagtcctccggcctgtactctctgtcctctgtcgtgacagtgccttccagctctctgggcacccagacctacatctgcaacgtgaaccacaagccttccaataccaaggtggacaagagagtggaacccaagtcctgc

SEQ ID NO: 139 - DNA, 675 bp, Synthetic construct
  source 1..675: mol_type = other DNA; note = IL-31 Fd
caggtgcagctggttcagtctggcgccgaagtgaagaaacccggcagcagcgtgaaggtgtcctgtaaagccagcggctacacccggaccagatactggatgcagtgggttcgacaggcccctggacaaggacttgagtggatgggagccatctatcctggcgagggcgatacccggtacagccagaaattcaagggcagagtgaccatcaccgccgacaagagcacaagcaccgcctacatggaactgagcagcctgagaagcgaggacaccgccgtgtactactgtgcctttcctgagggaccttacgccgctccttacggcatgcattattggggccagggcaccaccgtgaccgtttcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgc

SEQ ID NO: 140 - DNA, 675 bp, Synthetic construct
  source 1..675: mol_type = other DNA; note = IL-31 Fd
caggtgcagctggttcagtctggcgccgaagtgaagaaacccggcagcagcgtgaaggtgtcctgtaaagccagcggctacacccggaccagatactggatgcagtgggttcgacaggcccctggacaaggacttgagtggatgggagccatctatcctggcgagggcgatacccggtacagccagaaattcaagggcagagtgaccatcaccgccgacaagagcacaagcaccgcctacatggaactgagcagcctgagaagcgaggacaccgccgtgtactactgtgcctttcctgagggacagtacgccgctccttacggcatgcattattggggccagggcaccaccgtgaccgtttcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgc

SEQ ID NO: 141 - DNA, 675 bp, Synthetic construct
  source 1..675: mol_type = other DNA; note = IL-31 Fd
caggtgcagctggttcagtctggcgccgaagtgaagaaacccggcagcagcgtgaaggtgtcctgtaaagccagcggctacacccggaccagatactggatgcagtgggttcgacaggcccctggacaaggacttgagtggatgggagccatctatcctgccgagggcgatacccggtacagccagaaattcaagggcagagtgaccatcaccgccgacaagagcacaagcaccgcctacatggaactgagcagcctgagaagcgaggacaccgccgtgtactactgtgcctttcctgagggaccttacgccgctccttacggcatgcattattggggccagggcaccaccgtgaccgtttcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgc

SEQ ID NO: 142 - DNA, 663 bp, Synthetic construct
  source 1..663: mol_type = other DNA; note = IL-13 Fd
caagtcactctgcgcgaatcaggaccggccctggtcaagcccacccagaccctgaccctgacttgtactgtgtcgggtttctccctgtccgcgtatagcgtgaactggattagacagcctcccggaaaggccctggagtggcttgcgatgatctggggggacgggaagatcgtgtacaacagcgccctcaagagccgcctgaccatcagcaaggacacctctaagaatcaggtcgtgctgactatgaccaacatggatcccgtggacaccgccacctactactgtgctggggacggctactatccctacgccatggacaactggggacagggttccctggttactgtgtcctccgcttcgaccaagggcccgtcggtgtttccactcgccccgagctccaagagcacttcaggcggtactgcagccttgggatgcctcgtgaaggactacttccctgagccggtcaccgtgtcctggaacagcggcgccctgacttccggggtgcacaccttcccggccgtgctgcaatcctccggcctgtactcactgtcctcggtcgtgaccgtgccgagcagcagcctgggcacccagacctacatctgcaacgtgaaccacaagccctccaacaccaaggtcgataagagggtggaacccaagtcctgc

SEQ ID NO: 143 - protein, 164 aa, Synthetic construct
  source 1..164: note = |Q6EBC2|IL31_HUMAN Interleukin-31
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 144 - protein, 163 aa, Synthetic construct
  source 1..163: note = G7PJ78_MACFA Interleukin 31
MASHSGPATSVLFLLCCLGGWLTSHTLPVHFLQPSDIQKIVEELQSLSKMLLKDVKEDKGVLVSQNYTLPCLTPDAQPPNIIHSPAIRAYLKTIRQLDNKSVIDEIIEHLDKLIFQDAPETNISVPTDTHECKRFILTISQQFSECMDLALKSLTSGAQQATT

SEQ ID NO: 145 - protein, 125 aa, Synthetic construct
  source 1..125: note = HUMAN Interleukin-31
PSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHESKRFILTISQQFSECMDLALKSLTSG

SEQ ID NO: 146 - protein, 123 aa, Synthetic construct
  source 1..123: note = MACFA Interleukin 31
SDIQKIVEELQSLSKMLLKDVKEDKGVLVSQNYTLPCLTPDAQPPNIIHSPAIRAYLKTIRQLDNKSVIDEIIEHLDKLIFQDAPETNISVPTDTHESKRFILTISQQFSECMDLALKSLTSG

SEQ ID NO: 147 - protein, 225 aa, Synthetic construct
  source 1..225: note = TPP-13357 Heavy Chain
QVQLVQSGAEVKKPGSSVKVSCKASGYTLTRYWMQWVRQAPGQGLEWMGAIYPGDGDTRYSQKFKGRVTITADKSTSTAYMELSSLRSEDTAVYYCAFPEGYYAAPYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 148 - protein, 214 aa, Synthetic construct
  source 1..214: note = TPP-13357 Light Chain
DIQMTQSPSSLSASVGDRVTITCRASGNIHNYLAWYQQKPGKVPKLLIYNAKTLAEGVPSRFSGSRSETDYTLTISSLQPEDVATYYCQHFWSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 115 aa, Synthetic construct
  source 1..115: note = TPP-13357 Heavy Chain
QVQLVQSGAEVKKPGSSVKVSCKASGYTLTRYWMQWVRQAPGQGLEWMGAIYPGDGDTRYSQKFKGRVTITADKSTSTAYMELSSLRSEDTAVYYCAFPEGYYAAPYGMDYWGQG

SEQ ID NO: 150 - protein, 108 aa, Synthetic construct
  source 1..108: note = TPP-13357 Light Chain
DIQMTQSPSSLSASVGDRVTITCRASGNIHNYLAWYQQKPGKVPKLLIYNAKTLAEGVPSRFSGSRSETDYTLTISSLQPEDVATYYCQHFWSTPWTFGGGTKVEIKR

SEQ ID NO: 151 - protein, 115 aa, Synthetic construct
  source 1..115: note = TPP-17880 Heavy Chain
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQG

SEQ ID NO: 152 - protein, 108 aa, Synthetic construct
  source 1..108: note = TPP-17880 Light Chain
AIQMTQSPSSLSASVGDRVTITCRASRNIGNYLAWYQQKPGKVPKLLIYYAKTSASGVPSRFSGSRSGTDYTLTISSLQPEDVATYYCQHFWSTPWTFGGGTKVEIKR

SEQ ID NO: 153 - protein, 225 aa, Synthetic construct
  source 1..225: note = TPP-20012 Heavy Chain
QVQLVQSGAEVKKPGASVKVSCKASGYTLTRYWMQWVRQAPGQGLEWMGAIYPGDGDTRYSQKFQGRVTLTADKSTSTAYMELSSLRSEDTAVYYCAFPDGYYAAPYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 154 - protein, 214 aa, Synthetic construct
  source 1..214: note = TPP-20012 Light Chain
DIQMTQSPSSLSASVGDRVTITCRASGNIHNYLAWYQQKPGKVPKLLIYNAKTLADGVPSRFSGSRSETQYSLTISSLQPEDVATYYCQHFWSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 225 aa, Synthetic construct
  source 1..225: note = TPP-13348 Heavy Chain
QVQLVQSGAEVKKPGSSVKVSCKASGYTLTRYWMQWVRQAPGQGLEWMGAIYPGDGDTRYSQKFKGRVTITADKSTSTAYMELSSLRSEDTAVYYCAFPDGYYAAPYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 156 - protein, 214 aa, Synthetic construct
  source 1..214: note = TPP-13348 Light Chain
DIQMTQSPSSLSASVGDRVTITCRASGNIHNYLAWYQQKPGKVPKLLIYNAKTLADGVPSRFSGSRSETDYTLTISSLQPEDVATYYCQHFWSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 122 aa, Synthetic construct
  source 1..122: note = TPP-13357 VH
QVQLVQSGAEVKKPGSSVKVSCKASGYTLTRYWMQWVRQAPGQGLEWMGAIYPGDGDTRYSQKFKGRVTITADKSTSTAYMELSSLRSEDTAVYYCAFPEGYYAAPYGMDYWGQGTTVTVSS

SEQ ID NO: 158 - protein, 107 aa, Synthetic construct
  source 1..107: note = TPP-13357 VL
DIQMTQSPSSLSASVGDRVTITCRASGNIHNYLAWYQQKPGKVPKLLIYNAKTLAEGVPSRFSGSRSETDYTLTISSLQPEDVATYYCQHFWSTPWTFGGGTKVEIK

SEQ ID NO: 159 - protein, 122 aa, Synthetic construct
  source 1..122: note = TPP-13348 VH
QVQLVQSGAEVKKPGSSVKVSCKASGYTLTRYWMQWVRQAPGQGLEWMGAIYPGDGDTRYSQKFKGRVTITADKSTSTAYMELSSLRSEDTAVYYCAFPDGYYAAPYGMDYWGQGTTVTVSS

SEQ ID NO: 160 - protein, 107 aa, Synthetic construct
  source 1..107: note = TPP-13348 VL
DIQMTQSPSSLSASVGDRVTITCRASGNIHNYLAWYQQKPGKVPKLLIYNAKTLADGVPSRFSGSRSETDYTLTISSLQPEDVATYYCQHFWSTPWTFGGGTKVEIK

SEQ ID NO: 161 - DNA, 366 bp, Synthetic construct
  source 1..366: mol_type = other DNA; note = TPP-13348 VH DNA
caggtgcagctggttcagtctggcgccgaagtgaagaaacccggcagcagcgtgaaggtgtcctgtaaagccagcggctacaccctgaccagatactggatgcagtgggttcgacaggcccctggacaaggacttgagtggatgggagctatctaccctggcgacggcgatacccggtacagccagaaattcaagggcagagtgaccatcaccgccgacaagagcacaagcaccgcctacatggaactgagcagcctgagaagcgaggacaccgccgtgtactactgtgcctttcctgacggctactacgccgctccttacggcatggattattggggccagggcaccaccgtgaccgtttcttct

SEQ ID NO: 162 - DNA, 321 bp, Synthetic construct
  source 1..321: mol_type = other DNA; note = TPP-13348 VL DNA
gacatccagatgacacagagccctagcagcctgtctgcctctgtgggcgatagagtgaccatcacatgcagagccagcggcaacatccacaactacctggcctggtatcagcagaaacccggcaaggtgcccaagctgctgatctacaatgccaagacactggccgatggcgtgccaagcagattttccggcagcagaagcgagacagactacaccctgaccatcagcagcctgcagcctgaggatgtggccacctactactgccagcacttttggagcaccccttggacctttggcggaggcaccaaggtggaaatcaag

SEQ ID NO: 163 - DNA, 366 bp, Synthetic construct
  source 1..366: mol_type = other DNA; note = TPP-13357 VH DNA
caggtgcagctggttcagtctggcgccgaagtgaagaaacccggcagcagcgtgaaggtgtcctgtaaagccagcggctacaccctgaccagatactggatgcagtgggttcgacaggcccctggacaaggacttgagtggatgggagctatctaccctggcgacggcgatacccggtacagccagaaattcaagggcagagtgaccatcaccgccgacaagagcacaagcaccgcctacatggaactgagcagcctgagaagcgaggacaccgccgtgtactactgtgcctttcctgagggctactacgccgctccttacggcatggattattggggccagggcaccaccgtgaccgtttcttct

SEQ ID NO: 164 - DNA, 321 bp, Synthetic construct
  source 1..321: mol_type = other DNA; note = TPP-13357 VL DNA
gacatccagatgacacagagccctagcagcctgtctgcctctgtgggcgatagagtgaccatcacatgcagagccagcggcaacatccacaactacctggcctggtatcagcagaaacccggcaaggtgcccaagctgctgatctacaatgccaagacactggccgagggcgtgccaagcagattttctggcagcagaagcgagacagactacaccctgaccatcagcagcctgcagcctgaggatgtggccacctactactgccagcacttttggagcaccccttggacctttggcggaggcaccaaggtggaaatcaag

SEQ ID NO: 165 - DNA, 366 bp, Synthetic construct
  source 1..366: mol_type = other DNA; note = TPP-20012 VH DNA
caggtgcagctggttcagtctggcgccgaagttaagaaaccaggcgcctctgtgaaggtgtcctgcaaggcctctggctacaccctgaccagatactggatgcagtgggttcgacaggcccctggacaaggacttgagtggatgggagctatctaccctggcgacggcgatacccggtacagccagaaatttcagggcagagtgaccctgaccgccgacaagagcacaagcacagcctacatggaactgagcagcctgagaagcgaggacaccgccgtgtactactgtgcctttcctgacggctactacgccgctccttacggcatggattattggggccagggcaccctggtcaccgtttcttct

SEQ ID NO: 166 - DNA, 321 bp, Synthetic construct
  source 1..321: mol_type = other DNA; note = TPP-20012 VL DNA
gacatccagatgacacagagccctagcagcctgtctgcctctgtgggcgatagagtgaccatcacatgcagagccagcggcaacatccacaactacctggcctggtatcagcagaaacccggcaaggtgcccaagctgctgatctacaatgccaagacactggccgatggcgtgccaagcagattttccggcagcagaagcgagacacagtacagcctgacaatcagcagcctgcagcctgaggatgtggccacctactactgccagcacttttggagcaccccttggacctttggccagggcaccaaggtggaaatcaag

SEQ ID NO: 167 - DNA, 675 bp, Synthetic construct
  source 1..675: mol_type = other DNA; note = TPP-13348 Heavy Chain DNA
caggtgcagctggttcagtctggcgccgaagtgaagaaacccggcagcagcgtgaaggtgtcctgtaaagccagcggctacaccctgaccagatactggatgcagtgggttcgacaggcccctggacaaggacttgagtggatgggagctatctaccctggcgacggcgatacccggtacagccagaaattcaagggcagagtgaccatcaccgccgacaagagcacaagcaccgcctacatggaactgagcagcctgagaagcgaggacaccgccgtgtactactgtgcctttcctgacggctactacgccgctccttacggcatggattattggggccagggcaccaccgtgaccgtttcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgc

SEQ ID NO: 168 - DNA, 642 bp, Synthetic construct
  source 1..642: mol_type = other DNA; note = TPP-13348 Light Chain DNA
gacatccagatgacacagagccctagcagcctgtctgcctctgtgggcgatagagtgaccatcacatgcagagccagcggcaacatccacaactacctggcctggtatcagcagaaacccggcaaggtgcccaagctgctgatctacaatgccaagacactggccgatggcgtgccaagcagattttccggcagcagaagcgagacagactacaccctgaccatcagcagcctgcagcctgaggatgtggccacctactactgccagcacttttggagcaccccttggacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 169 - DNA, 675 bp, Synthetic construct
  source 1..675: mol_type = other DNA; note = TPP-13357 Heavy Chain DNA
caggtgcagctggttcagtctggcgccgaagtgaagaaacccggcagcagcgtgaaggtgtcctgtaaagccagcggctacaccctgaccagatactggatgcagtgggttcgacaggcccctggacaaggacttgagtggatgggagctatctaccctggcgacggcgatacccggtacagccagaaattcaagggcagagtgaccatcaccgccgacaagagcacaagcaccgcctacatggaactgagcagcctgagaagcgaggacaccgccgtgtactactgtgcctttcctgagggctactacgccgctccttacggcatggattattggggccagggcaccaccgtgaccgtttcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgc

SEQ ID NO: 170 - DNA, 642 bp, Synthetic construct
  source 1..642: mol_type = other DNA; note = TPP-13357 Light Chain DNA
gacatccagatgacacagagccctagcagcctgtctgcctctgtgggcgatagagtgaccatcacatgcagagccagcggcaacatccacaactacctggcctggtatcagcagaaacccggcaaggtgcccaagctgctgatctacaatgccaagacactggccgagggcgtgccaagcagattttctggcagcagaagcgagacagactacaccctgaccatcagcagcctgcagcctgaggatgtggccacctactactgccagcacttttggagcaccccttggacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 171 - DNA, 675 bp, Synthetic construct
  source 1..675: mol_type = other DNA; note = TPP-20012 Heavy Chain DNA
caggtgcagctggttcagtctggcgccgaagttaagaaaccaggcgcctctgtgaaggtgtcctgcaaggcctctggctacaccctgaccagatactggatgcagtgggttcgacaggcccctggacaaggacttgagtggatgggagctatctaccctggcgacggcgatacccggtacagccagaaatttcagggcagagtgaccctgaccgccgacaagagcacaagcacagcctacatggaactgagcagcctgagaagcgaggacaccgccgtgtactactgtgcctttcctgacggctactacgccgctccttacggcatggattattggggccagggcaccctggtcaccgtttcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgc

SEQ ID NO: 172 - DNA, 642 bp, Synthetic construct
  source 1..642: mol_type = other DNA; note = TPP-20012 Light Chain DNA
gacatccagatgacacagagccctagcagcctgtctgcctctgtgggcgatagagtgaccatcacatgcagagccagcggcaacatccacaactacctggcctggtatcagcagaaacccggcaaggtgcccaagctgctgatctacaatgccaagacactggccgatggcgtgccaagcagattttccggcagcagaagcgagacacagtacagcctgacaatcagcagcctgcagcctgaggatgtggccacctactactgccagcacttttggagcaccccttggacctttggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 173 - protein, 141 aa, synthetic construct
SHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHESKRFILTISQQFSECMDLALKSLTSGAQQATT

SEQ ID NO: 174 - protein, 720 aa, synthetic construct
EVQLVESGGGLVQPGGSLRLSCAASGFSSSSAHYMCWVRQAPGKGLEWIGCIYAGSRGSTYYASWAKGRFTISRDNSKNTVYLQMNSLRAEDTAVYYCARAAFVNRGVSWIWPYYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSAFQLTQSPSSLSASVGDRVTITCQASQSIDSWLAWYQQKPGKPPKLLIYQASKLASGVPSRFSGSGSGTDYTLTISSLQPEDFATYYCQTYYGGGHIGWGFGTGTKVTVLGGGGGSGGGGSGGGGSGGGGSQSQLVESGGGRVQPGGSLRLSCAASGFSFSANYWICWVRQAPGKGLEWIVCIYIGSRADTYYAPWAKGRFTISKDNSKNTVYLQMNSLRAEDTATYFCARFSSGIYDLDRFFLWGQGTQVTVSS

SEQ ID NO: 175 - protein, 216 aa, synthetic construct
DIQMTQSPSSLSARVGDRVTITCQASEDIESYLAWYQQKPGKAPKLLIYRASTLAYGVPSRFSGSGSGRDFTLTISSLQPEDFATYYCLGSSYSTGSNIFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 452 aa, synthetic construct
QVQLVQSGAEVKKPGSSVKVSCKASGYTRTRYWMQWVRQAPGQGLEWMGAIYPAEGDTRYSQKFKGRVTITADKSTSTAYMELSSLRSEDTAVYYCAFPEGPYAAPYGMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 177 - protein, 214 aa, synthetic construct; identical to SEQ ID NO: 9

SEQ ID NO: 178 - protein, 448 aa, synthetic construct
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 218 aa, synthetic construct; identical to SEQ ID NO: 19